SEQUENCE LISTING (92 sequences)

SEQ ID NO: 1 - DNA, 2151 bp, Homo sapiens
tgcgttggctgcggcctggcaccaaaggggcggccccggcggagagcggacccagtggcctcggcgattatggacccggccgaggcggtgctgcaagagaaggcactcaagtttatgaattcctcagagagagaagactgtaataatggcgaaccccctaggaagataataccagagaagaattcacttagacagacatacaacagctgtgccagactctgcttaaaccaagaaacagtatgtttagcaagcactgctatgaagactgagaattgtgtggccaaaacaaaacttgccaatggcacttccagtatgattgtgcccaagcaacggaaactctcagcaagctatgaaaaggaaaaggaactgtgtgtcaaatactttgagcagtggtcagagtcagatcaagtggaatttgtggaacatcttatatcccaaatgtgtcattaccaacatgggcacataaactcgtatcttaaacctatgttgcagagagatttcataactgctctgccagctcggggattggatcatatcgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttacagagaattcactgccgaagtgaaacaagcaaaggagtttactgtttacagtatgatgatcagaaaatagtaagcggccttcgagacaacacaatcaagatctgggataaaaacacattggaatgcaagcgaattctcacaggccatacaggttcagtcctctgtctccagtatgatgagagagtgatcataacaggatcatcggattccacggtcagagtgtgggatgtaaatacaggtgaaatgctaaacacgttgattcaccattgtgaagcagttctgcacttgcgtttcaataatggcatgatggtgacctgctccaaagatcgttccattgctgtatgggatatggcctccccaactgacattaccctccggagggtgctggtcggacaccgagctgctgtcaatgttgtagactttgatgacaagtacattgtttctgcatctggggatagaactataaaggtatggaacacaagtacttgtgaatttgtaaggaccttaaatggacacaaacgaggcattgcctgtttgcagtacagggacaggctggtagtgagtggctcatctgacaacactatcagattatgggacatagaatgtggtgcatgtttacgagtgttagaaggccatgaggaattggtgcgttgtattcgatttgataacaagaggatagtcagtggggcctatgatggaaaaattaaagtgtgggatcttgtggctgctttggacccccgtgctcctgcagggacactctgtctacggacccttgtggagcattccggaagagtttttcgactacagtttgatgaattccagattgtcagtagttcacatgatgacacaatcctcatctgggacttcctaaatgatccagctgcccaagctgaacccccccgttccccttctcgaacatacacctacatctccagataaataaccatacactgacctcatacttgcccaggacccattaaagttgcggtatttaacgtatctgccaataccaggatgagcaacaacagtaacaatcaaactactgcccagtttccctggactagccgaggagcagggctttgagactcctgttgggacacagttggtctgcagtcggcccaggacggtctactcagcacaactgactgcttcagtgctgctatcagaagatgtcttctatcaattgtgaatgattggaacttttaaacctcccctcctctcctcctttcacctctgcacctagttttttcccattggttccagacaaaggtgacttataaatatatttagtgttttgccagaaaaaaaaaa

SEQ ID NO: 2 - protein, 569 aa, Homo sapiens
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRSETSKGVYCLQYDDQKIVSGLRDNTIKIWDKNTLECKRILTGHTGSVLCLQYDERVIITGSSDSTVRVWDVNTGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLVAALDPRAPAGTLCLRTLVEHSGRVFRLQFDEFQIVSSSHDDTILIWDFLNDPAAQAEPPRSPSRTYTYISR

SEQ ID NO: 3 - DNA, 1476 bp, Homo sapiens
atggagagaaaggactttgagacatggcttgataacatttctgttacatttctttctctgacggacttgcagaaaaatgaaactctggatcacctgattagtctgagtggggcagtccagctcaggcatctctccaataacctagagactctcctcaagcgggacttcctcaaactccttcccctggagctcagtttttatttgttaaaatggctcgatcctcagactttactcacatgctgcctcgtctctaaacagtggaataaggtgataagtgcctgtacagaggtgtggcagactgcatgtaaaaatttgggctggcagatagatgattctgttcaggacgctttgcactggaagaaggtttatttgaaggctattttgagaatgaagcaactggaggaccatgaagcctttgaaacctcgtcattaattggacacagtgccagagtgtatgcactttactacaaagatggacttctctgtacagggtcagatgacttgtctgcaaagctgtgggatgtgagcacagggcagtgcgtttatggcatccagacccacacttgtgcagcggtgaagtttgatgaacagaagcttgtgacaggctcctttgacaacactgtggcttgctgggaatggagttccggagccaggacccagcactttcgggggcacacgggggcggtatttagcgtggactacaatgatgaactggatatcttggtgagcggctctgcagacttcactgtgaaagtatgggctttatctgctgggacatgcctgaacacactcaccgggcacacggaatgggtcaccaaggtagttttgcagaagtgcaaagtcaagtctctcttgcacagtcctggagactacatcctcttaagtgcagacaaatatgagattaagatttggccaattgggagagaaatcaactgtaagtgcttaaagacattgtctgtctctgaggatagaagtatctgcctgcagccaagacttcattttgatggcaaatacattgtctgtagttcagcacttggtctctaccagtgggactttgccagttatgatattctcagggtcatcaagactcctgagatagcaaacttggccttgcttggctttggagatatctttgccctgctgtttgacaaccgctacctgtacatcatggacttgcggacagagagcctgattagtcgctggcctctgccagagtacagggaatcaaagagaggctcaagcttcctggcaggcgaacatcctggctgaatggactggatgggcacaatgacacgggcttggtctttgccaccagcatgcctgaccacagtattcacctggtgttgtggaaggagcacggctgacaccatgagccaccaccgctgactgactttgggtgccggggctgcgggttttgggtgcacctctgcggcacgcgactgcatgaaccaaagttctcacctaatggtatcatca

SEQ ID NO: 4 - protein, 422 aa, Homo sapiens
MERKDFETWLDNISVTFLSLTDLQKNETLDHLISLSGAVQLRHLSNNLETLLKRDFLKLLPLELSFYLLKWLDPQTLLTCCLVSKQWNKVISACTEVWQTACKNLGWQIDDSVQDALHWKKVYLKAILRMKQLEDHEAFETSSLIGHSARVYALYYKDGLLCTGSDDLSAKLWDVSTGQCVYGIQTHTCAAVKFDEQKLVTGSFDNTVACWEWSSGARTQHFRGHTGAVFSVDYNDELDILVSGSADFTVKVWALSAGTCLNTLTGHTEWVTKVVLQKCKVKSLLHSPGDYILLSADKYEIKIWPIGREINCKCLKTLSVSEDRSICLQPRLHFDGKYIVCSSALGLYQWDFASYDILRVIKTPEIANLALLGFGDIFALLFDNRYLYIMDLRTESLISRWPLPEYRESKRGSSFLAGEHPG

SEQ ID NO: 5 - DNA, 1407 bp, Homo sapiens
cggggtggtgtgtgggggaagccgcccccggcagcaggatgaaacgaggaggaagagatagtgaccgtaattcatcagaagaaggaactgcagagaaatccaagaaactgaggactacaaatgagcattctcagacttgtgattggggtaatctccttcaggacattattctccaagtatttaaatatttgcctcttcttgaccgggctcatgcttcacaagtttgccgcaactggaaccaggtatttcacatgcctgacttgtggagatgttttgaatttgaactgaatcagccagctacatcttatttgaaagctacccatccagagctgatcaaacagattattaaaagacattcaaaccatctacaatatgtcagcttcaaggtggacagcagcaaggaatcagctgaagcagcttgtgatatactatcgcaacttgtgaattgctctttaaaaacacttggacttatttcaactgctcgaccaagctttatggatttaccaaagtctcactttatctctgcactgacagttgtgttcgtaaactccaaatccctgtcttcgcttaagatagatgatactccagtagatgatccatctctcaaagtactagtggccaacaatagtgatacactcaagctgttgaaaatgagcagctgtcctcatgtctctccagcaggtatcctttgtgtggctgatcagtgtcacggcttaagagaactagccctgaactaccacttattgagtgatgagttgttacttgcattgtcttctgaaaaacatgttcgattagaacatttgcgcattgatgtagtcagtgagaatcctggacagacacacttccatactattcagaagagtagctgggatgctttcatcagacattcacccaaagtgaacttagtgatgtatttttttttatatgaagaagaatttgaccccttctttcgctatgaaatacctgccacccatctgtactttgggagatcagtaagcaaagatgtgcttggccgtgtgggaatgacatgccctagactggttgaactagtagtgtgtgcaaatggattacggccacttgatgaagagttaattcgcattgcagaacgttgcaaaaatttgtcagctattggactaggggaatgtgaagtctcatgtagtgcctttgttgagtttgtgaagatgtgtggtggccgcctatctcaattatccattatggaagaagtactaattcctgaccaaaagtatagtttggagcagattcactgggaagtgtccaagcatcttggtagggtgtggtttcccgacatgatgcccacttggtaaaaactgcatgatgaatagcaccttaatttcaagcaaatgtattataattaaagttttatttgctgtaaaaaaaaaaaaaaaa

SEQ ID NO: 6 - protein, 428 aa, Homo sapiens
MKRGGRDSDRNSSEEGTAEKSKKLRTTNEHSQTCDWGNLLQDIILQVFKYLPLLDRAHASQVCRNWNQVFHMPDLWRCFEFELNQPATSYLKATHPELIKQIIKRHSNHLQYVSFKVDSSKESAEAACDILSQLVNCSLKTLGLISTARPSFMDLPKSHFISALTVVFVNSKSLSSLKIDDTPVDDPSLKVLVANNSDTLKLLKMSSCPHVSPAGILCVADQCHGLRELALNYHLLSDELLLALSSEKHVRLEHLRIDVVSENPGQTHFHTIQKSSWDAFIRHSPKVNLVMYFFLYEEEFDPFFRYEIPATHLYFGRSVSKDVLGRVGMTCPRLVELVVCANGLRPLDEELIRIAERCKNLSAIGLGECEVSCSAFVEFVKMCGGRLSQLSIMEEVLIPDQKYSLEQIHWEVSKHLGRVWFPDMMPTW

SEQ ID NO: 7 - DNA, 1444 bp, Homo sapiens
atggcgggaagcgagccgcgcagcggaacaaattcgccgccgccgcccttcagcgactggggccgcctggaggcggccatcctcagcggctggaagaccttctggcagtcagtgagcaaggatagggtggcgcgtacgacctcccgggaggaggtggatgaggcggccagcaccctgacgcggctgccgattgatgtacagctatatattttgtcctttctttcacctcatgatctgtgtcagttgggaagtacaaatcattattggaatgaaactgtaagaaatccaattctgtggagatactttttgttgagggatcttccttcttggtcttctgttgactggaagtctcttccatatctacaaatcttaaaaaagcctatatctgaggtctctgatggtgcattttttgactacatggcagtctatctaatgtgctgtccatacacaagaagagcttcaaaatccagccgtcctatgtatggagctgtcacttcttttttacactccctgatcattcccaatgaacctcgatttgctctgtttggaccacgtttggaacaattgaatacctctttggtgttgagcttgctgtcttcagaggaactttgcccaacagctggtttgcctcagaggcagattgatggtattggatcaggagtcaattttcagttgaacaaccaacataaattcaacattctaatcttatattcaactaccagaaaggaaagagatagagcaagggaagagcatacaagtgcagttaacaagatgttcagtcgacacaatgaaggtgatgatcgaccaggaagccggtacagtgtgattccacagattcaaaaactgtgtgaagttgtagatgggttcatctatgttgcaaatgctgaagctcataaaagacatgaatggcaagatgaattttctcatattatggcaatgacagatccagcctttgggtcttcgggaagaccattgttggttttatcttgtatttctcaaggggatgtaaaaagaatgccctgtttttatttggctcatgagctgcatctgaatcttctaaatcacccatggctggtccaggatacagaggctgaaactctgactggttttttgaatggcattgagtggattcttgaagaagtggaatctaagcgtgcaagatgattctcttttcagatcttgggaactgaaaccatttgaaatttattactaaggtcgtgatgtgaatatttgctcagtcagcccaccttgtcctgcctttttgcagataggctttcatttggacagctataactgctgtgttttttatattatttttactttttaccataaatcaattacaagaaaagagtttcagtcctagtatttagccccaaaatgaacctttaaacatttttttggtaatttttatattttctgtctttttaaaaatattaaattttgg

SEQ ID NO: 8 - protein, 472 aa, Homo sapiens [sequence omitted]

SEQ ID NO: 9 - DNA, 2076 bp, Homo sapiens
aggttgctcagctgcccccggagcggttcctccacctgaggcagacaccacctcggttggcatgagccggcgcccctgcagctgcgccctacggccaccccgctgctcctgcagcgccagccccagcgcagtgacagccgccgggcgccctcgaccctcggatagttgtaaagaagaaagttctaccctttctgtcaaaatgaagtgtgattttaattgtaaccatgttcattccggacttaaactggtaaaacctgatgacattggaagactagtttcctacacccctgcatatctggaaggttcctgtaaagactgcattaaagactatgaaaggctgtcatgtattgggtcaccgattgtgagccctaggattgtacaacttgaaactgaaagcaagcgcttgcataacaaggaaaatcaacatgtgcaacagacacttaatagtacaaatgaaatagaagcactagagaccagtagactttatgaagacagtggctattcctcattttctctacaaagtggcctcagtgaacatgaagaaggtagcctcctggaggagaatttcggtgacagtctacaatcctgcctgctacaaatacaaagcccagaccaatatcccaacaaaaacttgctgccagttcttcattttgaaaaagtggtttgttcaacattaaaaaagaatgcaaaacgaaatcctaaagtagatcgggagatgctgaaggaaattatagccagaggaaattttagactgcagaatataattggcagaaaaatgggcctagaatgtgtagatattctcagcgaactctttcgaaggggactcagacatgtcttagcaactattttagcacaactcagtgacatggacttaatcaatgtgtctaaagtgagcacaacttggaagaagatcctagaagatgataagggggcattccagttgtacagtaaagcaatacaaagagttaccgaaaacaacaataaattttcacctcatgcttcaaccagagaatatgttatgttcagaaccccactggcttctgttcagaaatcagcagcccagacttctctcaaaaaagatgctcaaaccaagttatccaatcaaggtgatcagaaaggttctacttatagtcgacacaatgaattctctgaggttgccaagacattgaaaaagaacgaaagcctcaaagcctgtattcgctgtaattcacctgcaaaatatgattgctatttacaacgggcaacctgcaaacgagaaggctgtggatttgattattgtacgaagtgtctctgtaattatcatactactaaagactgttcagatggcaagctcctcaaagccagttgtaaaataggtcccctgcctggtacaaagaaaagcaaaaagaatttacgaagattgtgatctcttattaaatcaattgttactgatcatgaatgttagttagaaaatgttaggttttaacttaaaaaaaattgtattgtgattttcaattttatgttgaaatcggtgtagtatcctgaggtttttttccccccagaagataaagaggatagacaacctcttaaaatatttttacaatttaatgagaaaaagtttaaaattctcaatacaaatcaaacaatttaaatattttaagaaaaaaggaaaagtagatagtgatactgagggtaaaaaaaaaattgattcaattttatggtaaaggaaacccatgcaattttacctagacagtcttaaatatgtctggttttccatctgttagcatttcagacattttatgttcctcttactcaattgataccaacagaaatatcaacttctggagtctattaaatgtgttgtcacctttctaaagctttttttcattgtgtgtatttcccaagaaagtatcctttgtaaaaacttgcttgttttccttatttctgaaatctgttttaatatttttgtatacatgtaaatatttctgtattttttatatgtcaaagaatatgtctcttgtatgtacatataaaaataaattttgctcaataaaattgtaagcttaaaaaaaaaaaaaaaaaactcgagactagtgc

SEQ ID NO: 10 - protein, 447 aa, Homo sapiens
MSRRPCSCALRPPRCSCSASPSAVTAAGRPRPSDSCKEESSTLSVKMKCDFNCNHVHSGLKLVKPDDIGRLVSYTPAYLEGSCKDCIKDYERLSCIGSPIVSPRIVQLETESKRLHNKENQHVQQTLNSTNEIEALETSRLYEDSGYSSFSLQSGLSEHEEGSLLEENFGDSLQSCLLQIQSPDQYPNKNLLPVLHFEKVVCSTLKKNAKRNPKVDREMLKEIIARGNFRLQNIIGRKMGLECVDILSELFRRGLRHVLATILAQLSDMDLINVSKVSTTWKKILEDDKGAFQLYSKAIQRVTENNNKFSPHASTREYVMFRTPLASVQKSAAQTSLKKDAQTKLSNQGDQKGSTYSRHNEFSEVAKTLKKNESLKACIRCNSPAKYDCYLQRATCKREGCGFDYCTKCLCNYHTTKDCSDGKLLKASCKIGPLPGTKKSKKNLRRL

SEQ ID NO: 11 - DNA, 1535 bp, Homo sapiens
gcgcgttcgggagcttcggccctgcgtaggaggcgggtgcaggtgtgggtgctgagccgcccgccgcctggagggggagacagcttcaggacacgcaggccgcagcgagggcccgggcccgggggatcccaggccatggacgctccccactccaaagcagccctggacagcattaacgagctgcccgataacatcctgctggagctgttcacgcacgtgcccgcccgccagctgctgctgaactgccgcctggtctgcagcctctggcgggacctcatcgacctcctgaccctctggaaacgcaagtgcctgcgaaagggcttcatcaccaaggactgggaccagcccgtggccgactggaaaatcttctacttcctacggagcctgcataggaacctcctgcgcaacccgtgtgctgaaaacgatatgtttgcatggcaaattgatttcaatggtggggaccgctggaaggtggatagcctccctggagcccacgggacagaatttcctgaccccaaagtcaagaagtcttttgtcacatcctacgaactgtgcctcaagtgggagctggtggaccttctagccgaccgctactgggaggagctactagacacattccggccggacatcgtggttaaggactggtttgctgccagagccgactgtggctgcacctaccaactcaaagtgcagctggcctcggctgactacttcgtgttggcctccttcgagcccccacctgtgaccatccaacagtggaacaatgccacatggacagaggtctcctacaccttctcagactacccccggggtgtccgctacatcctcttccagcatgggggcagggacacccagtactgggcaggctggtatgggccccgagtcaccaacagcagcattgtcgtcagccccaagatgaccaggaaccaggcctcgtccgaggctcagcctgggcagaagcatggacaggaggaggctgcccaatcgccctacggagctgttgtccagattttctgacagctgtccatcctgtgtctgggtcagccagaggttcctccaggcaggagctgagcatggggtgggcagtgaggtccctgtaccagcgactcctgccccggttcaaccctaccagcttgtggtaacttactgtcacatagctctgacgttttgttgtaataaatgttttcaggccgggcactgtggctcacgcctgtaatcccagcactttgggagaccgaggcaggtggatcacgaggtcaggagacagagaccatcctggccaacacggtgaaaccctgtctctactaaaaatacaaaaaattagccgggcgtggtggcgggcgcctgtagtcccagctactcgggaggctgatgcagaagaatggcgtgaacccggaaggcagagcttgcagtgagccgagatcacgccactgcactccagcctgggtgacagagcgagactctggctcataaaataataataataataaataaataaaaaataaatggttttcagtaaaaaaaaaaaaaaaaaa

SEQ ID NO: 12 - protein, 338 aa, Homo sapiens
ARSGASALRRRRVQVWVLSRPPPGGGDSFRTRRPQRGPGPGGSQAMDAPHSKAALDSINELPDNILLELFTHVPARQLLLNCRLVCSLWRDLIDLLTLWKRKCLRKGFITKDWDQPVADWKIFYFLRSLHRNLLRNPCAENDMFAWQIDFNGGDRWKVDSLPGAHGTEFPDPKVKKSFVTSYELCLKWELVDLLADRYWEELLDTFRPDIVVKDWFAARADCGCTYQLKVQLASADYFVLASFEPPPVTIQQWNNATWTEVSYTFSDYPRGVRYILFQHGGRDTQYWAGWYGPRVTNSSIVVSPKMTRNQASSEAQPGQKHGQEEAAQSPYGAVVQIF

SEQ ID NO: 13 - DNA, 1763 bp, Homo sapiens
tggaattcccatggaccatgtctaatacccgatttacaattacattgaactacaaggatcccctcactggagatgaagagaccttggcttcatatgggattgtttctggggacttgatatgtttgattcttcacgatgacattccaccgcctaatataccttcatccacagattcagagcattcttcactccagaacaatgagcaaccctctttggccaccagctccaatcagactagcatacaggatgaacaaccaagtgattcattccaaggacaggcagcccagtctggtgtttggaatgacgacagtatgttagggcctagtcaaaattttgaagctgagtcaattcaagataatgcgcatatggcagagggcacaggtttctatccctcagaacccctgctctgtagtgaatcggtggaagggcaagtgccacattcattagagaccttgtatcaatcagctgactgttctgatgccaatgatgcgttgatagtgttgatacatcttctcatgttggagtcaggttacatacctcagggcaccgaagccaaagcactgtccctgccggagaagtggaagttgagcggggtgtataagctgcagtacatgcatcatctctgcgagggcagctccgctactctcacctgtgtgcctttgggaaacctgattgttgtaaatgctacactaaaaatcaacaatgagattagaagtgtgaaaagattgcagctgctaccagaatcttttatttgcaaagagaaactaggggaaaatgtagccaacatatacaaagatcttcagaaactctctcgcctctttaaagaccagctggtgtatcctcttctggcttttacccgacaagcactgaacctaccaaatgtatttgggttggtcgtcctcccattggaactgaaactacggatcttccgacttctggatgttcgttccgtcttgtctttgtctgcggtttgtcgtgacctctttactgcttcaaatgacccactcctgtggaggtttttatatctgcgtgattttcgagacaatactgtcagagttcaagacacagattggaaagaactgtacaggaagaggcacatacaaagaaaagaatccccgaaagggcggtttgtgctgctcctgccatcgtcaacccacaccattccattctatcccaaccccttgcaccctaggccatttcctagctcccgccttcctccaggaattatcgggggtgaatatgaccaaagaccaacacttccctatgttggagacccaatcagttcactcattcctggtcctggggagacgcccagccagttacctccactgagaccacgctttgatccagttggcccacttccaggacctaaccccatcttgccagggcgaggcggccccaatgacagatttccctttagacccagcaggggtcggccaactgatggccgcctgtcattcatgtgattgatttgtaatttcatttctggagctccatttgtttttgtttctaaactacagatgtcactccttggggtgctgatctcgagtgttattttctgattgtggtgttgagagttgcactcccagaaaccttttaagagatacatttatagccctaggggtggtatgacccaaaggttcctctgtgacaaggttggccttgggaatagttggctgccaatctccctgctcttggttctcctctagattgaagtttgttttctgatgctgttcttaccagattaaaaaaaagtgtaaatt

SEQ ID NO: 14 - protein, 482 aa, Homo sapiens [sequence omitted]

SEQ ID NO: 15 - protein, 43 aa, Homo sapiens
LPARGLDHIAENILSYLDAKSLCAAELVCKEWYRVTSDGMLWK

SEQ ID NO: 16 - protein, 40 aa, Homo sapiens
LPLELSFYLLKWLDPQTLLTCCLVSKQWNKVISACTEVWQ

SEQ ID NO: 17 - protein, 39 aa, Homo sapiens
LLQDIILQVFKYLPLLDRAHASQVCRNWNQVFHMPDLWR

SEQ ID NO: 18 - protein, 39 aa, Homo sapiens
LPIDVQLYILSFLSPHDLCQLGSTNHYWNETVRNPILWR

SEQ ID NO: 19 - protein, 39 aa, Homo sapiens
LRHVLATILAQLSDMDLINVSKVSTTWKKILEDDKGAFQ

SEQ ID NO: 20 - protein, 40 aa, Homo sapiens
LPDNILLELFTHVPARQLLLNCRLVCSLWRDLIDLLTLWK

SEQ ID NO: 21 - protein, 39 aa, Homo sapiens
LPLELKLRIFRLLDVRSVLSLSAVCRDLFTASNDPLLWR

SEQ ID NO: 22 - protein, 39 aa, Homo sapiens
LPDELLLGIFSCLCLPELLKVSGVCKRWYRLASDESLWQ

SEQ ID NO: 23 - DNA, 1323 bp, Homo sapiens
acattttctaatgtttacagaatgaagaggaacagtttatctgttgagaataaaattgtccagttgtcaggagcagcgaaacagccaaaagttgggttctactcttctctcaaccagactcatacacacacggttcttctagactgggggagtttgcctcaccatgtagtattacaaatttttcagtatcttcctttactagatcgggcctgtgcatcttctgtatgtaggaggtggaatgaagtttttcatatttctgacctttggagaaagtttgaatttgaactgaaccagtcagctacttcatcttttaagtccactcatcctgatctcattcagcagatcattaaaaagcattttgctcatcttcagtatgtcagctttaaggttgacagtagcgctgagtcagcagaagctgcctgtgatatactctctcagctggtaaattgttccatccagaccttgggcttgatttcaacagccaagccaagtttcatgaatgtgtcggagtctcattttgtgtcagcacttacagttgtttttatcaactcaaaatcattatcatcaatcaaaattgaagatacaccagtggatgatccttcattgaagattcttgtggccaataatagtgacactctaagactcccaaagatgagtagctgtcctcatgtttcatctgatggaattctttgtgtagctgaccgttgtcaaggccttagagaactggcgttgaattattacatcctaactgatgaacttttccttgcactctcaagcgagactcatgttaaccttgaacatcttcgaattgatgttgtgagtgaaaatcctggacagattaaatttcatgctgttaaaaaacacagttgggatgcacttattaaacattcccctagagttaatgttgttatgcacttctttctatatgaagaggaattcgagacgttcttcaaagaagaaacccctgttactcacctttattttggtcgttcagtcagcaaagtggttttaggacgggtaggtctcaactgtcctcgactgattgagttagtggtgtgtgctaatgatcttcagcctcttgataatgaacttatttgtattgctgaacactgtacaaacctaacagccttgggcctcagcaaatgtgaagttagctgcagtgccttcatcaggtttgtaagactgtgtgagagaaggttaacacagctctctgtaatggaggaagttttgatccctgatgaggattatagcctagatgaaattcacactgaagtctccaaatacctgggaagagtatggttccctgatgtgatgcctctctgg

SEQ ID NO: 24 - protein, 434 aa, Homo sapiens
MKRNSLSVENKIVQLSGAAKQPKVGFYSSLNQTHTHTVLLDWGSLPHHVVLQIFQYLPLLDRACASSVCRRWNEVFHISDLWRKFEFELNQSATSSFKSTHPDLIQQIIKKHFAHLQYVSFKVDSSAESAEAACDILSQLVNCSIQTLGLISTAKPSFMNVSESHFVSALTVVFINSKSLSSIKIEDTPVDDPSLKILVANNSDTLRLPKMSSCPHVSSDGILCVADRCQGLRELALNYYILTDELFLALSSETHVNLEHLRIDVVSENPGQIKFHAVKKHSWDALIKHSPRVNVVMHFFLYEEEFETFFKEETPVTHLYFGRSVSKVVLGRVGLNCPRLIELVVCANDLQPLDNELICIAEHCTNLTALGLSKCEVSCSAFIRFVRLCERRLTQLSVMEEVLIPDEDYSLDEIHTEVSKYLGRVWFPDVMPLW

SEQ ID NO: 25 - DNA, 1970 bp, Homo sapiens
ggaaacgtcaaaattgggatagtcggcagttctggcccctgcagctggaggtaccctgagttctgagggtcgtagtgctgtttctggtattctcatcgcggtcacctctaccggtgtggacaagtaaagtttgaatcagcttctccatggcctgggcaccagttcccggctgagccattttccttttggctaaaagtccccgcccagaggccaattcgtcgcggcggcggtggagatcgcaggtcgctcaggcttgcagatgggtcaagggttgtggagagtggtcagaaaccagcagctgcaacaagaaggctacagtgagcaaggctacctcaccagagagcagagcaggagaatggctgcgagcaacatttctaacaccaatcatcgtaaacaagtccaaggaggcattgacatatatcatcttttgaaggcaaggaaatcgaaagaacaggaaggattcattaatttggaaatgttgcctcctgagctaagctttaccatcttgtcctacctgaatgcaactgacctttgcttggcttcatgtgtttggcaggaccttgcgaatgatgaacttctctggcaagggttgtgcaaatccacttggggtcactgttccatatacaataagaacccacctttaggattttcttttagaaaaktgtatatgcagctggatgaaggcagcctcacctttaatgccaacccagatgagggagtgaactactttatgtccaagggtatcctggatgattcgccaaaggaaatagcaaagtttatcttctgtacaagaacactaaattggaaaaaactgagaatctatcttgatgaaaggagagatgtcttggatgaccttgtaacattgcataattttagaaatcagttcttgccaaatgcactgagagaattttttcgtcatatccatgcccctgaagagcgtggagagtatcttgaaactcttataacaaagttctcacatagattctgtgcttgcaaccctgatttaatgcgagaacttggccttagtcctgatgctgtctatgtactgtgctactctttgattctactttccattgacctcactagccctcatgtgaagaataaaatgtcaaaaagggaatttattcgaaatacccgtcgcgctgctcaaaatattagtgaagattttgtagggcatctttatgacaatatctaccttattggccatgtggctgcataaaaagcacaattgctaggacttcagtttttacttcagactaaagctacccaaggacttagcagatatgggggttacatcagtgctggtcattgtagcctgagtatacaatcaagcttcagtgtgcaacctttttttcttttgccattttctattttagtaatttccttggggaactaaataattttgcagaatttttcctaattttgtttatcacgttttgcacaaagcagagccactgtctaacacagctgttaacgaatgataaactgacattatactctaaaagatggtgtatttgtgcattagatttgcctgaaaaactttatccatttccattctttatacaaataccatgtaatgtgtacatatttaactaaagagatttatagtcataattattttattgtaaagattttaactaaagtttttccttttctctcaaactgagttctgaaatttatttgattctgatctgaaactattgtctycgtaaaagttagatctgacttcagrcagaaaccaataccagcttccttttcctttaaactttgaagagtgttgatttgttactatattactatgcaaaactggcagttatttttataatataaatttataatttgattttttattttaaaaactgggttaatcaagtctcggtaagtcctttaaaccatttaggatttttaaaacatcaaaatttatgatttacattcataggaataaaataaaatatyattagaactctggt

SEQ ID NO: 26 - protein, 634 aa, Homo sapiens
  SITE all Xaa positions: Xaa=unknown amino acid residue
ETSKLGSAVLAPAAGGTLSSEGRSAVSGILIAVTSTGVDKSLNQLLHGLGTSSRLSHFPFGKSPPRGQFVAAAVEIAGRSGLQMGQGLWRVVRNQQLQQEGYSEQGYLTREQSRRMAASNISNTNHRKQVQGGIDIYHLLKARKSKEQEGFINLEMLPPELSFTILSYLNATDLCLASCVWQDLANDELLWQGLCKSTWGHCSIYNKNPPLGFSFRKXYMQLDEGSLTFNANPDEGVNYFMSKGILDDSPKEIAKFIFCTRTLNWKKLRIYLDERRDVLDDLVTLHNFRNQFLPNALREFFRHIHAPEERGEYLETLITKFSHRFCACNPDLMRELGLSPDAVYVLCYSLILLSIDLTSPHVKNKMSKREFIRNTRRAAQNISEDFVGHLYDNIYLIGHVAAKAQLLGLQFLLQTKATQGLSRYGGYISAGHCSLSIQSSFSVQPFFLLPFSILVISLGNIILQNFSFCLSRFAQSRATVHSCRMINHYTLKDGVFVHICLKNFIHFHSLYKYHVMCTYLTKEIYSHNYFIVKILTKVFPFLSNVLKFIFSETIVXVKVRSDFRQKPIPASFSFKLRVLICYYITMQNWQLFLYKFIIFFILKTGLIKSRVLTIDFNIKIYDLHSENKIXLELW

SEQ ID NO: 27 - DNA, 4168 bp, Homo sapiens
gatggcggcggcagcagtcgacagcgcgatggaggtggtgccggcgctggcggaggaggccgcgccggaggtagcgggcctcagctgcctcgtcaacctgccgggtgaggtgctggagtacatcctgtgctgcggctcgctgacggccgccgacatcggccgtgtctccagcacctgccggcggctgcgcgagctgtgccagagcagcgggaaggtgtggaaggagcagttccgggtgaggtggccttcccttatgaaacactacagccccaccgactacgtcaattggttggaagagtataaagttcggcaaaaagctgggttagaagcgcggaagattgtagcctcgttctcaaagaggttcttttcagagcacgttccttgtaatggcttcagtgacattgagaaccttgaaggaccagagattttttttgaggatgaactggtgtgtatcctaaatatggaaggaagaaaagctttgacctggaaatactacgcaaaaaaaattctttactacctgcggcaacagaagatcttaaataatcttaaggcctttcttcagcagccagatgactatgagtcgtatcttgaaggtgctgtatatattgaccagtactgcaatcctctctccgacatcagcctcaaagacatccaggcccaaattgacagcatcgtggagcttgtttgcaaaacccttcggggcataaacagtcgccaccccagcttggccttcaaggcaggtgaatcatccatgataatggaaatagaactccagagccaggtgctggatgccatgaactatgtcctttacgaccaactgaagttcaaggggaatcgaatggattactataatgccctcaacttatatatgcatcaggttttgattcgcagaacaggaatcccaatcagcatgtctctgctctatttgacaattgctcggcagttgggagtcccactggagcctgtcaacttcccaagtcacttcttattaaggtggtgccaaggcgcagaaggggcgaccctggacatctttgactacatctacatagatgcttttgggaaaggcaagcagctgacagtgaaagaatgcgagtacttgatcggccagcacgtgactgcagcactgtatggggtggtcaatgtcaagaaggtgttacagagaatggtgggaaacctgttaagcctggggaagcgggaaggcatcgaccagtcataccagctcctgagagactcgctggatctctatctggcaatgtacccggaccaggtgcagcttctcctcctccaagccaggctttacttccacctgggaatctggccagagaaggtgcttgacatcctccagcacatccaaaccctagacccggggcagcacggggcggtgggctacctggtgcagcacactctagagcacattgagcgcaaaaaggaggaggtgggcgtagaggtgaagctgcgctccgatgagaagcacagagatgtctgctactccatcgggctcattatgaagcataagaggtatggctataactgtgtgatctacggctgggaccccacctgcatgatgggacacgagtggatccggaacatgaacgtccacagcctgccgcacggccaccaccagcctttctataacgtgctggtggaggacggctcctgtcgatacgcagcccaagaaaacttggaatataacgtggagcctcaagaaatctcacaccctgacgtgggacgctatttctcagagtttactggcactcactacatcccaaacgcagagctggagatccggtatccagaagatctggagtttgtctatgaaacggtgcagaatatttacagtgcaaagaaagagaacatagatgagtaaagtctagagaggacattgcacctttgctgctgctgctatcttccaagagaacgggactccggaagaagacgtctccacggagccctcgggacctgctgcaccaggaaagccactccaccagtagtgctggttgcctcctactaagtttaaataccgtgtgctcttccccagctgcaaagacaatgttgctctccgcctacactagtgaattaatctgaaaggcactgtgtcagtggcatggcttgtatgcttgtcctgtggtgacagtttgtgacattctgtcttcatgaggtctcacagtcgacgctcctgtaatcattctttgtattcactccattcccctgtctgtctgcatttgtctcagaacatttccttggctggacagatggggttatgcatttgcaataatttccttctgatttctctgtggaacgtgttcggtcccgagtgaggactgtgtgtctttttaccctgaagttagttgcatattcagaggtaaagttgtgtgctatcttggcagcatcttagagatggagacattaacaagctaatggtaattagaatcatttgaatttatttttttctaatatgtgaaacacagatttcaagtgttttatcttttttttttaaatttaaatgggaatataacacagttttcccttccatattcctctcttgagtttatgcacatctctataaatcattagttttctattttattacataaaattcttttagaaaatgcaaatagtgaactttgtgaatggatttttccatactcatctacaattcctccattttaaatgactacttttattttttaatttaaaaaatctacttcagtatcatgagtaggtcttacatcagtgatgggttctttttgtagtgagacatacaaatctgatgttaatgtttgctcttagaagtcatactccatggtcttcaaagaccaaaaaatgaggttttgcctttgtaatcaggaaaaaaaaaaattaatgaaccttaaaaaaaaaaaaaaaggttttgaagggaaaaaaagtggtttcacacctcttgttattccttagagtcacttcaaggcctgtttgaatgtggcaggttagaaagagagagaatgtctttcatttgaagagtgttggacttgtgtgaaaggagatgtgcgtgttggaatctgcttttccaagccgccagggtcctgacggcagcaggacgaagcctgttgtggcgtcttctgggaaagcctgaccgtgtgttcggacggcactggctcctttccgaagttctcagtaactgagcccagagtaactgcacgcctttgtgcagctctggagctccaccaactctcggcctgccagttctcaagcgagctaatcttgtcattaatcgatagaagctaacttccgaagttaggacctagttactttgctctcaacatttaaaataatgcagttgctctagtgaatggggcgttaggggcctgtctctgcacctgtctgtccatctgcatgcagtattctcacccatgttgaatgcctgctgcttgtttaccctttggaaaccctggggtgaccaaggtttggaaagccacctgagaccacttcatagcaagggaaggctttaagcagttactagaaagagatggggatttggcccctggctcctccagcctgaatgagctatttaatccactgtccatgttcctcatcagtcaaatccaaagtcaaaggatttgaacctgcatctggaaacgtaaccactcacagcacctggcccgccaaggttgggaggattgtacactactttcatttaaaggggaaagtttgataatacggaattaattaatatgaatgagatgcattaataagaacctgagcatgctgagagttgcaattgttggttttctggtttgattgatttccttttttcttagacacatcaaagtcaagaaagatggttttacctttactgacccagctgtacatatgtatctagactgtttttaaatgtctttcttcatgaatgcttcatggggctccaggaagcctgtatcacctgtgtaagttggtatttgggcactttatatttttctaaaaacgtgttttggatcctgtactctaataaatcataagtttctttttaaaaattttccaaaacttttctccattttaaaaagccctgttataaacgttgaactttcacaatgttaaaatgttaaatatttggatatagcaacttcttttctcttcaaatgaatgccaagatttttttgtacaatgattaataaatggaacttatccagag

SEQ ID NO: 28 - protein, 621 aa, Homo sapiens
MAAAAVDSAMEVVPALAEEAAPEVAGLSCLVNLPGEVLEYILCCGSLTAADIGRVSSTCRRLRELCQSSGKVWKEQFRVRWPSLMKHYSPTDYVNWLEEYKVRQKAGLEARKIVASFSKRFFSEHVPCNGFSDIENLEGPEIFFEDELVCILNMEGRKALTWKYYAKKILYYLRQQKILNNLKAFLQQPDDYESYLEGAVYIDQYCNPLSDISLKDIQAQIDSIVELVCKTLRGINSRHPSLAFKAGESSMIMEIELQSQVLDAMNYVLYDQLKFKGNRMDYYNALNLYMHQVLIRRTGIPISMSLLYLTIARQLGVPLEPVNFPSHFLLRWCQGAEGATLDIFDYIYIDAFGKGKQLTVKECEYLIGQHVTAALYGVVNVKKVLQRMVGNLLSLGKREGIDQSYQLLRDSLDLYLAMYPDQVQLLLLQARLYFHLGIWPEKVLDILQHIQTLDPGQHGAVGYLVQHTLEHIERKKEEVGVEVKLRSDEKHRDVCYSIGLIMKHKRYGYNCVIYGWDPTCMMGHEWIRNMNVHSLPHGHHQPFYNVLVEDGSCRYAAQENLEYNVEPQEISHPDVGRYFSEFTGTHYIPNAELEIRYPEDLEFVYETVQNIYSAKKENIDE

SEQ ID NO: 29 - DNA, 278 bp, Homo sapiens
  modified_base all n positions: n=a, c, g or t
ccgtagtactggnttccggcgggctggtgaggaatggagccggtagntgcttgcggcgagtcccgggntcctccgtagacccgcgganaccttcgtgttgagtaacctggcggaggtggtggagcgtgtgctcaccttcctgcccgccaaggcgttgctgcgggtggcctgcgtgtgccgcttatggagggagtgtgtgcgcagagtattgcggacccatcggagcgtaacctggatctccgcaggcctggcggaggccggccacctggnggggcatt

SEQ ID NO: 30 - protein, 91 aa, Homo sapiens
  SITE all Xaa positions: Xaa=unknown amino acid residue
RSTGFRRAGEEWSRXLAASPGXLRRPAXTFVLSNLAEVVERVLTFLPAKALLRVACVCRLWRECVRRVLRTHRSVTWISAGLAEAGHLXGH

SEQ ID NO: 31 - DNA, 592 bp, Homo sapiens
gcggccgcgcccggtgcagcaacagcagcagcagcccccgcagcagccgccgccgcagccgccccagcagcagccgccccagcagcagcctccgccgccgccgcagcagcagcagcagcagcagcctccgccgccgccaccgccgcctccgccgctgcctcaggagcggaacaacgtcggcgagcgggatgatgatgtgcctgcagatatggttgcagaagaatcaggtcctggtgcacaaaatagtccataccaacttcgtagaaaaactcttttgccgaaaagaacagcgtgtcccacaaagaacagtatggagggcgcctcaacttcaactacagaaaactttggtcatcgtgcaaaacgtgcaagagtgtctggaaaatcacaagatctatcagcagcacctgctgaacagtatcttcaggagaaactgccagatgaagtggttctaaaaatcttctcttacttgctggaacaggatctttgtagagcagcttgtgtatgtaaacgcttcagtgaacttgctaatgatcccaatttgtggaaacgattatatatggaagtatttgaatatactcgccctatgatgcat

SEQ ID NO: 32 - protein, 197 aa, Homo sapiens
RPRPVQQQQQQPPQQPPPQPPQQQPPQQQPPPPPQQQQQQQPPPPPPPPPPLPQERNNVGERDDDVPADMVAEESGPGAQNSPYQLRRKTLLPKRTACPTKNSMEGASTSTTENFGHRAKRARVSGKSQDLSAAPAEQYLQEKLPDEVVLKIFSYLLEQDLCRAACVCKRFSELANDPNLWKRLYMEVFEYTRPMMH

SEQ ID NO: 33 - DNA, 537 bp, Homo sapiens
gcggccgcggcccggactccgcggtgggcgagcgccctgtgaggtgaccatggaggctggtggcctccccttggagctgtggcgcatgatcttagcctacttgcaccttcccgacctgggccgctgcagcctggtatgcagggcctggtatgaactgatcctcagtctcgacagcacccgctggcggcagctgtgtctgggttgcaccgagtgccgccatcccaattggcccaaccagccagatgtggagcctgagtcttggagagaagccttcaagcagcattaccttgcatccaagacatggaccaagaatgccttggacttggagtcttccatctgcttttctctattccgccggaggagggaacgacgtaccctgagtgttgggccaggccgtgagtttgacagcctgggcagtgccttggccatggccagcctgtatgaccgaattgtgctcttcccaggtgtgtacgaagagcaaggtgaaatcatcttgaaggtgcctgtggagattgtagggcaggggaagttgggtga

SEQ ID NO: 34 - protein, 178 aa, Homo sapiens
RPRPGLRGGRAPCEVTMEAGGLPLELWRMILAYLHLPDLGRCSLVCRAWYELILSLDSTRWRQLCLGCTECRHPNWPNQPDVEPESWREAFKQHYLASKTWTKNALDLESSICFSLFRRRRERRTLSVGPGREFDSLGSALAMASLYDRIVLFPGVYEEQGEIILKVPVEIVGQGKLG

SEQ ID NO: 35 - DNA, 751 bp, Homo sapiens
gagaccgagacggcgccgctgaccctagagtcgctgcccaccgatcccctgctcctcatcttatcctttttggactatcgggatctaatcaactgttgttatgtcagtcgaagattaagccagctatcaagtcatgatccgctgtggagaagacattgcaaaaaatactggctgatatctgaggaagagaaaacacagaagaatcagtgttggaaatctctcttcatagatacttactctgatgtaggaagatacattgaccattatgctgctattaaaaaggcctcgggaatgatctcaagaaatatttggagcccaggtgtcctcggatgggttttatctctgaaagaggggtgctcgagaggaagacctcgatgctgtggaagcgcagattgggctgcaagtttcctggacgattatcgatgttcataccgaattcacaatggacagaagttagttggttcctggggttattgggaagcatggcactgtctaatcactatcgttctgaagatttgttagacgtcgatacagctgccggagattccagcagagacagggactgaaatactgtctccctttaacttttgcatacatactggtttgagtcagtacatagcagtggaagctgcagagggttgaaacaaaaatgaagttttctaccaatgtcagacagtagaacgtgtgtttaaatatggcattaagatgtgttctgatggttgtataaatggcatgcattaggtattttcag

SEQ ID NO: 36 - protein, 247 aa, Homo sapiens [sequence omitted]

SEQ ID NO: 37 - DNA, 368 bp, Homo sapiens
  modified_base all n positions: n=a, c, g or t
ggctccggtttccgggccggcgggtggccgctcaccatgcccggnaagcaccagcatttccaggaacctgaggtcggctgctgcgggaaatacttcctgtttggcttcaacattgtcttctgggtgctgggagccctgttcctggctatcggcctctgggcctggggtgagaagggcgttctctcgaacatctcagcgctgacagatctgggaggccttgaccccgtgtggcttgtttgtggtagttggaggcgtcatgtcggtgctgggctttgctgggctgcaattggggccctccgggagaacaccttcctgctcaagtttttctncgngttcctcggtctcatcttcttcctggagctggcaac

SEQ ID NO: 38 - protein, 122 aa, Homo sapiens
  SITE all Xaa positions: Xaa=unknown amino acid residue
GSGFRAGGWPLTMPGKHQHFQEPEVGCCGKYFLFGFNIVFWVLGALFLAIGLWAWGEKGVLSNISALTDLGGLDPVWLVCGSWRRHVGAGLCWAAIGALRENTFLLKFFXXFLGLIFFLELA

SEQ ID NO: 39 - DNA, 774 bp, Homo sapiens
gcggcggccgccgccgcgtacctggacgagctgcccgagccgctgctgctgcgcgtgctggccgcactgccggccgccgagctggtgcaggcctgccgcctggtgtgcctgcgctggaaggagctggtggacggcgccccgctgtggctgctcaagtgccagcaggaggggctggtgcccgagggcggcgtggaggaggagcgcgaccactggcagcagttctacttcctgagcaagcggcgccgcaaccttctgcgtaacccgtgtggggaagaggacttggaaggctggtgtgacgtggagcatggtggggacggctggagggtggaggagctgcctggagacagtggggtggagttcacccacgatgagagcgtcaagaagtacttcgcctcctcctttgagtggtgtcgcaaagcacaggtcattgacctgcaggctgagggctactgggaggagctgctggacacgactcagccggccatcgtggtgaaggactggtactcgggccgcagcgacgctggttgcctctacgagctcaccgttaagctactgtccgagcacgagaacgtgctggctgagttcagcagcgggcaggtggcagtgccccaagacagtgacggcgggggctggatggagatctcccacaccttcaccgactacgggccgggcgtccgcttcgtccgcttcgagcacggggggcagggctccgtctactggaagggctggttcggggcccgggtgaccaacagcagcgtgtgggtagaaccctga

SEQ ID NO: 40 - protein, 257 aa, Homo sapiens
AAAAAAYLDELPEPLLLRVLAALPAAELVQACRLVCLRWKELVDGAPLWLLKCQQEGLVPEGGVEEERDHWQQFYFLSKRRRNLLRNPCGEEDLEGWCDVEHGGDGWRVEELPGDSGVEFTHDESVKKYFASSFEWCRKAQVIDLQAEGYWEELLDTTQPAIVVKDWYSGRSDAGCLYELTVKLLSEHENVLAEFSSGQVAVPQDSDGGGWMEISHTFTDYGPGVRFVRFEHGGQGSVYWKGWFGARVTNSSVWVEP

SEQ ID NO: 41 - DNA, 957 bp, Homo sapiens
atgggcgagaaggcggtccctttgctaaggaggaggcgggtgaagagaagctgcccttcttgtggctcggagcttggggttgaagagaagagggggaaaggaaatccgatttccatccagttgttccccccagagctggtggagcatatcatctcattcctcccagtcagagaccttgttgccctcggccagacctgccgctacttccacgaagtgtgcgatggggaaggcgtgtggagacgcatctgtcgcagactcagtccgcgcctccaagatcaggacacgaagggcctgtatttccaggcatttggaggccgccgccgatgtctcagcaagagcgtggcccccttgctagcccacggctaccgccgcttcttgcccaccaaggatcacgtcttcattcttgactacgtggggaccctcttcttcctcaaaaatgccctggtctccaccctcggccagatgcagtggaagcgggcctgtcgctatgttgtgttgtgtcgtggagccaaggattttgcctcggacccaaggtgtgacacagtttaccgtaaatacctctacgtcttggccactcgggagccgcaggaagtggtgggtaccaccagcagccgggcctgtgactgtgttgaggtctatctgcagtctagtgggcagcgggtcttcaagatgacattccaccactcaatgaccttcaagcagatcgtgctggttggtcaggagacccagcgggctctactgctcctcacagaggaaggaaagatctactctttggtagtgaatgagacccagcttgaccagccacgctcctacacggttcagctggccctgaggaaggtgtcccactacctgcctcacctgcgcgtggcctgcatgacttccaaccagagcagcaccctctacgtcacagatcctattctgtgctcttggctacaaccaccttggcctggtggatga

SEQ ID NO: 42 - protein, 318 aa, Homo sapiens
MGEKAVPLLRRRRVKRSCPSCGSELGVEEKRGKGNPISIQLFPPELVEHIISFLPVRDLVALGQTCRYFHEVCDGEGVWRRICRRLSPRLQDQDTKGLYFQAFGGRRRCLSKSVAPLLAHGYRRFLPTKDHVFILDYVGTLFFLKNALVSTLGQMQWKRACRYVVLCRGAKDFASDPRCDTVYRKYLYVLATREPQEVVGTTSSRACDCVEVYLQSSGQRVFKMTFHHSMTFKQIVLVGQETQRALLLLTEEGKIYSLVVNETQLDQPRSYTVQLALRKVSHYLPHLRVACMTSNQSSTLYVTDPILCSWLQPPWPGG

SEQ ID NO: 43 - DNA, 1590 bp, Homo sapiens
cgagggggaagcgaaggaaggggaagaggaagggaaaagcgagcgagaggggcaaggcggaagaggaagcagggcggaagggaagcccgggccgcagacggcgaaggaggcagcgggccgggggctgaggcgggagcgaggacacgcccaagagaggaagcagagggaggcggaagcgtggaggaaggggcgagaggcatcatcaaaggagatgaggggagcgtaggggccgggaaagaggcacaaggaagaaagtatgggaaggaggaatggagggtcagggctaggcggcgggagggcgccaggccgggaagagtacaaggacaaggaggtcaggtttgggcctacatcccggggacaggggcggccatggcggcggcagccagggaggaggaggaggaggcggctcgggagtcagccgcctgcccggctgcggggccagcgctctggcgcctgccggaagtgctgctgctgcacatgtgctcctacctcgacatgcgggccctcggccgcctggcccaggtgtaccgctggctgtggcacttcaccaactgcgacctgctccggcgccagatagcctgggcctcgctcaactccggcttcacgcggctcggcaccaacctgatgaccagtgtcccagtgaaggtgtctcagaactggatagtggggtgctgccgagaggggattctgctgaagtggagatgcagtcagatgccctggatgcagctagaggatgatgctttgtacatatcccaggctaatttcatcctggcctaccagttccgtccagatggtgccagcttgaaccgtcagcctctgggagtctctgctgggcatgatgaggacgtttgccactttgtgctggccacctcgcatattgtcagtgcaggaggagatgggaagattggccttggtaagattcacagcaccttcgctgccaagtactgggctcatgaacaggaggtgaactgtgtggattgcaaagggggcatcatatcatttggctccagggacaggacggccaaggtgtggcctttggcctcaggccagctggggcagtgtttatacaccatccagactgaagaccaaatctggtctgttgctatcaggccattactcagctcttttgtgacagggacggcttgttgtgggcacttctcacccctgaaaatctgggacctcaacagtgggcagctgatgacacacttggacagagactttcccccaagggctggggtgctggatgtcatatatgagtcccctttcgcactgctctcctgtggctatgacacctatgttcgctactgggactgccgcaccagtgtccggaaatgtgtcatggagtgggaggagccccacaacagcaccctgtactgcctgcagacagatggcaaccacttgcttgccacaggttcctccttctatagcgttgtacggctgtgggaccggcaccaaagggcctgcccgcacaccttcccgctgacgtcgacccgcctcggcagccctgtgtactgcctgcatctcaccaccaagcatctctatgctgcgctgtcttacaacctccacgtcctggatattcaaaacccgtga

SEQ ID NO: 44 - protein, 529 aa, Homo sapiens
RGGSEGRGRGREKRARGARRKRKQGGREARAADGEGGSGPGAEAGARTRPREEAEGGGSVEEGARGIIKGDEGSVGAGKEAQGRKYGKEEWRVRARRREGARPGRVQGQGGQVWAYIPGTGAAMAAAAREEEEEAARESAACPAAGPALWRLPEVLLLHMCSYLDMRALGRLAQVYRWLWHFTNCDLLRRQIAWASLNSGFTRLGTNLMTSVPVKVSQNWIVGCCREGILLKWRCSQMPWMQLEDDALYISQANFILAYQFRPDGASLNRQPLGVSAGHDEDVCHFVLATSHIVSAGGDGKIGLGKIHSTFAAKYWAHEQEVNCVDCKGGIISFGSRDRTAKVWPLASGQLGQCLYTIQTEDQIWSVAIRPLLSSFVTGTACCGHFSPLKIWDLNSGQLMTHLDRDFPPRAGVLDVIYESPFALLSCGYDTYVRYWDCRTSVRKCVMEWEEPHNSTLYCLQTDGNHLLATGSSFYSVVRLWDRHQRACPHTFPLTSTRLGSPVYCLHLTTKHLYAALSYNLHVLDIQNP

SEQ ID NO: 45 - DNA, 1214 bp, Homo sapiens
gcattgctataattttactatactctcatctaaatctaaaatcagtcttcaaaataaaaacaaattgtcctttgccaaaaatttttttaatcgcacaattaattgacattaactgccaattctttttggctaattgactaattttaacttctgtgttgcttttccagaggcatggctattgcaccttgggagaagcctttaatcggttagacttctcaagtgcaattcaagatatccgaacgttcaattatgtggtcaaactgttgcagctaattgcaaaatcccagttaacttcattgagtggcgtggcacagaagaattacttcaacattttggataaaatcgttcaaaaggttcttgatgaccaccacaatcctcgcttaatcaaagatcttctgcaagacctaagctctaccctctgcattcttattagaggagtagggaagtctgtattagtgggaaacatcaatatttggatttgccgattagaaactattctcgcctggcaacaacagctacaggatcttcagatgactaagcaagtgaacaatggcctcaccctcagtgaccttcctctgcacatgctgaacaacatcctataccggttctcagacggatgggacatcatcaccttaggccaggtgacccccacgttgtatatgcttagtgaagacagacagctgtggaagaagctttgtcagtaccattttgctgaaaagcagttttgtagacatttgatcctttcagaaaaaggtcatattgaatggaagttgatgtactttgcacttcagaaacattacccagcgaaggagcagtacggagacacactgcatttctgtcggcactgcagcattctcttttggaaggactcaggacacccctgcacggcggccgaccctgacagctgcttcacgcctgtgtctccgcagcacttcatcgacctcttcaagttttaagggctgcccctgccatccctattggagattgtgaatcctgctgtctgtgcagggctcatagtgagtgttctgtgaggtgggtggagactcctcggaagcccctgcttccagaaagcctgggaagaactgcccttctgcaaaggggggactgcatggttgcattttcatcactgaaagtcagaggccaaggaaatcatttctacttctttaaaaactccttctaagcatattaaaatgtgaaattttgcgtactctctc

SEQ ID NO: 46 - protein, 272 aa, Homo sapiens
LILTSVLLFQRHGYCTLGEAFNRLDFSSAIQDIRTFNYVVKLLQLIAKSQLTSLSGVAQKNYFNILDKIVQKVLDDHHNPRLIKDLLQDLSSTLCILIRGVGKSVLVGNINIWICRLETILAWQQQLQDLQMTKQVNNGLTLSDLPLHMLNNILYRFSDGWDIITLGQVTPTLYMLSEDRQLWKKLCQYHFAEKQFCRHLILSEKGHIEWKLMYFALQKHYPAKEQYGDTLHFCRHCSILFWKDSGHPCTAADPDSCFTPVSPQHFIDLFKF

SEQ ID NO: 47 - DNA, 4059 bp, Homo sapiens
agtacggcagtgagggcaaaggcagctcgagcatctcatctgacgtgagttcaagtacagatcacacgcccactaaagcccagaagaatgtggctaccagcgaagactccgacctgagcatgcgcacactgagcacgcccagcccagccctgatatgtccaccgaatctcccaggatttcagaatggaaggggctcgtccacctcctcgtcctccatcaccggggagacggtggccatggtgcactccccgcccccgacccgcctcacacacccgctcatccggctcgcctccagaccccagaaggagcaggccagcatagaccggctcccggaccactccatggtgcagatcttctccttcctgcccaccaaccagctgtgccgctgcgcgcgagtgtgccgccgctggtacaacctggcctgggacccgcggctctggaggactatccgcctgacgggcgagaccatcaacgtggaccgcgccctcaaggtgctgacccgcagactctgccaggacacccccaacgtgtgtctcatgctggaaaccgtaactgtcagtggctgcaggcggctcacagaccgagggctgtacaccatcgcccagtgctgccccgaactgaggcgactggaagtctcaggctgttacaatatctccaacgaggccgtctttgatgtggtgtccctctgccctaatctggagcacctggatgtgtcaggatgctccaaagtgacctgcatcagcttgacccgggaggcctccattaaactgtcacccttgcatggcaaacagatttccatccgctacctggacatgacggactgcttcgtgctggaggacgaaggcctgcacaccatcgcggcgcactgcacgcagctcacccacctctacctgcgccgctgcgtccgcctgaccgacgaaggcctgcgctacctggtgatctactgcgcctccatcaaggagctgagcgtcagcgactgccgcttcgtcagcgacttcggcctgcgggagatcgccaagctggagtcccgcctgcggtacctgagcatcgcgcactgcggccgggtcaccgacgtgggcatccgctacgtggccaagtactgcagcaagctgcgctacctcaacgcgaggggctgcgagggcatcacggaccacggtgtggagtacctcgccaagaactgcaccaaactcaaatccctggatatcggcaaatgccctttggtatccgacacgggcctggagtgcctggccctgaactgcttcaacctcaagcggctcagcctcaagtcctgcgagagcatcaccggccagggcttgcagatcgtggccgccaactgctttgacctccagacgctgaatgtccaggactgcgaggtctccgtggaggccctgcgctttgtcaaacgccactgcaagcgctgcgtcatcgagcacaccaacccggctttcttctgaagggacagagttcatccggcgttgtattcacacaaacctgaacaaagcaaatttttttaaaagcagcgtatgtaagcaccgacacccactcaaaacagctctttcttccgggaaggttattaggaatctggcctttatttttcctcatttctcatgggcaacagaggccaaagaaacgaagcaagacaaacagcaaacaggcattttggtcaggtcatttgtaggcagtttctcttctcacaaaagatgtacttaagcaggctgatcgctgttccttgagcaaggcgcttactctcctccgctcaggcccccaaggccgccctttccctcgcacacaggccccacccccacagttccacgccccccccccaaggccacaccctccctccctagagcagcagcgaggatccatcatcagaatcacagtgctctccagacctcctctctaaactgcttcattgacctaagtcactctcttcaatcccacacccatggacattcttgtcaactcaataccatagcactttgcataggcaaaatacttttcaggcctttttaaaaaattcattacagcaaacagctggggaaggacatgcagtcctcccccagctctgtcaatgactatgaccttggccaaagcacttcactgctctgggctgcagcttccagcactgaatcagaggccacacagcccaaagattagcttcatgtccattatagcattgagggagcagagatacccatacacagaagcaccttggcatagagcacccaggcatcgacctcttccaggagaactgattctgtggatggatgtgatttcaggagattgtgcagtgccagcatcagtgcataaagggtcctgtatgtcctttggctgcaaatcacccacttccctgtgtttcagtgggagaatttcctctcccacctcctcacatcctcttttgccaggctggatgctgtcgtctctgtacacaaatactttctgcattcccccctccacaccatcctagcgaggcaccagcacacctaatcacagcaaagcccagatccccccatcagttgcttttactcagtgttttcaaataggagtaaaggcccttgcaatttttaattaacaagcaaggcccaagggaacacatgtcctcaaaagtttttctgatccctcgccttgcacacctggcatgcatcaggcacatctgtcctacagctggcagagacagatgcctcggttctttgtcattcagattgcatttgacctcttctcatctatttatttctttatacatccagacttcatcacatgaagcctattggggttaagtttgtaagtgtttaattgtgcaaattgccaccctgtgtacctcctccatgtctgtctgcgtgttttccaccaaagaatgcaaagcagacttccaggtgtttaaattctgttcactcaacaatgccagatgaatggaagagggaacacactgagatgacttagactctggtccaccaaccagacccttggaaaggaatactaaaatcattacaaggtatggattttaaatggatgaaacttcaaattatcttatttggatagaagtctatattctagcctcatttgcatgaagtcagatagccagaagaaattccattgctggttttcacgaaattcacttgtcttttgctaataaacacatggccctttcccagattattctctagccaagccccacctttgttacgttgaaatccctcatttattttcttctcaaaatgcccattatccaaatgcagaacctctgcatctccaagccagttatgctgaatttgtcaaacttagacacccttgacaactgcactcctactgtaggctcctgtgcatactgtcgtcttctgtgggggatggagaggttagtgtgatgaggtggtgtctgcccaggaggtttctttcaaacatcatggcctcccatccaatcaacatcatcaaattacatgtgtaatcaaggctctgtgccatgggggaaatgaatcatttagctaggccaggatctagtgaaagccacagagtttaaaaccatgaaagaagttgaaggcagcattcctcagctctgtgacttgtgaccctatttgaagtttcaggatttgggtgtcacaaaggattgtccctaatccttggccctggggtcttccgagtgagctggtttaatactctgagaatgagcagggagatccagagaatgaatccctgaccgcatcacctaaactgtcttccaaacatgagacaaagctgactgttcacactgattgcccagcacataccgtcttgccagtttcttcttttctcccagtctcctgttcatccattctgttctcccttggggtgggaatctatgatggaggttactggggaaacagctcagcagatttttggagaccaaaccaaaggtctcactaggaaatttatctgttttaaaacattgcttccttcctggctctgctaaattgaatgctcattgtttgttgttgttgttttttaattctaatgttcaaatcactgcgtgctgtatgaatctagaaagccttaatttactaccaagaaataaagcaatatgttcgt

SEQ ID NO: 48 - protein, 483 aa, Homo sapiens
YGSEGKGSSSISSDVSSSTDHTPTKAQKNVATSEDSDLSMRTLSTPSPALICPPNLPGFQNGRGSSTSSSSITGETVAMVHSPPPTRLTHPLIRLASRPQKEQASIDRLPDHSMVQIFSFLPTNQLCRCARVCRRWYNLAWDPRLWRTIRLTGETINVDRALKVLTRRLCQDTPNVCLMLETVTVSGCRRLTDRGLYTIAQCCPELRRLEVSGCYNISNEAVFDVVSLCPNLEHLDVSGCSKVTCISLTREASIKLSPLHGKQISIRYLDMTDCFVLEDEGLHTIAAHCTQLTHLYLRRCVRLTDEGLRYLVIYCASIKELSVSDCRFVSDFGLREIAKLESRLRYLSIAHCGRVTDVGIRYVAKYCSKLRYLNARGCEGITDHGVEYLAKNCTKLKSLDIGKCPLVSDTGLECLALNCFNLKRLSLKSCESITGQGLQIVAANCFDLQTLNVQDCEVSVEALRFVKRHCKRCVIEHTNPAFF

SEQ ID NO: 49 - DNA, 850 bp, Homo sapiens
tgcggccgcgcccgcacccgcaccggcacccacgcccacgcccgaggaagggcccgacgcgggctggggagaccgcattcccttggaaatcctggtgcagattttcgggttgttggtggcggcggacggccccatgcccttcctgggcagggctgcgcgcgtgtgccgccgctggcaggaggccgcttcccaacccgcgctctggcacaccgtgaccctgtcgtccccgctggtcggccggcctgccaagggcggggtcaaggcggagaagaagctccttgcttccctggagtggcttatgcccaatcggttttcacagctccagaggctgaccctcatccactggaagtctcaggtacaccccgtgttgaagctggtaggtgagtgctgtcctcggctcactttcctcaagctctccggctgccacggtgtgactgctgacgctctggtcatgctagccaaagcctgctgccagctccatagcctggacctacagcactccatggtggagtccacagctgtggtgagcttcttggaggaggcagggtcccgaatgcgcaagttgtggctgacctacagctcccagacgacagccatcctgggcgcattgctgggcagctgctgcccccagctccaggtcctggaggtgagcaccggcatcaaccgtaatagcattccccttcagctgcctgtcgaggctctgcagaaaggctgccctcagctccaggtgctgcggctgttgaacctgatgtggctgcccaagcctccgggacgaggggtggctcccggaccaggcttccctagcctagaggagctctgcctggcgagctcaacctgcaactttgtgagc

SEQ ID NO: 50 - protein, 283 aa, Homo sapiens
AAAPAPAPAPTPTPEEGPDAGWGDRIPLEILVQIFGLLVAADGPMPFLGRAARVCRRWQEAASQPALWHTVTLSSPLVGRPAKGGVKAEKKLLASLEWLMPNRFSQLQRLTLIHWKSQVHPVLKLVGECCPRLTFLKLSGCHGVTADALVMLAKACCQLHSLDLQHSMVESTAVVSFLEEAGSRMRKLWLTYSSQTTAILGALLGSCCPQLQVLEVSTGINRNSIPLQLPVEALQKGCPQLQVLRLLNLMWLPKPPGRGVAPGPGFPSLEELCLASSTCNFVS

SEQ ID NO: 51 - DNA, 1777 bp, Homo sapiens
  modified_base all n positions: n=a, c, g or t
acaacactgctctcagaaggatactgcagaactccttagaggtcttagcctatggaatcatgctgaagagcgacagaarttttttaaatattccgtggatgaaaagtcagataaagaagcagaagtgtcagaacactccacaggtataacccatcttcctcctgaggtaatgctgtcaattttcagctatcttaatcctcaagagttatgtcgatgcagtcaagtaagcatgaaatggtctcagctgacaaaaacgggatcgctttggaaacatctttaccctgttcattgggccagaggtgactggtatagtggtcccgcaactgaacttgatactgaacctgatgatgaatgggtgaaaaataggaaagatgaaagtcgtgcttttcatgagtgggatgaagatgctgacattgatgaatctgaagagtctgcggaggaatcaattgctatcagcattgcacaaatggaaaaacgtttactccatggcttaattcataacgttctaccatatgttggtacttctgtaaaaaccttagtattagcatacagctctgcagtttccagcaaaatggttaggcagattttagagctttgtcctaacctggagcatctggatcttacccagactgacatttcagattctgcatttgacagttggtcttggcttggttgctgccagagtcttcggcatcttgatctgtctggttgtgagaaaatcacagatgtggccctagagaagatttccagagctcttggaattctgacatctcatcaaagtggctttttgaaaacatctacaagcaaaattacttcaactgcgtggaaaaataaagacattaccatgcagtccaccaagcagtatgcctgtttgcacgatttaactaacaagggcattggagaagaaatagataatgaacacccctggactaagcctgtttcttctgagaatttcacttctccttatgtgtggatgttagatgctgaagatttggctgatattgaagatactgtggaatggagacatagaaatgttgaaagtctttgtgtaatggaaacagcatccaactttagttgttccacctctggttgttttagtaaggacattgttggactaaggactagtgtctgttggcagcagcattgtgcttctccagcctttgcgtattgtggtcactcattttgttgtacaggaacagctttaagaactatgtcatcactcccagaatcttctgcaatgtgtagaaaagcagcaaggactagattgcctaggggaaaagacttaatttactttgggagtgaaaaatctgatcaagagactggacgtgtacttctgtttctcagtttatctggatgttatcagatcacagaccatggtctcagggttttgactctgggaggagggctgccttatttggagcaccttaatctctctggttgtcttactataactggtgcaggcctgcaggatttggtttcagcatgtccttctctgaatgatgaatacttttactactgtgacaacattaacggtcctcatgctgataccgccagtggatgccagaatttgcagtgtggttttcgagcctgctgccgctctggcgaatgacccttgacttctgatctttgtctacttcatttagctgagcaggctttctttcatgcactttactcatagcacatttcttgtgttaaccatccctttttgagcgtgacttgttttgggcccattnyttacaacttcagaaatcttaattaccagtgrattgtaatgttg

SEQ ID NO: 52 - protein, 590 aa, Homo sapiens
  SITE all Xaa positions: Xaa=unknown amino acid residue
QHCSQKDTAELLRGLSLWNHAEERQKFFKYSVDEKSDKEAEVSEHSTGITHLPPEVMLSIFSYLNPQELCRCSQVSMKWSQLTKTGSLWKHLYPVHWARGDWYSGPATELDTEPDDEWVKNRKDESRAFHEWDEDADIDESEESAEESIAISIAQMEKRLLHGLIHNVLPYVGTSVKTLVLAYSSAVSSKMVRQILELCPNLEHLDLTQTDISDSAFDSWSWLGCCQSLRHLDLSGCEKITDVALEKISRALGILTSHQSGFLKTSTSKITSTAWKNKDITMQSTKQYACLHDLTNKGIGEEIDNEHPWTKPVSSENFTSPYVWMLDAEDLADIEDTVEWRHRNVESLCVMETASNFSCSTSGCFSKDIVGLRTSVCWQQHCASPAFAYCGHSFCCTGTALRTMSSLPESSAMCRKAARTRLPRGKDLIYFGSEKSDQETGRVLLFLSLSGCYQITDHGLRVLTLGGGLPYLEHLNLSGCLTITGAGLQDLVSACPSLNDEYFYYCDNINGPHADTASGCQNLQCGFRACCRSGEPLTSDLCLLHLAEQAFFHALYSHISCVNHPFLSVTCFGPIXYNFRNLNYQXIVML

SEQ ID NO: 53 - DNA, 1681 bp, Homo sapiens
  modified_base all n positions: n=a, c, g or t
ttttactgtacacagttgatgtattttgatgctgggcctgtctggtctgtcttgaggattattaacctttagaggtatcagagaagcaaatgggtactggtgaggctgctcattagggaagagggcaaaaggagcactagctaggtcagagccatgtttcaggtcacaatgtgatgtcagatgttgcttataaatcctttcttgtcttcgccattcttaaatcttgataggtgcctgttgggaaactgtaaatgcctttcccaatggagaatcaacagattgggtgatggtggagtcggtcaggaagactcaggtcttctagaggaaaggatgcctcatcaccccttnggcccaggcagctgctgtcagagaatgacacagcacctgcacagtcgctgtccacttcctgccactgctgtcggtggggtgacgggagcaaagtaggcgtggactttgacatgagggagctgagcccgcatccgcttgatgcctgcacgggtaacctgctggcagtcgtacagctcgaggcgctccaggcctcggcagttctctaggtgtyccagggccacatcagtgatgaggaggcagttgtccaactccagtacccgcagcctctcatggccacaggtactgttgctcaggtgcaggatcccatcatctgkgatgagttcacagtgggacaggctcagggcttgcagtttaggacagtgaatggagagctggatgagtgtgctgtcggttatcaggatgcawtcttcaagatccatcttctccaattcgtggcaattccgagctaaaagtgtaaaacctgcgtcagtcaaatgggagcatcgggcagcctccaaaatttgcagtcgcggacagttcaaacccagggctgtaagagaggcatctgtgaggttgctgcaacccgaaaggcagagagcctgtagccggtgacagcccctgcatatctgcaccacaccttcatccgtgatacgtgagcaggactgcaagttgaggctcacaagctcatggcagtaattctgaatgtgtttcagagcttcatcttctaactgtgtgcagcccctcaggagcagggctttcaggcctcgacaacctcgcaccagtgcctcgatgccatccttcgtgatctgatcacaccaagagaggttcaggtactccaggtttcggcagccctcactgatccccttcaaggagctgtttgtaatagacacacaggaggtcagawccagatgtttcagcttggaacagaatctgctaaggctataacacgtgctgtcagtgatttttgtgcatccattgaggttcaaatgttcaatgtttcggcagttctgtgcaaaggtcttcaaggaggaatccccaacaccaatgcagcctcgcaagctgagcttcctcaggaatccaacgcatcgcttcgagatattttccaccactcgaccctctacatctatttgaaagttaaaaagatctattctttgccagttgcttccatccagggctaagatgttccaagccttggaaatctgtgcacatcggcacaaagttactatatccaagaaggaaaatattcttaacagaagttctttgggtaactttttgttaataaggccttcatcattgtttgagaaaaccatggccgaagagccgcgagcgagcccacagcccgaagtcacacggc

SEQ ID NO: 54 - protein, 437 aa, Homo sapiens
  SITE all Xaa positions: Xaa=unknown amino acid residue
RVTSGCGLARGSSAMVFSNNDEGLINKKLPKELLLRIFSFLDIVTLCRCAQISKAWNILALDGSNWQRIDLFNFQIDVEGRVVENISKRCVGFLRKLSLRGCIGVGDSSLKTFAQNCRNIEHLNLNGCTKITDSTCYSLSRFCSKLKHLXLTSCVSITNSSLKGISEGCRNLEYLNLSWCDQITKDGIEALVRGCRGLKALLLRGCTQLEDEALKHIQNYCHELVSLNLQSCSRITDEGVVQICRGCHRLQALCLSGCSNLTDASLTALGLNCPRLQILEAARCSHLTDAGFTLLARNCHELEKMDLEXCILITDSTLIQLSIHCPKLQALSLSHCELIXDDGILHLSNSTCGHERLRVLELDNCLLITDVALXHLENCRGLERLELYDCQQVTRAGIKRMRAQLPHVKVHAYFAPVTPPTAVAGSGQRLCRCCVIL

SEQ ID NO: 55 - DNA, 1866 bp, Homo sapiens
atgtcaccggtctttcccatgttaacagttctgaccatgttttattatatatgccttcggcgccgagccaggacagctacaagaggagaaatgatgaacacccatagagctatagaatcaaacagccagacttcccctctcaatgcagaggtagtccagtatgccaaagaagtagtggatttcagttcccattatggaagtgagaatagtatgtcctatactatgtggaatttggctggtgtaccaaatgtattcccaagttctggtgactttactcagacagctgtgtttcgaacttatgggacatggtgggatcagtgtcctagtgcttccttgccattcaagaggacgccacctaattttcagagccaggactatgtggaacttacttttgaacaacaggtgtatcctacagctgtacatgttctagaaacctatcatcccggagcagtcattagaattctcgcttgttctgcaaatccttattccccaaatccaccagctgaagtaagatgggagattctttggtcagagagacctacgaaggtgaatgcttcccaagctcgccagtttaaaccttgtattaagcagataaatttccccacaaatcttatacgactggaagtaaatagttctcttctggaatattacactgaattagatgcagttgtgctacatggtgtgaaggacaagccagtgctttctctcaagacttcacttattgacatgaatgatatagaagatgatgcctatgcagaaaaggatggttgtggaatggacagtcttaacaaaaagtttagcagtgctgtcctcggggaagggccaaataatgggtattttgataaactaccttatgagcttattcagctgattctgaatcatcttacactaccagacctgtgtagattagcacagacttgcaaactactgagccagcattgctgtgatcctctgcaatacatccacctcaatctgcaaccatactgggcaaaactagatgacacttctctggaatttctacagtctcgctgcactcttgtccagtggcttaatttatcttggactggcaatagaggcttcatctctgttgcaggatttagcaggtttctgaaggtttgtggatccgaattagtacgccttgaattgtcttgcagccactttcttaatgaaacttgcttagaagttatttctgagatgtgtccaaatctacaggccttaaatctctcctcctgtgataagctaccacctcaagctttcaaccacattgccaagttatgcagccttaaacgacttgttctctatcgaacaaaagtagagcaaacagcactgctcagcattttgaacttctgttcagagcttcagcacctcagtttaggcagttgtgtcatgattgaagactatgatgtgatagctagcatgataggagccaagtgtaaaaaactccggaccctggatctgtggagatgtaagaatattactgagaatggaatagcagaactggcttctgggtgtccactactggaggagcttgaccttggctggtgcccaactctgcagagcagcaccgggtgcttcaccagactggcacaccagctcccaaacttgcaaaaactctttcttacagctaatagatctgtgtgtgacacagacattgatgaattggcatgtaattgtaccaggttacagcagctggacatattaggaacaagaatggtaagtccggcatccttaagaaaactcctggaatcttgtaaagatctttctttacttgatgtgtccttctgttcgcagattgataacagagctgtgctagaactgaatgcaagctttccaaaagtgttcataaaaaagagctttactcagtga

SEQ ID NO: 56 - protein, 621 aa, Homo sapiens
MSPVFPMLTVLTMFYYICLRRRARTATRGEMMNTHRAIESNSQTSPLNAEVVQYAKEVVDFSSHYGSENSMSYTMWNLAGVPNVFPSSGDFTQTAVFRTYGTWWDQCPSASLPFKRTPPNFQSQDYVELTFEQQVYPTAVHVLETYHPGAVIRILACSANPYSPNPPAEVRWEILWSERPTKVNASQARQFKPCIKQINFPTNLIRLEVNSSLLEYYTELDAVVLHGVKDKPVLSLKTSLIDMNDIEDDAYAEKDGCGMDSLNKKFSSAVLGEGPNNGYFDKLPYELIQLILNHLTLPDLCRLAQTCKLLSQHCCDPLQYIHLNLQPYWAKLDDTSLEFLQSRCTLVQWLNLSWTGNRGFISVAGFSRFLKVCGSELVRLELSCSHFLNETCLEVISEMCPNLQALNLSSCDKLPPQAFNHIAKLCSLKRLVLYRTKVEQTALLSILNFCSELQHLSLGSCVMIEDYDVIASMIGAKCKKLRTLDLWRCKNITENGIAELASGCPLLEELDLGWCPTLQSSTGCFTRLAHQLPNLQKLFLTANRSVCDTDIDELACNCTRLQQLDILGTRMVSPASLRKLLESCKDLSLLDVSFCSQIDNRAVLELNASFPKVFIKKSFTQ

SEQ ID NO: 57 - DNA, 984 bp, Homo sapiens
atgcaacttgtacctgatatagagttcaagattacttatacccggtctccagatggtgatggcgttggaaacagctacattgaagataatgatgatgacagcaaaatggcagatctcttgtcctacttccagcagcaactcacatttcaggagtctgtgcttaaactgtgtcagcctgagcttgagagcagtcagattcacatatcagtgctgccaatggaggtcctgatgtacatcttccgatgggtggtgtctagtgacttggacctcagatcattggagcagttgtcgctggtgtgcagaggattctacatctgtgccagagaccctgaaatatggcgtctggcctgcttgaaagtttggggcagaagctgtattaaacttgttccgtacacgtcctggagagagatgtttttagaacggcctcgtgttcggtttgatggcgtgtatatcagtaaaaccacatatattcgtcaaggggaacagtctcttgatggtttctatagagcctggcaccaagtggaatattacaggtacataagattctttcctgatggccatgtgatgatgttgacaacccctgaagagcctcagtccattgttccacgtttaagaactaggaataccaggactgatgcaattctactgggtcactatcgcttgtcacaagacacagacaatcagaccaaagtatttgctgtaataactaagaaaaaagaagaaaaaccacttgactataaatacagatattttcgtcgtgtccctgtacaagaagcagatcagagttttcatgtggggctacagctatgttccagtggtcaccagaggttcaacaaactcatctggatacatcattcttgtcacattacttacaaatcaactggtgagactgcagtcagtgcttttgagattgacaagatgtacacccccttgttcttcgccagagtaaggagctacacagctttctcagaaaggcctctgtag

SEQ ID NO: 58 - protein, 327 aa, Homo sapiens
MQLVPDIEFKITYTRSPDGDGVGNSYIEDNDDDSKMADLLSYFQQQLTFQESVLKLCQPELESSQIHISVLPMEVLMYIFRWVVSSDLDLRSLEQLSLVCRGFYICARDPEIWRLACLKVWGRSCIKLVPYTSWREMFLERPRVRFDGVYISKTTYIRQGEQSLDGFYRAWHQVEYYRYIRFFPDGHVMMLTTPEEPQSIVPRLRTRNTRTDAILLGHYRLSQDTDNQTKVFAVITKKKEEKPLDYKYRYFRRVPVQEADQSFHVGLQLCSSGHQRFNKLIWIHHSCHITYKSTGETAVSAFEIDKMYTPLFFARVRSYTAFSERPL

SEQ ID NO: 59 - DNA, 765 bp, Homo sapiens
  modified_base all n positions: n=a, c, g or t
gcagccctggatcctgacttagagaatgatgatttctttgtcagaaagactggggctttccatgcaaatccatatgttctccgagcttttgaagactttagaaagttctctgagcaagatgattctgtagagcgagatataattttacagtgtagagaaggtgaacttgtacttccggatttggaaaaagatgatatgattgttcgccgaatcccagcacagaagaaagaagtgccgctgtctggggccccagatagataccacccagtcccttttcccgaaccctggactcttcctccagaaattcaagcaaaatttctctgtgtacttgaaaggacatgcccatccaaagaaaaaagtaatagctgtagaatattagttccttcatatcggcagaagaaagatgacatgctgacacgtaagattcagtcctggaaactgggaactaccgtgcctcccatcagtttcacncctggcccctgcagtgaggctgacttgaagagatgggaggccatccgggaggccagcagactcaggcacaagaaaaggctgatggtggagagactctttcaaaagatttatggtgagaatgggagtaagtccatgagtgatgtcagcgcagaagatgttcaaaacttgcgtcagctgcgttacgaggagatgcagaaaataaaatcacaattaaaagaacaagatcagaaatggcaggatgaccttgcaaaatggaaagatcgtcgaaaaagttacacttcagatctgcagaag

SEQ ID NO: 60 - protein, 255 aa, Homo sapiens
AALDPDLENDDFFVRKTGAFHANPYVLRAFEDFRKFSEQDDSVERDIILQCREGELVLPDLEKDDMIVRRIPAQKKEVPLSGAPDRYHPVPFPEPWTLPPEIQAKFLCVLERTCPSKEKSNSCRILVPSYRQKKDDMLTRKIQSWKLGTTVPPISFTPGPCSEADLKRWEAIREASRLRHKKRLMVERLFQKIYGENGSKSMSDVSAEDVQNLRQLRYEEMQKIKSQLKEQDQKWQDDLAKWKDRRKSYTSDLQK

SEQ ID NO: 61 - protein, 36 aa, Homo sapiens
LPPELSFTILSYLNATDLCLASCVWQDLANDELLWQ

SEQ ID NO: 62 - protein, 42 aa, Homo sapiens
LPGEVLEYILCCGSLTAADIGRVSSTCRRLRELCQSSGKVWK

SEQ ID NO: 63 - protein, 44 aa, Homo sapiens
LAEVVERVLTFLPAKALLRVACVCRLWRECVRRVLRTHRSVTWI

SEQ ID NO: 64 - protein, 39 aa, Homo sapiens
LPDEVVLKIFSYLLEQDLCRAACVCKRFSELANDPNLWK

SEQ ID NO: 65 - protein, 41 aa, Homo sapiens
LPLELWRMILAYLHLPDLGRCSLVCRAWYELILSLDSTRWR

SEQ ID NO: 66 - protein, 39 aa, Homo sapiens
LPTDPLLLILSFLDYRDLINCCYVSRRLSQLSSHDPLWR

SEQ ID NO: 67 - protein, 40 aa, Homo sapiens
LPEPLLLRVLAALPAAELVQACRLVCLRWKELVDGAPLWL

SEQ ID NO: 68 - protein, 40 aa, Homo sapiens
LFPPELVEHIISFLPVRDLVALGQTCRYFHEVCDGEGVWR

SEQ ID NO: 69 - protein, 44 aa, Homo sapiens
LPEVLLLHMCSYLDMRALGRLAQVYRWLWHFTNCDLLRRQIAWA

SEQ ID NO: 70 - protein, 40 aa, Homo sapiens
LPLHMLNNILYRFSDGWDIITLGQVTPTLYMLSEDRQLWK

SEQ ID NO: 71 - protein, 39 aa, Homo sapiens
LPDHSMVQIFSFLPTNQLCRCARVCRRWYNLAWDPRLWR

SEQ ID NO: 72 - protein, 44 aa, Homo sapiens
IPLEILVQIFGLLVAADGPMPFLGRAARVCRRWQEAASQPALWH

SEQ ID NO: 73 - protein, 39 aa, Homo sapiens
LPPEVMLSIFSYLNPQELCRCSQVSMKWSQLTKTGSLWK

SEQ ID NO: 74 - protein, 39 aa, Homo sapiens
LPKELLLRIFSFLDIVTLCRCAQISKAWNILALDGSNWQ

SEQ ID NO: 75 - protein, 48 aa, Homo sapiens
LPYELIQLILNHLTLPDLCRLAQTCKLLSQHCCDPLQYIHLNLQPYWA

SEQ ID NO: 76 - protein, 44 aa, Homo sapiens
LPMEVLMYIFRWVVSSDLDLRSLEQLSLVCRGFYICARDPEIWR

SEQ ID NO: 77 - protein, 49 aa, Homo sapiens
LPPEIQAKFLCVLERTCPSKEKSNSCRILVPSYRQKKDDMLTRKIQSWK

SEQ ID NO: 78 - protein, 39 aa, Homo sapiens
LPHHVVLQIFQYLPLLDRACASSVCRRWNEVFHISDLWR

SEQ ID NO: 79 - protein, 43 aa, Homo sapiens
LWAWGEKGVLSNISALTDLGGLDPVWLVCGSWRRHVGAGLCWA

SEQ ID NO: 80 - DNA, 59 bp, Artificial Sequence
  Description of Artificial Sequence Oligonucleotide
agtagtaacaaaggtcaaagacagttgactgtatcgtcgaggatgccttcaattaagtt

SEQ ID NO: 81 - DNA, 58 bp, Artificial Sequence
  Description of Artificial Sequence Oligonucleotide
gcggttacttacttagagctcgacgtcttacttacttagctcacttctcttcacacca

SEQ ID NO: 82 - protein, 12 aa, Homo sapiens
CDGEKDTYSYLA

SEQ ID NO: 83 - protein, 25 aa, Homo sapiens
CESSFSLNMNFSSKRTKFKITTSMQ

SEQ ID NO: 84 - protein, 12 aa, Homo sapiens
CEEAQVRKENQW

SEQ ID NO: 85 - protein, 19 aa, Homo sapiens
  Phosphorylation 8: Phosothreonine
NAGSVEQTPKKPGLRRRQT

SEQ ID NO: 86 - DNA, 17 bp, Artificial Sequence
  Description of Artificial Sequence Oligonucleotide
cctgggggatgttctca

SEQ ID NO: 87 - DNA, 17 bp, Artificial Sequence
  Description of Artificial Sequence Oligonucleotide
ggcttccgggcatttag

SEQ ID NO: 88 - DNA, 17 bp, Artificial Sequence
  Description of Artificial Sequence Oligonucleotide
catctggcacgattcca

SEQ ID NO: 89 - DNA, 17 bp, Artificial Sequence
  Description of Artificial Sequence Oligonucleotide
ccgctcatcgtatgaca

SEQ ID NO: 90 - protein, 19 aa, Homo sapiens
  Phosphorylation 8: Phosotyrosine
AEIGVGAYGTVYKARDPHS

SEQ ID NO: 91 - protein, 20 aa, Homo sapiens
  Phosphorylation 9: Phosothreonine
NNAGSVEWTPKKPGLRRRQT

SEQ ID NO: 92 - protein, 15 aa, Homo sapiens
QIYYSDKDDEEFEYR

(19) United States
(12) Patent Application Publication
(10) Pub. No.: US 2005/0214879 A1
(43) Pub. Date: Sep. 29, 2005

(54) Novel ubiquitin ligases as therapeutic targets

(75) Inventor: Michele Pagano, New York, NY (US)

Correspondence Address: JONES DAY, 222 EAST 41ST ST, NEW YORK, NY, 10017, US

(73) Assignee: New York University

(21) Appl. No.: 11/073,470

(22) Filed: Mar. 4, 2005

Related U.S. Application Data
(63) Continuation of application No. 10/042,417, filed on Jan. 7, 2002.
(63) Continuation-in-part of application No. 10/632,150, filed on Jul. 30, 2003.
(60) Provisional application No. 60/260,179, filed on Jan. 5, 2001.

(51) Int. Cl. G01N 33/574; C12Q 1/37

(52) U.S. Cl. 435/7.23; 435/23

(57) ABSTRACT

The present invention relates to the discovery, identification and characterization of nucleotides that encode novel substrate-targeting subunits of ubiquitin ligases. The invention encompasses nucleotides encoding novel substrate-targeting subunits of ubiquitin ligases: FBP1, FBP2, FBP3, FBP4, FBP5, FBP6, FBP7, FBP8, FBP9, FBP10, FBP11, FBP12, FBP13, FBP14, FBP15, FBP16, FBP17, FBP18, FBP19, FBP20, FBP21, FBP22, FBP23, FBP24, and FBP25, transgenic mice, knock-out mice, host cell expression systems and proteins encoded by the nucleotides of the present invention. The present invention relates to screening assays that use the novel substrate-targeting subunits to identify potential therapeutic agents such as small molecules, compounds or derivatives and analogues of the novel ubiquitin ligases which modulate activity of the novel ubiquitin ligases for the treatment of proliferative and differentiative disorders, such as cancer, major opportunistic infections, immune disorders, certain cardiovascular diseases, and inflammatory disorders. The invention further encompasses therapeutic protocols and pharmaceutical compositions designed to target ubiquitin ligases and their substrates for the treatment of proliferative disorders.

This application claims priority under 35 U.S.C. § 119(e) to U.S. Application No. 60/260,179, filed Jan. 5, 2001, the contents of which are incorporated herein by reference in their entirety.

### 1. INTRODUCTION

The present invention relates to the discovery, identification and characterization of nucleotide sequences that encode novel substrate-targeting subunits of ubiquitin ligases. The invention encompasses nucleic acid molecules comprising nucleotide sequences encoding novel substrate-targeting subunits of ubiquitin ligases: FBP1, FBP2, FBP3a, FBP3b, FBP4, FBP5, FBP6, FBP7, FBP8, FBP11, FBP12, FBP13, FBP14, FBP15, FBP17, FBP18, FBP20, FBP21) FBP22, FBP23, AND FBP25, transgenic mice, knock-out mice, host cell expression systems and proteins encoded by the nucleotides of the present invention. The present invention relates to screening assays to identify potential therapeutic agents such as small molecules, compounds or derivatives and analogues of the novel ubiquitin ligases which modulate activity of the novel ubiquitin ligases for the treatment of proliferative and differentiative disorders, such as cancer, major opportunistic infections, immune disorders, certain cardiovascular diseases, and inflammatory disorders. The invention further encompasses therapeutic protocols and pharmaceutical compositions designed to target ubiquitin ligases and their substrates for the treatment of proliferative disorders.

### 2. BACKGROUND OF THE INVENTION

2.1 Cell Cycle Regulatory Proteins

The eukaryotic cell cycle is regulated by a family of serine/threonine protein kinases called cyclin dependent kinases (Cdks) because their activity requires the association with regulatory subunits named Cyclins (Hunter & Pines, 1994, Cell 79:573). Cdks also associate with Cdk inhibitors (Ckis) which mediate cell cycle arrest in response to various antiproliferative signals. So far, based on their sequence homology, two families of Ckis have been identified in mammalian cells: the Cip/Kip family, which includes p21, p27 and p57; and the Ink family, which includes p15, p16, p18, and p20 (Sherr & Roberts, 1999, Genes & Dev. 13: 1501).

2.2 The Ubiquitin Pathway

Ubiquitin-mediated proteolysis is an important pathway of non-lysosomal protein degradation which controls the timed destruction of many cellular regulatory proteins including, p27, p53, p300, cyclins, E2F, STAT-1, c-Myc, c-Jun, EGF receptor, IkBα, NFkB and β-catenin (reviewed in Pagano, 1997, FASEB J. 11:1067). Ubiquitin is an evolutionary highly conserved 76-amino acid polypeptide which is abundantly present in all eukaryotic cells. The ubiquitin pathway leads to the covalent attachment of a poly-ubiquitin chain to target substrates which are then degraded by the multi-catalytic proteasome complex (see Pagano, supra, for a recent review). Many of the steps regulating protein ubiquitination are known. Initially the ubiquitin activating enzyme (E1), forms a high energy thioester with ubiquitin which is, in turn, transferred to a reactive cysteine residue of one of many ubiquitin conjugating enzymes (Ubcs or E2s). The final transfer of ubiquitin to an e-amino group of a reactive lysine residue in the target protein occurs in a reaction that may or may not require an ubiquitin ligase (E3) protein. The large number of ubiquitin ligases ensures the high level of substrate specificity.

2.3 The Ubiquitin Pathway and the Regulation of the G1 Phase by F Box Proteins

Genetic and biochemical studies in several organisms have shown that the G1 phase of the cell cycle is regulated by the ubiquitin pathway. Proteolysis of cyclins, Ckis and other G1 regulatory proteins is controlled in yeast by the ubiquitin conjugating enzyme Ubc3 (also called Cdc34) and by an E3 ubiquitin ligase formed by three subunits: Cdc53, Skp1 and one of many F box proteins (reviewed in E. Patton et al., 1998, TIG. 14:6). The F box proteins (FBPs) are so called because they contain a motif, the F box, that was first identified in Cyclin F, and that is necessary for FBP interaction with Skp1 (Bai, et al., 1996, Cell 86:263). In addition, F box proteins also contain either WD-40 domains or Leucine-Rich Repeats (LRR) protein-protein interaction domains. Cdc53 (also called Cul A) and Skp1 appear to participate in the formation of at least three distinct E3, each containing a different F box protein. Because these ligases are similar protein modules composed of Skp1, Cul A, and an F box protein, they have been named SCF. The interaction of the ligase with its substrates occurs via the F box subunit. The three SCFs identified so far in S. cerevisiae are: SCF^{Cdc4} (which recruits the Ckis Sic1 and Far1, the replication factor Cdc6, and the transcriptional activator Gcn4, as substrates through the F box protein Cdc4), SCF^{Grr1} (which recruits the G1 cyclins Cln1 and Cln2 as substrates through the F box protein GRR1), and SCF^{Met30} (which recruits the G1 cyclin Cln3 as a substrate throughout the F box protein MET30; see Pagano and Patton, supra, for recent reviews).

The intracellular level of the human Cki p27, a cell cycle regulated cyclin-dependent kinase (Cdk) inhibitor, is mainly regulated by degradation and it is known that the ubiquitin system controls p27 degradation (Pagano et al., 1995, Science 269:682). Similarly, degradation of other G1 human regulatory proteins (Cyclin E, Cyclin D1, p21, E2F, β-catenin) is controlled by the ubiquitin-pathway (reviewed in M. Pagano, supra). Yet, the specific enzymes involved in the degradation of G1 regulatory proteins have not been identified. A family of 6 genes (CUL1, 2, 3, 4a, 4b, and 5) homologous to S. cerevisiae cul A have been identified by searching the EST database (Kipreos, et al., 1996, Cell 85:829). Human Skp1 and the F box protein Skp2 (that contains five LRRs) were identified as two proteins associated in vivo with Cyclin A and thus designated as S-phase kinase-associated protein 1 and 2 (Zhang, et al., 1995, Cell 82:915). It has been demonstrated that phosphorylated p27 is specifically recognized by Skp2. Skp1 and Skp2 are also found to associate with Cul-1 and ROC1/Rbx1 to form an SCF ubiquitin ligase complex, SCF^{Skp2} ubiquitin ligase complex. While studies establish that p27 is targeted for degradation by the SCF^{Skp2} ubiquitin ligase complex, key factors involved in the degradation were unknown. It had been hypothesized that Nedd8, a highly conserved ubiquitin-like protein that is ligated to different cullins, is a necessary component for ligation of p27 (Podust, et al., 2000, Proc. Natl. Acad. Sci. USA 97:4579).

The highly conserved Suc1 (suppressor of Cdc2 mutation)/Cks(cyclin-dependent kinase subunit) family of cell cycle regulatory proteins binds to some cyclin-dependent kinases and phosphorylated proteins and is essential for cell cycle progression. Suc1 (Hayles, et al., 1986, Mol. Gen. Genet. 202:291) and Cks1 (Hadwiger, et al., 1989, Mol. Cell Biol. 9:2034) were discovered in fission and budding yeast, respectively, as essential gene products that interact with cyclin-dependent kinases. Homologues from different species share extensive sequence conservation, and the two human homologues can functionally substitute for Cks1 in budding yeast (Richardson, et al. 1990, Genes and Dev. 4:1332). Crystal structures of the two human homologues and the fission yeast Suc1 have shown that they share a four-stranded β-sheet involved in binding to a Cdk catalytic subunit (Bourne, et al., 1996, Cell 84:863; Pines, J., 1996, Curr. Biol. 11:1399). In addition, they share a highly conserved phosphate-binding site, positioned on a surface contiguous to the Cdk catalytic site in the Cks-Cdk complex (Bourne, et al., 1996, Cell 84:863).

Cks proteins are involved in several cell cycle transitions, including the G1 to S-phase transition, entry into mitosis and exit from mitosis (Pines, J., 1996, Curr. Biol. 11:1399), but the molecular basis for their different actions is not well understood. With the exception of Cln2/Cln3-Cdk1 complexes from budding yeast being activated by Cks1 (Reynard, et al., 2000, Mol. Cell Biol. 20:5858), Cks proteins do not directly affect the catalytic activity of the cyclin-dependent kinase. However, Cks proteins can promote multi-site phosphorylations of some substrates by cyclin-dependent kinases. It has been proposed that by simultaneously binding to a partially phosphorylated protein and to a Cdk, Cks proteins increase the affinity of the kinase for the substrate and thus accelerate subsequent multiple phosphorylations (Pines, J., 1996, Curr. Biol. 11:1399). Indeed, Cks proteins promote Cdk-catalyzed multiple phosphorylations of subunits of the cyclosome/APC (Patra, D. & Dunphy, W. G., 1998, Genes Dev. 12:2549; Shteinberg, M. & Hershko, A., 1999, Biochem. Biophys. Res. Commun. 257:12), as well as G2/M regulators such as Cdc25, Myt1 and Wee1 (Patra, et al., 1999, J. Biol. Chem. 274:36839).

2.4 Deregulation of the Ubiquitin Pathway in Cancer and Other Proliferative Disorders

Cancer develops when cells multiply too quickly. Cell proliferation is determined by the net balance of positive and negative signals. When positive signals overcome or when negative signals are absent, the cells multiply too quickly and cancer develops.

Ordinarily cells precisely control the amount of any given protein and eliminate the excess or any unwanted protein. To do so, the cell specifically tags the undesired protein with a long chain of molecules called ubiquitin. These molecules are then recognized and destroyed by a complex named proteasome. However, all this mechanism goes awry in tumors leading to the excessive accumulation of positive signals (oncogenic proteins), or resulting in the abnormal degradation of negative regulators (tumor suppressor proteins). Thus, without tumor suppressor proteins or in the presence of too much oncogenic proteins, cells multiply ceaselessly, forming tumors (reviewed by Ciechanover, 1998, EMBO J. 17: 7151; Spataro, 1998, Br. J. Cancer 77: 448). For example, abnormal ubiquitin-mediated degradation of the p53 tumor suppressor (reviewed by J. Brown and M. Pagano, 1997, Biochim. Biophys. Acta1332: 1), the putative oncogene β-catenin (reviewed by Peifer, 1997, Science 275:1752) and the Cki p27 (reviewed in Ciechanover, supra; Spataro, supra; Lloyd, 1999, Am. J. Pathol. 154: 313) have been correlated with tumorgenesis, opening to the hypothesis that some genes encoding ubiquitinating enzymes may be mutated in tumors.

Initial evidence indicates that human F-box proteins play a role in the ubiquitination of G1 regulatory proteins as their homologues do in yeast (see below). Unchecked degradation of cell cycle regulatory proteins has been observed in certain tumors and it is possible that deregulated ubiquitin ligase play a role in the altered degradation of cell cycle regulators. A well understood example is that of Mdm2, a ubiquitin ligase whose overexpression induces low levels of its substrate, the tumor suppressor p53.

### 3. SUMMARY OF THE INVENTION

The present invention relates to novel F box proteins and therapeutic protocols and pharmaceutical compositions designed to target the novel F box proteins and their interactions with substrates for the treatment of proliferative and differentiative disorders. The present invention also relates to screening assays to identify substrates of the novel F box proteins and to identify agents which modulate or target the novel ubiquitin ligases and interactions with their substrates. The invention further relates to screening assays based on the identification of novel substrates of known F box proteins, such as the two novel substrates of the known F box protein Skp2, E2F and p27. The screening assays of the present invention may be used to identify potential therapeutic agents for the treatment of proliferative or differentiative disorders and other disorders that related to levels of expression or enzymatic activity of F box proteins.

The invention is based in part, on the Applicants' discovery, identification and characterization of nucleic acids comprising nucleotide sequences that encode novel ubiquitin ligases with F box motifs. These twenty-six novel substrate-targeting subunits of ubiquitin ligase complexes, FBP1, FBP2, FBP3a, FBP3b, FBP4, FBP5, FBP6, FBP7, FBP8, FBP9, FBP10, FBP11, FBP12, FBP13, FBP14, FBP15, FBP16, FBP17, FBP18, FBP19, FBP20, FBP21, FBP22, FBP23, FBP24, and FBP25, described herein, were first identified based on their interaction with components of the ubiquitin ligase complex (FBP1, FBP2, FBP3a, FBP4, FBP5, FBP6 and FBP7) or by sequence comparison of these proteins with nucleotide sequences present in DNA databases (FBP3b, FBP8, FBP9, FBP10, FBP11, FBP12, FBP13, FBP14, FBP15, FBP16, FBP17, FBP18, FBP19, FBP20, FBP21, FBP22, FBP23, FBP24, and FBP25). These novel substrate-targeting subunits of ubiquitin ligase complexes each contain an F box motif through which they interact with the other components of the ubiquitin ligase complex. In addition, some of these FBPs contain WD-40 domains and LRRs (which appear to be involved in their interaction with substrates), while other FBPs contain potential protein-protein interaction modules not yet identified in FBPs, such as leucine zippers, ring fingers, helix-loop-helix motifs, proline rich motifs and SH2 domains. The invention is also based, in part, on the Applicants' discovery and identification of FBP specific substrates p27 and β-catenin and on methods to identify novel FBP substrates. Some of the genes encoding the novel F box proteins were also mapped to chromosome sites frequently altered in breast, prostate and ovarian cancer, nasopharyngeal and small cell lung carcinomas, gastric hepatocarcinomas, Burkitt's lymphoma and parathyroid adenomas. Finally, the invention is also based, in part, on the Applicants' generation of transgenic mice expressing wild type or dominant negative versions of FBP proteins and on the generation of FBP knock-out mice.

The invention encompasses the following nucleotide sequences, host cells expressing such nucleotide sequences, and the expression products of such nucleotide sequences: (a) nucleotide sequences that encode mammalian FBP1, FBP2, FBP3a, FBP3b, FBP4, FBP5, FBP6, FBP7, FBP8, FBP11, FBP12, FBP13, FBP14, FBP15, FBP17, FBP18, FBP20, FBP21, FBP22, FBP23, and FBP25, including the human nucleotides, and their gene products; (b) nucleotides that encode portions of the novel substrate-targeting subunits of ubiquitin ligase complexes, and the polypeptide products specified by such nucleotide sequences, including but not limited to F box motifs, the substrate binding domains; WD-40 domains; and leucine rich repeats, etc.; (c) nucleotides that encode mutants of the novel ubiquitin ligases in which all or part of the domain is deleted or altered, and the polypeptide products specified by such nucleotide sequences; (d) nucleotides that encode fusion proteins containing the novel ubiquitin ligases or one of its domains fused to another polypeptide.

The invention further encompasses agonists and antagonists of the novel substrate-targeting subunits of ubiquitin ligase complexes, including small molecules, large molecules, mutants that compete with native F box binding proteins, and antibodies as well as nucleotide sequences that can be used to inhibit ubiquitin ligase gene expression (e.g., antisense and ribozyme molecules, and gene regulatory or replacement constructs) or to enhance ubiquitin ligase gene expression (e.g., expression constructs that place the ubiquitin ligase gene under the control of a strong promoter system), and transgenic animals that express a ubiquitin ligase transgene or knock-outs that do not express the novel ubiquitin ligases.

Further, the present invention also relates to methods for the use of the genes and/or gene products of novel substrate-targeting subunits of ubiquitin ligase complexes for the identification of compounds which modulate, i.e., act as agonists or antagonists, of ubiquitin ligase activity. Such compounds can be used as agents to control proliferative or differentiative disorders, e.g. cancer. In particular, the present invention encompasses methods to inhibit the interaction between β-catenin and FBP1 or p27 and Skp2. In fact, agents able to block these interactions can be used to modulate cell proliferation and/or growth.

Still further, the invention encompasses screening methods to identify derivatives and analogues of the novel substrate-targeting subunits of ubiquitin ligase complexes which modulate the activity of the novel ligases as potential therapeutics for proliferative or differentiative disorders. The invention provides methods of screening for proteins that interact with novel components of the ubiquitin ligase complex, including FBP1, FBP2, FBP3a, FBP3b, FBP4, FBP5, FBP6, FBP7, FBP8, FBP9, FBP10, FBP11, FBP12, FBP13, FBP14, FBP15, FBP16, FBP17, FBP18, FBP19, FBP20, FBP21, FBP22, FBP23, FBP24, and FBP25 or derivatives, fragments or domains thereof, such as the F box motif. In accordance with the invention, the screening methods may utilize known assays to identify protein-protein interactions including phage display assays or the yeast two-hybrid assay system or variations thereof.

In addition, the present invention is directed to methods that utilize FBP gene sequences and/or FBP gene product sequences for the diagnostic evaluation, genetic testing and/or prognosis of an FBP-related disorder, such as a proliferative disorder. For example, the invention relates to methods for diagnosing FBP-related disorders, e.g., proliferative disorders, wherein such methods can comprise measuring FBP gene expression in a patient sample, or detecting an FBP mutation that correlates with the presence or development of such a disorder, in the genome of a mammal suspected of exhibiting such a disorder. In particular, the invention encompasses methods for determining if a subject (e.g., a human patient) is a risk for a disorder characterized by one or more of: (i) a mutation of an FBP gene encoding a protein represented in part A of FIGS. 3-28, or a homologues thereof; (ii) the mis-expression of an FBP gene; (iii) the mis-expression of an FBP protein.

The invention is illustrated by way of working examples which demonstrate the identification and characterization of the novel substrate-targeting subunits of ubiquitin ligase complexes. The working examples of the present invention further demonstrate the identification of the specific interaction of (i) FBP1 with β-catenin and (ii) the known FBP, Skp2, with the cell-cycle regulatory proteins E2F and p27 and the cell cycle protein Cks1. These interactions suggest that β-catenin is a specific substrate of FBP1, while E2F and p27 are substrates of Skp2 and Cks1 is a mediator for Skp2 and p27. In fact, the working examples of the present invention further demonstrate that β-catenin is a specific substrate of FBP1, while p27 is substrates of Skp2 and Cks1 binds to both p27 and Skp2. The identification of proteins interacting with the novel FBPs will be possible using the methods described herein or with a different approach.

3.1 Definitions

As used herein, the term “F-box motif” refers to a stretch of approximately 40 amino acid that was identified as being necessary for the interaction of F-box containing proteins with Skp1. The consensus sequence of an F-box motif is described in Bai et al., 1996, Cell 86:263-274, incorporated herein by reference in its entirety.

As used herein the term “F-box protein” (FBP) refers to peptide, polypeptide or protein which contains an F-box motif.

Although, FBPs are substrate-targeting subunits of ubiquitin ligase complexes, as used herein the term “ubiquitin ligase” refers to a peptide, polypeptide or protein that contains an F-box motif and interacts with Skp1.

As used herein, the term “functionally equivalent to an FBP gene product” refers to a gene product that exhibits at least one of the biological activities of the endogenous FBP gene product. For example, a functionally equivalent FBP gene product is one that is capable of interacting with Skp1 so as to become associated with a ubiquitin ligase complex. Such a ubiquitin ligase complex may be capable of ubiquitinating a specific cell-cycle regulatory protein, such as a cyclin or cki protein.

As used herein, the term “to target” means to inhibit, block or prevent gene expression, enzymatic activity, or interaction with other cellular factors.

As used herein, the term “therapeutic agent” refers to any molecule, compound or treatment that alleviates of assists in the treatment of a proliferative disorder or related disorder.

As used herein, the terms “WD-40 domain”, “Leucine Rich Repeat”, “Leucine Zipper”, “Ring finger”, “Helix-loop-helix motif”, “Proline rich motif”, and “SH2 domain” refer to domains potentially involved in mediating protein-protein interactions. The “WD-40 domain” refers to a consensus sequence of forty amino acid repeats which is rich in tryptophan and aspartic acid residues and is commonly found in the beta subunits of trimeric G proteins (see Neer et al., 1994 Nature 371:297-300 and references therein, which are incorporated herein by reference in their entirety). An “LRR” or a “Leucine Rich Repeat” is a leucine rich sequence also known to be involved in mediating protein-protein interactions (see Kobe & Deisenhofer, 1994, Trends. Biochem. Sci. 19:415-421 which are incorporated herein by reference in their entirety). A “leucine zipper” domain refers to a domain comprising a stretch of amino acids with a leucine residue in every seventh position which is present in a large family of transcription factors (see Landshultz et al., 1988, Science 240:1759-64; see also Sudol et al., 1996, Trends Biochem. 21:1-3, and Koch et al., 1991, Science 252:668-74).

### 4. BRIEF DESCRIPTION OF THE FIGURES

FIG. 1. Alignment of the conserved F-box motif amino acid residues in the human F-box proteins FBP1 (SEQ ID NO:15), FBP2 (SEQ ID NO:16), FBP3a (SEQ ID NO:17), FBP3b (SEQ ID NO:78), FBP4 (SEQ ID NO:18), FBP5 (SEQ ID NO:19), FBP6 (SEQ ID NO:20), FBP7 (SEQ ID NO:21), Skp2 (SEQ ID NO:22), FBP8 (SEQ ID NO:61) FBP9 (SEQ ID NO:62), FBP10 (SEQ ID NO:63), FBP11 (SEQ ID NO:64), FBP12 (SEQ ID NO:65), FBP13 (SEQ ID NO:79); FBP14 (SEQ ID NO:66); FBP15 (SEQ ID NO:67), FBP16 (SEQ ID NO:68), FBP17 (SEQ ID NO:69), FBP18 (SEQ ID NO:70), FBP19 (SEQ ID NO:71), FBP20 (SEQ ID NO:72), FBP21 (SEQ ID NO:73), FBP22 (SEQ ID NO:74), FBP23 (SEQ ID NO:75), FBP24 (SEQ ID NO:76), FBP25 (SEQ ID NO:77). Alignment of the F-boxes of a previously known FBP, Skp2, with the F-boxes of FBPs identified through a two-hybrid screen (designated by the pound symbol) or BLAST searches (designated by a cross) was performed using the Clustal W method (MacVector(tm)) followed by manual re-adjustment. Identical residues in at least 15 F-boxes are shaded in dark gray, while similar residues are shaded in light gray. One asterisk indicates the presence in the cDNA of a STOP codon followed by a polyA tail, while potential full length clones are designated with two asterisks. The asterisks on the bottom of the figure indicate the amino acid residues mutated in FBP3a (see FIG. 29).

FIG. 2. Schematic representation of FBPs. Putative protein-protein interaction domains in human FBPs are represented (see key-box for explanation). FBPs identified by a two-hybrid screen are designated by the pound symbol, FBPs identified through BLAST searches by a cross. The double slash indicates that the corresponding cDNAs are incomplete at the 5′ end; the asterisks indicate the presence in the cDNA of a STOP codon followed by a polyA tail.

FIG. 3A-B. A. Amino acid sequence of human F-box protein FBP1 (SEQ ID NO:2). B. Corresponding cDNA (SEQ ID NO:1).

FIG. 4A-B. A. Amino acid sequence of human F-box protein FBP2 (SEQ ID NO:4). B. Corresponding cDNA (SEQ ID NO:3).

FIG. 5A-B. A. Amino acid sequence of human F-box protein FBP3a (SEQ ID NO:6). B. Corresponding cDNA (SEQ ID NO:5).

FIG. 6A-B. A. Amino acid sequence of human F-box protein FBP3b (SEQ ID NO:24). B. Corresponding cDNA (SEQ ID NO:23).

FIG. 7A-B. A. Amino acid sequence of human F-box protein FBP4 (SEQ ID NO:8). B. Corresponding cDNA (SEQ ID NO:7).

FIG. 8A-B. A. Amino acid sequence of human F-box protein FBP5 (SEQ ID NO:10). B. Corresponding cDNA (SEQ ID NO:9).

FIG. 9A-B. A. Amino acid sequence of human F-box protein FBP6 (SEQ ID NO:12). B. Corresponding cDNA (SEQ ID NO:11).

FIG. 10A-B. A. Amino acid sequence of human F-box protein FBP7 (SEQ ID NO:14). B. Corresponding cDNA (SEQ ID NO:13).

FIG. 11A-B. A. Amino acid sequence of human F-box protein FBP8 (SEQ ID NO:26). B. Corresponding cDNA (SEQ ID NO:25).

FIG. 12A-B. A. Amino acid sequence of human F-box protein FBP9 (SEQ ID NO:28). B. Corresponding cDNA (SEQ ID NO:27).

FIG. 13A-B. A. Amino acid sequence of human F-box protein FBP10 (SEQ ID NO:30). B. Corresponding cDNA (SEQ ID NO:29).

FIG. 14A-B. A. Amino acid sequence of human F-box protein FBP11 (SEQ ID NO:32). B. Corresponding cDNA (SEQ ID NO:31).

FIG. 15A-B. A. Amino acid sequence of human F-box protein FBP12 (SEQ ID NO:34). B. Corresponding cDNA (SEQ ID NO:33).

FIG. 16A-B. A. Amino acid sequence of human F-box protein FBP13 (SEQ ID NO:36). B. Corresponding cDNA (SEQ ID NO:35).

FIG. 17A-B. A. Amino acid sequence of human F-box protein FBP14 (SEQ ID NO:38). B. Corresponding cDNA (SEQ ID NO:37).

FIG. 18A-B. A. Amino acid sequence of human F-box protein FBP15 (SEQ ID NO:40). B. Corresponding cDNA (SEQ ID NO:39).

FIG. 19A-B. A. Amino acid sequence of human F-box protein FBP16 (SEQ ID NO:42). B. Corresponding cDNA (SEQ ID NO:41).

FIG. 20A-B. A. Amino acid sequence of human F-box protein FBP17 (SEQ ID NO:44). B. Corresponding cDNA (SEQ ID NO:43).

FIG. 21A-B. A. Amino acid sequence of human F-box protein FBP18 (SEQ ID NO:46). B. Corresponding cDNA (SEQ ID NO:45).

FIG. 22A-B. A. Amino acid sequence of human F-box protein FBP19 (SEQ ID NO:48). B. Corresponding cDNA (SEQ ID NO:47).

FIG. 23A-B. A. Amino acid sequence of human F-box protein FBP20 (SEQ ID NO:50). B. Corresponding cDNA (SEQ ID NO:49).

FIG. 24A-B. A. Amino acid sequence of human F-box protein FBP21 (SEQ ID NO:52). B. Corresponding cDNA (SEQ ID NO:51).

FIG. 25A-B. A. Amino acid sequence of human F-box protein FBP22 (SEQ ID NO:54). B. Corresponding cDNA (SEQ ID NO:53).

FIG. 26A-B. A. Amino acid sequence of human F-box protein FBP23 (SEQ ID NO:56). B. Corresponding cDNA (SEQ ID NO:55).

FIG. 27A-B. A. Amino acid sequence of human F-box protein FBP24 (SEQ ID NO:58). B. Corresponding cDNA (SEQ ID NO:57).

FIG. 28A-B. A. Amino acid sequence of human F-box protein FBP25 (SEQ ID NO:60). B. Corresponding cDNA (SEQ ID NO:59).

FIG. 29. FBPs interact specifically with Skp1 through their F-box. The cDNAs of FBPs (wild type and mutants) were transcribed and translated in vitro (IVT) in the presence of 35S-methionine. Similar amounts of IVT proteins (indicated at the top of each lane) were subjected to a histidine-tagged pull-down assay using Nickel-agarose beads to which either His-tagged-Skp1 (lanes 1, 3, 4, 6-10, 12, 15, 17, 19 and 21), His-tagged-Elongin C (lanes 2, 5, 11, 14, 16, 18, 19 and 22), or His-tagged p27 (lane 12) were pre-bound. Bound IVT proteins were analyzed by SDS-PAGE and autoradiography. The arrows on the left side of the panels point to the indicated FBPs. The apparent molecular weights of the protein standards are indicated on the right side of the panels.

FIG. 30. FBP1, FBP2, FBP3a, FBP4 and FBP7 form novel SCFs with endogenous Skp1 and Cul1 in vivo. HeLa cells were transfected with mammalian expression plasmids encoding Flag-tagged versions of FBP1 (lane 1), (ΔF)FBP1 (lane 2), FBP4 (lane 3), FBP7 (lane 5), FBP2 (lane 7), (ΔF)FBP2 (lane 8), FBP3a (lane 9), (ΔF)FBP3a (lane 10), or with an empty vector (lanes 4 and 6). Cells were lysed and extracts were subjected to immunoprecipitation with a rabbit anti-Flag antibody (lanes 1-8). Immunoprecipitates were then immunoblotted with a mouse anti-Cul1 monoclonal antibody, a rabbit anti-Skp1 polyclonal antibody or a rabbit anti-Cul2 polyclonal antibody, as indicated. The last lane contains 25 μg of extracts from non-transfected HeLa cells; lane 9 contains recombinant Cul1, Skp1, or Cul2 proteins used as markers. The slower migrating bands detected with the antibodies to Cul1 and Cul2 are likely generated by the covalent attachment of a ubiquitin-like molecule to these two cullins, as already described for the yeast cullin Cdc53 and mammalian Cul4a.

FIG. 31. FBP1, FBP2, FBP3a, FBP4 and FBP7 associate with a ubiquitin ligase activity. HeLa cells were transfected with mammalian expression plasmids encoding human Skp1, Cul1 and Flag-tagged versions of FBP1 (lane 3), (ΔF)FBP1 (lane 4), FBP2 (lanes 2 and 5), (ΔF)FBP2 (lane 6), FBP7 (lane 7), FBP3a (lanes 8 and 13), (ΔF)FBP3a (lane 9), a non relevant Flag-tagged protein (Irf3, lane 10), FBP4 (lanes 11 and 12) or with an empty vector (lane 1). Cells were lysed and extracts were subjected to immunoprecipitation with a rabbit anti-Flag antibody. Immunoprecipitates were incubated in the presence of purified recombinant E1 and Ubc4 (lanes 1-11) or Ubc2 (lanes (12 and 13) and a reaction mix containing biotynilated ubiquitin. Reaction in lane 2 contained also NEM. Ubiquitinated proteins were visualized by blotting with HRP-streptavidin. The bracket on the left side of the panels marks a smear of ubiquitinated proteins produced in the reaction, the asterisk indicates ubiquitin conjugated with E1 that were resistant to boiling.

FIG. 32. Subcellular localization of FBPs. HeLa cells were transfected with mammalian expression plasmids encoding Flag-tagged versions of FBP1 (a-b), FBP2 (c-d), FBP3a (e-f), FBP4 (g-h), (DF)FBP2 (i-j), or (ΔF)FBP3a (k-l). After 24 hours, cells were subjected to immunofluorescence with a rabbit anti-Flag antibody (a, c, e, g, i, k) to stain FBPs and bisbenzimide (b, d, f, h, j, l ) to stain nuclei.

FIG. 33. Abundance of FBP transcripts in human tissues. Membranes containing electrophoretically fractionated poly(A)+mRNA from different human tissues were hybridized with specific probes prepared form FBP1, FBP2, FBP3a, FBP4, SKP2, and β-ACTIN cDNAs. The arrows on the left side of the figure point to the major transcripts as described in the text.

FIG. 34A-E. FISH localization of FBP genes. Purified phage DNA containing a genomic probe was labeled with digoxygenin dUTP and detected with Cy3-conjugated antibodies. The signals corresponding to the locus of the genomic probe (red) are seen against the DAPI-Actimomycin D stained normal human chromosomes (blue-white). Panel A shows localization of FBP1 to 10q24, B shows localization of FBP2 to 9q34, C shows localization of FBP3a to 13q22, D shows localization of FBP4 to 5p 12, and E shows localization of FBP5 to 6q25-26. Arrows point to FBP-specific FISH signals.

FIG. 35A-C. FBP1 associates with β-catenin. A. Extracts from baculovirus-infected insect cells expressing either β-catenin alone (lane 1) or in combination with Flag-tagged FBP1 (lane 2) were immunoprecipitated (IP) with a rabbit anti-Flag antibody (rα-Flag), followed by immunoblotting with anti-Flag (mα-Flag) and anti-β-catenin mouse antibodies, as indicated. Lanes 3 and 4 contain 25 μg of extracts from infected insect cells immunoblotted with the same antibodies. B. Extracts from baculovirus-infected insect cells expressing cyclin D1, Flag-FBP1 in the absence (lanes 1-3) or in the presence of Skp1 (lanes 4-6) were immunoprecipitated with normal rabbit IgG (r-IgG, lanes 1 and 4), rabbit anti-Flag antibody® α-Flag, lanes 2 and 5), or rabbit anti-cyclin D1 antibody® α-D1, lanes 3 and 6). Immunoprecipitates were then immunoblotted with anti-Flag (mα-Flag) and cyclin D1 (m α-D1) mouse antibodies, as indicated. The last lane contains 25 μg of a representative extract from infected insect cells immunoblotted with the same antibodies. C. 293 cells were transfected with mammalian expression plasmids encoding HA-tagged β-catenin alone or in combination with either Flag-tagged FBP1 or Flag-tagged (ΔF)FBP1. Cells were lysed and extracts were subjected to immunoprecipitation with a rabbit anti-Flag antibody® α-Flag, lanes 4-6) and immunoblotted with rat anti-HA (α-HA) and mouse anti-Flag (m α-Flag) antibodies, as indicated. The first three lanes contain 25 μg of extracts from transfected 293 cells immunoblotted with the same antibodies. Transfecting high levels of β-catenin expression vector, the associations of β-catenin with FBP1 and (ΔF)FBP1 could be determined independently of β-catenin levels.

FIG. 36A-B. Stabilization of β-catenin by a dominant negative (ΔF)FBP1 mutant. A. Human 293 cells were transfected with mammalian expression plasmids encoding HA-tagged β-catenin alone or in combination with either Flag-tagged (ΔF)FBP1 or Flag-tagged (ΔF)FBP2. Cells were lysed and extracts were subjected to immunoblotting with rat anti-HA and rabbit anti-Flag® α-Flag) antibody, as indicated. B. Pulse chase analysis of β-catenin turnover rate. HA-tagged β-catenin in combination with either an empty vector, FBP1, or (ΔF)FBP1 was co-transfected in 293 cells. 24 hours later cells were labeled with 35S-methionine for 30 minutes and chased with medium for the indicated times. Extracts were then subjected to immunoprecipitation with a rat anti-HA antibody.

FIG. 37A-C. Binding of phosphorylated p27 to Skp2. A. A panel of in vitro translated [35S]FBPs were used in binding reactions with beads coupled to the phospho-peptide NAGSVEQT*PKKPGLRRRQT, corresponding to the carboxy terminus of the human p27 with a phosphothreonine at position 187 (T*). Beads were washed with RIPA buffer and bound proteins were eluted and subjected to electrophoresis and autoradiography (Upper Panel). Bottom Panel: 10% of the in vitro translated [35S]FBP inputs. B. HeLa cell extracts were incubated with beads coupled to the phospho-p27 peptide (lane 2), an identical except unphosphorylated p27 peptide (lane 1) or the control phospho-peptide AEIGVGAY*GTVYKARDPHS, corresponding to an amino terminal peptide of human Cdk4 with a phosphotyrosine at position 17 (Y*) (lane 3). Beads were washed with RIPA buffer and bound proteins were immunoblotted with antibodies to the proteins indicated on the left of each panel. A portion of the HeLa extract (25 μg) was used as a control (lane 4). The slower migrating band in Cul1 is likely generated by the covalent attachment of a ubiquitin-like molecule, as already described for other cullins 48. C. One μl of in vitro translated [35S] wild type p27 (WT, lanes 1-4) or p27(T187A) mutant (T187A, lanes 5-6) were incubated for 30 minutes at 30¼C in 10 μl of kinase buffer. Where indicated, ˜2.5 pmole of recombinant purified cyclin E/Cdk2 or ˜1 pmole Skp2 (in Skp1/Skp2 complex) were added. Samples were then incubated with 6 μl of Protein-A beads to which antibodies to Skp2 had been covalently linked. Beads were washed with RIPA buffer and bound proteins subjected to electrophoresis and autoradiography. Lanes 1-6: Skp2-bound proteins; Lanes 7 and 8: 7.5% of the in vitro translated [35S] protein inputs.

FIG. 38. In vivo binding of Skp2 to p27. Extracts from HeLa cells (lanes 1-2 and 5-6) or IMR90 fibroblasts (lanes 9-10) were immunoprecipitated with different affinity purified (AP) antibodies to Skp2 or with purified control IgG fractions. Lane 1: extract immunoprecipitated with a goat IgG (G-IgG); lane 2: with an AP goat antibody to an N-terminal Skp2 peptide (G-α-Skp2,); lanes 5 and 9: with a rabbit IgG (R-IgG); lanes 6 and 10: with an AP rabbit antibody to Skp2 (R-α-Skp2). Immunoprecipitates were immunoblotted with antibodies to the proteins indicated on the left of each panel. Lanes 1-4 in the bottom panel were immunoblotted with a phospho-site p27 specific antibody. Lanes 3, 7, and 11 contain 25 μg of cell extracts; Lanes 4, 8, and 12 contain the relevant recombinant proteins used as markers. The altered migration of some markers is due to the presence of tags on the recombinant proteins.

FIG. 39A-B. Skp2 and cyclin E/Cdk2 complex are rate-limiting for p27 ubiquitination in G1 extracts. A. In vitro ubiquitin ligation (lanes 1-12 and 17-20) and degradation (lanes 13-16) of p27 were carried out with extracts from asynchronously growing (Asyn. ext., lanes 2-3) or G1-arrested (G1 ext., lanes 4-20) HeLa cells. Lane 1 contains no extract. Recombinant purified proteins were supplemented as indicated. Reactions were performed using wild-type p27 (lanes 1-18) or p27(T187A) mutant (T187A, lanes 19-20). Lanes 1-8,9-12, and 17-20 are from three separate experiments. The bracket on the left side of the panels marks a ladder of bands >27,000 corresponding to polyubiquitinated p27. The asterisk indicates a non-specific band present in most samples. B. Immunoblot analysis of levels of Skp2 and p27 in extracts from asynchronous (lane 1) or G1-arrested (lane 2) HeLa cells.

FIG. 40A-C. Skp2 is required for p27-ubiquitin ligation activity. A. Immunodepletion. Extracts from asynchronous HeLa cells were untreated (lane 2) or immunodepleted with pre-immune serum (lane 3), anti-Skp2 antibody pre-incubated with 2 μg of purified GST (lane 4), or anti-Skp2 antibody pre-incubated with 2 μg of purified GST-Skp2 (lane 5). Lane 1 contains no extract. Samples (30 μg of protein) were assayed for p27 ubiquitination in the presence of cyclin E/Cdk2. The bracket on the left side of the panels marks a ladder of bands >27,000 corresponding to polyubiquitinated p27. The asterisk indicates a non-specific band present in all samples. B. Reconstitution. The restoration of p27 ubiquitination activity in Skp2-immunodepleted extracts was tested by the addition of the indicated purified proteins. All samples contained 30 μg of Skp2-depleted extract (Skp2-dep1. ext.) and cyclin E/Cdk2. C. Immunopurification. Extracts from asynchronous HeLa cells were immunoprecipitated with a rabbit anti-Skp2 antibody (lanes 3 and 5) or pre-immune serum (PI, lanes 2 and 4). Total extract (lane 1) and immuno-beads (lanes 2-5) were added with p27, recombinant purified cyclin E/Cdk2 and ubiquitination reaction mix. Samples in lanes 4 and 5 were supplemented with recombinant purified E1 and Ubc3. All samples were then assayed for p27 ubiquitination.

FIG. 41A-B. In vivo role of Skp2 in p27 degradation. A. Stabilization of p27 by a dominant negative (AF)Skp2 mutant in vivo. NIH-3T3 cells were transfected with mammalian expression vectors encoding human p27 alone (lane 2), p27 in combination with either (ΔF)Skp2 (lane 3), or (ΔF)FBP1 (lane 4). Lane 1: untransfected cells. Cells were lysed and extracts were subjected to immunoblotting with antibodies to p27, Skp2 or Flag [to detect Flag-tagged (ΔF)FBP1]. Exogenous human p27 protein migrates more slowly than the endogenous murine p27. B. Pulse chase analysis of p27 turnover rate. Human p27 in combination with either an empty vector, or (ΔF)Skp2 was transfected in NIH-3T3 cells. Twenty-four hours later, cells were labeled with [35S]-methionine for 20 minutes and chased with medium for the indicated times. Extracts were then subjected to immunoprecipitation with a mouse anti-p27 antibody.

FIG. 42. Stabilization of cellular p27 by antisense oligonucleotides targeting SKP2 mRNA. HeLa cells were treated for 16-18 hours with two different anti-sense oligodeoxynucleotides (AS) targeting two different regions of SKP2 mRNA. Lanes 2, 6, 12 and 16: AS targeting the N-terminal SKP2 region (NT); Lanes 4 and 8: AS targeting the C-terminal SKP2 region (CT); Lanes 1, 3, 5, 7 11 and 15: control oligodeoxynucleotides pairs (Ctrl). Lanes 1-4, and 5-8 are from two separate experiments. Lanes 11-12 and 15-16: HeLa cells were blocked in G1/S with either Hydroxyurea or Aphidicolin treatment respectively, for 24 hours. Cells were then transfected with oligodeoxynucleotides, lysed after 12 hours (before cells had re-entered G1) and immunoblotted with antibodies to Skp2 (top panels) and p27 (bottom panels). Lanes 9 and 13: Untransfected HeLa cells; Lanes 10 and 14: Untransfected HeLa cells treated with drugs as transfected cells.

FIG. 43A-C. Timing of Skp2 action in the process of p27 degradation. A. IMR90 fibroblasts were synchronized in G0/G1 by serum deprivation, reactivated with serum, and sampled at the indicated intervals. Protein extracts were analyzed by immunoblot with the antibodies to the indicated proteins. The Skp2 doublet was likely generated by phosphorylation since was consistently observed using a 12.5% gel only when cell lysis was performed in the presence of okadaic acid. B. HeLa cells blocked in mitosis with nocodazole were shaken off, released in fresh medium and sampled at the indicated intervals. Protein extracts were analyzed by immunoblotting with the antibodies to the indicated proteins. C. Extracts from G1 (3 hours after release from nocodazole block) (lane 1) and S-phase (12 hours after release from the nocodazole block) (lane 2) HeLa cells were either immunoprecipitated with an anti-p27 antibody (top two panels) or with an anti-Skp2 antibody (bottom three panels) and then immunoblotted with the antibodies to the indicated proteins.

FIG. 44. The heat-stable factor is sensitive to trypsin action. Heat-treated Fraction 1 (˜0.1 mg/ml) was incubated at 37° C. for 60 min with 50 mM Tris-HCl (pH 8.0) either in the absence (lane 1) or in the presence of 0.6 mg/ml of TPCK-treated trypsin (Sigma T8642) (lane 2). Trypsin action was terminated by the addition of 2 mg/ml of soybean trypsin inhibitor (STI). In lane 3, STI was added 5 min prior to a similar incubation with trypsin. Subsequently, samples corresponding to ˜50 ng of heat-treated Fraction 1 were assayed for the stimulation of p27-ubiquitin ligation.

FIG. 45A-C. The heat-stable factor is not Nedd8 and is required following the modification of Cul-1 by Nedd8. A. Purified Nedd8 does not replace the factor in the stimulation of p27-ubiquitin ligation. Where indicated, ˜50 ng of heat-treated Fraction 1 or 100 ng of purified recombinant human Nedd8 were added to the p27-MeUb ligation assay. B. Ligation of Nedd8 to Cul-1. Cul-1/ROC1 (3 μl) was incubated with Nedd8 (10 μg) and purified Nedd8-conjugating enzymes (20 μl) in a 100-μl reaction mixture containing Tris (pH 7.6), MgCl₂, ATP, phosphocreatine, creatine phosphokinase, DTT, glycerol and STI at concentrations similar to those described for the p27-ubiquitin ligation assay. A control preparation of Cul1/ROC1 was incubated under similar conditions, but without Nedd8 conjugating enzymes. Following incubation at 30° C. for 2 hours, samples of control (lane 1) or Nedd8-modified (lane 2) preparations were separated on an 8% polyacrylamide-SDS gel and immunoblotted with an anti-Cul-1 antibody (Zymed). C. SCF^{Skp2} complex containing Nedd8-modified Cul-1 still requires the factor from Fraction 1 for p27-ubiquitin ligation. p27-MeUb ligation was assayed, except that ³⁵S-labeled p27 was replaced by bacterially expressed purified p27 (20 ng), and Cul-1/ROC1 was replaced by 2 μl of the unmodified or Nedd8-modified Cul-1/ROC1 preparations. Following incubation (30° C., 60 min), samples were separated on a 12.5% polyacrylamide-SDS gel, transferred to nitrocellulose and blotted with an anti-p27 monoclonal antibody (Transduction Laboratories). A cross-reacting protein is labeled by an asterisk.

FIG. 46A, B. Purification of the factor required for p27-ubiquitin ligation and its identification as Cks1. A. Last step of purification by gel filtration chromatography. The peak of active material from the MonoS step was applied to a Superdex 75 HR 10/30 column (Pharmacia) equilibrated with 20 mM Tris-HCl (pH 7.2), 150 mM NaCl, 1 mM DTT and 01% Brij-35. Samples of 0.5 ml were collected at a flow rate of 0.4 ml/min. Column fractions were concentrated to a volume of 50 μl by centrifuge ultrafiltration (Centricon-10, Amicon). Samples of 0.004 μl of column fractions were assayed for activity to stimulate p27-ubiquitin ligation. Results were quantified by phosphorimager analysis and were expressed as the percentage of ³⁵S-p27 converted to ubiquitin conjugates. Arrows at top indicate the elution position of molecular mass marker proteins (kDa). B. Silver staining of samples of 2.5 μl from the indicated fractions of the Superdex 75 column, resolved on a 16% polyacrylamide-SDS gel. Numbers on the right indicate the migration position of molecular mass marker proteins (kDa).

FIG. 47. All bacterially expressed Cks/Suc1 proteins stimulate the multi-phosphorylation of the Cdc27 subunit of the cyclosome/APC. Cyclosomes from S-phase HeLa cells were partially purified and incubated with 500 units of Suc1-free Cdk1/cyclin B (Shteinberg, M. & Hershko, A., 1999, Biochem. Biophys. Res. Commun. 257:12; Yudkovsky, et al., 2000, Biochem. Biophys. Res. Commun. 271:299). Where indicated, 10 ng/μl of the corresponding Cks/Suc1 protein was supplemented. The samples were subjected to immunoblotting with a monoclonal antibody directed against human Cdc27 (Transduction Laboratories).

FIG. 48A, B. Identification of the factor required for p27-ubiquitin ligation as Cks1. A. The ligation of ³⁵S-p27 to MeUb was assayed. Where indicated, Fraction 1 (5 μg protein) or heat-treated Fraction 1 (˜50 ng) were added. The bracket on the left side of the panels marks a ladder of bands>27,000 Da corresponding to polyubiquitinated p27. B. Cks1, but not other Cks proteins, is required for p27-ubiquitin ligation. Where indicated, the following proteins were added: “Factor”, 0.02 μl of pooled fractions # 28-29 from the peak of the Superdex column, which is the last step of purification of the factor required for p27 ubiquitinylation; “Cks1 IVT”, 0.3 μl of in-vitro translated Cks1; “Cks2 IVT”, 0.3 μl of in vitro-translated Cks2; “Retic. lys.”, 0.3 μl of reticulocyte lysate translation mix; Cks1, Cks2 and Suc1, 2 ng of the corresponding bacterially expressed, purified proteins. In vitro-translated ³⁵S-labeled Cks1 and Cks2 in lanes 3 and 4 are not visible since they migrated off the gel.

FIG. 49A-D. Cks1 increases the binding of phosphorylated p27 to Skp2. A. Cks1 does not affect the phosphorylation of p27 by Cdk2/cyclin E. Purified p27 was phosphorylated with the only difference that the mixtures were incubated at 20° C. for the time periods indicated. Where indicated, 2 ng of purified Cks1 was added. Samples of 1 μl were taken for SDS-polyacrylamide gel electrophoresis and autoradiography. B. Cks1 acts at a stage subsequent to the phosphorylation of p27. ³²P purified p27 was prepared Where indicated, 0.02 μl of “Factor” (purified as in FIG. 1b, lane 2) or 1 ng of purified recombinant human Cks1 were added. Using this purified system, we have not observed conjugates with MeUb larger than the di-ubiquitinylated form, as opposed to the 4-5 conjugates observed using in vitro-translated ³⁵S-p27 (compare with FIG. 1). Possibly, ubiquitin is ligated to only two Lys residues in p27, and the larger conjugates may contain short polyubiquitin chains (derived from ubiquitin present in reticulocyte lysates) terminated by MeUb. C. Cks1 increases the binding of p27 to Skp2/Skp1, dependent upon phosphorylation of Thr-187. The binding of ³⁵S-labeled wild-type (WT) or Thr-187-Ala mutant p27 (T187A) to Skp2/Skp1 was determined. Where indicated, 1 ng of purified Cks1 was added to the incubation. Inputs show 5% of the starting material. D. Cks1 increases the binding of ³²P-p27 to Skp2/Skp1. The experiment was similar to that described in 2c, except that ³⁵S-p27 was replaced by ³²P-labeled purified p27.

FIG. 50A-D. Binding of Cks1 to Skp2 and phosphorylated p27. A. Cks1 but not Cks2 binds to Skp2/Skp1. The binding of ³⁵S-labeled Cks1 or Cks2 to Skp2/Skp1 was assayed by a procedure similar to that described for the binding of p27 to Skp2/Skp1, except that Cdk2/cyclin E, ATP and the ATP-regenerating system were omitted. Where indicated, 1 μl of Skp2/Skp1 was added. B. Cks1 does not bind to Skp1. The binding of ³⁵S-Cks1 to His₆-Skp1 or to the Skp2/His₆-Skp1 complex (1 μl each) was determined as described in 3a, except that Ni-NTA-agarose beads (Quiagen, 10 μl) were used for precipitation. In both 3a and 3b, inputs show 5% of the starting material. C. Cks1 stimulates the binding of Skp2 to p27 phosphopeptide. Sepharose beads to which a peptide corresponding to 19 C-terminal amino acid residues of p27 (“p27 beads”), or to a similar peptide containing phosphorylated Thr187 (“P-p27 beads”) were prepared as described in Carrano, et al., 1999, Nat. Cell Biol 1:193. In vitro-translated ³⁵S-Skp2 (3 μl) was mixed with 15 μl of the corresponding beads in the absence (lanes 1 and 3) or in the presence of 10 ng (lane 4) or 100 ng (lanes 2 and 5) of Cks1. Following rotation at 4° C. for 2 hours, beads were washed 4 times with RIPA buffer. D. Cks1 binds to p27 phosphopeptide. ³⁵S-Cks1 (2 μl) was mixed with the indicated beads, and beads were treated as in FIG. 3c. Inputs show 10% of the starting material.

FIG. 51A-C. Western blot analysis of Skp2/E2F interaction assay. Details of the Western Blot experiments are given in the Example in Section 9.

### 5. DETAILED DESCRIPTION OF THE INVENTION

The present invention relates to novel F-box proteins and to novel substrates of F-box proteins. The present invention relates to screening assays designed to identify substrates of the novel F-box proteins and to identify small molecules and compounds which modulate the interaction and/or activity of the F-box proteins and their substrates.

The present invention relates to screening assays to identify substrates of the novel F-box proteins and to identify potential therapeutic agents. The present invention further relates to screening assays based on the identification of novel substrates of both novel and known F-box proteins. The screening assays of the present invention may be used to identify potential therapeutic agents which may be used in protocols and as pharmaceutical compositions designed to target the novel ubiquitin ligases and interactions with their substrates for the treatment of proliferative disorders. In one particular embodiment the present invention relates to screening assays and potential therapeutic agents which target the interaction of FBP with novel substrates β-catenin, p27 and E2F as identified by Applicants.

The invention further encompasses the use of nucleotides encoding the novel F-box proteins, proteins and peptides, as well as antibodies to the novel ubiquitin ligases (which can, for example, act as agonists or antagonists), antagonists that inhibit ubiquitin ligase activity or expression, or agonists that activate ubiquitin ligase activity or increase its expression. In addition, nucleotides encoding the novel ubiquitin ligases and proteins are useful for the identification of compounds which regulate or mimic their activity and therefore are potentially effective in the treatment of cancer and tumorigenesis.

In particular, the invention described in the subsections below encompasses FBP1, FBP2, FBP3a, FBP3b, FBP4, FBP5, FBP6, FBP7, FBP8, FBP9, FBP10, FBP11, FBP12, FBP13, FBP14, FBP15, FBP16, FBP17, FBP18, FBP19, FBP20, FBP21, FBP22, FBP23, FBP24, and FBP25 polypeptides or peptides corresponding to functional domains of the novel ubiquitin ligases (e.g., the F-box motif, the substrate binding domain, and leucine-rich repeats), mutated, truncated or deleted (e.g. with one or more functional domains or portions thereof deleted), ubiquitin ligase fusion proteins, nucleotide sequences encoding such products, and host cell expression systems that can produce such ubiquitin ligase products.

The present invention provides methods of screening for peptides and proteins that interact with novel components of the ubiquitin ligase complex, including FBP1, FBP2, FBP3a, FBP3b, FBP4, FBP5, FBP6, FBP7, FBP8, FBP9, FBP10, FBP11, FBP12, FBP13, FBP14, FBP15, FBP16, FBP17, FBP18, FBP19, FBP20, FBP21, FBP22, FBP23, FBP24, and FBP25 or derivatives, fragments or analogs thereof. Preferably, the method of screening is a yeast two-hybrid assay system or a variation thereof, as further described below. Derivatives (e.g., fragments) and analogs of a protein can be assayed for binding to a binding partner by any method known in the art, for example, the modified yeast two-hybrid assay system described below, immunoprecipitation with an antibody that binds to the protein in a complex followed by analysis by size fractionation of the immunoprecipitated proteins (e.g., by denaturing or nondenaturing polyacrylamide gel electrophoresis), Western analysis, non-denaturing gel electrophoresis, etc.

The present invention relates to screening assays to identify agents which modulate the activity of the novel ubiquitin ligases. The invention encompasses both in vivo and in vitro assays to screen small molecules, compounds, recombinant proteins, peptides, nucleic acids, antibodies etc. which modulate the activity of the novel ubiquitin ligases and thus, identify potential therapeutic agents for the treatment of proliferative or differentiative disorders. In one embodiment, the present invention provides methods of screening for proteins that interact with the novel ubiquitin ligases.

The invention also encompasses antibodies and anti-idiotypic antibodies, antagonists and agonists, as well as compounds or nucleotide constructs that inhibit expression of the ubiquitin ligase gene (transcription factor inhibitors, antisense and ribozyme molecules, or gene or regulatory sequence replacement constructs), or promote expression of the ubiquitin ligase (e.g., expression constructs in which ubiquitin ligase coding sequences are operatively associated with expression control elements such as promoters, promoter/enhancers, etc.). The invention also relates to host cells and animals genetically engineered to express the human (or mutants thereof) or to inhibit or “knock-out” expression of the animal's endogenous ubiquitin ligase.

Finally, the ubiquitin ligase protein products and fusion protein products, (i.e., fusions of the proteins or a domain of the protein, e.g., F-box motif), antibodies and anti-idiotypic antibodies (including Fab fragments), antagonists or agonists (including compounds that modulate the ubiquitization pathway can be used for therapy of proliferative or differentiative diseases. Thus, the invention also encompasses pharmaceutical formulations and methods for treating cancer and tumorigenesis.

Various aspects of the invention are described in greater detail in the subsections below.

5.1 FBP Genes

The invention provides nucleic acid molecules comprising seven novel nucleotide sequences, and fragments thereof, FBP1, FBP2, FBP3a, FBP4, FBP5, FBP6, and FBP7, nucleic acids which are novel genes identified by the interaction of their gene products with Skp1, a component of the ubiquitin ligase complex. The invention further provides fourteen novel nucleic acid molecules comprising the nucleotide sequences of FBP1, FBP2, FBP3a, FBP3b, FBP4, FBP5, FBP6, FBP7, FBP8, FBP11, FBP12, FBP13, FBP14, FBP15, FBP17, FBP18, FBP20, FBP21, FBP22, FBP23, FBP24, and FBP25, which Nucleic acid sequences of the identified FBP genes are described herein.

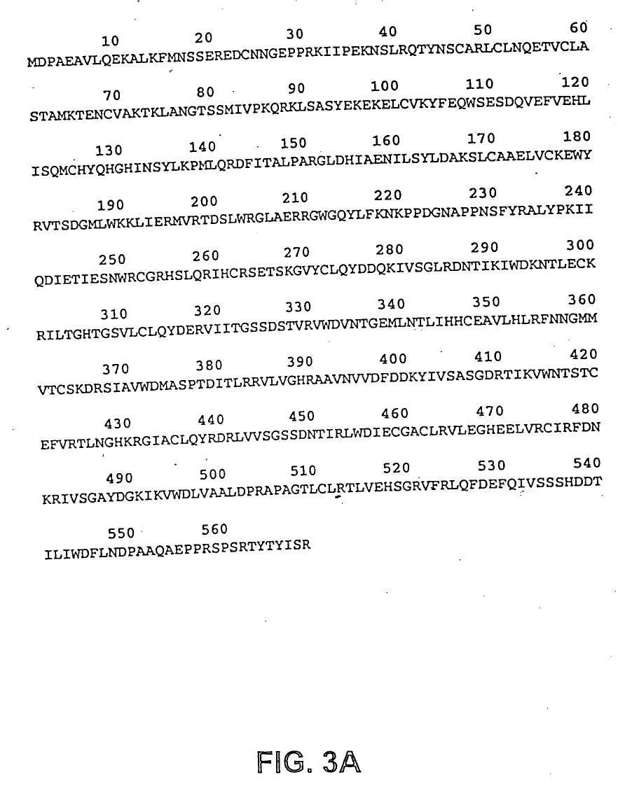

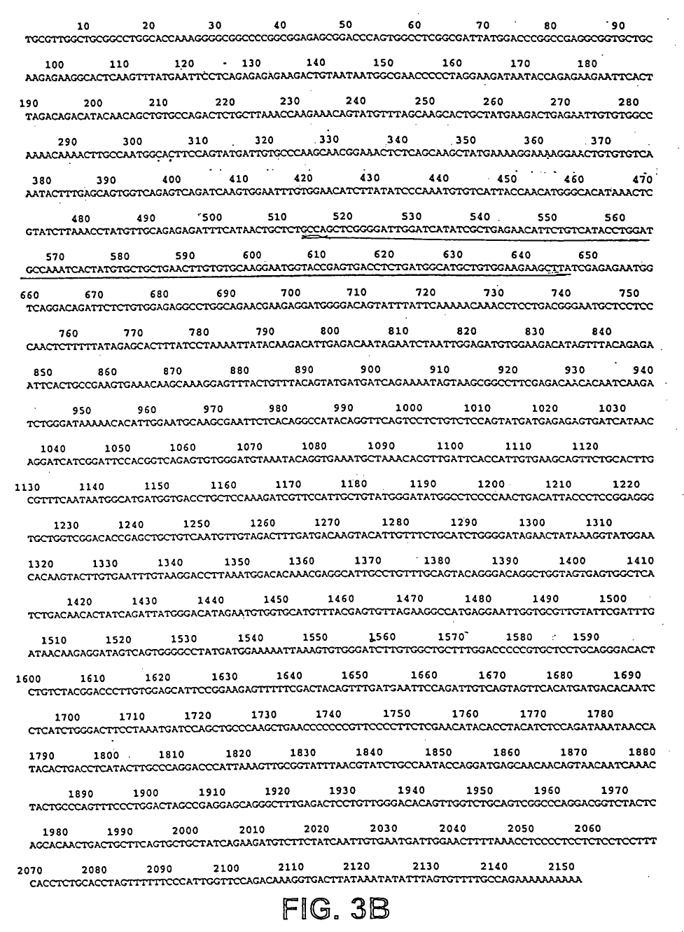

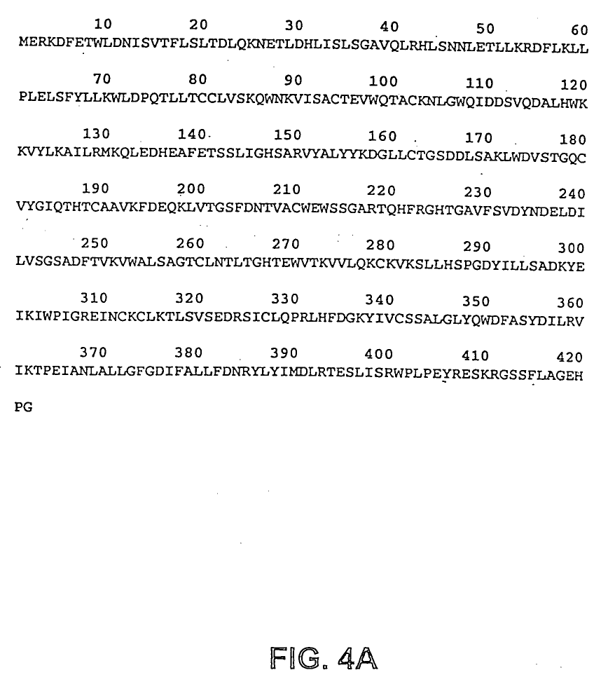

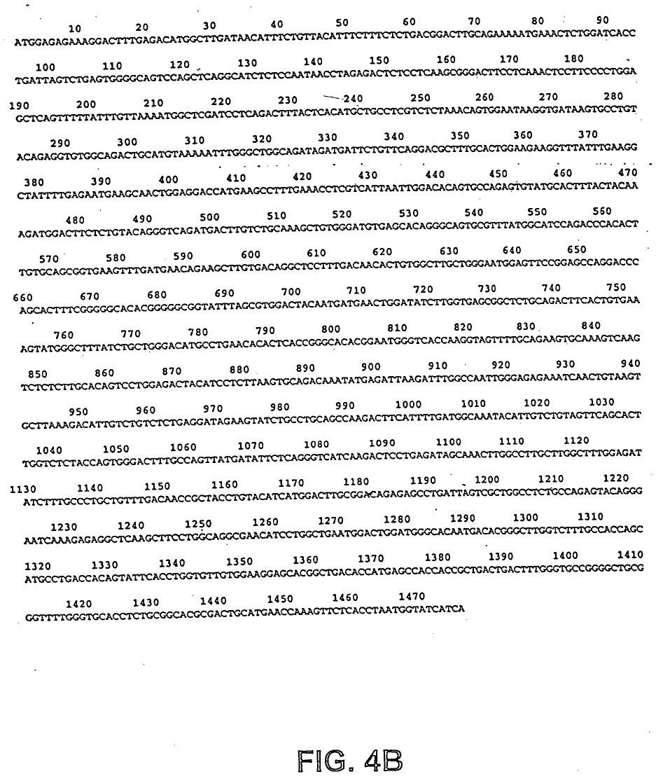

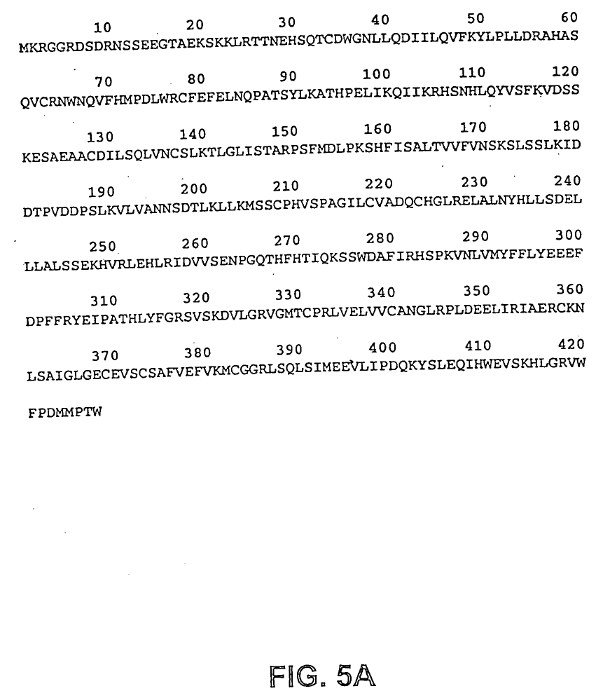

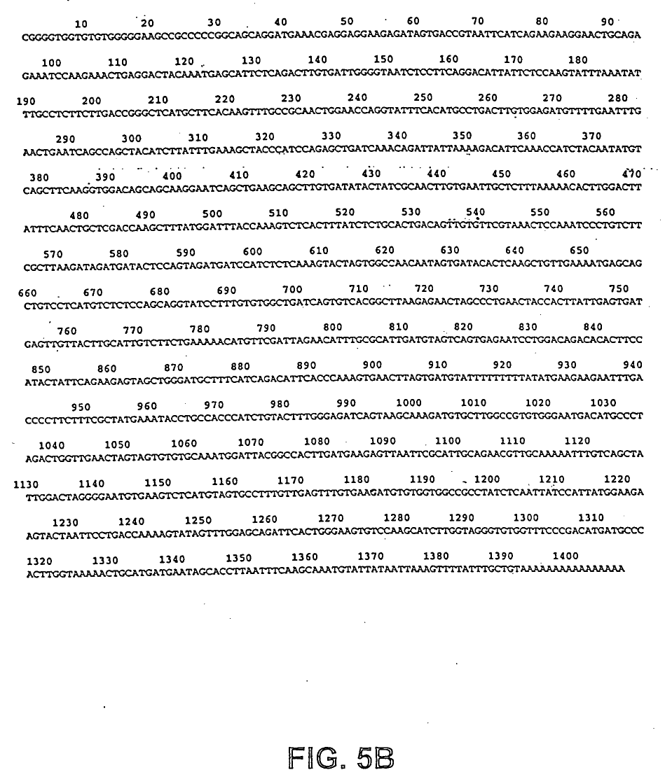

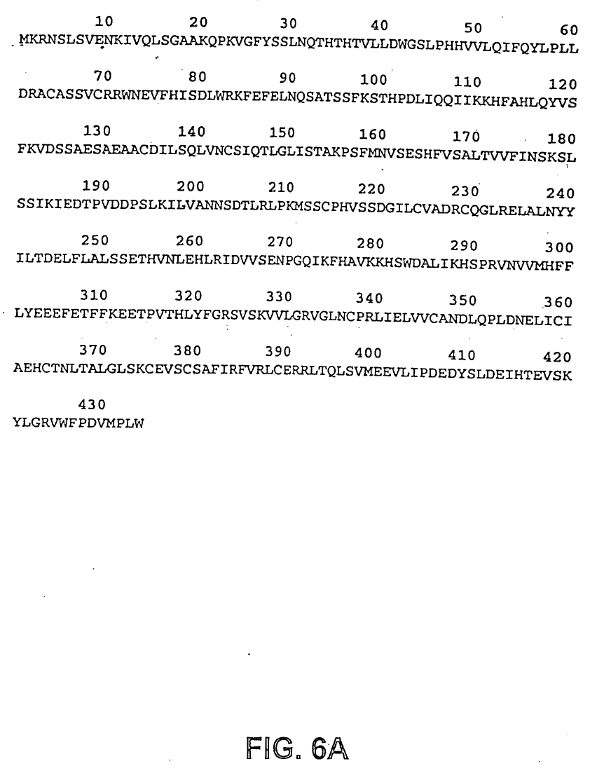

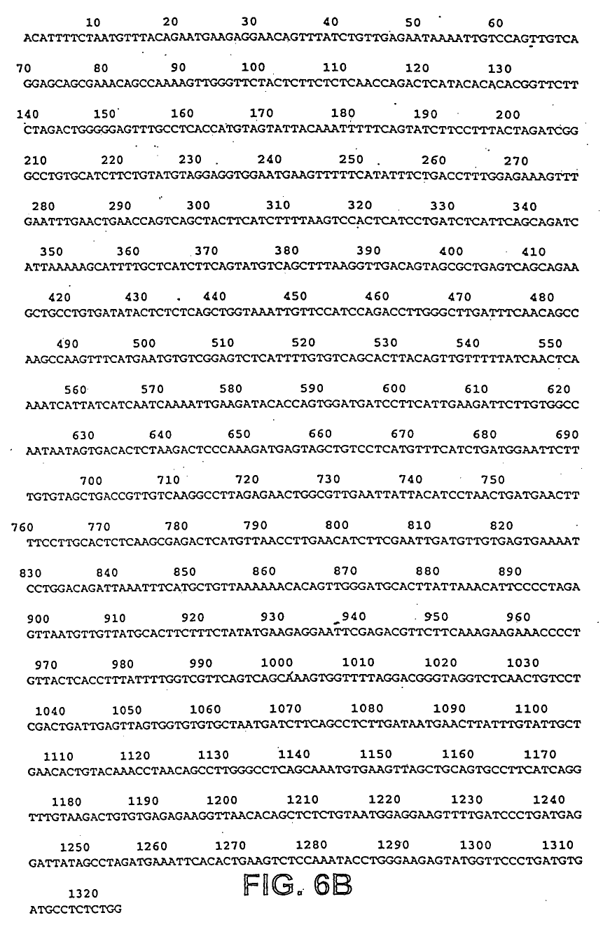

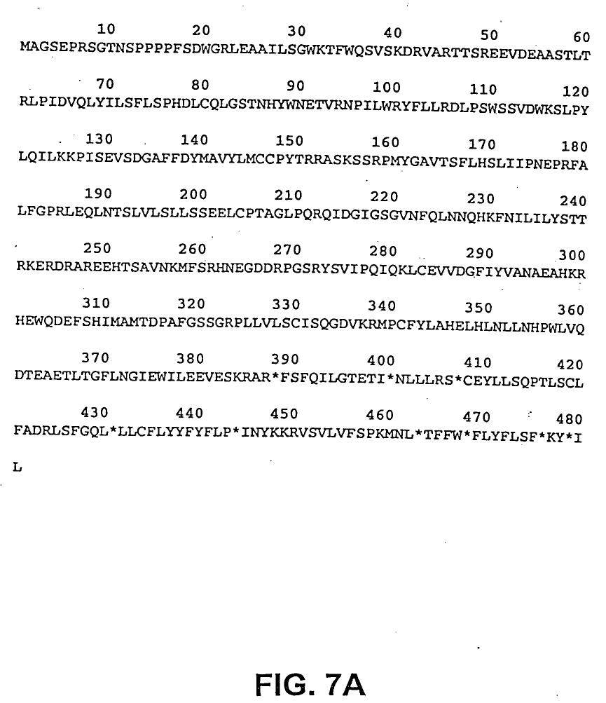

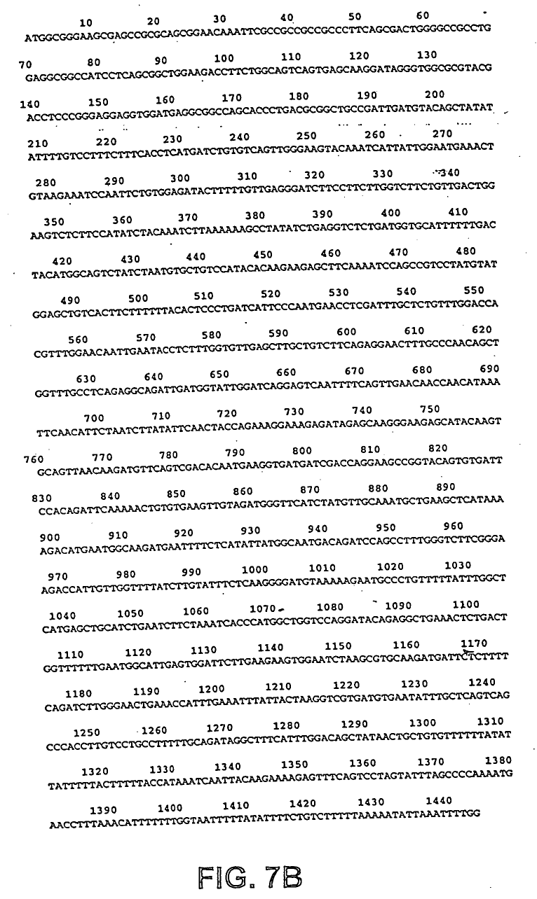

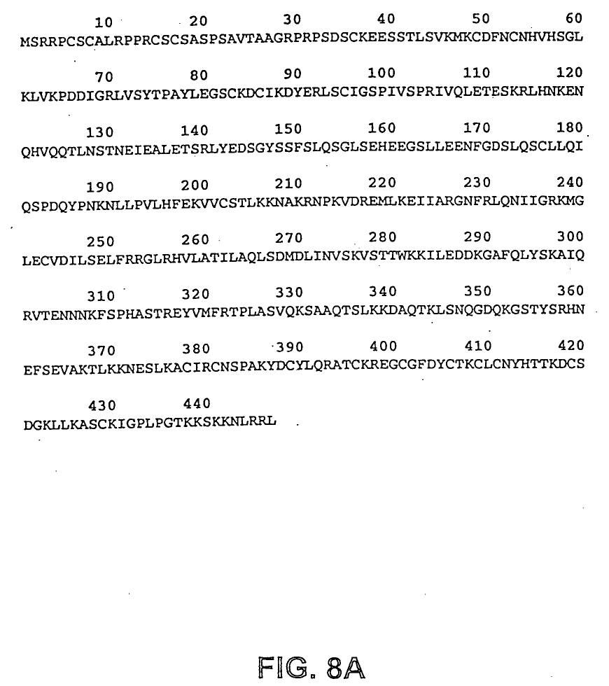

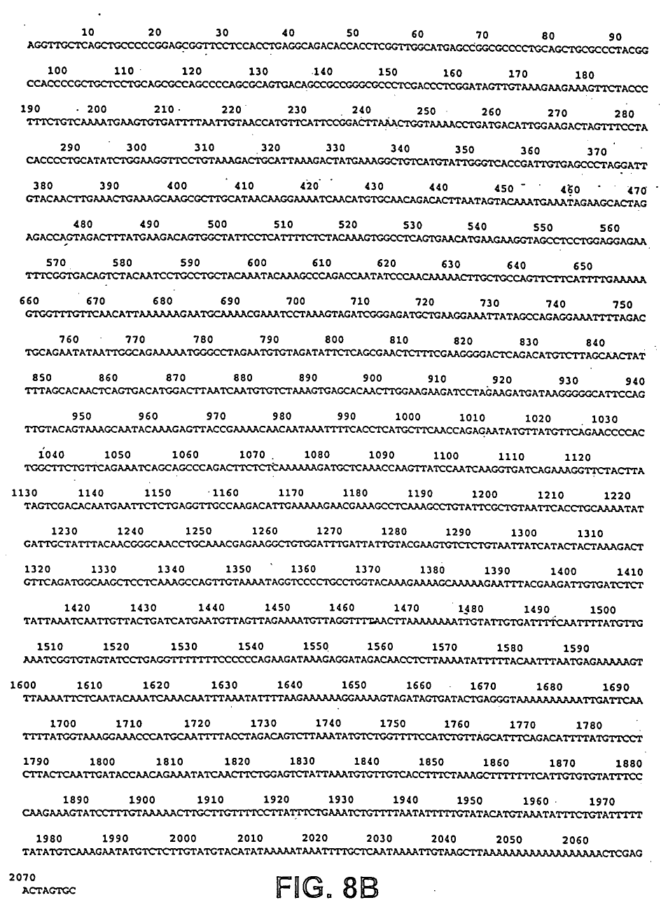

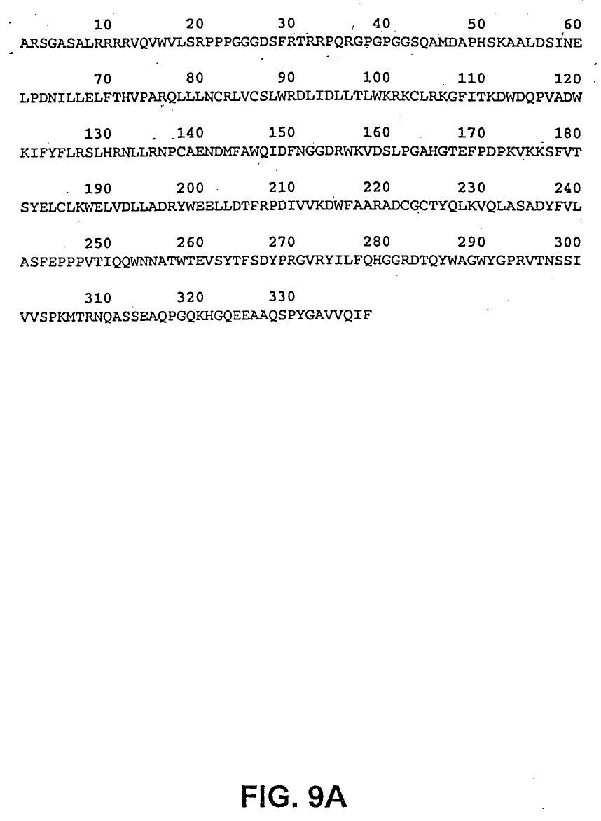

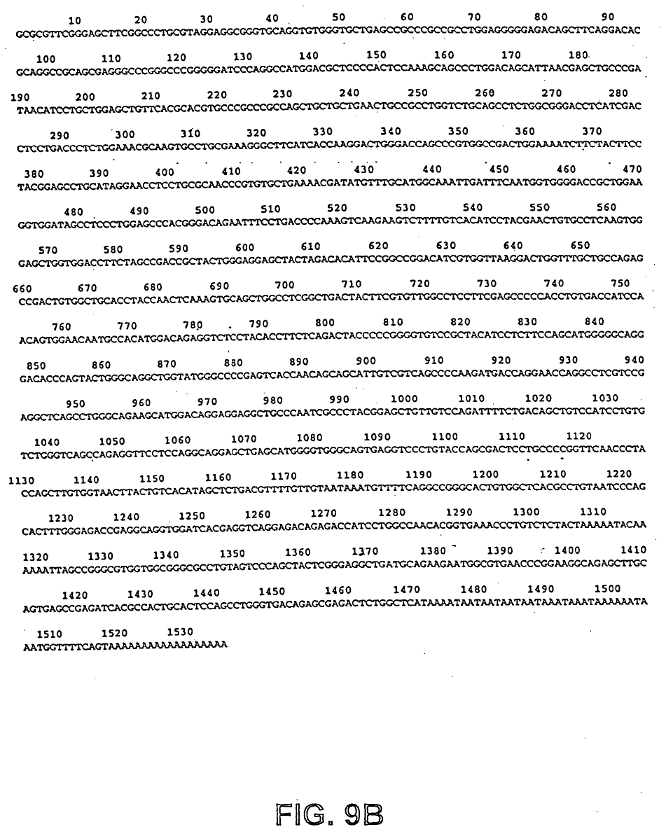

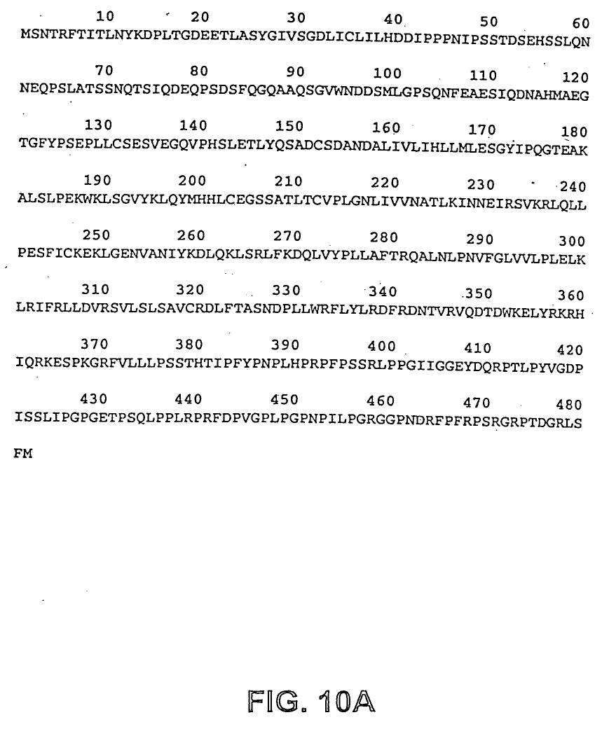

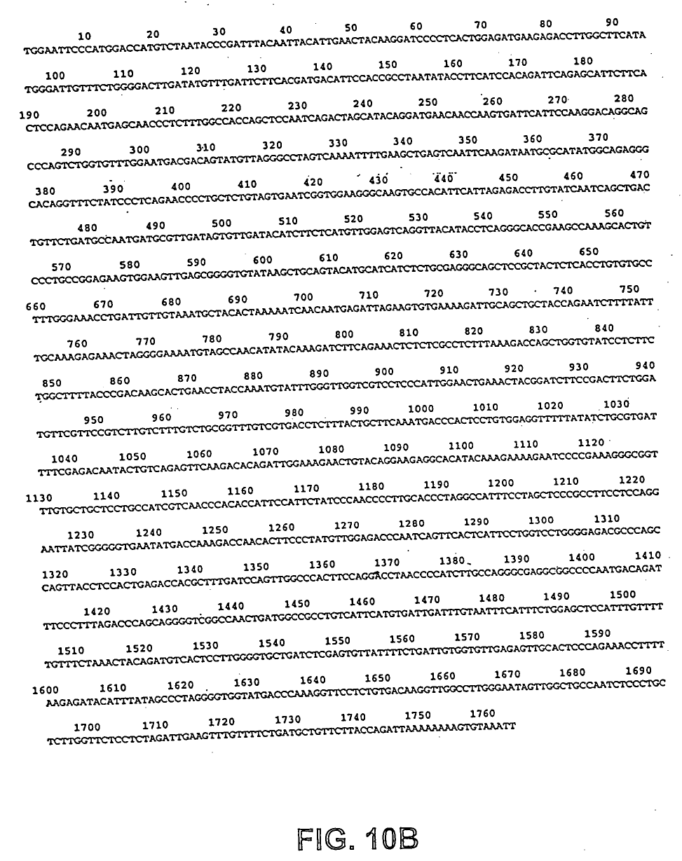

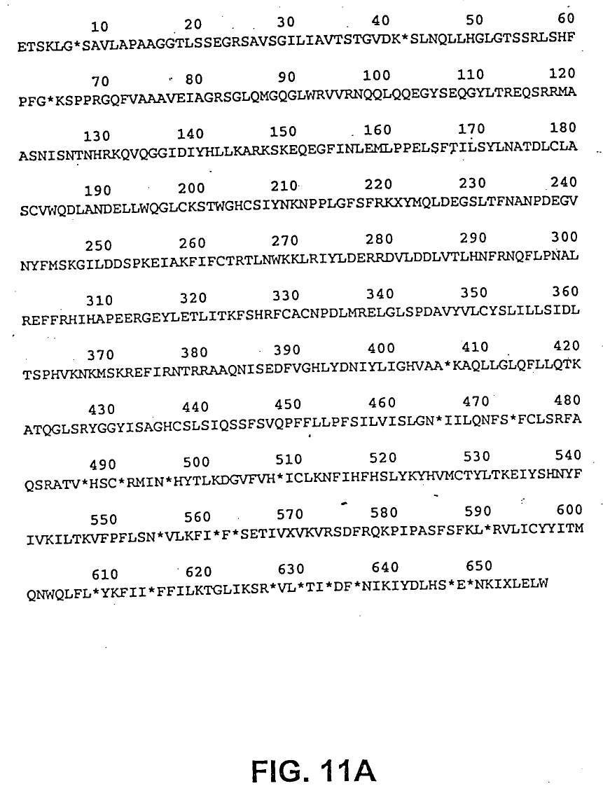

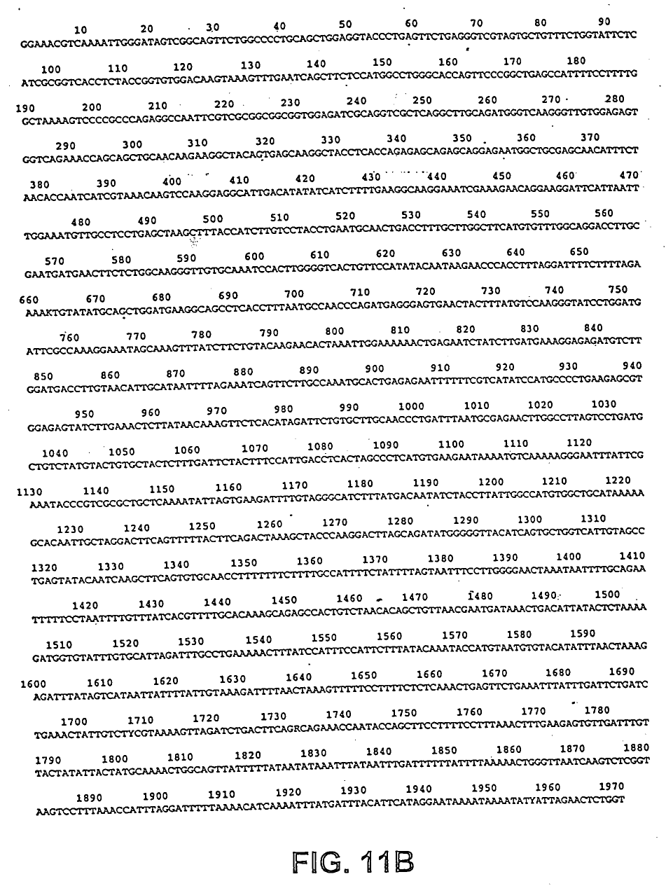

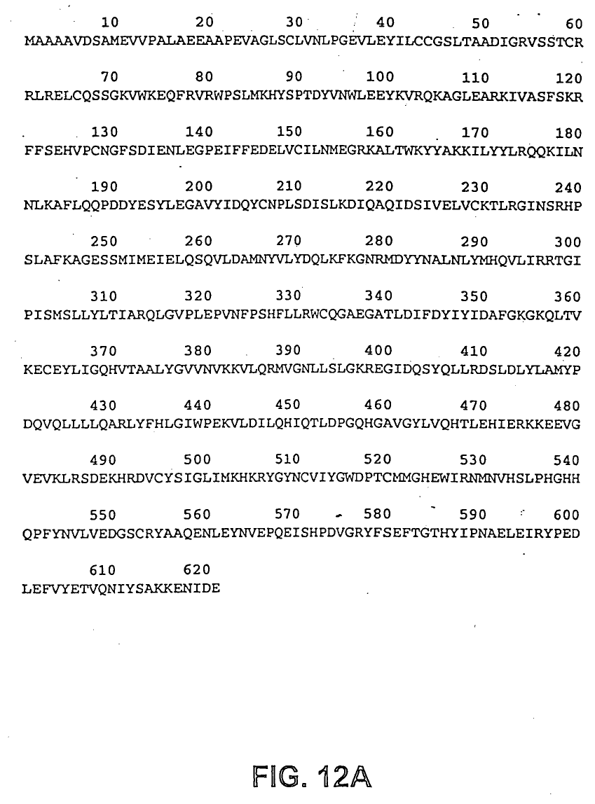

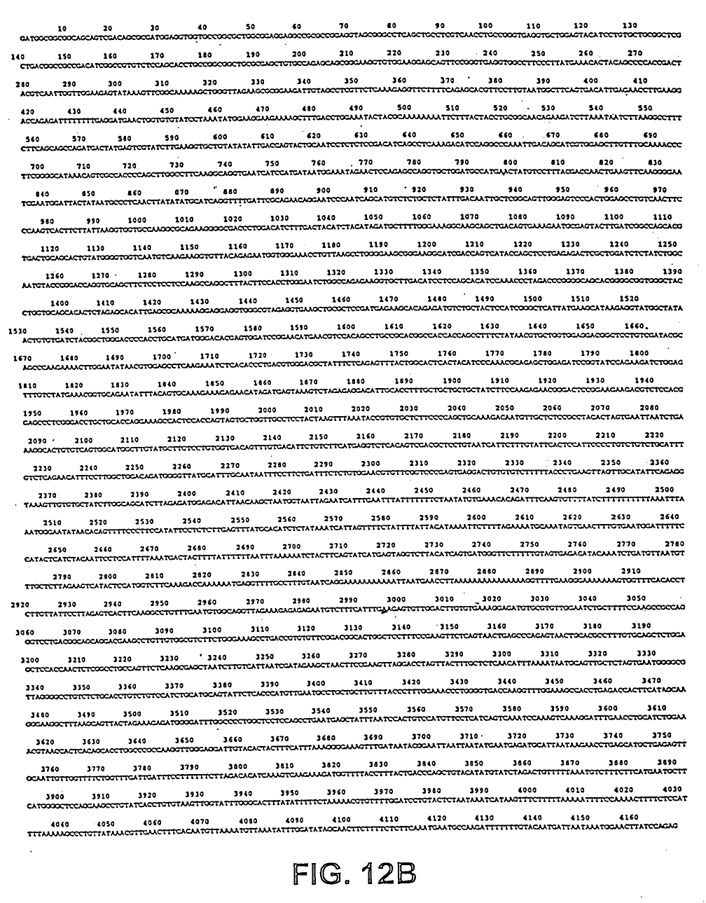

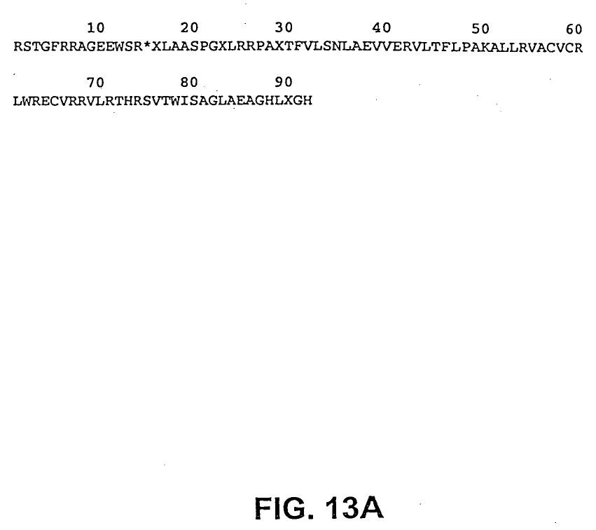

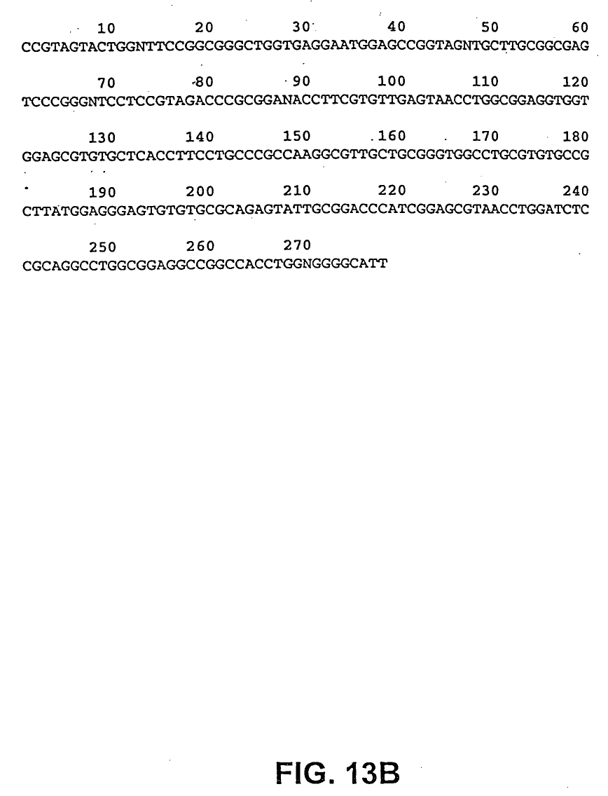

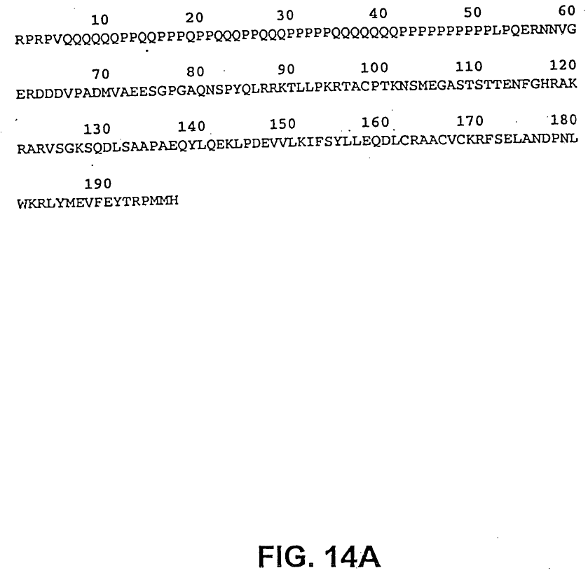

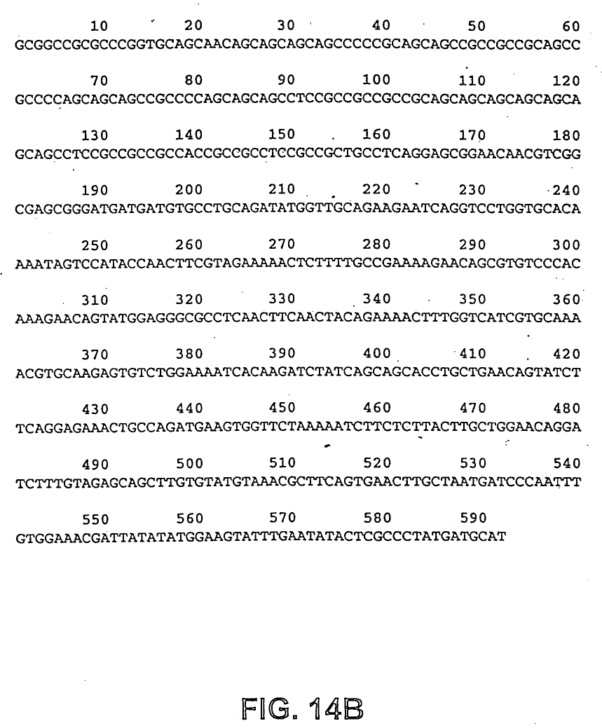

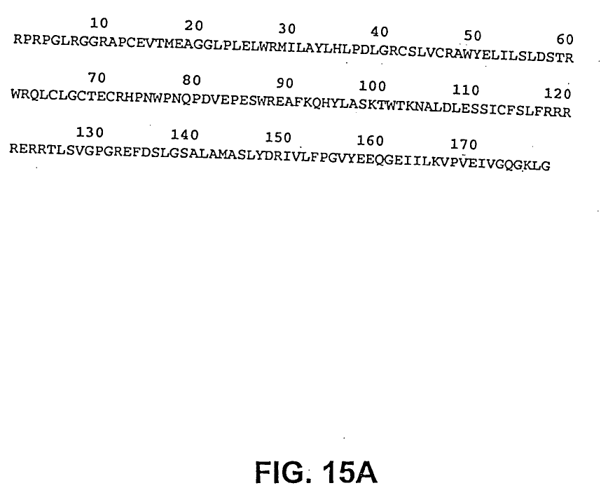

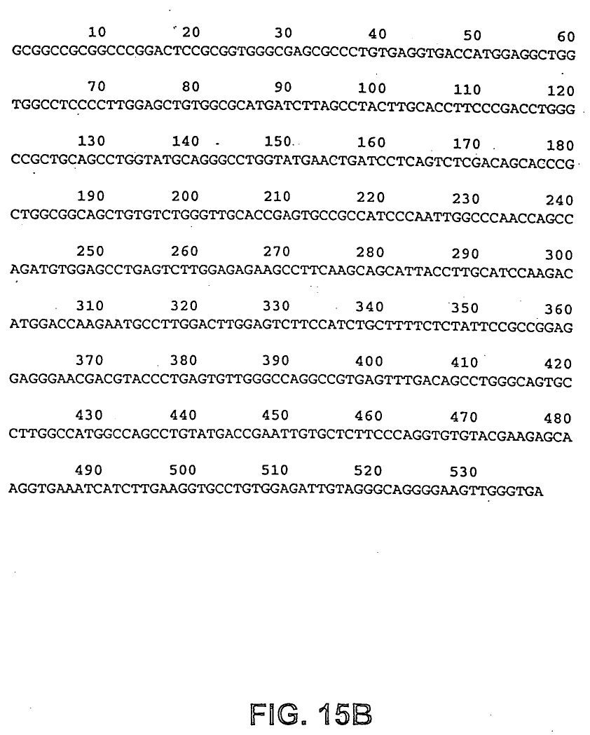

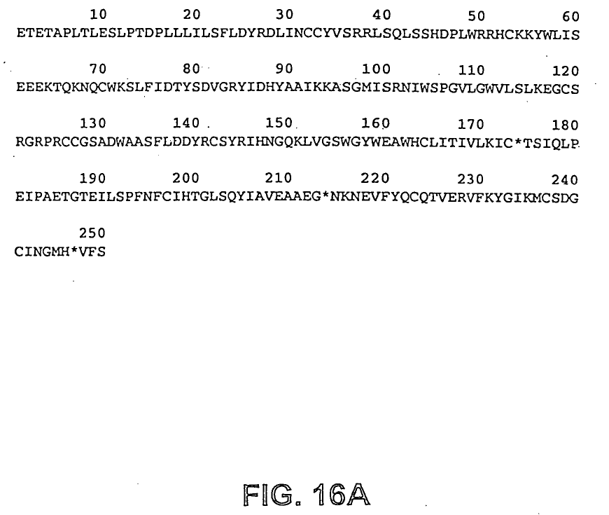

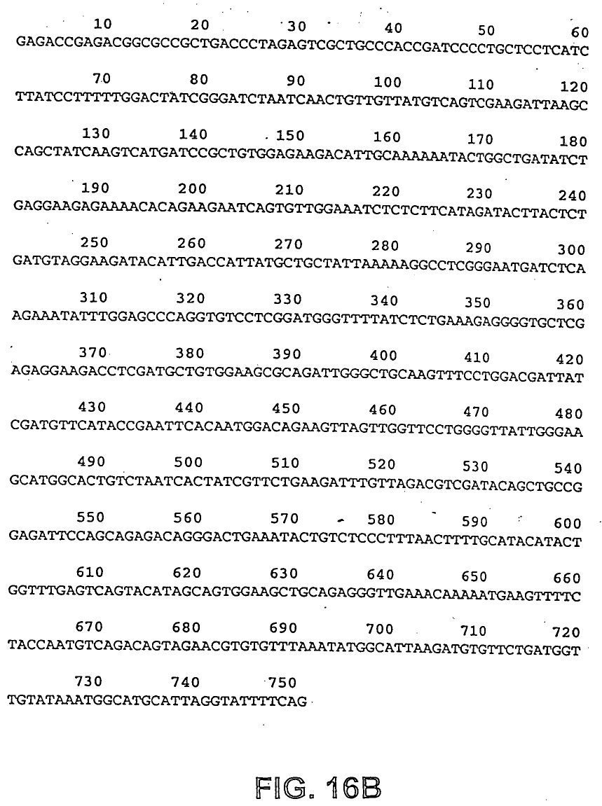

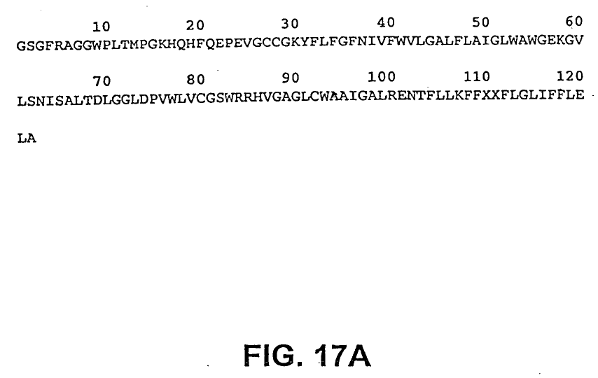

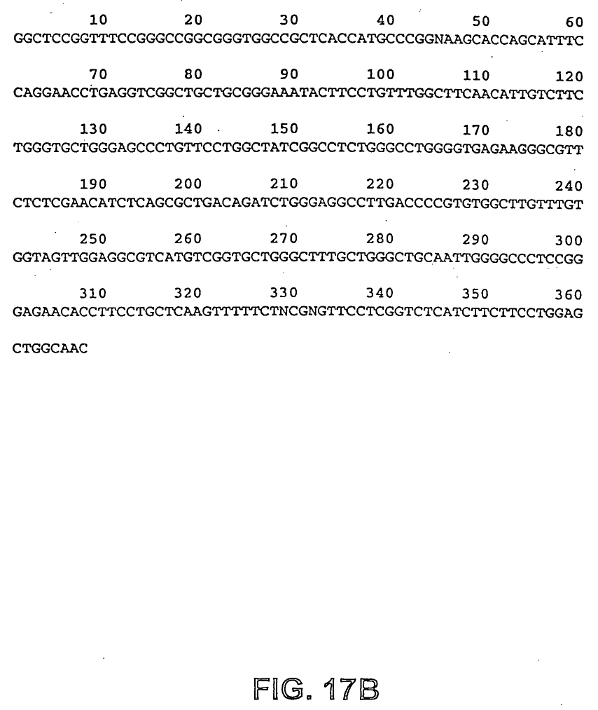

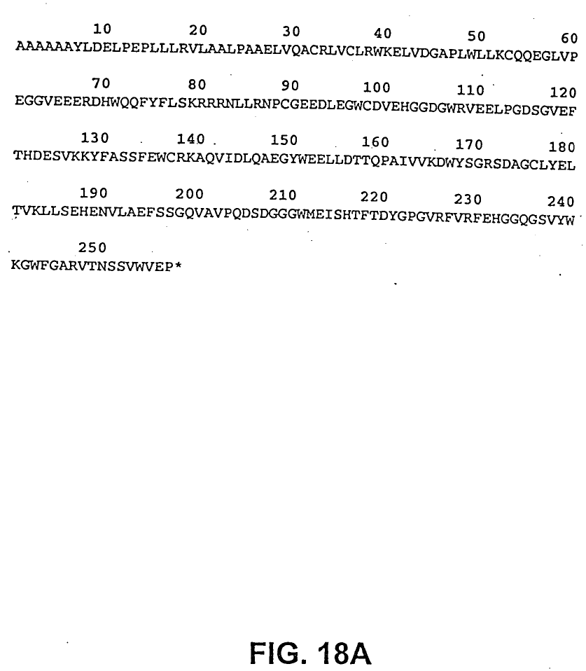

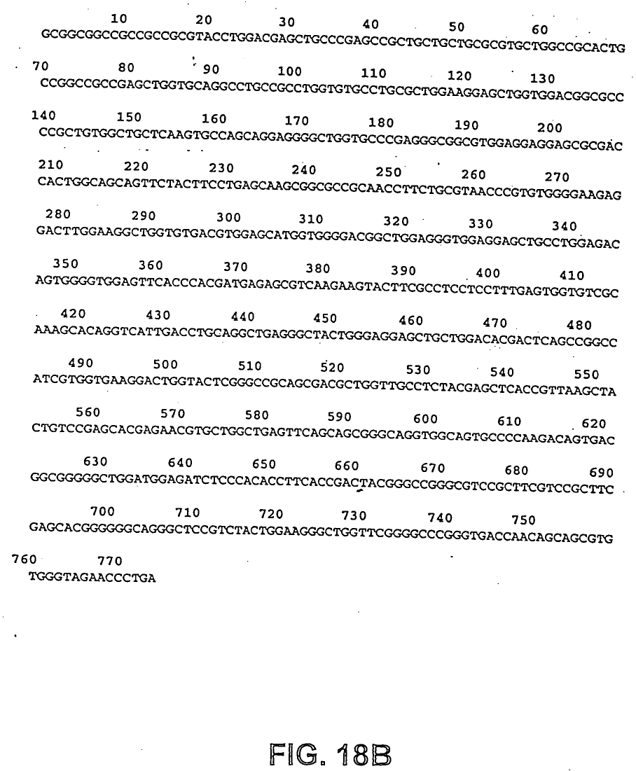

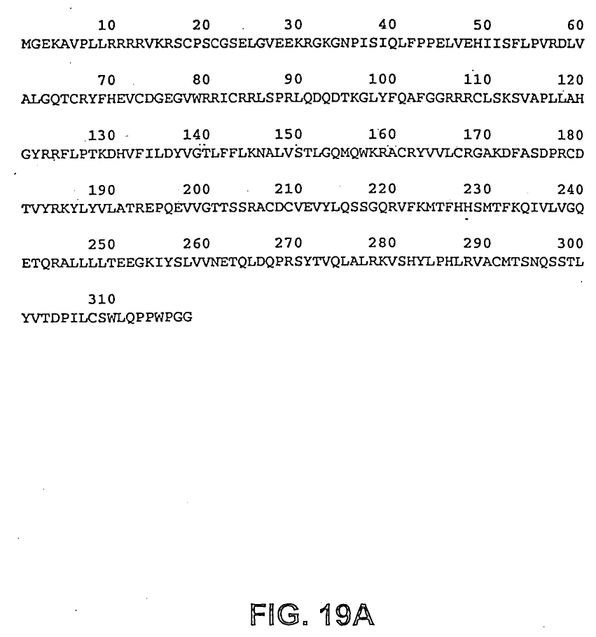

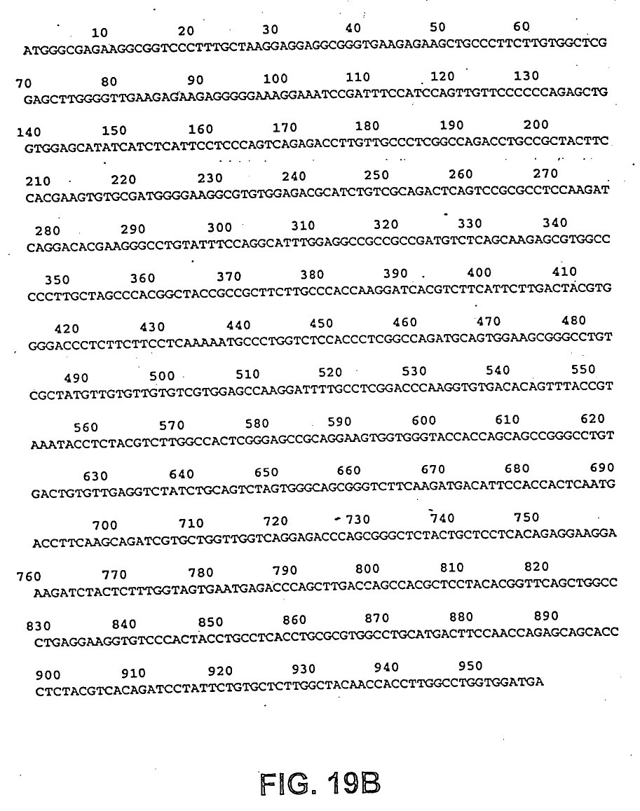

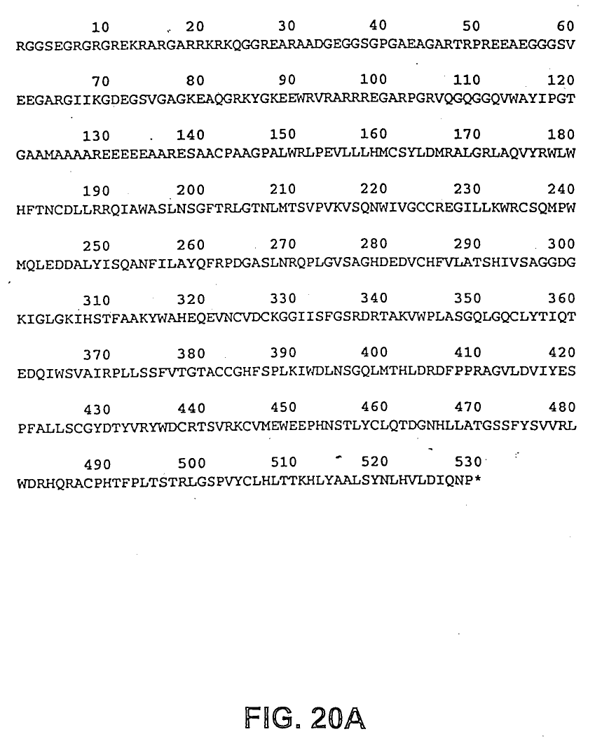

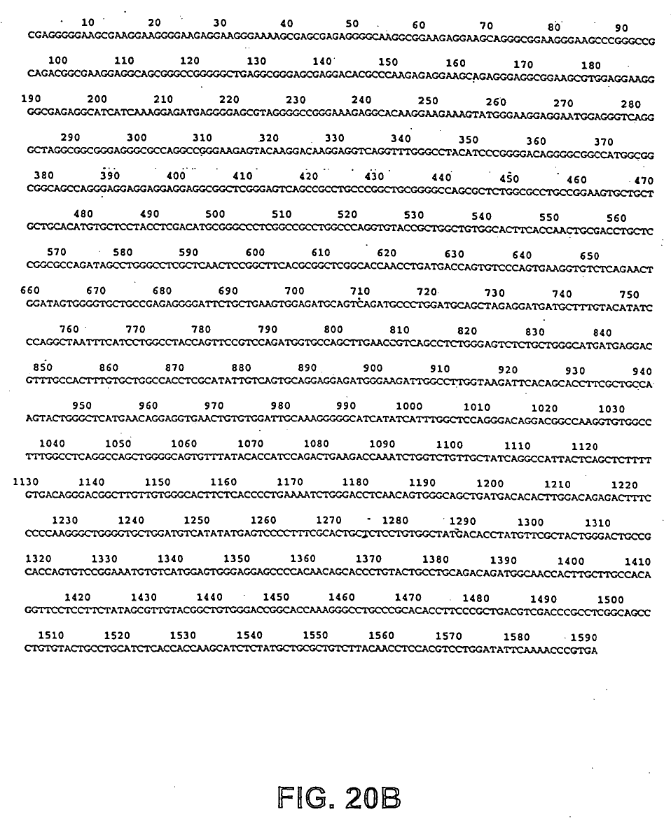

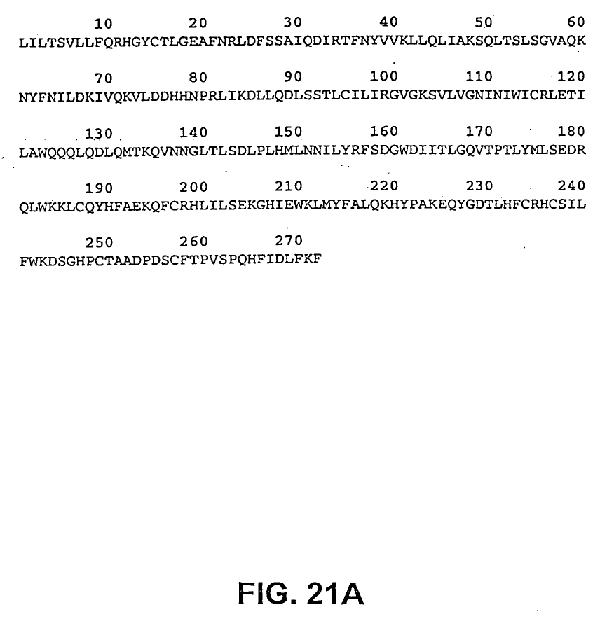

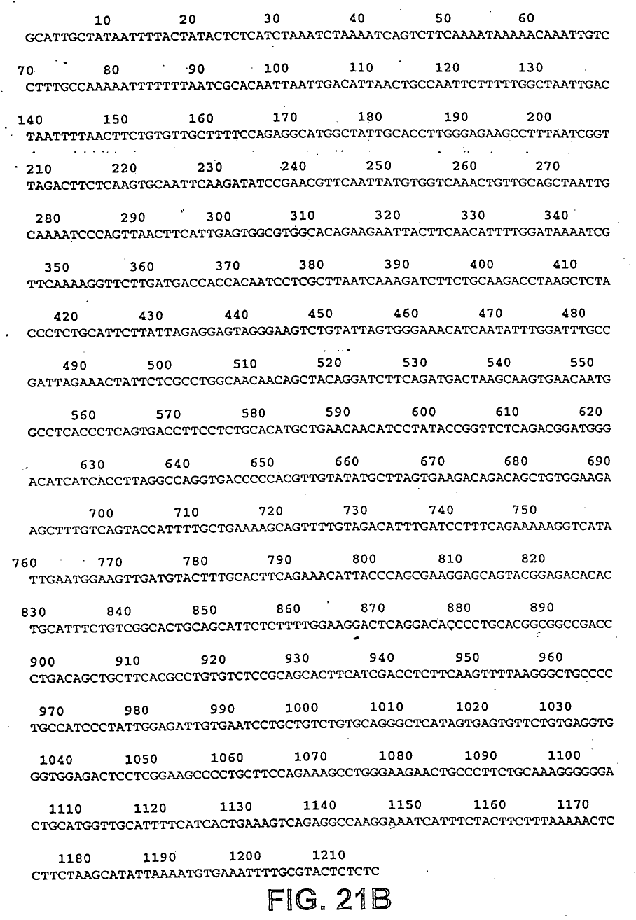

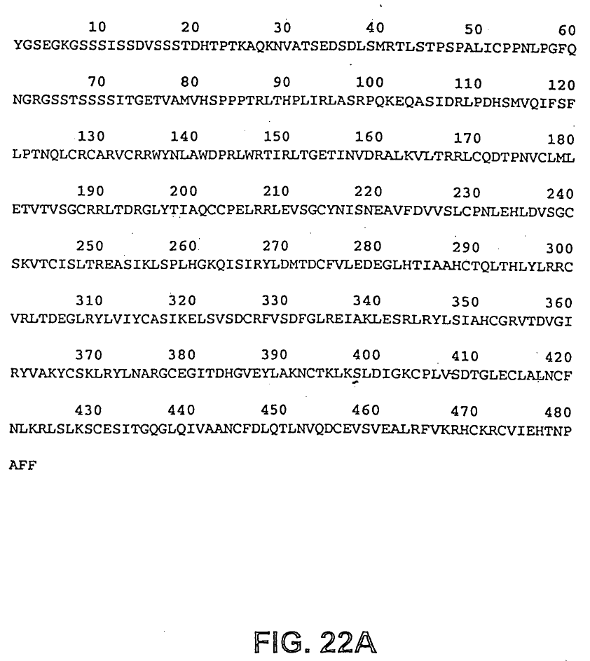

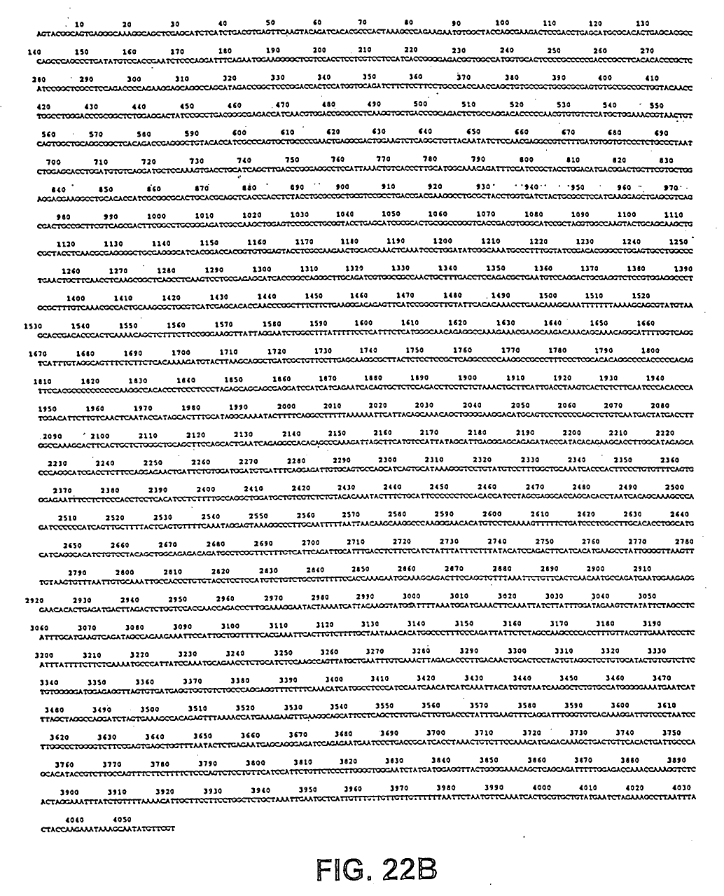

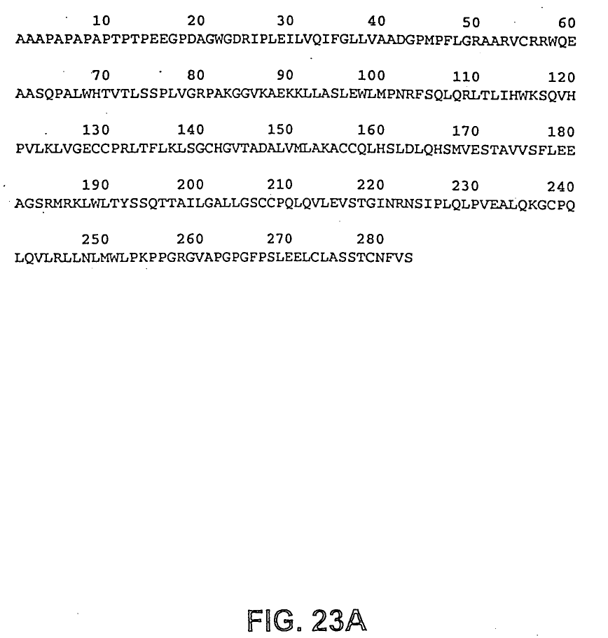

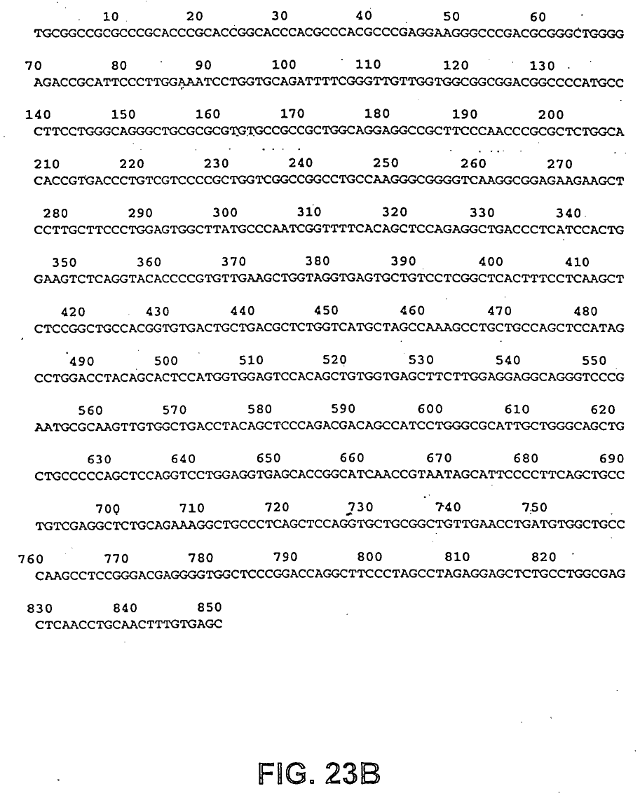

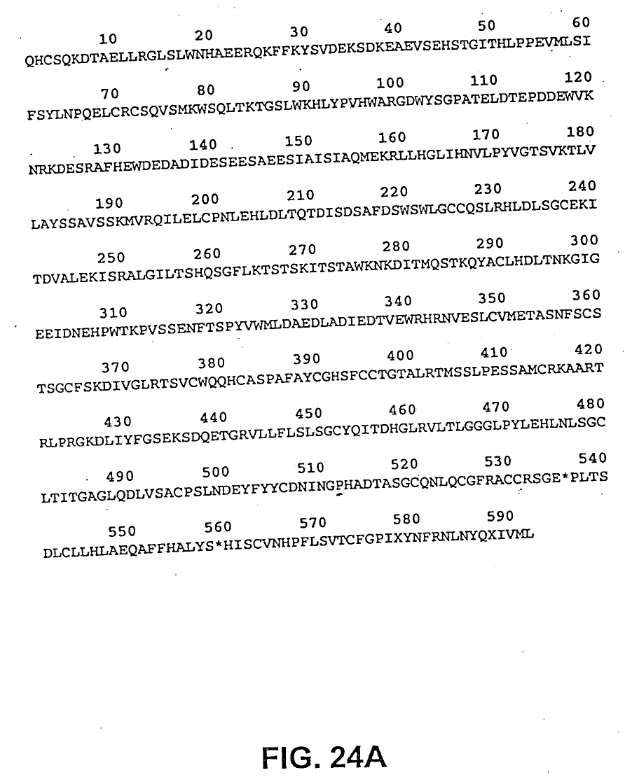

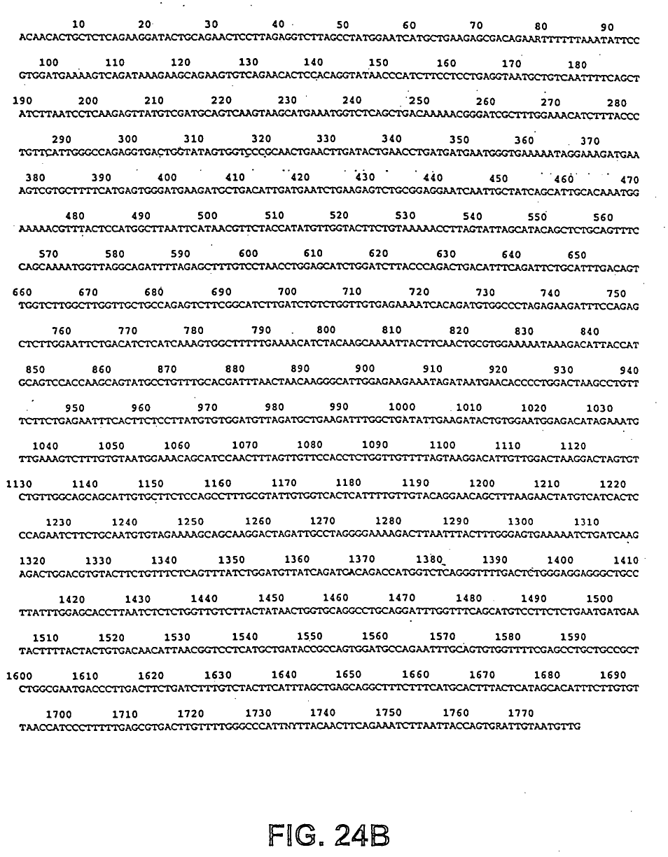

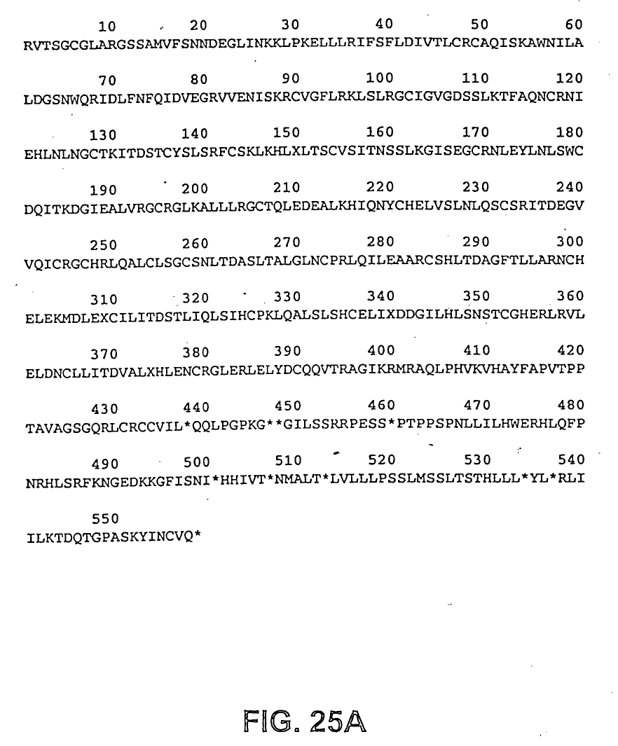

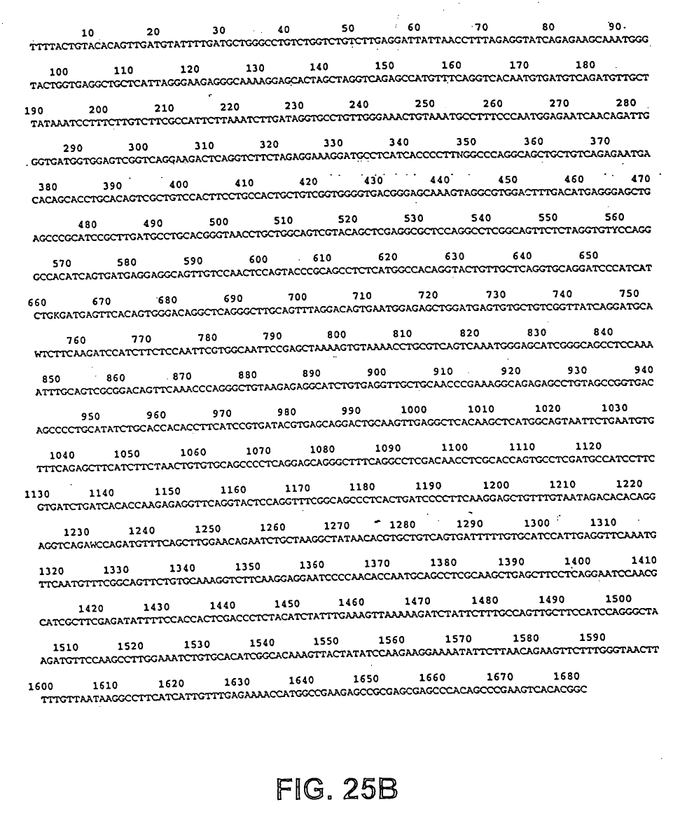

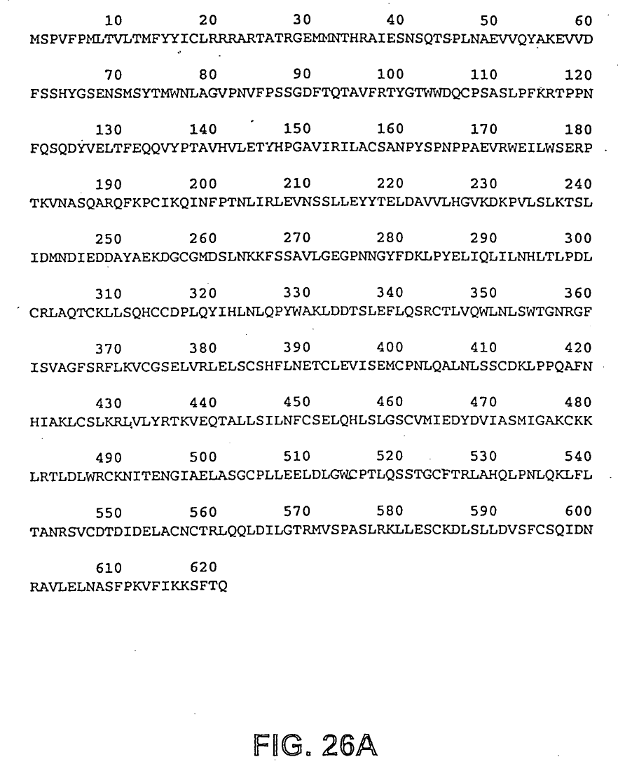

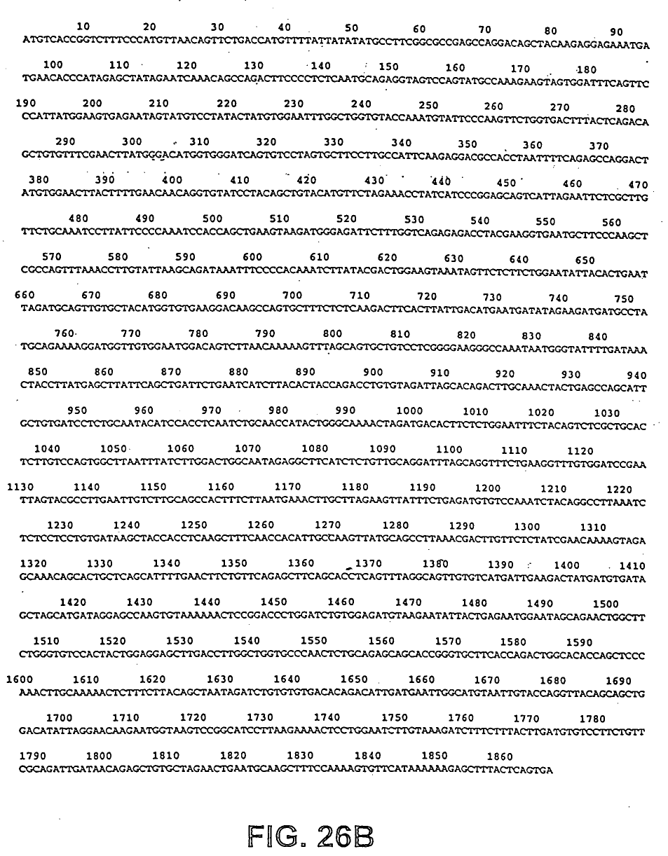

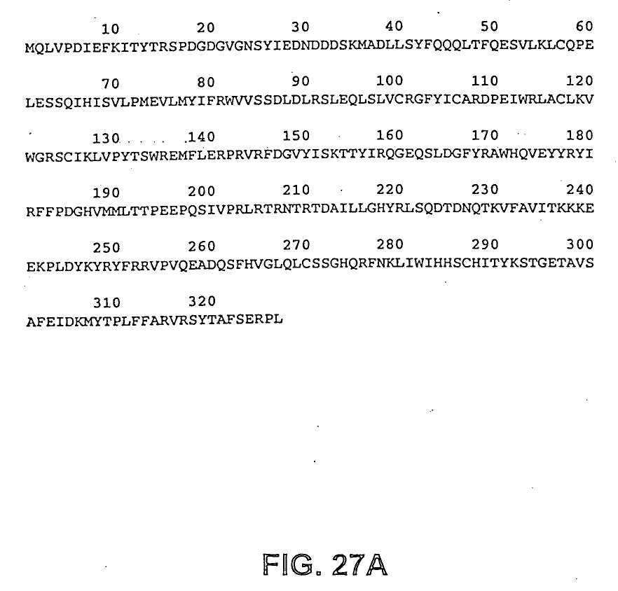

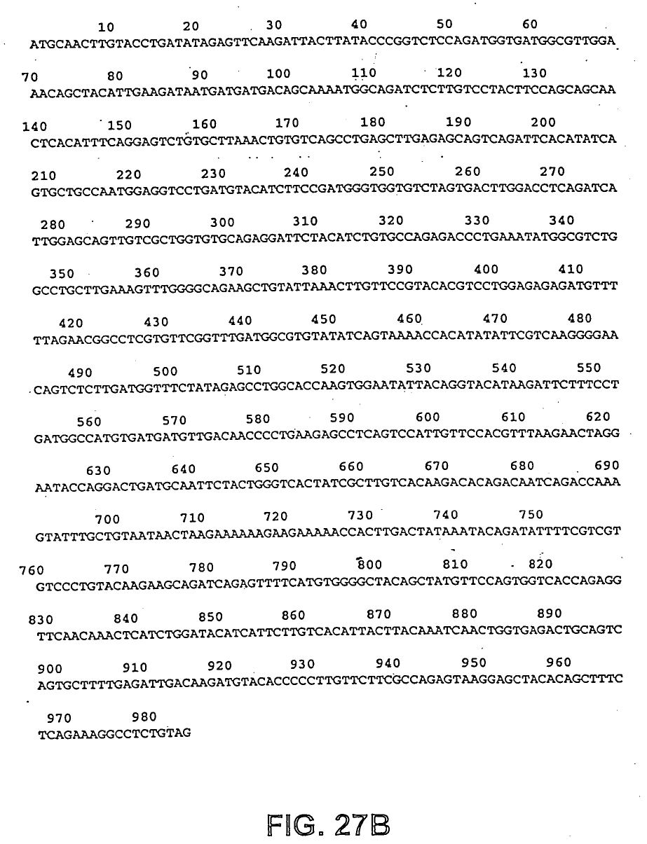

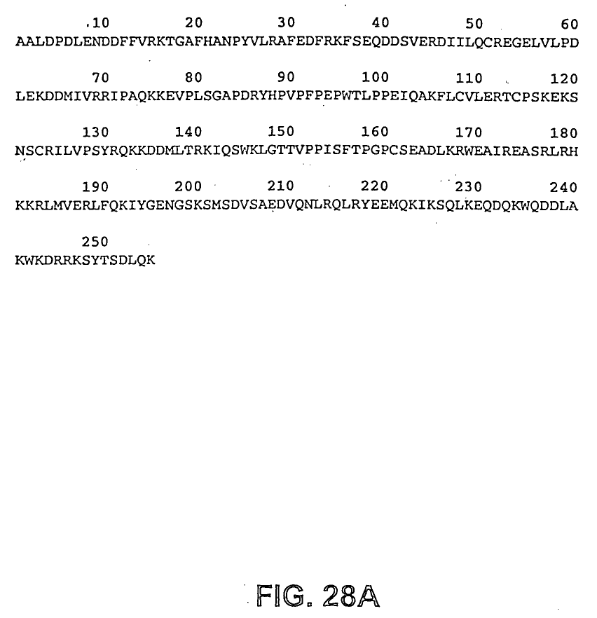

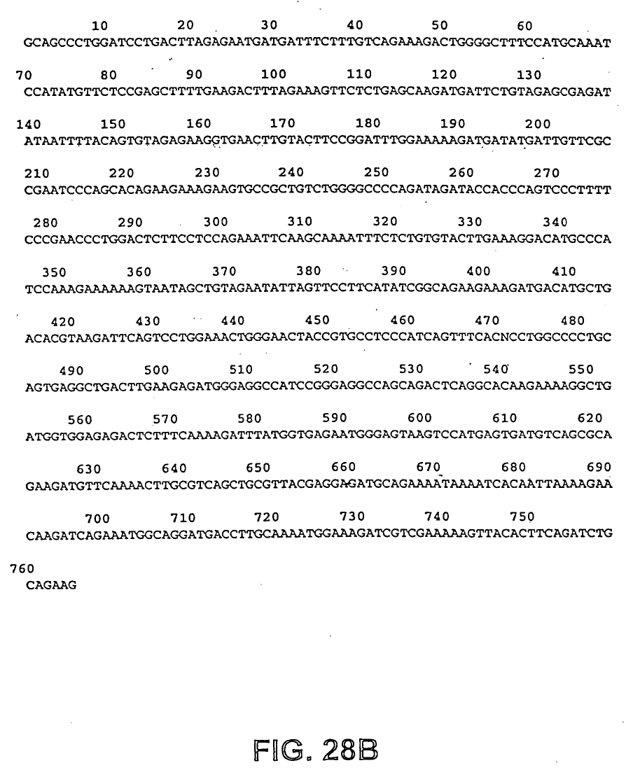

As used herein, “an FBP gene” refers to:
(a) a nucleic acid molecule containing the DNA sequences of FBP1, shown in FIG. 3 (SEQ ID NO:1), the DNA sequences of FBP2, shown in FIG. 4 (SEQ ID NO:3), the DNA sequences of FBP3a, shown in FIG. 5 (SEQ ID NO:5), the DNA sequences of FBP3b, shown in FIG. 6 (SEQ ID NO:23), the DNA sequences of FBP4, shown in FIG. 7 (SEQ ID NO:7), the DNA sequences of FBP5, shown in FIG. 8 (SEQ ID NO:9), the DNA sequences of FBP6, shown in FIG. 9 (SEQ ID NO:11), the DNA sequences of FBP7, shown in FIG. 10 (SEQ ID NO:13), the DNA sequences of FBP8, shown in FIG. 11 (SEQ ID NO:25), the DNA sequences of FBP9, shown in FIG. 12 (SEQ ID NO:27), the DNA sequences of FBP10, shown in FIG. 13 (SEQ ID NO:29), the DNA sequences of FBP11, shown in FIG. 14 (SEQ ID NO:31), the DNA sequences of FBP12, shown in FIG. 15 (SEQ ID NO:33), the DNA sequences of FBP13, shown in FIG. 16 (SEQ ID NO:35), the DNA sequences of FBP14, shown in FIG. 17 (SEQ ID NO:37), the DNA sequences of FBP15, shown in FIG. 18 (SEQ ID NO:39), the DNA sequences of FBP16, shown in FIG. 19 (SEQ ID NO:41), the DNA sequences of FBP17, shown in FIG. 20 (SEQ ID NO:43), the DNA sequences of FBP18, shown in FIG. 21 (SEQ ID NO:45), the DNA sequences of FBP19, shown in FIG. 22 (SEQ ID NO:47), the DNA sequences of FBP20, shown in FIG. 23 (SEQ ID NO:49), the DNA sequences of FBP21, shown in FIG. 24 (SEQ ID NO:51), the DNA sequences of FBP22, shown in FIG. 25 (SEQ ID NO:53), the DNA sequences of FBP23, shown in FIG. 26 (SEQ ID NO:55), the DNA sequences of FBP24, shown in FIG. 27 (SEQ ID NO:57), the DNA sequences of FBP25, shown in FIG. 28 (SEQ ID NO:59).
(b) any DNA sequence that encodes a polypeptide containing: the amino acid sequence of FBP1 shown in FIG. 3A (SEQ ID NO:2), the amino acid sequence of FBP2, shown in FIG. 4A (SEQ ID NO:4), the amino acid sequence of FBP3a shown in FIG. 5A (SEQ ID NO:6), the amino acid sequence of FBP3b shown in FIG. 6A (SEQ ID NO:24), the amino acid sequence of FBP4 shown in FIG. 7A (SEQ ID NO:8), the amino acid sequence of FBP5 shown in FIG. 8A (SEQ ID NO:10), or the amino acid sequence of FBP6 shown in FIG. 9A (SEQ ID NO:12), the amino acid sequences of FBP7, shown in FIG. 10 (SEQ ID NO:14), the amino acid sequences of FBP8, shown in FIG. 11 (SEQ ID NO:26), the amino acid sequences of FBP9, shown in FIG. 12 (SEQ ID NO:28), the amino acid sequences of FBP10, shown in FIG. 13 (SEQ ID NO:30), the amino acid sequences of FBP11, shown in FIG. 14 (SEQ ID NO:32), the amino acid sequences of FBP12, shown in FIG. 15 (SEQ ID NO:34), the amino acid sequences of FBP13, shown in FIG. 16 (SEQ ID NO:36), the amino acid sequences of FBP14, shown in FIG. 17 (SEQ ID NO:38), the amino acid sequences of FBP15, shown in FIG. 18 (SEQ ID NO:40), the amino acid sequences of FBP16, shown in FIG. 19 (SEQ ID NO:42), the amino acid sequences of FBP17, shown in FIG. 20 (SEQ ID NO:44), the amino acid sequences of FBP18, shown in FIG. 21 (SEQ ID NO:46), the amino acid sequences of FBP19, shown in FIG. 22 (SEQ ID NO:48), the amino acid sequences of FBP20, shown in FIG. 23 (SEQ ID NO:50), the amino acid sequences of FBP21, shown in FIG. 24 (SEQ ID NO:52), the amino acid sequences of FBP22, shown in FIG. 25 (SEQ ID NO:54), the amino acid sequences of FBP23, shown in FIG. 26 (SEQ ID NO:56), the amino acid sequences of FBP24, shown in FIG. 27 (SEQ ID NO:58), the amino acid sequences of FBP25, shown in FIG. 28 (SEQ ID NO:60).
(c) any DNA sequence that hybridizes to the complement of the DNA sequences that encode any of the amino acid sequences of (SEQ ID NO: 2, 4, 6, 8, 10, 12 or 14) or FIG. 15 under highly stringent conditions, e.g., hybridization to filter-bound DNA in 0.5 M NaHPO4, 7% sodium dodecyl sulfate (SDS), 1 mM EDTA at 65 C, and washing in 0.1×SSC/0.1% SDS at 68 C (Ausubel F. M. et al., eds., 1989, Current Protocols in Molecular Biology, Vol. 1, Green Publishing Associates, Inc., and John Wiley & sons, Inc., New York, at p. 2.10.3); and/or
(d) any DNA sequence that hybridizes to the complement of the DNA sequences that encode any of the amino acid sequences in (SEQ ID NO: 2, 4, 6, 8, 10, 12 or 14) or FIG. 15, under less stringent conditions, such as moderately stringent conditions, e.g., washing in 0.2×SSC/0.1% SDS at 42 C (Ausubel et al., 1989, supra), and encodes a gene product functionally equivalent to an FBP gene product.

It is understood that the FBP gene sequences of the present invention do not encompass the previously described genes encoding other mammalian F-box proteins, Skp2, Elongin A, Cyclin F, mouse Md6, (see Pagano, 1997, supra; Zhang et al., 1995, supra; Bai et al., 1996, supra; Skowyra et al., 1997, supra). It is further understood that the nucleic acid molecules of the invention do not include nucleic acid molecules that consist solely of the nucleotide sequence in GenBank Accession Nos. AC002428, AI457595, AI105408, H66467, T47217, H38755, THC274684, AI750732, AA976979, AI571815, T57296, Z44228, Z45230, N42405, AA018063, AI751015, AI400663, T74432, AA402415, AI826000, AI590138, AF174602, Z45775, AF174599, THC288870, AI017603, AF174598, THC260994, AI475671, AA768343, AF174595, THC240016, N70417, T10511, AF174603, EST04915, AA147429, AI192344, AF174594, AI147207, AI279712, AA593015, AA644633, AA335703, N26196, AF174604, AF053356, AF174606, AA836036, AA853045, AI479142, AA772788, AA039454, AA397652, AA463756, AA007384, AA749085, AI640599, THC253263, AB020647, THC295423, AA434109, AA370939, AA215393, THC271423, AF052097, THC288182, AL049953, CAB37981, AL022395, AL031178, THC197682, and THC205131.

FBP sequences of the present invention are derived from a eukaryotic genome, preferably a mammalian genome, and more preferably a human or murine genome. Thus, the nucleotide sequences of the present invention do not encompass those derived from yeast genomes. In a specific embodiment, the nucleotides of the present invention encompass any DNA sequence derived from a mammalian genome which hybridizes under highly stringent conditions to SEQ ID NO: 1, 3, 5, 7, 9, 11 or 13, or to DNA sequence shown in FIG. 14, encodes a gene product which contains an F-box motif and binds to Skp1. In a specific embodiment, the nucleotides of the present invention encompass any DNA sequence derived from a mammalian genome which hybridize under highly stringent conditions to SEQ ID NO: 1, 3, 5, 7, 9, 11 or 13 encodes a gene product which contains an F-box motif and another domain selected from the group comprising WD-40, leucine rich region, leucine zipper motif, or other protein-protein interaction domain, and binds to Skp-1 and is at least 300 or 400 nucleotides in length.

FBP sequences can include, for example, either eukaryotic genomic DNA (cDNA) or cDNA sequences. When referring to a nucleic acid which encodes a given amino acid sequence, therefore, it is to be understood that the nucleic acid need not only be a cDNA molecule, but can also, for example, refer to a cDNA sequence from which an mRNA species is transcribed that is processed to encode the given amino acid sequence.

As used herein, an FBP gene may also refer to degenerate variants of DNA sequences (a) through (d).

The invention also includes nucleic acid molecules derived from mammalian nucleic acids, preferably DNA molecules, that hybridize to, and are therefore the complements of, the DNA sequences (a) through (d), in the preceding paragraph. Such hybridization conditions may be highly stringent or less highly stringent, as described above. In instances wherein the nucleic acid molecules are deoxyoligonucleotides (“oligos”), highly stringent conditions may refer, e.g., to washing in 6×SSC/0.05% sodium pyrophosphate at 37 C (for 14-base oligos), 48 C (for 17-base oligos), 55 C (for 20-base oligos), and 60 C (for 23-base oligos). These nucleic acid molecules may encode or act as FBP gene antisense molecules, useful, for example, in FBP gene regulation (for and/or as antisense primers in amplification reactions of FBP gene nucleic acid sequences). With respect to FBP gene regulation, such techniques can be used to regulate, for example, an FBP-regulated pathway, in order to block cell proliferation associated with cancer. Further, such sequences may be used as part of ribozyme and/or triple helix sequences, also useful for FBP gene regulation. Still further, such molecules may be used as components of diagnostic methods whereby, for example, the presence of a particular FBP allele responsible for causing an FBP-related disorder, e.g., proliferative or differentiative disorders such as tumorigenesis or cancer, may be detected.

The invention also encompasses:
(a) DNA vectors that contain any of the foregoing FBP coding sequences and/or their complements (i.e., antisense);
(b) DNA expression vectors that contain any of the foregoing FBP coding sequences operatively associated with a regulatory element that directs the expression of the coding sequences; and
(c) genetically engineered host cells that contain any of the foregoing FBP coding sequences operatively associated with a regulatory element that directs the expression of the coding sequences in the host cell.

As used herein, regulatory elements include but are not limited to inducible and non-inducible promoters, enhancers, operators and other elements known to those skilled in the art that drive and regulate expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast-mating factors.

The invention further includes fragments of any of the DNA sequences disclosed herein.

In one embodiment, the FBP gene sequences of the invention are mammalian gene sequences, with human sequences being preferred.

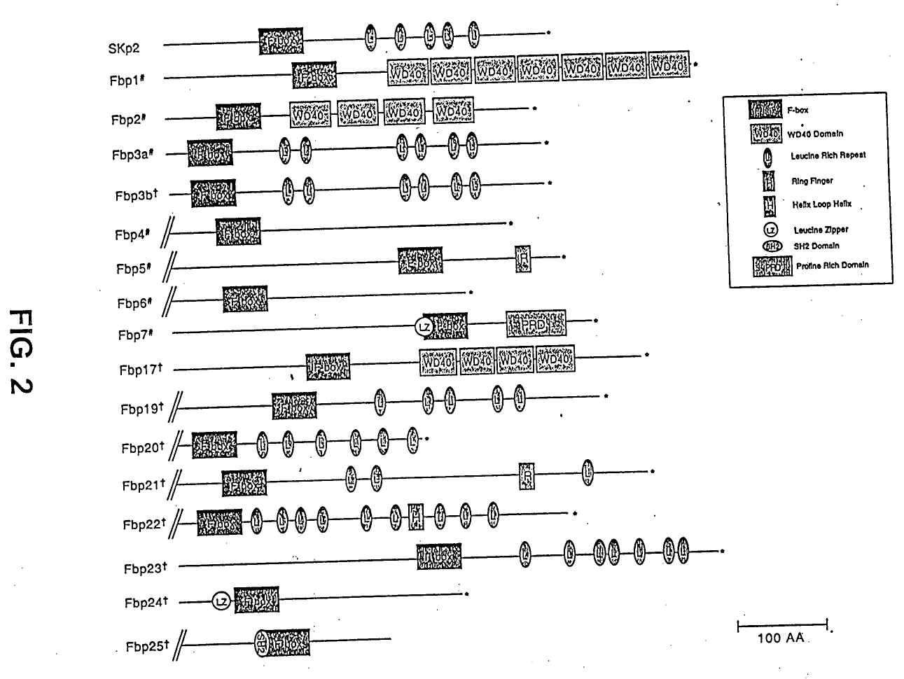

In yet another embodiment, the FBP gene sequences of the invention are gene sequences encoding FBP gene products containing polypeptide portions corresponding to (that is, polypeptide portions exhibiting amino acid sequence similarity to) the amino acid sequence depicted in FIGS. 2, 4-9 or 15, wherein the corresponding portion exhibits greater than about 50% amino acid identity with the depicted sequence, averaged across the FBP gene product's entire length.

In specific embodiments, F-box encoding nucleic acids comprise the cDNA sequences of SEQ ID NOs: 1, 3, 5, 23, 7, 9, 11, 13, 15, 25, 27, 29, 31, 33, 35, 37, 39, 41, 43, 45, 47, 49, 51, 53, 55, 57, or 59, nucleotide sequence of FIGS. 3B, 4B, 5B, 6B, 7B, 8B, 9B, 10B, 11B, 12B, 13B, 14B, 15B, 16B, 17B, 18B, 19B, 20B, 21B, 22B, 23B, 24B, 25B, 26B, 27B, or 28B, respectively, or the coding regions thereof, or nucleic acids encoding an F-box protein (e.g., a protein having the sequence of SEQ ID NOs: 2, 4, 6, 24, 8, 10, 12, 14, 26, 28, 30, 32, 34, 36, 38, 40, 42, 44, 46, 48, 50, 52, 54, 56, 68, or 60, or as shown in FIGS. 3A, 4A, 5A, 6A, 7A, 8A, 9A, 10A, 11A, 12A, 13A, 14A, 15A, 16A, 17A, 18A, 19A, 20A, 21A, 22A, 23A, 24A, 25A, 26A, 27A, or 28A, respectively).

The invention further provides nucleotide fragments of nucleotide sequences encoding FBP1, FBP2, FBP3a, FBP4, FBP5, FBP6, or FBP7 (SEQ ID NOs: 1, 3, 5, 7, 9, 11 and 13, respectively) of the invention. Such fragments consist of at least 8 nucleotides (i.e., a hybridizable portion) of an FBP gene sequence; in other embodiments, the nucleic acids consist of at least 25 (continuous) nucleotides, 50 nucleotides, 100 nucleotides, 150 nucleotides, or 200 nucleotides of an F-box sequence, or a full-length F-box coding sequence. In another embodiment, the nucleic acids are smaller than 35, 200 or 500 nucleotides in length. Nucleic acids can be single or double stranded. The invention also relates to nucleic acids hybridizable to or complementary to the foregoing sequences. In specific aspects, nucleic acids are provided which comprise a sequence complementary to at least 10, 25, 50, 100, or 200 nucleotides or the entire coding region of an F-box gene.

The invention further relates to the human genomic nucleotide sequences of nucleic acids. In specific embodiments, F-box encoding nucleic acids comprise the genomic sequences of SEQ ID NOs:1, 3, 5, 7, 9, 11 or 13 or the coding regions thereof, or nucleic acids encoding an FBP protein (e.g., a protein having the sequence of SEQ ID Nos: 2, 4, 6, 8, 10, 12 or 14). The invention provides purified nucleic acids consisting of at least 8 nucleotides (i.e., a hybridizable portion) of an FBP gene sequence; in other embodiments, the nucleic acids consist of at least 25 (continuous) nucleotides, 50 nucleotides, 100 nucleotides, 150 nucleotides, or 200 nucleotides of an FBP gene sequence or a full-length FBP gene coding sequence. In another embodiment, the nucleic acids are smaller than 35, 200 or 500 nucleotides in length. Nucleic acids can be single or double stranded. The invention also relates to nucleic acids hybridizable to or complementary to the foregoing sequences. In specific aspects, nucleic acids are provided which comprise a sequence complementary to at least 10, 25, 50, 100, or 200 nucleotides or the entire coding region of an FBP gene sequence.

In addition to the human FBP nucleotide sequences disclosed herein, other FBP gene sequences can be identified and readily isolated, without undue experimentation, by molecular biological techniques well known in the art, used in conjunction with the FBP gene sequences disclosed herein. For example, additional human FBP gene sequences at the same or at different genetic loci as those disclosed in SEQ ID Nos: 1, 3, 5, 7, 9, 11 or 13 can be isolated readily. There can exist, for example, genes at other genetic or physical loci within the human genome that encode proteins that have extensive homology to one or more domains of the FBP gene products and that encode gene products functionally equivalent to an FBP gene product. Further, homologous FBP gene sequences present in other species can be identified and isolated readily.

The FBP nucleotide sequences of the invention further include nucleotide sequences that encode polypeptides having at least 30%, 35%, 40%, 45%, 50%, 55%, 60%, 65%, 70%, 75%, 80%, 85%, 90%, 95%, 98%, or higher amino acid sequence identity to the polypeptides encoded by the FBP nucleotide sequences of SEQ ID No. 1, 3, 5, 7, 9, 11 or 13.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of overlapping positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

With respect to identification and isolation of FBP gene sequences present at the same genetic or physical locus as those sequences disclosed herein, such sequences can, for example, be obtained readily by utilizing standard sequencing and bacterial artificial chromosome (BAC) technologies.

With respect to the cloning of an FBP gene homologue in human or other species (e.g., mouse), the isolated FBP gene sequences disclosed herein may be labeled and used to screen a cDNA library constructed from mRNA obtained from appropriate cells or tissues (e.g., brain tissues) derived from the organism (e.g., mouse) of interest. The hybridization conditions used should be of a lower stringency when the cDNA library is derived from an organism different from the type of organism from which the labeled sequence was derived.

Alternatively, the labeled fragment may be used to screen a genomic library derived from the organism of interest, again, using appropriately stringent conditions. Low stringency conditions are well known to those of skill in the art, and will vary predictably depending on the specific organisms from which the library and the labeled sequences are derived. For guidance regarding such conditions see, for example, Sambrook, et al., 1989, Molecular Cloning, A Laboratory Manual, Second Edition, Cold Spring Harbor Press, N.Y.; and Ausubel, et al., supra. Further, an FBP gene homologue may be isolated from, for example, human nucleic acid, by performing PCR using two degenerate oligonucleotide primer pools designed on the basis of amino acid sequences within any FBP gene product disclosed herein.

The PCR product may be subcloned and sequenced to ensure that the amplified sequences represent the sequences of an FBP gene nucleic acid sequence. The PCR fragment may then be used to isolate a full length cDNA clone by a variety of methods. For example, the amplified fragment may be labeled and used to screen a bacteriophage cDNA library. Alternatively, the labeled fragment may be used to isolate genomic clones via the screening of a genomic library.

PCR technology may also be utilized to isolate full length cDNA sequences. For example, RNA may be isolated, following standard procedures, from an appropriate cellular or tissue source (i.e., one known, or suspected, to express the FBP gene, such as, for example, blood samples or brain tissue samples obtained through biopsy or post-mortem). A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of cloning strategies that may be used, see e.g., Sambrook et al., supra.

FBP gene sequences may additionally be used to identify mutant FBP gene alleles. Such mutant alleles may be isolated from individuals either known or proposed to have a genotype that contributes to the symptoms of an FBP gene disorder, such as proliferative or differentiative disorders involved in tumorigenesis or causing cancer, for example. Mutant alleles and mutant allele products may then be utilized in the therapeutic, diagnostic and prognostic systems described below. Additionally, such FBP gene sequences can be used to detect FBP gene regulatory (e.g., promoter) defects which can be associated with an FBP disorder, such as proliferative or differentiative disorders involved in tumorigenesis or causing cancer, for example.

FBP alleles may be identified by single strand conformational polymorphism (SSCP) mutation detection techniques, Southern blot, and/or PCR amplification techniques. Primers can routinely be designed to amplify overlapping regions of the whole FBP sequence including the promoter region. In one embodiment, primers are designed to cover the exon-intron boundaries such that, first, coding regions can be scanned for mutations. Genomic DNA isolated from lymphocytes of normal and affected individuals is used as PCR template. PCR products from normal and affected individuals are compared, either by single strand conformational polymorphism (SSCP) mutation detection techniques and/or by sequencing. SSCP analysis can be performed as follows: 100 ng of genomic DNA is amplified in a 10 μl reaction, adding 10 pmols of each primer, 0.5 U of Taq DNA polymerase (Promega), 1 μCi of α-[³²P]dCTP (NEN; specific activity, 3000 Ci/mmol), in 2.5 μM dNTPs (Pharmacia), 10 mM Tris-HCl (pH 8.8), 50 mM KCl, 1 mM MgCl2, 0.01% gelatin, final concentration. Thirty cycles of denaturation (94° C.), annealing (56° C. to 64° C., depending on primer melting temperature), and extension (72° C.) is carried out in a thermal-cycler (MJ Research, Boston, Mass., USA), followed by a 7 min final extension at 72° C. Two microliters of the reaction mixture is diluted in 0.1% SDS, 10 mM EDTA and then mixed 1:1 with a sequencing stop solution containing 20 mM NaOH. Samples are heated at 95 C for 5 min, chilled on ice for 3 min and then 3 l will be loaded onto a 6% acrylamide/TBE gel containing 5% (v/v) glycerol. Gels are run at 8 W for 12-15 h at room temperature. Autoradiography is performed by exposure to film at −70 C with intensifying screens for different periods of time. The mutations responsible for the loss or alteration of function of the mutant FBP gene product can then be ascertained.

Alternatively, a cDNA of a mutant FBP gene may be isolated, for example, using PCR. In this case, the first cDNA strand may be synthesized by hybridizing an oligo-dT oligonucleotide to mRNA isolated from tissue known or suspected to be expressed in an individual putatively carrying the mutant FBP allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant FBP allele to that of the normal FBP allele, the mutation(s) responsible for the loss or alteration of function of the mutant FBP gene product can be ascertained.

Alternatively, a genomic library can be constructed using DNA obtained from an individual suspected of or known to carry a mutant FBP allele, or a cDNA library can be constructed using RNA from a tissue known, or suspected, to express a mutant FBP allele. An unimpaired FBP gene or any suitable fragment thereof may then be labeled and used as a probe to identify the corresponding mutant FBP allele in such libraries. Clones containing the mutant FBP gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant FBP allele in an individual suspected of or known to carry such a mutant allele. In this manner, gene products made by the putatively mutant tissue may be expressed and screened using standard antibody screening techniques in conjunction with antibodies raised against the normal FBP gene product, as described, below, in Section 5.3. (For screening techniques, see, for example, Harlow and Lane, eds., 1988, “Antibodies: A Laboratory Manual”, Cold Spring Harbor Press, Cold Spring Harbor.)

Nucleic acids encoding derivatives and analogs of FBP proteins, and FBP antisense nucleic acids can be isolated by the methods recited above. As used herein, a “nucleic acid encoding a fragment or portion of an F-box protein” shall be construed as referring to a nucleic acid encoding only the recited fragment or portion of the FBP and not the other contiguous portions of the FBP protein as a continuous sequence.

Fragments of FBP gene nucleic acids comprising regions conserved between (i.e., with homology to) other FBP gene nucleic acids, of the same or different species, are also provided. Nucleic acids encoding one or more FBP domains can be isolated by the methods recited above.

In cases where an FBP mutation results in an expressed gene product with altered function (e.g., as a result of a missense or a frameshift mutation), a polyclonal set of anti-FBP gene product antibodies are likely to cross-react with the mutant FBP gene product. Library clones detected via their reaction with such labeled antibodies can be purified and subjected to sequence analysis according to methods well known to those of skill in the art.

5.2 Proteins and Polypeptides of FBP Genes

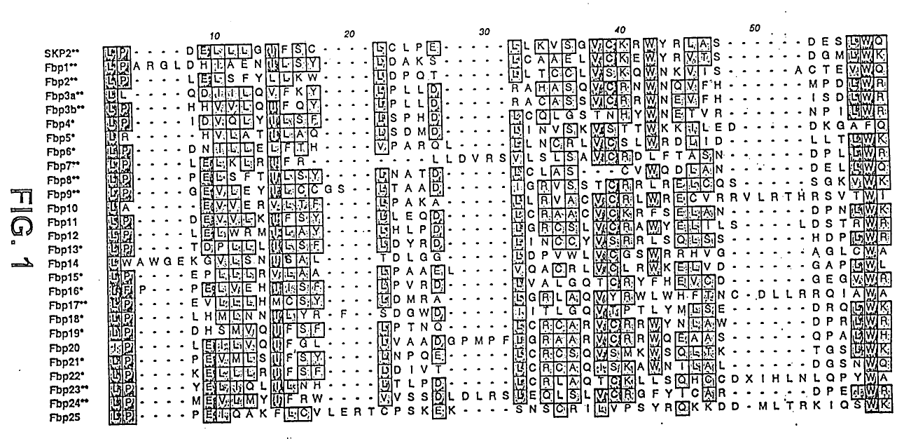

The amino acid sequences depicted in FIGS. 1, 2, and parts B of FIGS. 3 to 28 represent FBP gene products. The FBP1 gene product, sometimes referred to herein as a “FBP1 protein”, includes those gene products encoded by the FBP1 gene sequences described in Section 5.1, above. Likewise, the FBP2, FBP3a, FBP3b, FBP4, FBP5, FBP6, FBP7, FBP8, FBP9, FBP10, FBP1, FBP12, FBP13, FBP14, FBP15, FBP16, FBP17, FBP18, FBP19, FBP20, FBP21, FBP22, FBP23, FBP24, and FBP25 gene products, referred to herein as an FBP2, FBP3a, FBP3b, FBP4, FBP5, FBP6, FBP7, FBP8, FBP9, FBP10, FBP11, FBP12, FBP13, FBP14, FBP15, FBP16, FBP17, FBP18, FBP19, FBP20, FBP21, FBP22, FBP23, FBP24, and FBP25 proteins, include those gene products encoded by the FBP2, FBP3, FBP4, FBP5, FBP6, FBP7, FBP8, FBP9, FBP10, FBP11, FBP12, FBP13, FBP14, FBP15, FBP16, FBP17, FBP18, FBP19, FBP20, FBP21, FBP22, FBP23, FBP24, and FBP25 genes. In accordance with the present invention, the nucleic acid sequences encoding the FBP gene products are derived from eukaryotic genomes, including mammalian genomes. In a preferred embodiment the nucleic acid sequences encoding the FBP gene products are derived from human or murine genomes.

FBP gene products, or peptide fragments thereof, can be prepared for a variety of uses. For example, such gene products, or peptide fragments thereof, can be used for the generation of antibodies, in diagnostic and prognostic assays, or for the identification of other cellular or extracellular gene products involved in the ubiquitination pathway and thereby implicated in the regulation of cell cycle and proliferative disorders.

In addition, FBP gene products of the present invention may include proteins that represent functionally equivalent (see Section 5.1 for a definition) gene products. FBP gene products of the invention do not encompass the previously identified mammalian F-box proteins Skp2, Cyclin F, Elongin A, or mouse Md6 (see Pagano, 1997, supra; Zhang et al., 1995 supra; Bai et al., 1996 supra; Skowyra et al., 1997, supra).

Functionally equivalent FBP gene products may contain deletions, including internal deletions, additions, including additions yielding fusion proteins, or substitutions of amino acid residues within and/or adjacent to the amino acid sequence encoded by the FBP gene sequences described, above, in Section 5.1, but that result in a “silent” change, in that the change produces a functionally equivalent FBP gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Alternatively, where alteration of function is desired, deletion or non-conservative alterations can be engineered to produce altered FBP gene products. Such alterations can, for example, alter one or more of the biological functions of the FBP gene product. Further, such alterations can be selected so as to generate FBP gene products that are better suited for expression, scale up, etc. in the host cells chosen. For example, cysteine residues can be deleted or substituted with another amino acid residue in order to eliminate disulfide bridges.

The FBP gene products, peptide fragments thereof and fusion proteins thereof, may be produced by recombinant DNA technology using techniques well known in the art. Thus, methods for preparing the FBP gene polypeptides, peptides, fusion peptide and fusion polypeptides of the invention by expressing nucleic acid containing FBP gene sequences are described herein. Methods that are well known to those skilled in the art can be used to construct expression vectors containing FBP gene product coding sequences and appropriate transcriptional and translational control signals. These methods include, for example, in vitro recombinant DNA techniques, synthetic techniques, and in vivo genetic recombination. See, for example, the techniques described in Sambrook, et al., supra, and Ausubel, et al., supra. Alternatively, RNA capable of encoding FBP gene product sequences may be chemically synthesized using, for example, synthesizers. See, for example, the techniques described in “Oligonucleotide Synthesis”, 1984, Gait, ed., IRL Press, Oxford.

A variety of host-expression vector systems may be utilized to express the FBP gene coding sequences of the invention. Such host-expression systems represent vehicles by which the coding sequences of interest may be produced and subsequently purified, but also represent cells that may, when transformed or transfected with the appropriate nucleotide coding sequences, exhibit the FBP gene product of the invention in situ. These include but are not limited to microorganisms such as bacteria (e.g., E. coli, B. subtilis) transformed with recombinant bacteriophage DNA, plasmid DNA or cosmid DNA expression vectors containing FBP gene product coding sequences; yeast (e.g., Saccharomyces, Pichia) transformed with recombinant yeast expression vectors containing the FBP gene product coding sequences; insect cell systems infected with recombinant virus expression vectors (e.g., baculovirus) containing the FBP gene product coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing FBP gene product coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the FBP gene product being expressed. For example, when a large quantity of such a protein is to be produced, for the generation of pharmaceutical compositions of FBP protein or for raising antibodies to FBP protein, for example, vectors that direct the expression of high levels of fusion protein products that are readily purified may be desirable. Such vectors include, but are not limited, to the E. coli expression vector pUR278 (Ruther et al., 1983, EMBO J. 2, 1791), in which the FBP gene product coding sequence may be ligated individually into the vector in frame with the lac Z coding region so that a fusion protein is produced; pIN vectors (Inouye and Inouye, 1985, Nucleic Acids Res. 13, 3101-3109; Van Heeke and Schuster, 1989, J. Biol. Chem. 264, 5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica, nuclear polyhedrosis virus (AcNPV) is used as a vector to express foreign genes. The virus grows in Spodoptera frugiperda cells. The FBP gene coding sequence may be cloned individually into non-essential regions (for example the polyhedrin gene) of the virus and placed under control of an AcNPV promoter (for example the polyhedrin promoter). Successful insertion of FBP gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., see Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the FBP gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing FBP gene product in infected hosts. (e.g., See Logan and Shenk, 1984, Proc. Natl. Acad. Sci. USA 81, 3655-3659). Specific initiation signals may also be required for efficient translation of inserted FBP gene product coding sequences. These signals include the ATG initiation codon and adjacent sequences. In cases where an entire FBP gene, including its own initiation codon and adjacent sequences, is inserted into the appropriate expression vector, no additional translational control signals may be needed. However, in cases where only a portion of the FBP gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner, et al., 1987, Methods in Enzymol. 153, 516-544).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, and WI38.

For long-term, high-yield production of recombinant proteins, stable expression is preferred. For example, cell lines that stably express the FBP gene product may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method may advantageously be used to engineer cell lines that express the FBP gene product. Such engineered cell lines may be particularly useful in screening and evaluation of compounds that affect the endogenous activity of the FBP gene product.

A number of selection systems may be used, including but not limited to the herpes simplex virus thymidine kinase (Wigler, et al., 1977, Cell 11:223), hypoxanthine-guanine phosphoribosyltransferase (Szybalska and Szybalski, 1962, Proc. Natl. Acad. Sci. USA 48, 2026), and adenine phosphoribosyltransferase (Lowy, et al., 1980, Cell 22, 817) genes can be employed in tk-, hgprt- or aprt-cells, respectively. Also, antimetabolite resistance can be used as the basis of selection for the following genes: dhfr, which confers resistance to methotrexate (Wigler, et al., 1980, Natl. Acad. Sci. USA 77, 3567; O'Hare, et al., 1981, Proc. Natl. Acad. Sci. USA 78, 1527); gpt, which confers resistance to mycophenolic acid (Mulligan and Berg, 1981, Proc. Natl. Acad. Sci. USA 78, 2072); neo, which confers resistance to the aminoglycoside G-418 (Colberre-Garapin, et al., 1981, J. Mol. Biol. 150, 1); and hygro, which confers resistance to hygromycin (Santerre, et al., 1984, Gene 30, 147).

Alternatively, any fusion protein may be readily purified by utilizing an antibody specific for the fusion protein being expressed. For example, a system described by Janknecht, et al. allows for the ready purification of non-denatured fusion proteins expressed in human cell lines (Janknecht, et al., 1991, Proc. Natl. Acad. Sci. USA 88, 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni2+ nitriloacetic acid-agarose columns and histidine-tagged proteins are selectively eluted with imidazole-containing buffers.

The FBP gene products can also be expressed in transgenic animals. Animals of any species, including, but not limited to, mice, rats, rabbits, guinea pigs, pigs, micro-pigs, goats, sheep, and non-human primates, e.g., baboons, monkeys, and chimpanzees may be used to generate FBP transgenic animals. The term “transgenic,” as used herein, refers to animals expressing FBP gene sequences from a different species (e.g., mice expressing human FBP sequences), as well as animals that have been genetically engineered to overexpress endogenous (i.e., same species) FBP sequences or animals that have been genetically engineered to no longer express endogenous FBP gene sequences (i.e., “knock-out” animals), and their progeny.

In particular, the present invention relates to FBP1 knockout mice. The present invention also relates to transgenic mice which express human wild-type FBP1 and Skp2 gene sequences in addition to mice engineered to express human mutant FBP1 and Skp2 gene sequences deleted of their F-box domains. Any technique known in the art may be used to introduce an FBP gene transgene into animals to produce the founder lines of transgenic animals. Such techniques include, but are not limited to pronuclear microinjection (Hoppe and Wagner, 1989, U.S. Pat. No. 4,873,191); retrovirus mediated gene transfer into germ lines (Van der Putten, et al., 1985, Proc. Natl. Acad. Sci., USA 82, 6148-6152); gene targeting in embryonic stem cells (Thompson, et al., 1989, Cell 56, 313-321); electroporation of embryos (Lo, 1983, Mol. Cell. Biol. 3, 1803-1814); and sperm-mediated gene transfer (Lavitrano et al., 1989, Cell 57, 717-723) (For a review of such techniques, see Gordon, 1989, Transgenic Animals, Intl. Rev. Cytol. 115, 171-229)

Any technique known in the art may be used to produce transgenic animal clones containing an FBP transgene, for example, nuclear transfer into enucleated oocytes of nuclei from cultured embryonic, fetal or adult cells induced to quiescence (Campbell, et al., 1996, Nature 380, 64-66; Wilmut, et al., Nature 385, 810-813).

The present invention provides for transgenic animals that carry an FBP transgene in all their cells, as well as animals that carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, et al., 1992, Proc. Natl. Acad. Sci. USA 89, 6232-6236). The regulatory sequences required for such a cell-type specific activation will depend upon the particular cell type of interest, and will be apparent to those of skill in the art. Examples of regulatory sequences that can be used to direct tissue-specific expression of an FBP transgene include, but are not limited to, the elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:42S-51S); the insulin gene control region which is active in pancreatic beta cells (Hanahan, 1985, Nature 315:115-122); immunoglobulin gene control region which is active in lymphoid cells (Grosschedl et al., 1984, Cell 38:647-658; Adams et al., 1985, Nature 318:533-538; Alexander et al., 1987, Mol. Cell. Biol. 7:1436-1444): albumin gene control region which is active in liver (Pinkert et al., 1987, Genes and Devel. 1:268-276) alpha-fetoprotein gene control region which is active in liver (Krumlauf et al., 1985, Mol. Cell. Biol. 5:1639-1648; Hammer et al., 1987, Science 235:53-58); alpha-1-antitrypsin gene control region which is active in liver (Kelsey et al., 1987, Genes and Devel. 1:161-171); beta-globin gene control region which is active in myeloid cells (Magram et al., 1985, Nature 315:338-340; Kollias et al., 1986, Cell 46:89-94); myelin basic protein gene control region which is active in oligodendrocyte cells in the brain (Readhead et al., 1987, Cell 48:703-712); myosin light chain-2 gene control region which is active in skeletal muscle (Shani, 1985, Nature 314:283-286); and gonadotropic releasing hormone gene control region which is active in the hypothalamus (Mason et al., 1986, Science 234:1372-1378). Promoters isolated from the genome of viruses that grow in mammalian cells, (e.g., vaccinia virus 7.5K, SV40, HSV, adenoviruses MLP, MMTV, LTR and CMV promoters) may be used, as well as promoters produced by recombinant DNA or synthetic techniques.

When it is desired that the FBP gene transgene be integrated into the chromosomal site of the endogenous FBP gene, gene targeting is preferred. Briefly, when such a technique is to be utilized, vectors containing some nucleotide sequences homologous to the endogenous FBP gene are designed for the purpose of integrating, via homologous recombination with chromosomal sequences, into and disrupting the function of the nucleotide sequence of the endogenous FBP gene. The transgene may also be selectively introduced into a particular cell type, thus inactivating the endogenous FBP gene in only that cell type, by following, for example, the teaching of Gu, et al. (Gu, et al., 1994, Science 265, 103-106). The regulatory sequences required for such a cell-type specific inactivation will depend upon the particular cell type of interest, and will be apparent to those of skill in the art.

Once transgenic animals have been generated, the expression of the recombinant FBP gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include but are not limited to Northern blot analysis of tissue samples obtained from the animal, in situ hybridization analysis, and RT-PCR (reverse transcriptase PCR). Samples of FBP gene-expressing tissue, may also be evaluated immunocytochemically using antibodies specific for the FBP transgene product.

Transgenic mice harboring tissue-directed transgenes can be used to test the effects of FBP gene expression the intact animal. In one embodiment, transgenic mice harboring a human FBP1 transgene in the mammary gland can be used to assess the role of FBPs in mouse mammary development and tumorigenesis. In another embodiment, transgenic mice can be generated that overexpress the human FBP1 dominant negative mutant form (F-box deleted) in the mammary gland. In a specific embodiment, for example, the MMTV LTR promoter (mouse mammary tumor virus long terminal repeat) can be used to direct integration of the transgene in the mammary gland. An MMTV/FBP1 fusion gene can be constructed by fusing sequences of the MMTV LTR promoter to nucleotide sequences upstream of the first ATG of FBP1 gene. An SV40 polyadenylation region can also be fused to sequences downstream of the FBP1 coding region. Transgenic mice are generated by methods well known in the art (Gordon, 1989, Transgenic Animals, Intl. Rev. Cytol. 115, 171-229). Briefly, immature B6D2F1 female mice are superovulated and mated to CD-1 males. The following morning the females are examined for the presence of vaginal plugs, and fertilized ova are recovered and microinjected with a plasmid vector. Approximately 2000 copies of the material are microinjected into each pronucleus. Screening of founder animals is performed by extraction of DNA from spleen and Southern hybridization using the MMTV/FBP1 as a probe. Screening of offspring is performed by PCR of tail DNA. Once transgenic pedigrees are established, the expression pattern of the transgene is determined by Northern blot and RT-PCR analysis in different organs in order to correlate it with subsequent pathological changes.

The resulting transgenic animals can then be examined for the role of FBP genes in tumorigenesis. In one embodiment, for example, FBP transgenes can be constructed for use as a breast cancer model. Overexpression of FBP1 genes in such mice is expected to increase β-catenin ubiquitination and degradation, resulting in a tumor suppressor phenotype. Conversely, overexpression of the FBP1 deletion mutant is expected to result in stabilization of β-catenin and induce proliferation of mammary gland epithelium. These phenotypes can be tested in both female and male transgenic mice, by assays such as those described in Sections 5.4, 5.5 and 7.

In another specific embodiment, transgenic mice are generated that express FBP1 transgenes in T-lymphocytes. In this embodiment, a CD2/FBP1 fusion gene is constructed by fusion of the CD2 promoter, which drives expression in both CD4 positive and negative T-cells, to sequences located upstream of the first ATG of an FBP gene, e.g., the wild-type and mutant FBP1 genes. The construct can also contain an SV40 polyadenylation region downstream of the FBP gene. After generation and testing of transgenic mice, as described above, the expression of the FBP transgene is examined. The transgene is expressed in thymus and spleen. Overexpression of wild-type FBP1 is expected to result in a phenotype. For example, possible expected phenotypes of FBP1 transgenic mice include increased degradation of IKBα, increased activation of NFKB, or increased cell proliferation. Conversely, overexpression of the dominant negative mutant, FBP1, lacking the F-box domain, can be expected to have the opposite effect, for example, increased stability of IKBα, decreased activation of NFKB, or decreased cell proliferation. Such transgenic phenotypes can be tested by assays such as those used in Section 5.4 and 5.5.

In another specific embodiment, the SKP2 gene is expressed in T-lymphocytes of trangenic mice. Conversely, the F-box deletion form acts as dominant negative, stabilizing p27 and inhibiting T-cell activation. Construction of the CD2/SKP2-fusion genes and production of transgenic mice are as described above for CD2/FBP fusion genes, using wild-type and mutant SKP2 cDNA, instead of FBP1 cDNA, controlled by the CD2 promoter. Founders and their progeny are analyzed for the presence and expression of the SKP2 transgene and the mutant SKP2 transgene. Expression of the transgene in spleen and thymus is analyzed by Northern blot and RT-PCR

In another specific embodiment, transgenic mice are constructed by inactivation of the FBP1 locus in mice. Inactivation of the FBP1 locus in mice by homologous recombination involves four stages: 1) the construction of the targeting vector for FBP1; 2) the generation of ES +/−cells; 3) the production of knock-out mice; and 4) the characterization of the phenotype. A 129 SV mouse genomic phage library is used to identify and isolate the mouse FBP1 gene. Bacteriophages are plated at an appropriate density and an imprint of the pattern of plaques can be obtained by gently layering a nylon membrane onto the surface of agarose dishes. Bacteriophage particles and DNA are transferred to the filter by capillary action in an exact replica of the pattern of plaques. After denaturation, the DNA is bound to the filter by baking and then hybridized with ³²P-labeled-FBP1 cDNA. Excess probe is washed away and the filters were then exposed for autoradiography. Hybridizing plaques, identified by aligning the film with the original agar plate, were picked for a secondary and a tertiary screening to obtain a pure plaque preparation. Using this method, positive phage which span the region of interest, for example, the region encoding the F-box, are isolated. Using PCR, Southern hybridization, restriction mapping, subcloning and DNA sequencing the partial structure of the wild-type FBP1 gene can be determined.

To inactivate the Fbp1 locus by homologous recombination, a gene targeting vector in which exon 3 in the Fbp1 locus is replaced by a selectable marker, for example, the neoR gene, in an antisense orientation can be constructed. Exon 3 encodes the F-box motif which is known to be critical for Fbp1 interaction with Skp1. The targeting construct possesses a short and a long arm of homology flanking a selectable marker gene. One of the vector arms is relatively short (2 kb) to ensure efficient amplification since homologous recombinant ES clones will be screened by PCR. The other arm is >6 kb to maximize the frequency of homologous recombination. A thymidine kinase (tk) gene, included at the end of the long homology arm of the vector provides an additional negative selection marker (using gancylovir) against ES clones which randomly integrate the targeting vector. Since homologous recombination occurs frequently using linear DNA, the targeting vector is linearized prior to transfection of ES cells. Following electroporation and double drug selection of embryonic stem cell clones, PCR and Southern analysis is used to determine whether homologous recombination has occurred at the FBP1 locus. Screening by PCR is advantageous because a larger number of colonies can be analyzed with this method than with Southern analysis. In addition, PCR screening allows rapid elimination of negative clones thus to avoid feeding and subsequently freezing all the clones while recombinants are identified. This PCR strategy for detection of homologous recombinants is based on the use of a primer pair chosen such that one primer anneals to a sequence specific to the targeting construct, e.g., sequences of the neomycin gene or other selectable marker, and not in the endogenous locus, and the other primer anneals to a region outside the construct, but within the endogenous locus. Southern analysis is used to confirm that a homologous recombination event has occurred (both at the short arm of homology and at the long arm of homology) and that no gene duplication events have occurred during the recombination.

Such FBP1 knockout mice can be used to test the role of Fbp1 in cellular regulation and control of proliferation. In one embodiment, phenotype of such mice lacking Fbp1 is cellular hyperplasia and increased tumor formation. In another embodiment, FBP1 null mice phenotypes include, but are not limited to, increased β-catenin activity, stabilization of β-catenin, increased cellular proliferation, accumulation of IK-Ba, decreased NF-KB activity, deficient immune response, inflammation, or increased cell death or apoptotic activity. Alternatively, a deletion of the of the FBP1 gene can result in an embryonic lethality. In this case, heterozygous mice at the FBP1 allele can be tested using the above assays, and embryos of null FBP mice can be tested using the assays described above.

Transgenic mice bearing FBP transgenes can also be used to screen for compounds capable of modulating the expression of the FBP gene and/or the synthesis or activity of the FBP1 gene or gene product. Such compounds and methods for screening are described.

5.3 Generation of Antibodies to F-Box Proteins and Their Derivatives

According to the invention, F-box motif, its fragments or other derivatives, or analogs thereof, may be used as an immunogen to generate antibodies which immunospecifically bind such an immunogen. Such antibodies include but are not limited to polyclonal, monoclonal, chimeric, single chain, Fab fragments, and an Fab expression library. In a specific embodiment, antibodies to a human FBP protein are produced. In another embodiment, antibodies to a domain (e.g., the F-box domain or the substrate-binding domain) of an FBP are produced.

Various procedures known in the art may be used for the production of polyclonal antibodies to an FBP or derivative or analog. In a particular embodiment, rabbit polyclonal antibodies to an epitope of an FBP encoded by a sequence of FBP1, FBP2, FBP3a, FBP3b, FBP4, FBP5, FBP6, FBP7, FBP8, FBP9, FBP10, FBP11, FBP12, FBP13, FBP14, FBP15, FBP16, FBP17, FBP18, FBP19, FBP20, FBP21, FBP22, FBP23, FBP24, and FBP25, or a subsequence thereof, can be obtained (Pagano, M., 1995, “From peptide to purified antibody”, in Cell Cycle: Materials and Methods. M. Pagano, ed. Spring-Verlag. 217-281). For the production of antibody, various host animals can be immunized by injection with the native FBP, or a synthetic version, or derivative (e.g., fragment) thereof, including but not limited to rabbits, mice, rats, etc. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies directed toward an FBP sequence or analog thereof, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for FBP together with genes from a human antibody molecule of appropriate biological activity can be used; such antibodies are within the scope of this invention.

According to the invention, techniques described for the production of single chain antibodies (U.S. Pat. No. 4,946,778) can be adapted to produce FBP-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for FBPs, derivatives, or analogs.

Antibody fragments which contain the idiotype of the molecule can be generated by known techniques. For example, such fragments include but are not limited to: the F(ab′)2 fragment which can be produced by pepsin digestion of the antibody molecule; the Fab′ fragments which can be generated by reducing the disulfide bridges of the F(ab′)2 fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay). For example, to select antibodies which recognize a specific domain of an FBP, one may assay generated hybridomas for a product which binds to an FBP fragment containing such domain. For selection of an antibody that specifically binds a first FBP homolog but which does not specifically bind a different FBP homolog, one can select on the basis of positive binding to the first FBP homolog and a lack of binding to the second FBP homolog.

Antibodies specific to a domain of an FBP are also provided, such as an F-box motif.

The foregoing antibodies can be used in methods known in the art relating to the localization and activity of the FBP sequences of the invention, e.g., for imaging these proteins, measuring levels thereof in appropriate physiological samples, in diagnostic methods, etc.

In another embodiment of the invention (see infra), anti-FBP antibodies and fragments thereof containing the binding domain are used as therapeutics.

5.4 Screening Assays for the Identification of Agents that Interact with F-Box Proteins and/or Interfere with Their Enzymatic Activities

Novel components of the ubiquitin ligase complex, including FBP1, FBP2, FBP3a, FBP3b, FBP4, FBP5, FBP6, FBP7, FBP8, FBP9, FBP10, FBP11, FBP12, FBP13, FBP14, FBP15, FBP16, FBP17, FBP18, FBP19, FBP20, FBP21, FBP22, FBP23, FBP24, and FBP25, interact with cellular proteins to regulate cellular proliferation. One aspect of the present invention provides methods for assaying and screening fragments, derivatives and analogs of the novel components to identify polypeptides or peptides or other compounds that interact with the novel ubiquitin ligases such as potential substrates of ubiquitin ligase activity. The present invention also provides screening assays to identify compounds that modulate or inhibit the interaction of the novel FBPs with other subunits or numbers of the ubiquitin ligase complex, such as Skp1, or ubiquitinating enzymes with which the novel FBPs interact.

In yet another embodiment, the assays of the present invention may be used to identify polypeptides or peptides or other compounds which inhibit or modulate the interaction between the novel ubiquitin ligases or known (e.g., Skp1) components of the ubiquitin ligase complex with novel or known substrates. By way of example, but not by limitation, the screening assays described herein may be used to identify peptides or proteins that interfere with the interaction between known ubiquitin ligase component, Skp2, and its novel substrate, p27. In another example, compounds that interfere with the interaction between FBP1 and its novel substrate, β-catenin, are identified using the screening assay. In another example, compounds that interfere with the interaction between Skp2 and another putative substrate, E2F, are identified using the screening assay. In yet another example, compounds that interfere with the interaction between FBP1 and another putative substrate, IKBα, are identified using the screening assay.

In yet another embodiment, the assays of the present invention may be used to identify polypeptides or peptides which inhibit or activate the enzymatic activators of the novel FBPs.

5.4.1 Assays for Protein-Protein Interactions

Derivatives, analogs and fragments of proteins that interact with the novel components of the ubiquitin ligase complex of the present invention can be identified by means of a yeast two hybrid assay system (Fields and Song, 1989, Nature 340:245-246 and U.S. Pat. No. 5,283,173). Because the interactions are screened for in yeast, the intermolecular protein interactions detected in this system occur under physiological conditions that mimic the conditions in mammalian cells (Chien et al., 1991, Proc. Natl. Acad. Sci. U.S.A. 88:9578-9581).

Identification of interacting proteins by the improved yeast two hybrid system is based upon the detection of expression of a reporter gene, the transcription of which is dependent upon the reconstitution of a transcriptional regulator by the interaction of two proteins, each fused to one half of the transcriptional regulator. The “bait” (i.e., the novel components of the ubiquitin ligase complex of the present invention or derivatives or analogs thereof) and “prey” (proteins to be tested for ability to interact with the bait) proteins are expressed as fusion proteins to a DNA binding domain, and to a transcriptional regulatory domain, respectively, or vice versa. In various specific embodiments, the prey has a complexity of at least about 50, about 100, about 500, about 1,000, about 5,000, about 10,000, or about 50,000; or has a complexity in the range of about 25 to about 100,000, about 100 to about 100,000, about 50,000 to about 100,000, or about 100,000 to about 500,000. For example, the prey population can be one or more nucleic acids encoding mutants of a protein (e.g., as generated by site-directed mutagenesis or another method of making mutations in a nucleotide sequence). Preferably, the prey populations are proteins encoded by DNA, e.g., cDNA or genomic DNA or synthetically-generated DNA. For example, the populations can be expressed from chimeric genes comprising cDNA sequences from an un-characterized sample of a population of cDNA from mRNA.

In a specific embodiment, recombinant biological libraries expressing random peptides can be used as the source of prey nucleic acids.

In general, proteins of the bait and prey populations are provided as fusion (chimeric) proteins (preferably by recombinant expression of a chimeric coding sequence) comprising each protein contiguous to a pre-selected sequence. For one population, the pre-selected sequence is a DNA binding domain. The DNA binding domain can be any DNA binding domain, as long as it specifically recognizes a DNA sequence within a promoter. For example, the DNA binding domain is of a transcriptional activator or inhibitor. For the other population, the pre-selected sequence is an activator or inhibitor domain of a transcriptional activator or inhibitor, respectively. The regulatory domain alone (not as a fusion to a protein sequence) and the DNA-binding domain alone (not as a fusion to a protein sequence) preferably do not detectably interact (so as to avoid false positives in the assay). The assay system further includes a reporter gene operably linked to a promoter that contains a binding site for the DNA binding domain of the transcriptional activator (or inhibitor). Accordingly, in the present method of the present invention, binding of a ubiquitin ligase fusion protein to a prey fusion protein leads to reconstitution of a transcriptional activator (or inhibitor) which activates (or inhibits) expression of the reporter gene. The activation (or inhibition) of transcription of the reporter gene occurs intracellularly, e.g., in prokaryotic or eukaryotic cells, preferably in cell culture.

The promoter that is operably linked to the reporter gene nucleotide sequence can be a native or non-native promoter of the nucleotide sequence, and the DNA binding site(s) that are recognized by the DNA binding domain portion of the fusion protein can be native to the promoter (if the promoter normally contains such binding site(s)) or non-native to the promoter.

Alternatively, the transcriptional activation binding site of the desired gene(s) can be deleted and replaced with GAL4 binding sites (Bartel et al., 1993, BioTechniques 14:920-924, Chasman et al., 1989, Mol. Cell. Biol. 9:4746-4749). The reporter gene preferably contains the sequence encoding a detectable or selectable marker, the expression of which is regulated by the transcriptional activator, such that the marker is either turned on or off in the cell in response to the presence of a specific interaction. Preferably, the assay is carried out in the absence of background levels of the transcriptional activator (e.g., in a cell that is mutant or otherwise lacking in the transcriptional activator).

The activation domain and DNA binding domain used in the assay can be from a wide variety of transcriptional activator proteins, as long as these transcriptional activators have separable binding and transcriptional activation domains. For example, the GAL4 protein of S. cerevisiae (Ma et al., 1987, Cell 48:847-853), the GCN4 protein of S. cerevisiae (Hope & Struhl, 1986, Cell 46:885-894), the ARD1 protein of S. cerevisiae (Thukral et al., 1989, Mol. Cell. Biol. 9:2360-2369), and the human estrogen receptor (Kumar et al., 1987, Cell 51:941-951), have separable DNA binding and activation domains. The DNA binding domain and activation domain that are employed in the fusion proteins need not be from the same transcriptional activator. In a specific embodiment, a GAL4 or LEXA DNA binding domain is employed. In another specific embodiment, a GAL4 or herpes simplex virus VP16 (Triezenberg et al., 1988, Genes Dev. 2:730-742) activation domain is employed. In a specific embodiment, amino acids 1-147 of GALA (Ma et al., 1987, Cell 48:847-853; Ptashne et al., 1990, Nature 346:329-331) is the DNA binding domain, and amino acids 411-455 of VP16 (Triezenberg et al., 1988, Genes Dev. 2:730-742; Cress et al., 1991, Science 251:87-90) comprise the activation domain.

In a preferred embodiment, the yeast transcription factor GAL4 is reconstituted by protein-protein interaction and the host strain is mutant for GAL4. In another embodiment, the DNA-binding domain is Ace1N and/or the activation domain is Ace1, the DNA binding and activation domains of the Ace1 protein, respectively. Ace1 is a yeast protein that activates transcription from the CUP1 operon in the presence of divalent copper. CUP1 encodes metallothionein, which chelates copper, and the expression of CUP1 protein allows growth in the presence of copper, which is otherwise toxic to the host cells. The reporter gene can also be a CUP1-lacZ fusion that expresses the enzyme beta-galactosidase (detectable by routine chromogenic assay) upon binding of a reconstituted Ace1N transcriptional activator (see Chaudhuri et al., 1995, FEBS Letters 357:221-226). In another specific embodiment, the DNA binding domain of the human estrogen receptor is used, with a reporter gene driven by one or three estrogen receptor response elements (Le Douarin et al., 1995, Nucl. Acids. Res. 23:876-878). The DNA binding domain and the transcriptional activator/inhibitor domain each preferably has a nuclear localization signal (see Ylikomi et al., 1992, EMBO J. 11:3681-3694, Dingwall and Laskey, 1991, TIBS 16:479-481) functional in the cell in which the fusion proteins are to be expressed.

To facilitate isolation of the encoded proteins, the fusion constructs can further contain sequences encoding affinity tags such as glutathione-S-transferase or maltose-binding protein or an epitope of an available antibody, for affinity purification (e.g., binding to glutathione, maltose, or a particular antibody specific for the epitope, respectively) (Allen et al., 1995, TIBS 20:511-516). In another embodiment, the fusion constructs further comprise bacterial promoter sequences for recombinant production of the fusion protein in bacterial cells.

The host cell in which the interaction assay occurs can be any cell, prokaryotic or eukaryotic, in which transcription of the reporter gene can occur and be detected, including, but not limited to, mammalian (e.g., monkey, mouse, rat, human, bovine), chicken, bacterial, or insect cells, and is preferably a yeast cell. Expression constructs encoding and capable of expressing the binding domain fusion proteins, the transcriptional activation domain fusion proteins, and the reporter gene product(s) are provided within the host cell, by mating of cells containing the expression constructs, or by cell fusion, transformation, electroporation, microinjection, etc.

Various vectors and host strains for expression of the two fusion protein populations in yeast are known and can be used (see e.g., U.S. Pat. No. 5,1468,614; Bartel et al., 1993, “Using the two-hybrid system to detect protein-protein interactions” In: Cellular Interactions in Development, Hartley, ed., Practical Approach Series xviii, IRL Press at Oxford University Press, New York, N.Y., pp. 153-179; Fields and Stemglanz, 1994, Trends In Genetics 10:286-292).

If not already lacking in endogenous reporter gene activity, cells mutant in the reporter gene may be selected by known methods, or the cells can be made mutant in the target reporter gene by known gene-disruption methods prior to introducing the reporter gene (Rothstein, 1983, Meth. Enzymol. 101:202-211).

In a specific embodiment, plasmids encoding the different fusion protein populations can be introduced simultaneously into a single host cell (e.g., a haploid yeast cell) containing one or more reporter genes, by co-transformation, to conduct the assay for protein-protein interactions. Or, preferably, the two fusion protein populations are introduced into a single cell either by mating (e.g., for yeast cells) or cell fusions (e.g., of mammalian cells). In a mating type assay, conjugation of haploid yeast cells of opposite mating type that have been transformed with a binding domain fusion expression construct (preferably a plasmid) and an activation (or inhibitor) domain fusion expression construct (preferably a plasmid), respectively, will deliver both constructs into the same diploid cell. The mating type of a yeast strain may be manipulated by transformation with the HO gene (Herskowitz and Jensen, 1991, Meth. Enzymol. 194:132-146).

In a preferred embodiment, a yeast interaction mating assay is employed using two different types of host cells, strain-type a and alpha of the yeast Saccharomyces cerevisiae. The host cell preferably contains at least two reporter genes, each with one or more binding sites for the DNA-binding domain (e.g., of a transcriptional activator). The activator domain and DNA binding domain are each parts of chimeric proteins formed from the two respective populations of proteins. One strain of host cells, for example the a strain, contains fusions of the library of nucleotide sequences with the DNA-binding domain of a transcriptional activator, such as GAL4. The hybrid proteins expressed in this set of host cells are capable of recognizing the DNA-binding site in the promoter or enhancer region in the reporter gene construct. The second set of yeast host cells, for example, the alpha strain, contains nucleotide sequences encoding fusions of a library of DNA sequences fused to the activation domain of a transcriptional activator.

In another embodiment, the fusion constructs are introduced directly into the yeast chromosome via homologous recombination. The homologous recombination for these purposes is mediated through yeast sequences that are not essential for vegetative growth of yeast, e.g., the MER2, MER1, ZIPI, REC102, or ME14 gene.

Bacteriophage vectors can also be used to express the DNA binding domain and/or activation domain fusion proteins. Libraries can generally be prepared faster and more easily from bacteriophage vectors than from plasmid vectors.

In a specific embodiment, the present invention provides a method of detecting one or more protein-protein interactions comprising (a) recombinantly expressing a novel ubiquitin ligase component of the present invention or a derivative or analog thereof in a first population of yeast cells being of a first mating type and comprising a first fusion protein containing the sequence of a novel ubiquitin ligase component of the present invention and a DNA binding domain, wherein said first population of yeast cells contains a first nucleotide sequence operably linked to a promoter driven by one or more DNA binding sites recognized by said DNA binding domain such that an interaction of said first fusion protein with a second fusion protein, said second fusion protein comprising a transcriptional activation domain, results in increased transcription of said first nucleotide sequence; (b) negatively selecting to eliminate those yeast cells in said first population in which said increased transcription of said first nucleotide sequence occurs in the absence of said second fusion protein; (c) recombinantly expressing in a second population of yeast cells of a second mating type different from said first mating type, a plurality of said second fusion proteins, each second fusion protein comprising a sequence of a fragment, derivative or analog of a protein and an activation domain of a transcriptional activator, in which the activation domain is the same in each said second fusion protein; (d) mating said first population of yeast cells with said second population of yeast cells to form a third population of diploid yeast cells, wherein said third population of diploid yeast cells contains a second nucleotide sequence operably linked to a promoter driven by a DNA binding site recognized by said DNA binding domain such that an interaction of a first fusion protein with a second fusion protein results in increased transcription of said second nucleotide sequence, in which the first and second nucleotide sequences can be the same or different; and (e) detecting said increased transcription of said first and/or second nucleotide sequence, thereby detecting an interaction between a first fusion protein and a second fusion protein.

5.4.2 Assays to Identify F-Box Protein Interactions with Known Proteins Including Potential Substrates

The cellular abundance of cell-cycle regulatory proteins, such as members of the cyclin family or the Cki inhibitory proteins, is regulated by the ubiquitin pathway. The enzymes responsible for the ubiquitination of mammalian cell cycle regulation are not known. In yeast, SCF complexes represent the ubiquitin ligases for cell cycle regulators. The F-box component of the ubiquitin ligase complexes, such as the novel F-box proteins of the invention, determines the specificity of the target of the ubiquitin ligase complex. The invention therefore provides assays to screen known molecules for specific binding to F-box protein nucleic acids, proteins, or derivatives under conditions conducive to binding, and then molecules that specifically bind to the FBP protein are identified.

In a specific embodiment, the invention provides a method for studying the interaction between the F-box protein FBP1 and the Cul1/Skp1 complex, and its role in regulating the stability of β-catenin. Protein-protein interactions can be probed in vivo and in vitro using antibodies specific to these proteins, as described in detail in the experiments in Section 7.

In another specific embodiment, methods for detecting the interaction between Skp2 and p27, a cell cycle regulated cyclin-dependent kinase (Cdk) inhibitor, are provided, as described in Section 8. The interaction between Skp2 and p27 may be targeted to identify modulators of Skp2 activity, including its interaction with cell cycle regulators, such as p27. The ubiquitination of Skp2-specific substrates, such as p27 may be used as a means of measuring the ability of a test compound to modulate Skp2 activity. In another embodiment of the screening assays of the present invention, immunodepletion assays, as described in Section 8, can be used to identify modulators of the Skp2/p27 interaction. In particular, Section 8 describes a method for detection of ubiquitination activity in vitro using p27 as a substrate, which can also be used to identify modulators of the Skp2-dependent ubiquitination of p27. In another embodiment of the screening assays of the present invention, antisense oligonucleotides, as described in Section 5.7.1, can be used as inhibitors of the Skp2 activity. Such identified modulators of p27 ubiquitination/degradation and of the Skp2/p27 interaction can be useful in anti-cancer therapies.

In another specific embodiment, methods for detecting the interaction between Skp2 and Cks1 and Skp2, Cks1, and p27 are provided. The interaction between Skp2 and Cks 1, and Skp2, Cks1 and p27 may be targeted to identify modulators of Skp2 activity, including its interaction with molecules involved in the cell cycle, such as Cks1 and p27. The ubiquitination of Skp2-specific substrates, such as p27 may be used as a means of measuring the ability of a test compound to modulate Skp2 activity in the presence or absence of Cks1. Section 9 describes another embodiment of the screening assays of the present invention for detection of ubiquitination activity by Skp2 with or without Cks1 in vitro using p27 or a phospho-peptide corresponding to the carboxy terminus of p27 with or without a phosphothreonine at position 187 as a substrate, which can also be used to identify modulators of the Skp2-dependent ubiquitination of p27. In another embodiment of the screening assays of the present invention, antisense oligonucleotides, as described in Section 5.7.1, can be used as inhibitors of the Skp2 activity. Such identified modulators of p27 ubiquitination/degradation and of the Skp2/Cks1/p27 interaction can be useful in anti-cancer therapies.

In another specific embodiment, the invention provides for a method for detecting the interaction between the F-box protein Skp2 and E2F-1, a transcription factor involved in cell cycle progression. Insect cells can be infected with baculoviruses co-expressing Skp2 and E2F-1, and cell extracts can be prepared and analyzed for protein-protein interactions. As described in detail in Section 10, this assay has been used successfully to identify potential targets, such as E2F, for known F-box proteins, such as Skp2. This assay can be used to identify other Skp2 targets, as well as targets for novel F-box proteins.

The invention further provides methods for screening ubiquitin ligase complexes having novel F-box proteins (or fragments thereof) as one of their components for ubiquitin ligase activity using known cell-cycle regulatory molecules as potential substrates for ubiquitination. For example, cells engineered to express FBP nucleic acids can be used to recombinantly produce FBP proteins either wild-type or dominant negative mutants in cells that also express a putative ubiquitin-ligase substrate molecule. Such candidates for substrates of the novel FBP of the present invention include, but are not limited to, such potential substrates as IKBα, β-catenin, myc, E2F-1, p27, p21, cyclin A, cyclin B, cycD1, cyclin E and p53. Then the extracts can be used to test the association of F-box proteins with their substrates, (by Western blot immunoassays) and whether the presence of the FBP increases or decreases the level of the potential substrates.

5.5 Assays for the Identification of Compounds that Modulate the Activity of F-Box Proteins

The present invention relates to in vitro and in vivo assay systems described in the subsections below, which can be used to identify compounds or compositions that modulate the interaction of known FBPs with novel substrates and novel components of the ubiquitin ligase complex. The screening assays of the present invention may also be used to identify compounds or compositions that modulate the interaction of novel FBPs with their identified substrates and components of the ubiquitin ligase complex.

Methods to screen potential agents for their ability to disrupt or moderate FBP expression and activity can be designed based on the Applicants' discovery of novel FBPs and their interaction with other components of the ubiquitin ligase complex as well as its known and potential substrates. For example, candidate compounds can be screened for their ability to modulate the interaction of an FBP and Skp1, or the specific interactions of Skp2 with E2F-1, Skp2 with Cks1, Skp2 with Cks1 and p27, or the FBP1/Cul1/Skp1 complex with β-catenin. In principle, many methods known to those of skill in the art, can be readily adapted in designed the assays of the present invention.

The screening assays of the present invention also encompass high-throughput screens and assays to identify modulators of FBP expression and activity. In accordance with this embodiment, the systems described below may be formulated into kits. To this end, cells expressing FBP and components of the ubiquitination ligase complex and the ubiquitination pathway, or cell lysates, thereof can be packaged in a variety of containers, e.g., vials, tubes, microtitre well plates, bottles, and the like. Other reagents can be included in separate containers and provided with the kit; e.g., positive control samples, negative control samples, buffers, cell culture media, etc.

The invention provides screening methodologies useful in the identification of proteins and other compounds which bind to, or otherwise directly interact with, the FBP genes and their gene products. Screening methodologies are well known in the art (see e.g., PCT International Publication No. WO 96/34099, published Oct. 31, 1996, which is incorporated by reference herein in its entirety). The proteins and compounds include endogenous cellular components which interact with the identified genes and proteins in vivo and which, therefore, may provide new targets for pharmaceutical and therapeutic interventions, as well as recombinant, synthetic, and otherwise exogenous compounds which may have binding capacity and, therefore, may be candidates for pharmaceutical agents. Thus, in one series of embodiments, cell lysates or tissue homogenates may be screened for proteins or other compounds which bind to one of the normal or mutant FBP genes and FBP proteins.

Alternatively, any of a variety of exogenous compounds, both naturally occurring and/or synthetic (e.g., libraries of small molecules or peptides), may be screened for binding capacity. All of these methods comprise the step of mixing an FBP protein or fragment with test compounds, allowing time for any binding to occur, and assaying for any bound complexes. All such methods are enabled by the present disclosure of substantially pure FBP proteins, substantially pure functional domain fragments, fusion proteins, antibodies, and methods of making and using the same.

5.5.1 Assays for F-Box Protein Agonists and Antagonists

FBP nucleic acids, F-box proteins, and derivatives can be used in screening assays to detect molecules that specifically bind to FBP nucleic acids, proteins, or derivatives and thus have potential use as agonists or antagonists of FBPs, in particular, molecules that thus affect cell proliferation. In a preferred embodiment, such assays are performed to screen for molecules with potential utility as anti-cancer drugs or lead compounds for drug development. The invention thus provides assays to detect molecules that specifically bind to FBP nucleic acids, proteins, or derivatives. For example, recombinant cells expressing FBP nucleic acids can be used to recombinantly produce FBP proteins in these assays, to screen for molecules that bind to an FBP protein. Similar methods can be used to screen for molecules that bind to FBP derivatives or nucleic acids. Methods that can be used to carry out the foregoing are commonly known in the art. The assays of the present invention may be first optimized on a small scale (i.e., in test tubes), and then scaled up for high-throughput assays. The screening assays of the present may be performed in vitro, i.e. in test tubes, using purified components or cell lysates. The screening assays of the present invention may also be carried out in intact cells in culture and in animal models. In accordance with the present invention, test compounds which are shown to modulate the activity of the FBP as described herein in vitro, will further be assayed in vivo, including cultured cells and animal models to determine if the test compound has the similar effects in vivo and to determine the effects of the test compound on cell cycle progression, the accumulation or degradation of positive and negative regulators, cellular proliferation etc.

In accordance with the present invention, screening assays may be designed to detect molecules which act as agonists or antagonists of the activity of the novel F-box proteins. In accordance with this aspect of the invention, the test compound may be added to an assay system to measure its effect on the activity of the novel FBP, i.e., ubiquitination of its substrates, interaction with other components of the ubiquitin ligase complex, etc. These assays should be conducted both in the presence and absence of the test compound.

In accordance with the present invention, ubiquitination activity of a novel FBP in the presence or absence of a test compound can be measured in vitro using purified components of the ubiquitination pathway or may be measured using crude cellular extracts obtained from tissue culture cells or tissue samples. In another embodiment of the aspect of the present invention the screening may be performed by adding the test agent to in vitro translation systems such as a rabbit reticulocyte lysate (RRL) system and then proceeding with the established analysis. As another alternative, purified or partially purified components which have been determined to interact with one another by the methods described above can be placed under conditions in which the interaction between them would normally occur, with and without the addition of the test agent, and the procedures previously established to analyze the interaction can be used to assess the impact of the test agent. In this approach, the purified or partially purified components may be prepared by fractionation of extracts of cells expressing the components of the ubiquitin ligase complex and pathway, or they may be obtained by expression of cloned genes or cDNAs or fragments thereof, optionally followed by purification of the expressed material.

Within the broad category of in vitro selection methods, several types of method are likely to be particularly convenient and/or useful for screening test agents. These include but are not limited to methods which measure a binding interaction between two or more components of the ubiquitin ligase complex or interaction with the target substrate, methods which measure the activity of an enzyme which is one of the interacting components, and methods which measure the activity or expression of “reporter” protein, that is, an enzyme or other detectable or selectable protein, which has been placed under the control of one of the components.

Binding interactions between two or more components can be measured in a variety of ways. One approach is to label one of the components with an easily detectable label, place it together with the other component(s) in conditions under which they would normally interact, perform a separation step which separates bound labeled component from unbound labeled component, and then measure the amount of bound component. The effect of a test agent included in the binding reaction can be determined by comparing the amount of labeled component which binds in the presence of this agent to the amount which binds in its absence.

In another embodiment, screening can be carried out by contacting the library members with an FBP protein (or nucleic acid or derivative) immobilized on a solid phase and harvesting those library members that bind to the protein (or nucleic acid or derivative). Examples of such screening methods, termed “panning” techniques are described by way of example in Parmley & Smith, 1988, Gene 73:305-318; Fowlkes et al., 1992, BioTechniques 13:422-427; PCT Publication No. WO 94/18318; and in references cited hereinabove.

In another embodiment, the two-hybrid system for selecting interacting proteins or peptides in yeast (Fields & Song, 1989, Nature 340:245-246; Chien et al., 1991, Proc. Natl. Acad. Sci. USA 88:9578-9582) can be used to identify molecules that specifically bind to an FBP protein or derivative.

Alternatively, test methods may rely on measurements of enzyme activity, such as ubiquitination of the target substrate. Once a substrate of a novel FBP is identified or a novel putative substrate of a known FBP is identified, such as the novel substrates of Skp2, E2F and p27, these components may be used in assays to determine the effect of a test compound on the ubiquitin ligase activity of the ubiquitin ligase complex.

In one embodiment, the screening assays may be conducted with a purified system in the presence and absence of test compound. Purified substrate is incubated together with purified ubiquitin ligase complex, ubiquitin conjugating enzymes, ubiquitin activating enzymes and ubiquitin in the presence or in the absence of test compound. Ubiquitination of the substrate is analyzed by immunoassay (see Pagano et al., 1995, Science 269:682-685). Briefly, ubiquitination of the substrate can be performed in vitro in reactions containing 50-200 ng of proteins in 50 mM Tris pH 7.5, 5 mM MgCl2, 2 mM ATPγ-S, 0.1 mM DTT and 5 μM of biotinylated ubiquitin. Total reactions (30 μl) can be incubated at 25° C. for up to 3 hours in the presence or absence of test compound and then loaded on an 8% SDS gel or a 4-20% gradient gel for analysis. The gels are run and proteins are electrophoretically transferred to nitrocellulose. Ubiquitination of the substrate can be detected by immunoblotting. Ubiquitinated substrates can be visualized using Extravidin-HRP (Sigma), or by using a substrate-specific antibody, and the ECL detection system (NEN).

In another embodiment, ubiquitination of the substrate may be assayed in intact cells in culture or in animal models in the presence and absence of the test compound. For example, the test compound may be administered directly to an animal model or to crude extracts obtained from animal tissue samples to measure ubiquitination of the substrate in the presence and absence of the test compounds. For these assays, host cells to which the test compound is added may be genetically engineered to express the FBP components of the ubiquitin ligase pathway and the target substrate, the expression of which may be transient, induced or constitutive, or stable. For the purposes of the screening methods of the present invention, a wide variety of host cells may be used including, but not limited to, tissue culture cells, mammalian cells, yeast cells, and bacteria. Each cell type has its own set of advantages and drawbacks. Mammalian cells such as primary cultures of human tissue cells may be a preferred cell type in which to carry out the assays of the present invention, however these cell types are sometimes difficult to cultivate. Bacteria and yeast are relatively easy to cultivate but process proteins differently than mammalian cells. This ubiquitination assay may be conducted as follows: first, the extracts are prepared from human or animal tissue. To prepare animal tissue samples preserving ubiquitinating enzymes, 1 g of tissue can be sectioned and homogenized at 15,000 r.p.m. with a Brinkmann Polytron homogenizer (PT 3000, Westbury, N.Y.) in 1 ml of ice-cold double-distilled water. The sample is frozen and thawed 3 times. The lysate is spun down at 15,000 r.p.m. in a Beckman JA-20.1 rotor (Beckman Instruments, Palo Alto, Calif.) for 45 min at 4° C. The supernatant is retrieved and frozen at −80° C. This method of preparation of total extract preserves ubiquitinating enzymes (Loda et al. 1997, Nature Medicine 3:231-234, incorporated by reference herein in its entirety).

Purified recombinant substrate is added to the assay system and incubated at 37° C. for different times in 30 μl of ubiquitination mix containing 100 μg of protein tissue homogenates, 50 mM Tris-HCl (pH 8.0), 5 mM MgCl2, and 1 mM DTT, 2 mM ATP, 10 mM creatine phosphokinase, 10 mM creatine phosphate and 5 μM biotinylated ubiquitin. The substrate is then re-purified with antibodies or affinity chromatography. Ubiquitination of the substrate is measured by immunoassays with either antibodies specific to the substrates or with Extravidin-HRP.

In addition, Drosophila can be used as a model system in order to detect genes that phenotypically interact with FBP. For example, overexpression of FBP in Drosophila eye leads to a smaller and rougher eye. Mutagenesis of the fly genome can be performed, followed by selecting flies in which the mutagenesis has resulted in suppression or enhancement of the small rough eye phenotype; the mutated genes in such flies are likely to encode proteins that interact/bind with FBP. Active compounds identified with methods described above will be tested in cultured cells and/or animal models to test the effect of blocking in vivo FBP activity (e.g. effects on cell proliferation, accumulation of substrates, etc.).

In various other embodiments, screening the can be accomplished by one of many commonly known methods. See, e.g., the following references, which disclose screening of peptide libraries: Parmley & Smith, 1989, Adv. Exp. Med. Biol. 251:215-218; Scott & Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar & Pabo, 1993, Science 263:671-673; and PCT Publication No. WO 94/18318.

Compounds, peptides, and small molecules can be used in screening assays to identify candidate agonists and antagonists. In one embodiment, peptide libraries may be used to screen for agonists or antagonists of the FBP of the present invention diversity libraries, such as random or combinatorial peptide or non-peptide libraries can be screened for molecules that specifically bind to FBP. Many libraries are known in the art that can be used, e.g., chemically synthesized libraries, recombinant (e.g., phage display libraries), and in vitro translation-based libraries.

Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott & Smith, 1990, Science 249:386-390; Devlin et al., 1990, Science, 249:404-406; Christian, et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

In vitro translation-based libraries include but are not limited to those described in PCT Publication No. WO 91/05058 dated Apr. 18, 1991; and Mattheakis et al., 1994, Proc. Natl. Acad. Sci. USA 91:9022-9026.

By way of examples of non-peptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

5.5.2 Assays for the Identification of Compounds that Modulate the Interaction of F-Box Proteins with Other Proteins

Once a substrate or interacting protein is identified, as described in detail in Section 5.4, then one can assay for modulators of the F-box protein interaction with such a protein. The present invention provides for methods of detecting agonists and antagonists of such interactions.

In one embodiment, the invention encompasses methods to identify modulators, such as inhibitors or agonists, of the interaction between the F-box protein Skp2 and E2F-1, identified in Section 7 and FIG. 10. Such methods comprise both in vivo and in vitro assays for modulator activity. For example, in an in vivo assay, insect cells can be co-infected with baculoviruses co-expressing Skp2 and E2F-1 as well as potential modulators of the Skp2/E2F-1 interaction. The screening methods of the present invention encompass in vitro assays which measure the ability of a test compound to inhibit the enzymatic activity of Skp2 as described above in Section 5.5.1. Cell extracts can be prepared and analyzed for protein-protein interactions by gel electrophoresis and detected by immunoblotting, as described in detail in Section 7 and presented in FIG. 10. Alternatively, an in vitro protein-protein interaction assay can be used. Recombinant purified Skp2, E2F-1, and putative agonist or antagonist molecules can be incubated together, under conditions that allow binding to occur, such as 37 C for 30 minutes. Protein-protein complex formation can be detected by gel analysis, such as those described herein in Section 7. This assay can be used to identify modulators of interactions of known FBP, such as Skp2 with novel substrates.

In another embodiment, the invention provides for a method for identification of modulators of F-box protein/Skp1 interaction. Such agonist and antagonists can be identified in vivo or in vitro. For example, in an in vitro assay to identify modulators of F-box protein/Skp1 interactions, purified Skp1 and the novel FBP can be incubated together, under conditions that allow binding occur, such as 37C for 30 minutes. In a parallel reaction, a potential agonist or antagonist, as described above in Section 5.5.1, is added either before or during the box protein/Skp1 incubation. Protein-protein interactions can be detected by gel analysis, such as those described herein in Section 7. Modulators of FBP activities and interactions with other proteins can be used as therapeutics using the methods described herein, in Section 5.7.

These assays may be carried out utilizing any of the screening methods described herein, including the following in vitro assay. The screening can be performed by adding the test agent to intact cells which express components of the ubiquitin pathway, and then examining the component of interest by whatever procedure has been established. Alternatively, the screening can be performed by adding the test agent to in vitro translation reactions and then proceeding with the established analysis. As another alternative, purified or partially purified components which have been determined to interact with one another by the methods described above can be placed under conditions in which the interaction between them would normally occur, with and without the addition of the test agent, and the procedures previously established to analyze the interaction can be used to assess the impact of the test agent. In this approach, the purified or partially purified components may be prepared by fractionation of extracts of cells expressing the components of the ubiquitin ligase complex and pathway, or they may be obtained by expression of cloned genes or cDNAs or fragments thereof, optionally followed by purification of the expressed material.

Within the broad category of in vitro selection methods, several types of method are likely to be particularly convenient and/or useful for screening test agents. These include but are not limited to methods which measure a binding interaction between two or more components of the ubiquitin ligase complex or interaction with the target substrate, methods which measure the activity of an enzyme which is one of the interacting components, and methods which measure the activity or expression of “reporter” protein, that is, an enzyme or other detectable or selectable protein, which has been placed under the control of one of the components.

Binding interactions between two or more components can be measured in a variety of ways. One approach is to label one of the components with an easily detectable label, place it together with the other component(s) in conditions under which they would normally interact, perform a separation step which separates bound labeled component from unbound labeled component, and then measure the amount of bound component. The effect of a test agent included in the binding reaction can be determined by comparing the amount of labeled component which binds in the presence of this agent to the amount which binds in its absence.

The separation step in this type of procedure can be accomplished in various ways. In one approach, (one of) the binding partner(s) for the labeled component can be immobilized on a solid phase prior to the binding reaction, and unbound labeled component can be removed after the binding reaction by washing the solid phase. Attachment of the binding partner to the solid phase can be accomplished in various ways known to those skilled in the art, including but not limited to chemical cross-linking, non-specific adhesion to a plastic surface, interaction with an antibody attached to the solid phase, interaction between a ligand attached to the binding partner (such as biotin) and a ligand-binding protein (such as avidin or streptavidin) attached to the solid phase, and so on.

Alternatively, the separation step can be accomplished after the labeled component had been allowed to interact with its binding partner(s) in solution. If the size differences between the labeled component and its binding partner(s) permit such a separation, the separation can be achieved by passing the products of the binding reaction through an ultrafilter whose pores allow passage of unbound labeled component but not of its binding partner(s) or of labeled component bound to its partner(s). Separation can also be achieved using any reagent capable of capturing a binding partner of the labeled component from solution, such as an antibody against the binding partner, a ligand-binding protein which can interact with a ligand previously attached to the binding partner, and so on.

5.6 Methods and Compositions for Diagnostic Use of F-Box Proteins, Derivatives, and Modulators

Cell cycle regulators are the products of oncogenes (cyclins, β-catenin, etc.), or tumor suppressor genes (ckis, p53, etc.) The FBPs, part of ubiquitin ligase complexes, might therefore be products of oncogenes or tumor suppressor genes, depending on which cell cycle regulatory proteins for which they regulate cellular abundance.

FBP proteins, analogues, derivatives, and subsequences thereof, FBP nucleic acids (and sequences complementary thereto), anti-FBP antibodies, have uses in diagnostics. The FBP and FBP nucleic acids can be used in assays to detect, prognose, or diagnose proliferative or differentiative disorders, including tumorigenesis, carcinomas, adenomas etc. The novel FBP nucleic acids of the present invention are located at chromosome sites associated with karyotypic abnormalities and loss of heterozygosity. The FBP1 nucleic acid of the present invention is mapped and localized to chromosome position 10q24, the loss of which has been demonstrated in 10% of human prostate tumors and small cell lung carcinomas (SCLC), suggesting the presence of a tumor suppressor gene at this location. In addition, up to 7% of childhood acute T-cell leukemia is accompanied by a translocation involving 10q24 as a breakpoint, either t(10;14)(q24;q11) or t(7;10)(q35;q24). 9q34 region (where FBP2 is located) has been shown to be a site of loss of heterozygosity (LOH) in human ovarian and bladder cancers. The FBP2 nucleic acid of the present invention is mapped and localized to chromosome position 9q34 which has been shown to be a site of loss of heterozygosity (LOH) in human ovarian and bladder cancers. The FBP3 nucleic acid of the present invention is mapped and localized to chromosome position 13q22, a region known to contain a putative tumor suppressor gene with loss of heterozygosity in approx. 75% of human SCLC. The FBP4 nucleic acid of the present invention is mapped and localized to chromosome position 5p12, a region shown to be a site of karyotypic abnormalities in a variety of tumors, including human breast cancer and nasopharyngeal carcinomas. The FBP5 nucleic acid of the present invention is mapped and localized to chromosome position 6q25-26, a region shown to be a site of loss of heterozygosity in human ovarian, breast and gastric cancers hepatocarcinomas, Burkitt's lymphomas, gliomas, and parathyroid adenomas. The FBP7 nucleic acid of the present invention is mapped and localized to chromosome position 15q15 a region which contains a tumor suppressor gene associated with progression to a metastatic stage in breast and colon cancers and a loss of heterozygosity in parathyroid adenomas.

The molecules of the present invention can be used in assays, such as immunoassays, to detect, prognose, diagnose, or monitor various conditions, diseases, and disorders affecting FBP expression, or monitor the treatment thereof. In particular, such an immunoassay is carried out by a method comprising contacting a sample derived from a patient with an anti-FBP antibody under conditions such that immunospecific binding can occur, and detecting or measuring the amount of any immunospecific binding by the antibody. In a specific aspect, such binding of antibody, in tissue sections, can be used to detect aberrant FBP localization or aberrant (e.g., low or absent) levels of FBP. In a specific embodiment, antibody to FBP can be used to assay a patient tissue or serum sample for the presence of FBP where an aberrant level of FBP is an indication of a diseased condition. By “aberrant levels,” is meant increased or decreased levels relative to that present, or a standard level representing that present, in an analogous sample from a portion of the body or from a subject not having the disorder.

The immunoassays which can be used include but are not limited to competitive and non-competitive assay systems using techniques such as western blots, immunohisto-chemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few.

FBP genes and related nucleic acid sequences and subsequences, including complementary sequences, can also be used in hybridization assays. FBP nucleic acid sequences, or subsequences thereof comprising about at least 8 nucleotides, can be used as hybridization probes. Hybridization assays can be used to detect, prognose, diagnose, or monitor conditions, disorders, or disease states associated with aberrant changes in FBP expression and/or activity as described supra. In particular, such a hybridization assay is carried out by a method comprising contacting a sample containing nucleic acid with a nucleic acid probe capable of hybridizing to FBP DNA or RNA, under conditions such that hybridization can occur, and detecting or measuring any resulting hybridization.

In specific embodiments, diseases and disorders involving overproliferation of cells can be diagnosed, or their suspected presence can be screened for, or a predisposition to develop such disorders can be detected, by detecting decreased levels of FBP protein, FBP RNA, or FBP functional activity (e.g., ubiquitin ligase target binding activity, F-box domain binding activity, ubiquitin ligase activity etc.), or by detecting mutations in FBP RNA, DNA or FBP protein (e.g., translocations in FBP nucleic acids, truncations in the FBP gene or protein, changes in nucleotide or amino acid sequence relative to wild-type FBP) that cause decreased expression or activity of FBP. Such diseases and disorders include but are not limited to those described in Section 5.7.3. By way of example, levels of FBP protein can be detected by immunoassay, levels of FBP RNA can be detected by hybridization assays (e.g., Northern blots, in situ-hybridization), FBP activity can be assayed by measuring ubiquitin ligase activity in E3 ubiquitin ligase complexes formed in vivo or in vitro, F-box domain binding activity can be assayed by measuring binding to Skp1 protein by binding assays commonly known in the art, translocations, deletions and point mutations in FBP nucleic acids can be detected by Southern blotting, FISH, RFLP analysis, SSCP, PCR using primers that preferably generate a fragment spanning at least most of the FBP gene, sequencing of FBP genomic DNA or cDNA obtained from the patient, etc.

In a preferred embodiment, levels of FBP mRNA or protein in a patient sample are detected or measured, in which decreased levels indicate that the subject has, or has a predisposition to developing, a malignancy or hyperproliferative disorder; in which the decreased levels are relative to the levels present in an analogous sample from a portion of the body or from a subject not having the malignancy or hyperproliferative disorder, as the case may be.

In another specific embodiment, diseases and disorders involving a deficiency in cell proliferation or in which cell proliferation is desirable for treatment, are diagnosed, or their suspected presence can be screened for, or a predisposition to develop such disorders can be detected, by detecting increased levels of FBP protein, FBP RNA, or FBP functional activity (e.g., ubiquitin ligase activity, Skp1 binding activity, etc.), or by detecting mutations in FBP RNA, DNA or protein (e.g., translocations in FBP nucleic acids, truncations in the gene or protein, changes in nucleotide or amino acid sequence relative to wild-type FBP) that cause increased expression or activity of FBP. Such diseases and disorders include but are not limited to those described in Section 5.7.3. By way of example, levels of FBP protein, levels of FBP RNA, ubiquitin ligase activity, FBP binding activity, and the presence of translocations or point mutations can be determined as described above.

In a specific embodiment, levels of FBP mRNA or protein in a patient sample are detected or measured, in which increased levels indicate that the subject has, or has a predisposition to developing, a growth deficiency or degenerative or hypoproliferative disorder; in which the increased levels are relative to the levels present in an analogous sample from a portion of the body or from a subject not having the growth deficiency, degenerative, or hypoproliferative disorder, as the case may be.

Kits for diagnostic use are also provided, that comprise in one or more containers an anti-FBP antibody, and, optionally, a labeled binding partner to the antibody. Alternatively, the anti-FBP antibody can be labeled (with a detectable marker, e.g., a chemiluminescent, enzymatic, fluorescent, or radioactive moiety). A kit is also provided that comprises in one or more containers a nucleic acid probe capable of hybridizing to FBP RNA. In a specific embodiment, a kit can comprise in one or more containers a pair of primers (e.g., each in the size range of 6-30 nucleotides) that are capable of priming amplification [e.g., by polymerase chain reaction (see e.g., Innis et al., 1990, PCR Protocols, Academic Press, Inc., San Diego, Calif.), ligase chain reaction (see EP 320,308) use of Q replicase, cyclic probe reaction, or other methods known in the art] under appropriate reaction conditions of at least a portion of a FBP nucleic acid. A kit can optionally further comprise in a container a predetermined amount of a purified FBP protein or nucleic acid, e.g., for use as a standard or control.

5.7 Methods and Compositions for Therapeutic Use of F-Box Proteins, Derivatives, and Modulators

Described below are methods and compositions for the use of F-box proteins in the treatment of proliferative disorders and oncogenic disease symptoms may be ameliorated by compounds that activate or enhance FBP activity, and whereby proliferative disorders and cancer may be ameliorated.

In certain instances, compounds and methods that increase or enhance the activity of an FBP can be used to treat proliferative and oncogenic disease symptoms. Such a case may involve, for example, a proliferative disorder that is brought about, at least in part, by a reduced level of FBP gene expression, or an aberrant level of an FBP gene product's activity. For example, decreased activity or under-expression of an FBP component of a ubiquitin ligase complex whose substrate is a positive cell-cycle regulator, such as a member of the Cyclin family, will result in increased cell proliferation. As such, an increase in the level of gene expression and/or the activity of such FBP gene products would bring about the amelioration of proliferative disease symptoms.

In another instance, compounds that increase or enhance the activity of an FBP can be used to treat proliferative and oncogenic disease symptoms resulting from defects in the expression or activity of other genes and gene products involved in cell cycle control, such as FBP substrate molecules. For example, an increase in the expression or activity of a positive cell-cycle positive molecule, such as a member of the Cyclin family, may result in its over-activity and thereby lead to increased cell proliferation. Compounds that increase the expression or activity of the FBP component of a ubiquitin ligase complex whose substrate is such a cell-cycle positive regulator will lead to ubiquitination of the defective molecule, and thereby result in an increase in its degradation. Disease symptoms resulting from such a defect may be ameliorated by compounds that compensate the disorder by increased FBP activity. Techniques for increasing FBP gene expression levels or gene product activity levels are discussed in Section 5.7, below.

Alternatively, compounds and methods that reduce or inactivate FBP activity may be used therapeutically to ameliorate proliferative and oncogenic disease symptoms. For example, a proliferative disorder may be caused, at least in part, by a defective FBP gene or gene product that leads to its overactivity. Where such a defective gene product is a component of a ubiquitin ligase complex whose target is a cell-cycle inhibitor molecule, such as a Cki, an overactive FBP will lead to a decrease in the level of cell-cycle molecule and therefore an increase in cell proliferation. In such an instance, compounds and methods that reduce or inactivate FBP function may be used to treat the disease symptoms.

In another instance, compounds and methods that reduce the activity of an FBP can be used to treat disorders resulting from defects in the expression or activity of other genes and gene products involved in cell cycle control, such as FBP substrate molecules. For example, a defect in the expression or activity of a cell-cycle negative regulatory molecule, such as a Cki, may lead to its under-activity and thereby result in increased cell proliferation. Reduction in the level and/or activity of an FBP component whose substrate was such molecule would decrease the ubiquitination and thereby increase the level of such a defective molecule. Therefore, compounds and methods aimed at reducing the expression and/or activity of such FBP molecules could thereby be used in the treatment of disease symptoms by compensating for the defective gene or gene product.

Techniques for the reduction of target gene expression levels or target gene product activity levels are discussed in Section 5.7 below.

5.7.1 Therapeutic Use of Inhibitory Antisense, Ribozyme and Triple Helix Molecules and Identified Agonists and Antagonists

In another embodiment, symptoms of certain FBP disorders, such as such as proliferative or differentiative disorders causing tumorigenesis or cancer, may be ameliorated by decreasing the level of FBP gene expression and/or FBP gene product activity by using FBP gene sequences in conjunction with well-known antisense, gene “knock-out” ribozyme and/or triple helix methods to decrease the level of FBP gene expression. Among the compounds that may exhibit the ability to modulate the activity, expression or synthesis of the FBP gene, including the ability to ameliorate the symptoms of an FBP disorder, such as cancer, are antisense, ribozyme, and triple helix molecules. Such molecules may be designed to reduce or inhibit either unimpaired, or if appropriate, mutant target gene activity. Techniques for the production and use of such molecules are well known to those of skill in the art. For example, antisense targeting SKP2 mRNA stabilize the Skp2-substrate p27, as described in Section X (Figure X).

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of the FBP gene could be used in an antisense approach to inhibit translation of endogenous FBP mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

In an embodiment of the present invention, oligonucleotides complementary to the nucleic acids encoding the F-box motif as indicated in FIGS. 2 and 4-9.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86, 6553-6556; Lemaitre, et al., 1987, Proc. Natl. Acad. Sci. 84, 648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6, 958-976) or intercalating agents (see, e.g., Zon, 1988, Pharm. Res. 5, 539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate (S-ODNs), a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an-anomeric oligonucleotide. An-anomeric oligonucleotide forms specific double-stranded hybrids with complementary RNA in which, contrary to the usual-units, the strands run parallel to each other (Gautier, et al., 1987, Nucl. Acids Res. 15, 6625-6641). The oligonucleotide is a 2-O-methylribonucleotide (Inoue, et al., 1987, Nucl. Acids Res. 15, 6131-6148), or a chimeric RNA-DNA analogue (Inoue, et al., 1987, FEBS Lett. 215, 327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16, 3209), methylphosphonate oligonucleotides can be prepared by use of controlled pore glass polymer supports (Sarin, et al., 1988, Proc. Natl. Acad. Sci. U.S.A. 85, 7448-7451), etc.

While antisense nucleotides complementary to the target gene coding region sequence could be used, those complementary to the transcribed, untranslated region are most preferred.

In one embodiment of the present invention, gene expression downregulation is achieved because specific target mRNAs are digested by RNAse H after they have hybridized with the antisense phosphorothioate oligonucleotides (S-ODNs). Since no rules exist to predict which antisense S-ODNs will be more successful, the best strategy is completely empirical and consists of trying several antisense S-ODNs. Antisense phosphorothioate oligonucleotides (S-ODNs) will be designed to target specific regions of mRNAs of interest. Control S-ODNs consisting of scrambled sequences of the antisense S-ODNs will also be designed to assure identical nucleotide content and minimize differences potentially attributable to nucleic acid content. All S-ODNs will be synthesized by Oligos Etc. (Wilsonville, Oreg.). In order to test the effectiveness of the antisense molecules when applied to cells in culture, such as assays for research purposes or ex vivo gene therapy protocols, cells will be grown to 60-80% confluence on 100 mm tissue culture plates, rinsed with PBS and overlaid with lipofection mix consisting of 8 ml Opti-MEM, 52.8 l Lipofectin, and a final concentration of 200 nM S-ODNs. Lipofections will be carried out using Lipofectin Reagent and Opti-MEM (Gibco BRL). Cells will be incubated in the presence of the lipofection mix for 5 hours. Following incubation the medium will be replaced with complete DMEM. Cells will be harvested at different time points post-lipofection and protein levels will be analyzed by Western blot.

Antisense molecules should be targeted to cells that express the target gene, either directly to the subject in vivo or to cells in culture, such as in ex vivo gene therapy protocols. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290, 304-310), the promoter contained in the 3 long terminal repeat of Rous sarcoma virus (Yamamoto, et al., 1980, Cell 22, 787-797), the herpes thymidine kinase promoter (Wagner, et al., 1981, Proc. Natl. Acad. Sci. U.S.A. 78, 1441-1445), the regulatory sequences of the metallothionein gene (Brinster, et al., 1982, Nature 296, 39-42), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product (see, e.g., PCT International Publication WO90/11364, published Oct. 4, 1990; Sarver, et al., 1990, Science 247, 1222-1225). In an embodiment of the present invention, oligonucleotides which hybridize to the FBP gene are designed to be complementary to the nucleic acids encoding the F-box motif as indicated in FIGS. 2 and 4-9.

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff & Gerlach, 1988, Nature, 334, 585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been & Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al., 1985, Nature 317, 230-234; Thomas & Capecchi, 1987, Cell 51, 503-512; Thompson, et al., 1989, Cell 5, 313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited modifications to ES (embryonic stem) cells can be used to generate animal offspring with an inactive target gene (e.g., see Thomas & Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triple helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods such as those described, below, in Section 5.7.2 that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

5.7.2 Gene Replacement Therapy

With respect to an increase in the level of normal FBP gene expression and/or FBP gene product activity, FBP gene nucleic acid sequences, described, above, in Section 5.1 can, for example, be utilized for the treatment of proliferative disorders such as cancer. Such treatment can be administered, for example, in the form of gene replacement therapy. Specifically, one or more copies of a normal FBP gene or a portion of the FBP gene that directs the production of an FBP gene product exhibiting normal FBP gene function, may be inserted into the appropriate cells within a patient, using vectors that include, but are not limited to adenovirus, adeno-associated virus, and retrovirus vectors, in addition to other particles that introduce DNA into cells, such as liposomes.

For FBP genes that are expressed in all tissues or are preferentially expressed, such as FBP1 gene is expressed preferably in the brain, such gene replacement therapy techniques should be capable delivering FBP gene sequences to these cell types within patients. Thus, in one embodiment, techniques that are well known to those of skill in the art (see, e.g., PCT Publication No. WO89/10134, published Apr. 25, 1988) can be used to enable FBP gene sequences to cross the blood-brain barrier readily and to deliver the sequences to cells in the brain. With respect to delivery that is capable of crossing the blood-brain barrier, viral vectors such as, for example, those described above, are preferable.

In another embodiment, techniques for delivery involve direct administration of such FBP gene sequences to the site of the cells in which the FBP gene sequences are to be expressed.

Additional methods that may be utilized to increase the overall level of FBP gene expression and/or FBP gene product activity include the introduction of appropriate FBP-expressing cells, preferably autologous cells, into a patient at positions and in numbers that are sufficient to ameliorate the symptoms of an FBP disorder. Such cells may be either recombinant or non-recombinant.

Among the cells that can be administered to increase the overall level of FBP gene expression in a patient are cells that normally express the FBP gene.

Alternatively, cells, preferably autologous cells, can be engineered to express FBP gene sequences, and may then be introduced into a patient in positions appropriate for the amelioration of the symptoms of an FBP disorder or a proliferative or differentiative disorders, e.g., cancer and tumorigenesis. Alternately, cells that express an unimpaired FBP gene and that are from a MHC matched individual can be utilized, and may include, for example, brain cells. The expression of the FBP gene sequences is controlled by the appropriate gene regulatory sequences to allow such expression in the necessary cell types. Such gene regulatory sequences are well known to the skilled artisan. Such cell-based gene therapy techniques are well known to those skilled in the art, see, e.g., Anderson, U.S. Pat. No. 5,399,349.

When the cells to be administered are non-autologous cells, they can be administered using well known techniques that prevent a host immune response against the introduced cells from developing. For example, the cells may be introduced in an encapsulated form which, while allowing for an exchange of components with the immediate extracellular environment, does not allow the introduced cells to be recognized by the host immune system.

Additionally, compounds, such as those identified via techniques such as those described, above, in Section 5.5, that are capable of modulating FBP gene product activity can be administered using standard techniques that are well known to those of skill in the art. In instances in which the compounds to be administered are to involve an interaction with brain cells, the administration techniques should include well known ones that allow for a crossing of the blood-brain barrier.

5.7.3 Target Proliferative Cell Disorders

With respect to specific proliferative and oncogenic disease associated with ubiquitin ligase activity, the diseases that can be treated or prevented by the methods of the present invention include but are not limited to: human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

Diseases and disorders involving a deficiency in cell proliferation or in which cell proliferation is desired for treatment or prevention, and that can be treated or prevented by inhibiting FBP function, include but are not limited to degenerative disorders, growth deficiencies, hypoproliferative disorders, physical trauma, lesions, and wounds; for example, to promote wound healing, or to promote regeneration in degenerated, lesioned or injured tissues, etc. In a specific embodiment, nervous system disorders are treated. In another specific embodiment, a disorder that is not of the nervous system is treated.

5.8 Pharmaceutical Preparations and Methods of Administration

The compounds that are determined to affect FBP gene expression or gene product activity can be administered to a patient at therapeutically effective doses to treat or ameliorate a cell proliferative disorder. A therapeutically effective dose refers to that amount of the compound sufficient to result in amelioration of symptoms of such a disorder.

5.8.1 Effective Dose

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

5.8.2 Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

6. Example Identification and Characterization of Novel Ubiquitin Ligase F-Box Proteins and Genes

The following studies were carried out to identify novel F-box proteins which may act to recruit novel specific substrates to the ubiquitination pathways. Studies involving several organisms have shown that some FBPs play a crucial role in the controlled degradation of important cellular regulatory proteins (e.g., cyclins, cdk-inhibitors, β-catenin, IKBa, etc.). These FBPs are subunits of ubiquitin protein SCF ligases formed by three basic subunits: a cullin subunit (called Cdc53 in S. cerevisiae and Cul1 in humans); Skp1; and one of many FBPs. SCF ligases target ubiquitin conjugating enzymes (either Ubc3 or Ubc4) to specific substrates which are recruited by different FBPs. Schematically, the Ubc is bound to the ligase through the cullin subunit while the substrate interacts with the FBP subunit. Although FBPs can bind the cullin subunit directly, the presence of fourth subunit, Skp1, which simultaneously can bind the cullin-terminus and the F-box of the FBP, stabilizes the complex. Thus, the substrate specificity of the ubiquitin ligase complex is provided by the F-box subunit.

6.1 Materials and Methods Used for the Identification and Characterization of Novel F-Box Genes

Yeast Two-Hybrid Screening In order to clone the human genes encoding F-box proteins, proteins associated with Skp1 were identified using a modified yeast 2-hybrid system (Vidal et al., 1996, Proc. Nat. Acad. Sci., 93:10315-20; Vidal et al., 1996, Proc. Nat. Acad. Sci., 93:10321-26). This modified system takes advantage of using three reporter genes expressed from three different Gal4 binding site promoters, thereby decreasing the number of false positive interactions. This multiple reporter gene assay facilitates identification of true interactors.

Human Skp1 was used as a bait to search for proteins that interact with Skp1, such as novel F-box proteins and the putative human homolog of Cdc4. The plasmids pPC97-CYH2 and pPC86 plasmids, encoding the DNA binding domain (DB, aa 1-147) and the transcriptional activation domain (AD, aa 768-881) of yeast GAL4, and containing LEU2 and TRP1 as selectable markers, respectively, were used (Chevray and Nathans, 1992, Proc. Nat. Acad. Sci., 89:5789-93; Vidal et al., supra).

An in-frame fusion between Skp1 and DB was obtained by homologous recombination of the PCR product described below. The following 2 oligonucleotides were designed and obtained as purified primers from Gene Link Inc.: 5′-AGT-AGT-AAC-AAA-GGT-CAA-AGA-CAG-TTG-ACT-GTA-TCG-TCG-AGG-ATG-CCT-TCA-ATT AAG-TT (SEQ ID NO: 80); 3′-GCG-GTT-ACT-TAC-TTA-GAG-CTC-GAC-GTC-TTA-CTT-ACT-TAG-CTC-ACT-TCT-CTT-CAC-ACC-A (SEQ ID NO: 81). The 5′ primer corresponds to a sequence located in the DB of the pPC97-CYH2 plasmid (underlined) flanked by the 5′ sequence of the skp1 gene. The 3′ primer corresponds to a sequence located by polylinker of the pPC97—CYH2 plasmid (underlined) flanked by the 3′ sequence of the skp1 gene. These primers were used in a PCR reaction containing the following components: 100 ng DNA template (skp1 pET plasmid), 1 μM of each primer, 0.2 mM dNTP, 2 mM MgCl₂, 10 mM KCl, 20 mM Tris Cl pH 8.0, 0.1% Triton X-100, 6 mM (NH₄)₂ SO₄, 10 μg/ml nuclease-free BSA, 1 unit of Pfu DNA polymerase (4′ at 94° C., 1′ at 50 C, 10′ at 72° C. for 28 cycles). Approximately 100 ng of PCR product were transformed into yeast cells (MaV103 strain; Vidal et al., 1996, Proc. Natl. Acad. Sci. U.S.A. 93:10315-10320; Vidal et al., 1996, Proc. Natl. Acad. Sci. U.S.A. 93:10321-10326) in the presence or in the absence of 100 ng of pPC97-CYH2 plasmid previously digested with BglII and SalI. As a result of the homologous recombination, only yeast cells containing the pPC97-CYH2 plasmid homologously recombined with skp1 cDNA, grew in the absence of leucine. Six colonies were isolated and analyzed by immunoblotting for the expression of Skp1, as described (Vidal et al., supra). All 6 colonies, but not control colonies, expressed a Mr 36,000 fusion-protein that was recognized by our affinity purified anti-Skp1 antibody.

The AD fusions were generated by cloning cDNA fragments in the frame downstream of the AD domains and constructs were confirmed by sequencing, immunoblot, and interaction with Skp1. The pPC86-Skp2s (pPC86) include: pPC86-Skp2, and pPC86-Skp2-CT (aa 181-435 of Skp2). The first fusion represents our positive control since Skp2 is a known interactor of Skp1 (Zhang, et al, 1995, Cell, 82: 915-25); the latter fusion was used as a negative control since it lacked the P-box required for the interaction with Skp1.

MaV103 strain harboring the DB-skp1 fusions was transformed with an activated T-cell cDNA library (Alala 2; Hu, et al., Genes & Dev. 11: 2701-14) in pPC86 using the standard lithium acetate method. Transformants were first plated onto synthetic complete (SC)-Leu-Trp plates, followed by replica plating onto (SC)-Leu-Trp-His plates containing 20 mM 3-aminotriazole (3-AT) after 2 days. Yeast colonies grown out after additional 3-4 days of incubation were picked as primary positives and further tested in three reporter assays: i) growth on SC-Leu-Trp-His plates supplemented with 20 mM 3-AT; ii)-galactosidase activity; and iii) URA3 activation on SC-Leu-Trp plates containing 0.2% 5-fluoroortic acid, as a counterselection method. Of the 3×10⁶ yeast transformants screened AD plasmids were rescued from the fifteen selected positive colonies after all three. MaV103 cells were re-transformed with either rescued AD plasmids and the DBskpl fusion or rescued AD plasmid and the pPC97-CYH2vector without a cDNA insert as control. Eleven AD plasmids from colonies that repeatedly tested positive in all three reporter assays (very strong interactors) and four additional AD plasmids from clones that were positive on some but not all three reporter assays (strong interactors) were recovered and sequenced with the automated ABI 373 DNA sequencing system.

Cloning of full length FBPs Two of the clones encoding FBP4 and FBP5 appeared to be full-length, while full length clones of 4 other cDNAs encoding FBP1, FBP2, FBP3 and FBP7 were obtained with RACE using Marathon-Ready cDNA libraries (Clonthec, cat. # 7406, 7445, 7402) according to the manufacturer's instructions. A full-length clone encoding FBP6 was not obtained. Criteria for full length clones included at least two of the following: i) the identification of an ORF yielding a sequence related to known F-box proteins; ii) the presence of a consensus Kozak translation initiation sequence at a putative initiator methionine codon; iii) the identification of a stop codon in the same reading frame but upstream of the putative initiation codon; iv) the inability to further increase the size of the clone by RACE using three different cDNA libraries.

Analysis by Immunoblotting of Protein from Yeast Extracts Yeast cells were grown to mid-logarithmic phase, harvested, washed and resuspended in buffer (50 mM Tris pH 8.0, 20% glycerol, 1 mM EDTA, 0.1% Triton X-100, 5 mM MgCl2, 10 mM β-mercaptoethanol, 1 mM PMSF, 1 mg/ml Leupeptin, 1 mg/ml Pepstatin) at a cell density of about 109 cells/ml. Cells were disrupted by vortexing in the presence of glass beads for 10 min at 40C. Debris was pelleted by centrifugation at 12,000 RPM for 15 min at 40C. Approximately 50 g of proteins were subjected to immunoblot analysis as described (Vidal et al., 1996a, supra; Vidal et al., 1996b, supra).

DNA database searches and analysis of protein motifs ESTs (expressed sequence tags) with homology to FBP genes were identified using BLAST, PSI-BLAST (http://www.ncbi.nlm.nih.gov/BLAST/) and TGI Sequence Search (http://www.tigr.org/cgi-bin/BlastSearch/blast_tgi.cgi). ESTs that overlapped more than 95% in at least 100 bps were assembled into novel contiguous ORFs using Sequencher 3.0. Protein domains were identified with ProfileScan Server (http://www.isrec.isb-sib.ch/software/PFSCAN_form.html), BLOCKS Sercher (http://www.blocks.fhcrc.org/blocks_search.html) and IMB Jena (http://genome.imb-jena.de/cgi-bin/GDEWWW/menu.cgi).

Construction of F-box mutants Delta-F-box mutants [(ΔF)FBP1, residues 32-179; (ΔF)FBP2, residues 60-101; (ΔF)FBP3a, residues 40-76; (ΔF)FBP4, residues 55-98] were obtained by deletion with the appropriate restriction enzymes with conservation of the reading frame. (ΔF)Skp2 mutant was obtained by removing a DNA fragment (nucleotides 338-997) with BspEI and XbaI restriction enzymes, and replacing it with a PCR fragment containing nucleotides 457 to 997. The final construct encoded a protein lacking residues 113-152. The leucine 51-to-alanine FBP3a mutant [FBP3a(L51A)] and the tryptophan 76-to-alanine FBP3a mutant [FBP3a(W76A)] were generated by oligonucleotide-directed mutagenesis using the polymerase chain reaction of the QuikChange site-directed mutagenesis kit (Stratagene). All mutants were sequenced in their entirety.

Recombinant proteins cDNA fragments encoding the following human proteins: Flag-tagged FBP1, Flag-tagged (ΔF)FBP1, Flag-tagged FBP3a, Skp2, HA-tagged Cul1, HA-tagged Cul2, (β-catenin, His-tagged cyclin D1, Skp1, His-tagged Skp1, His-tagged Elongin C were inserted into the baculovirus expression vector pBacpak-8 (Clonetech) and cotransfected into Sf9 cells with linearized baculovirus DNA using the BaculoGold transfection kit (Pharmingen). Recombinant viruses were used to infect 5B cells and assayed for expression of their encoded protein by immunoblotting as described above. His-proteins were purified with Nickel-agarose (Invitrogen) according to the manufacturer's instructions.

Antibodies. Anti-Cul1 antibodies was generated by injecting rabbits and mice with the following amino acid peptide: (C)DGEKDTYSYLA (SEQ ID NO: 82). This peptide corresponds to the carboxy-terminus of human Cul1 and is not conserved in other cullins. Anti-Cul2 antibodies was generated by injecting rabbits with the following amino acid peptide: (C)ESSFSLNMNFSSKRTKFKITTSMQ (SEQ ID NO: 83). This peptide is located 87 amino acids from the carboxy-terminus of human Cul2 and is not conserved in other cullins. The anti-Skp1 antibody was generated by injecting rabbits with the peptide (C)EEAQVRKENQW (SEQ ID NO: 84), corresponding to the carboxy-terminus of human Skp1. The cysteine residues (C) were added in order to couple the peptides to keyhole limpet hemocyanin (KLH). All of the antibodies were generated, affinity-purified (AP) and characterized as described (Pagano, M., ed., 1995, “From Peptide to Purified Antibody”, in Cell Cycle: Materials and Methods, Spring-Verlag, 217-281). Briefly, peptides whose sequence showed high antigenic index (high hydrophilicity, good surface probability, good flexibility, and good secondary structure) were chosen. Rabbits and mice were injected with peptide-KLH mixed with complete Freund's adjuvant. Subsequently they were injected with the peptide in incomplete Freund's adjuvant, every 2 weeks, until a significant immunoreactivity was detected by immunoprecipitation of 35S-methionine labeled HeLa extract. These antisera recognized bands at the predicted size in both human extracts and a extracts containing recombinant proteins.

Monoclonal antibody (Mab) to Ubc3 was generated and characterized in collaboration with Zymed Inc. Mab to cyclin B (cat # sc-245) was from Santa Cruz; Mabs to p21 (cat # C24420) and p27 (cat # K25020) from Transduction lab. (Mabs) cyclin E, (Faha, 1993, J. of Virology 67: 2456); AP rabbit antibodies to human p27, Skp2, Cdk2 (Pagano, 1992, EMBO J. 11: 761), and cyclin A (Pagano, 1992, EMBO J. 11: 761), and phospho-site p27 specific antibody, were obtained or generated by standard methods. Where indicated, an AP goat antibody to an N-terminal Skp2 peptide (Santa Cruz, cat # sc-1567) was used. Rat anti-HA antibody was from Boehringer Mannheim (cat. #1867423), rabbit anti-HA antibody was from Santa Cruz (cat. # sc-805), mouse anti-Flag antibody was from Kodak (cat. # IB13010), rabbit anti-Flag antibody was from Zymed (cat. #71-5400), anti-Skp1 and anti-(β-catenin mouse antibodies were from Transduction Laboratories (cat. # C19220 and P46020, respectively). The preparation, purification and characterization of a Mab to human cyclin D1 (clone AM29, cat. #33-2500) was performed in collaboration with Zymed Inc. Antiserum to human cyclin D1 was produced as described(Ohtsubo et al., 1995, Mol Cell Biol, 15, 2612-2624).

Extract preparation and cell synchronization Protein extraction was performed as previously described (Pagano, 1993, J. Cell Biol. 121: 101) with the only difference that 1 μm okadaic acid was present in the lysis buffer. Human lung fibroblasts IMR-90 were synchronized in G0/G1 by serum starvation for 48 hours and the restimulated to re-enter the cell cycle by serum readdition. HeLa cells were synchronized by mitotic shake-off as described (Pagano, 1992, EMBO J. 11: 761). Synchronization was monitored by flow cytometry. For in vitro ubiquitination and degradation assays, G1 HeLa cells were obtained with a 48-hour lovastatin treatment and protein extraction performed as described below.

Immunoprecipitation and Immunoblotting. Cell extracts were prepared by addition of 3-5 volumes of standard lysis buffers (Pagano et al., 1992, Science 255, 1144-1147), and conditions for immunoprecipitation were as described (Jenkins and Xiong, 1995; Pagano et al., 1992a Science 255-1144-1147). Proteins were transfered from gel to a nitrocellulose membrance (Novex) by wet blotting as described (Tam et al., 1994 Oncogene 9, 2663). Filters were subjected to immunoblotting using a chemiluminescence (DuPont-NEN) detection system according to the manufacturer's instructions

Protein extraction for in vitro ubiquitination assay Logarithmically growing, HeLa-S3 cells were collected at a density of 6×10⁵ cells/ml. Approx. 4 ml of HeLa S3 cell pellet were suspended in 6 ml of ice-cold buffer consisting of 20 mM Tris-HCl (pH 7.2), 2 mM DTT, 0.25 mM EDTA, 10 μg/ml leupeptin, and 10 μg/ml pepstatin. The suspension was transferred to a cell nitrogen-disruption bomb (Parr, Moline, Ill., cat #4639) that had been rinsed thoroughly and chilled on ice before use. The bomb chamber was connected to a nitrogen tank and the pressure was brought slowly to 1000 psi. The chamber was left on ice under the same pressure for 30 minutes and then the pressure was released slowly. The material was transferred to an Eppendorf tube and centrifuged in a microcentrifuge at 10,000 g for 10 minutes. The supernatant (S-10) was divided into smaller samples and frozen at −800C.

In vitro ubiquitination The ubiquitination assay was performed as described (Lyapina, 1998, Proc Natl Acad Sci USA, 95: 7451). Briefly, immuno-beads containing Flag-tagged FBPs immunoprecipitated with anti-Flag antibody were added with purified recombinant human E1 and E2 enzymes (Ubc2, Ubc3 or Ubc4) to a reaction mix containing biotinylated-ubiquitin. Samples were then analyzed by blotting with HRP-streptavidin. E1 and E2 enzymes and biotinylated-ubiquitin were produced as described (Pagano, 1995, Science 269: 682).

Transient transfections cDNA fragments encoding the following human proteins: FBP1, (ΔF)FBP1, FBP2, (ΔF)FBP2, FBP3a, (ΔF)FBP3a, FBP3a(L51A), FBP3a(W76A), FBP4, (ΔF)FBP4, Skp2, (ΔF)Skp2, HA-tagged β-catenin, untagged β-catenin, Skp1, cyclin D1 were inserted into the mammalian expression vector pcDNA3 (Invitrogen) in frame with a Flag-tag at their C-terminus. Cells were transfected with FuGENE transfection reagent (Boehringer, cat. #1-814-443) according to the manufacture's instruction.

Immunofluorescence Transfected cell monolayers growing on glass coverslips were rinsed in PBS and fixed with 4% paraformaldehyde in PBS for 10 minutes at 4° C. followed by permeabilization for 10 minutes with 0.25% Triton X-100 in PBS. Other fixation protocols gave comparable results. Immunofluorescence stainings were performed using 1 μg/ml rabbit anti-Flag antibody as described (Pagano, 1994, Genes & Dev., 8:1627).

Northern Blot Analysis Northern blots were performed using human multiple-tissue mRNAs from Clontech Inc. Probes were radiolabeled with [alpha-32P] dCTP (Amersham Inc.) using a random primer DNA labeling kit (Gibco BRL) (2×106 cpm/ml). Washes were performed with 0.2×SSC, 0.1% SDS, at 55-60° C. FBP1 and FBP3a probes were two HindIII restriction fragments (nucleotides 1-571 and 1-450, respectively), FBP2, FBP4, and FBP1 probes were their respective full-length cDNAs, and β-ACTIN probe was from Clontech Inc.

Fluorescence in situ hybridixation (FISH) Genomic clones were isolated by high-stringency screening (65° C., 0.2×SSC, 0.1% SDS wash) of a λFIX II placenta human genomic library (Stratagene) with cDNA probes obtained from the 2-hybrid screening. Phage clones were confirmed by high-stringency Southern hybridization and partial sequence analysis. Purified whole phage DNA was labeled and FISH was performed as described (M. Pagano., ed., 1994, in Cell Cycle: Materials and Methods, 29).

6.2 Results

6.2.1 Characterization of Novel F-box Proteins and their Activity In Vivo

An improved version of the yeast two-hybrid system was used to search for interactors of human Skp1. The MaV103 yeast strain harboring the Gal4 DB-Skp1 fusion protein as bait was transformed with an activated T-cell cDNA library expressing Gal4 AD fusion proteins as prey. After initial selection and re-transformation steps, 3 different reporter assays were used to obtain 13 positive clones that specifically interact with human Skp1. After sequence analysis, the 13 rescued cDNAs were found to be derived from 7 different open reading frames all encoding FBPs. These novel FBPs were named as follows: FBP1, shown in FIG. 3 (SEQ ID NO:1); FBP2, shown in FIG. 4 (SEQ ID NO:3), FBP3a, shown in FIG. 5 (SEQ ID NO:5), FBP4, shown in FIG. 7 (SEQ ID NO:7), FBP5, shown in FIG. 8 (SEQ ID NO:9), FBP6, shown in FIG. 9 (SEQ ID NO:11), FBP7, shown in FIG. 10 (SEQ ID NO:13). One of the seven FBPs, FBP1 (SEQ ID NO:1) was also identified by others while our screen was in progress (Margottin et al., 1998, Molecular Cell, 1:565-74).

BLAST programs were used to search for predicted human proteins containing an F-box in databases available through the National Center for Biotechnology Information and The Institute for Genomic Research. The alignment of the F-box motifs from these predicted human FBPs is shown in FIG. 1. Nineteen previously uncharacterized human FBPs were identified by aligning available sequences (GenBank Accession Nos. AC002428, AI457595, AI105408, H66467, T47217, H38755, THC274684, AI750732, AA976979, AI571815, T57296, Z44228, Z45230, N42405, AA018063, AI751015, AI400663, T74432, AA402415, AI826000, AI590138, AF174602, Z45775, AF174599, THC288870, AI017603, AF174598, THC260994, AI475671, AA768343, AF174595, THC240016, N70417, T10511, AF174603, EST04915, AA147429, AI192344, AF174594, AI147207, AI279712, AA593015, AA644633, AA335703, N26196, AF174604, AF053356, AF174606, AA836036, AA853045, AI479142, AA772788, AA039454, AA397652, AA463756, AA007384, AA749085, AI640599, THC253263, AB020647, THC295423, AA434109, AA370939, AA215393, THC271423, AF052097, THC288182, AL049953, CAB37981, AL022395, AL031178, THC197682, and THC205131), with the nucleotide sequences derived from the F-box proteins disclosed above.

The nineteen previously uncharacterized FBP nucleotide sequences thus identified were named as follows: FBP3b, shown in FIG. 6 (SEQ ID NO:23); FBP8, shown in FIG. 11 (SEQ ID NO:25); FBP9, shown in FIG. 12 (SEQ ID NO:27); FBP10, shown in FIG. 13 (SEQ ID NO:29); FBP11, shown in FIG. 14 (SEQ ID NO:31); FBP12, shown in FIG. 15 (SEQ ID NO:33); FBP13, shown in FIG. 16 (SEQ ID NO:35); FBP14, shown in FIG. 17 (SEQ ID NO:37); FBP15, shown in FIG. 18 (SEQ ID NO:39); FBP16, shown in FIG. 19 (SEQ ID NO:41); FBP17, shown in FIG. 20 (SEQ ID NO:43); FBP18, shown in FIG. 21 (SEQ ID NO:45); FBP19, shown in FIG. 22 (SEQ ID NO:47); FBP20, shown in FIG. 23 (SEQ ID NO:49); FBP21, shown in FIG. 24 (SEQ ID NO:51); FBP22, shown in FIG. 25 (SEQ ID NO:53); FBP23, shown in FIG. 26 (SEQ ID NO:55); FBP24, shown in FIG. 27 (SEQ ID NO:57); and FBP25, shown in FIG. 28 (SEQ ID NO:59). The alignment of the F-box motifs from these predicted human FBPs is shown in FIG. 1A. Of these sequences, the nucleotide sequences of fourteen identified FBPs, FBP3b (SEQ ID NO:23), FBP8 (SEQ ID NO:25), FBP11 (SEQ ID NO:31), FBP12 (SEQ ID NO:33), FBP13 (SEQ ID NO:35), FBP14 (SEQ ID NO:37), FBP15 (SEQ ID NO:39), FBP17 (SEQ ID NO:43), FBP18 (SEQ ID NO:45), FBP20 (SEQ ID NO:49), FBP21 (SEQ ID NO:51), FBP22 (SEQ ID NO:53), FBP23 (SEQ ID NO:55), and FBP25 (SEQ ID NO:59) were not previously assembled and represent novel nucleic acid molecules. The five remaining sequences, FBP9 (SEQ ID NO:27), FBP10 (SEQ ID NO:29), FBP16 (SEQ ID NO:41), FBP19 (SEQ ID NO:47), and FBP24 (SEQ ID NO:57) were previously assembled and disclosed in the database, but were not previously recognized as F-box proteins.

Computer analysis of human FBPs revealed several interesting features (see the schematic representation of FBPs in FIG. 2. Three FBPs contain WD-40 domains; seven FBPs contain LRRs, and six FBPs contain other potential protein-protein interaction modules not yet identified in FBPs, such as leucine zippers, ring fingers, helix-loop-helix domains, proline rich motifs and SH2 domains.

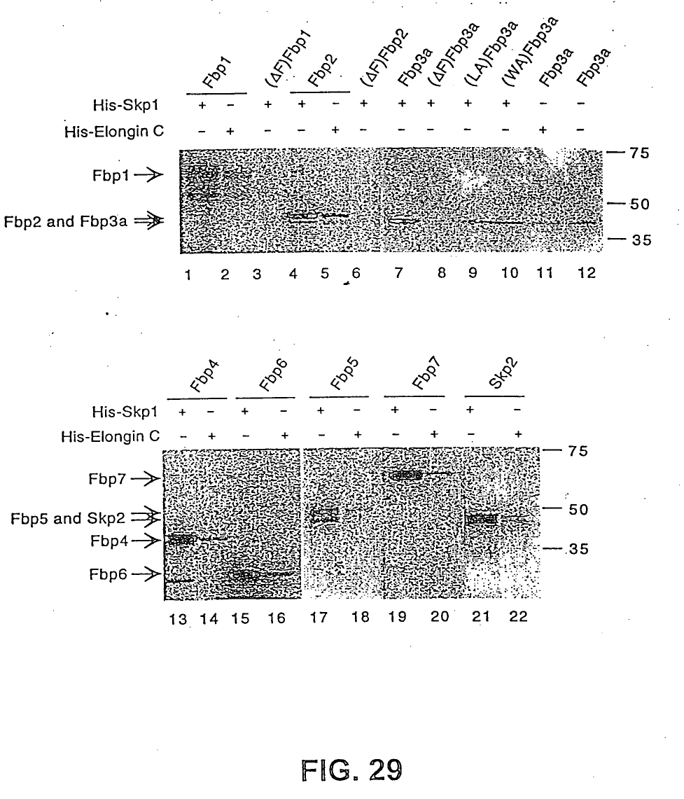

As examples of the human FBP family, a more detailed characterization of some FBPs was performed. To confirm the specificity of interaction between the novel FBPs and human Skp1, eight in vitro translated FBPs were tested for binding to His-tagged-Skp1 pre-bound to Nickel-agarose beads. As a control Elongin C was used, the only known human Skp1 homolog. All 7 FBPs were able to bind His-Skp1 beads but not to His-tagged-Elongin C beads (FIG. 29). The small amount of FBPs that bound to His-tagged-Elongin C beads very likely represents non-specific binding since it was also present when a non-relevant protein (His-tagged-p27) bound to Nickel-agarose beads was used in pull-down assays (see as an example, FIG. 29, lane 12).

F-box deletion mutants, (ΔF)FBP1, (ΔF)FBP2, (ΔF)FBP3a, and mutants containing single point mutations in conserved amino acid residues of the F-box, FBP3a(L51A) and FBP3a(W76A) were constructed. Mutants lacking the F-box and those with point mutations lost their ability to bind Skp1 (FIG. 29), confirming that human FBPs require the integrity of their F-box to specifically bind Skp1.

In order to determine whether FBP1, FBP2, FBP3a, FBP4 and FBP7 interact with human Skp1 and Cul1 in vivo (as Skp2 is known to do), flag-tagged-FBP1, -(ΔF)FBP1, -FBP2, -(ΔF)FBP2, -FBP3a, -(ΔF)FBP3a, -FBP4 and -FBP7 were expressed in HeLa cells from which cell extracts were made and subjected to immunoprecipitation with an anti-Flag antibody. As detected in immunoblots with specific antibodies to Cul1, Cul2 (another human cullin), and Skp1, the anti-Flag antibody co-precipitated Cul1 and Skp1, but not Cul2, exclusively in extracts from cells expressing wild-type FBPs (FIG. 29 and data not shown). These data indicate that as in yeast, the human Skp1/cullin complex forms a scaffold for many FBPs.

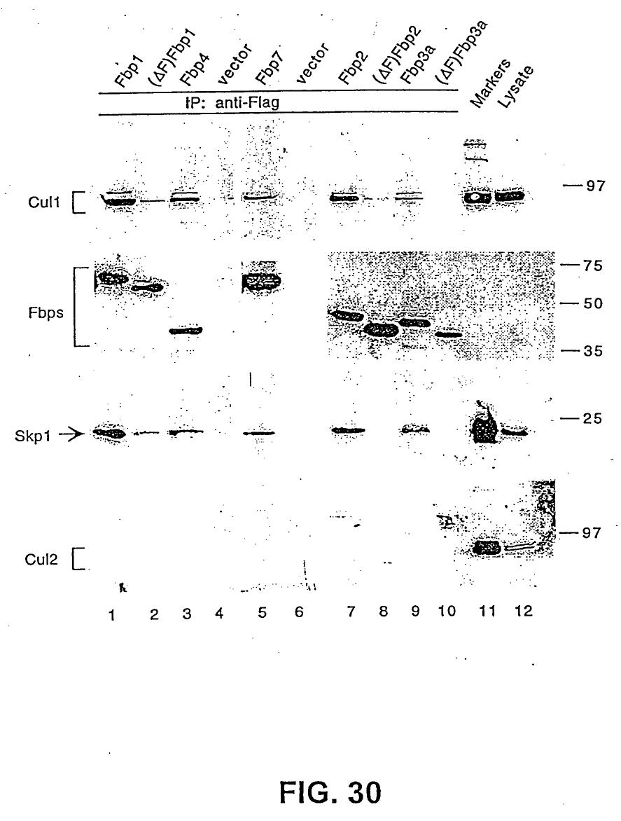

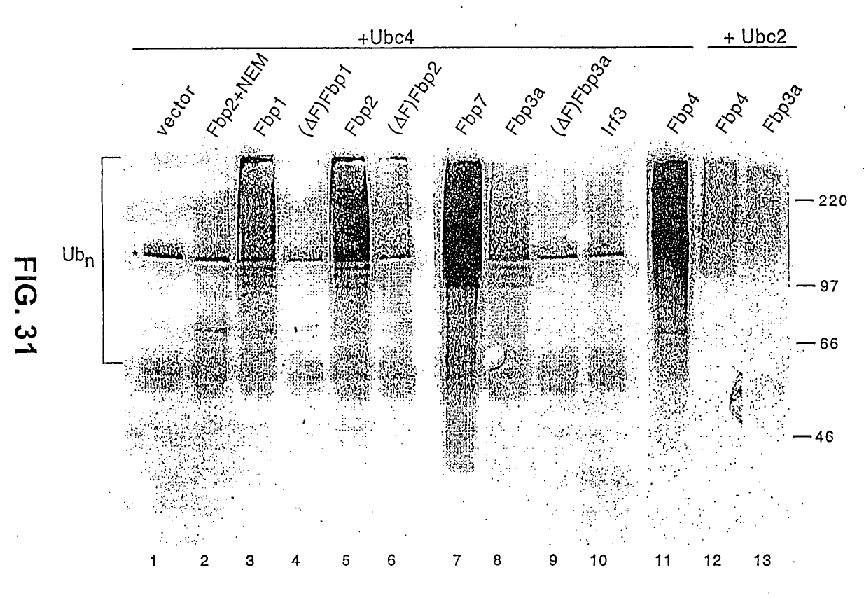

The binding of FBPs to the Skp1/Cul1 complex is consistent with the possibility that FBPs associate with a ubiquitin ligation activity. To test this possibility, Flag-tagged were expressed in HeLa cells, FBPs together with human Skp1 and Cul1. Extracts were subjected to immunoprecipitation with an anti-Flag antibody and assayed for ubiquitin ligase activity in the presence of the human ubiquitin-activating enzyme (E1) and a human Ubc. All of the wild type FBPs tested, but not FBP mutants, associated with a ubiquitin ligase activity which produced a high molecular weight smear characteristic of ubiquitinated proteins (FIG. 30). The ligase activity was N-ethylmaleimide (NEM) sensitive (FIG. 30, lane 2) and required the presence of both Ubc4 and E1. Results similar to those with Ubc4 were obtained using human Ubc3, whereas Ubc2 was unable to sustain the ubiquitin ligase activity of these SCFs (FIG. 30, lanes 12, 13).

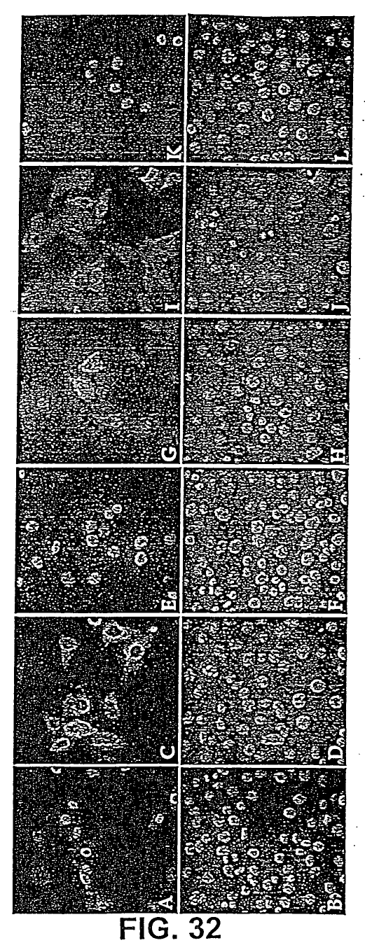

Using indirect immunofluorescence techniques, the subcellular distribution of FBP1, FBP2, FBP3a, FBP4 and FBP7 was studied in human cells. Flag-tagged-versions of these proteins were expressed in HeLa, U2OS, and 293T cells and subjected to immunofluorescent staining with an anti-Flag antibody. FBP1, FBP4 and FBP7 were found to be distributed both in the cytoplasm and in the nucleus, while FBP2 was detected mainly in the cytoplasm and FBP3a mainly in the nucleus. FIG. 32 shows, as an example, the subcellular localization of FBP1, FBP2, FBP3a, FBP4 observed in HeLa cells. The localization of (ΔF)FBP1, (ΔF)FBP2, (ΔF)FBP3a mutants was identical to those of the respective wild-type proteins (FIG. 32) demonstrating that the F-box and the F-box-dependent binding to Skp1 do not determine the subcellular localization of FBPs. Immunofluorescence stainings were in agreement with the results of biochemical subcellular fractionation.

6.2.2 Northern Blot Analysis of Novel Ubiquitin Ligase Gene Transcripts

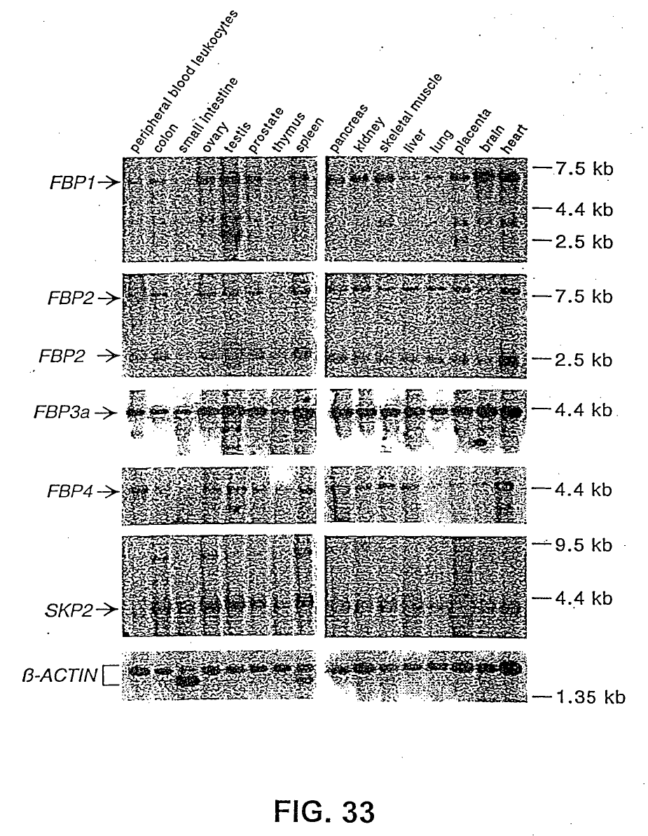

RNA blot analysis was performed on poly(A)+ mRNA from multiple normal human tissues (heart, brain, placenta, lung, liver, skeletal, muscle, kidney, pancreas, spleen, thymus, prostate, testis, ovary, small intestine, colon, peripheral blood leukocytes, see FIG. 33). FBP1 mRNA transcripts (a major band of ˜7-kb and two minor bands of ˜3.5- and ˜2.5 kb) were expressed in all of the 16 human tissues tested but were more prevalent in brain and testis. Testis was the only tissue expressing the smaller FBP1 mRNA forms in amounts equal to, if not in excess of, the 7 kb form. FBP2 transcripts (˜7.7-kb and ˜2.4-kb) were expressed in all tissues tested, yet the ratio of the FBP2 transcripts displayed some tissue differences. An approximately 4 kb FBP3a transcript was present in all tissues tested and two minor FBP3a forms of approximately 3 kb and 2 kb became visible, upon longer exposure, especially in the testis. An approximately 4.8 kb FBP4 transcript was expressed in all normal human tissues tested, but was particularly abundant in heart and pancreas. Finally, the pattern of expression of the new FBPs was compared to that of FBP1 whose mRNA species (a major band ˜4 kb and a minor band of ˜8.5 kb) were found in all tissues but was particularly abundant in placenta.

6.2.3 Chromosomal Localization of the Human FBP Genes

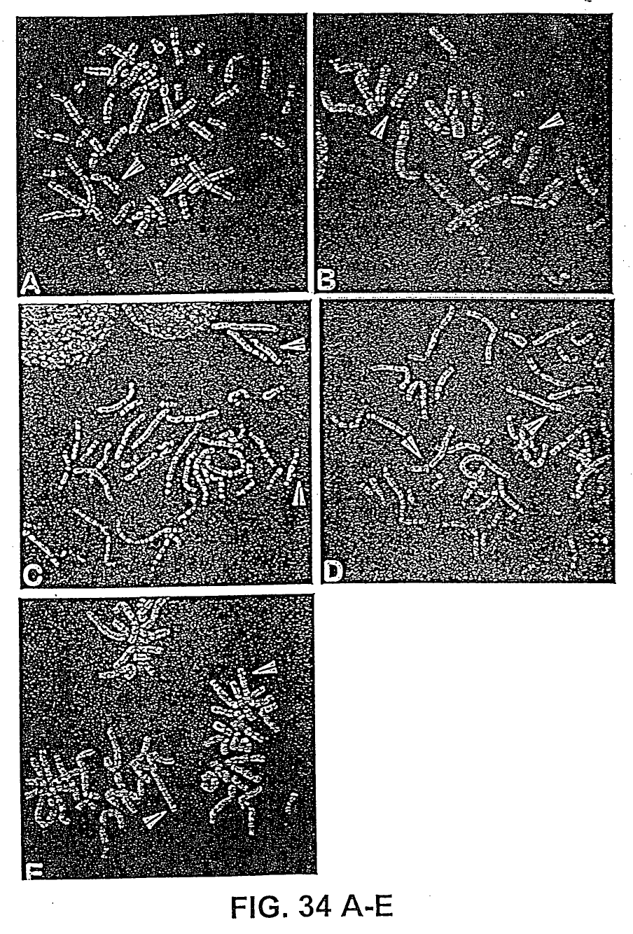

Unchecked degradation of cellular regulatory proteins (e.g., p53, p27, β-catenin) has been observed in certain tumors, suggesting the hypothesis that deregulated ubiquitin ligases play a role in this altered degradation (reviewed in A. Ciechanover, 1998, Embo J, 17: 7151). A well understood example is that of MDM2, a proto-oncogene encoding a ubiquitin ligase whose overexpression destabilize its substrate, the tumor suppressor p53 (reviewed by Brown and Pagano, 1997, Biochim Biophys Acta, 1332: 1, 1998). To map the chromosomal localization of the human FBP genes and to determine if these positions coincided with loci known to be altered in tumors or in inherited disease, fluorescence in situ hybridization (FISH) was used. The FBP1 gene was mapped and localized to 10q24 (FIG. 34A), FBP2 to 9q34 (FIG. 34B), FBP3a to 13q22 (FIG. 34C), FBP4 to 5p12 (FIG. 34D) and FBP5 to 6q25-26 (FIG. 34E). FBP genes (particularly FBP1, FBP3a, and FBP5) are localized to chromosomal loci frequently altered in tumors (for references and details see Online Mendelian Inheritance in Man database, http://www3.ncbi.nlm.nih.gov/omim/). In particular, loss of 10q24 (where FBP1 is located) has been demonstrated in approx. 10% of human prostate tumors and small cell lung carcinomas (SCLC), suggesting the presence of a tumor suppressor gene at this location. In addition, up to 7% of childhood acute T-cell leukemia is accompanied by a translocation involving 10q24 as a breakpoint, either t(10;14)(q24;q11) or t(7;10)(q35;q24). Although rarely, the 9q34 region (where FBP2 is located) has been shown to be a site of loss of heterozygosity (LOH) in human ovarian and bladder cancers. LOH is also observed in the region. Finally, 6q25-26 (where FBP5 is located) has been shown to be a site of loss of heterozygosity in human ovarian, breast and gastric cancers hepatocarcinomas, Burkitt's lymphomas, and parathyroid adenomas.

7. Example FBP1 Regulates the Stability of β-Catenin

Deregulation of β-catenin proteolysis is associated with malignant transformation. Xenopus Slimb and Drosophila FBP1 negatively regulate the Wnt/β-catenin signaling pathway (Jiang and Struhl, 1998, supra; Marikawa and Elinson, 1998). Since ubiquitin ligase complexes physically associate with their substrates, the studies in this Example were designed to determine whether FBP1 can interact with β-catenin. The results show that FBP1 forms a novel ubiquitin ligase complex that regulates the in vivo stability of β-catenin. Thus, the identification of FBP1 as a component of the novel ubiquitin ligase complex that ubiquitinates β-catenin, provides new targets that can be used in screens for agonists, antagonists, ligands, and novel substrates using the methods of the present invention. Molecules identified by these assays are potentially useful drugs as therapeutic agents against cancer and proliferative disorders.

7.1 Materials and Methods for Identification of FBP1 Function

Recombinant proteins, Construction of F-box mutants Antibodies, Transient transfections, Imunoprecipitation, Immunoblotting. Cell culture and Extract preparation Details of the methods are described in Section 6.1, supra.

7.2 Results

7.2.1 Human FBP1 Interacts With β-Catenin

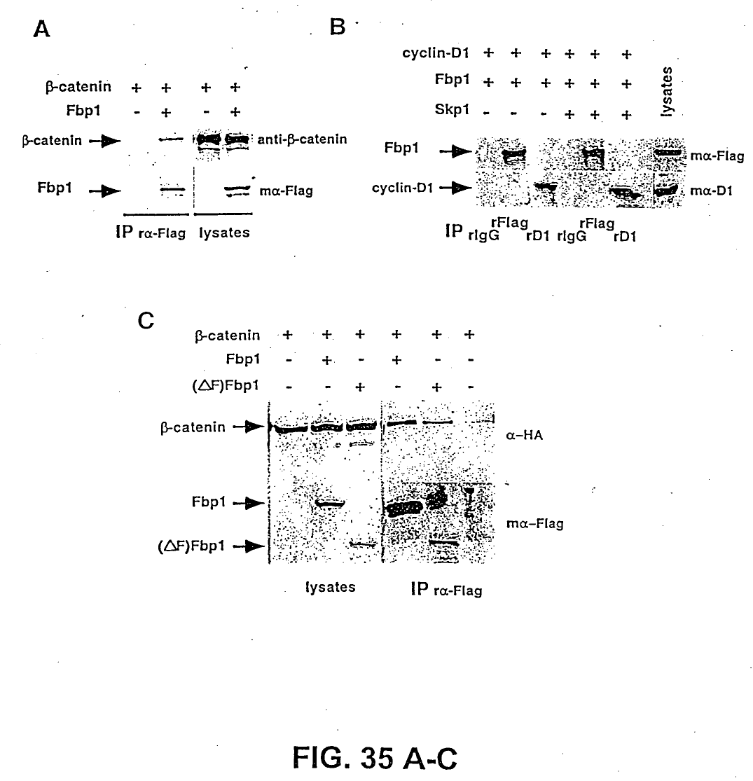

Flag-tagged FBP1 and β-catenin viruses were used to co-infect insect cells, and extracts were analyzed by immunoprecipitation followed by immunoblotting. β-catenin was co-immunoprecipitated by an anti-Flag antibody (FIG. 35A), indicating that in intact cells β-catenin and FBP1 physically interact. It has been shown that binding of the yeast FBP Cdc4 to its substrate Sic1 is stabilized by the presence of Skp1 (Skowyra et al., 1997, Cell, 91, 209-219). Simultaneous expression of human Skp1 had no effect on the strength of the interaction between FBP1 and β-catenin. To test the specificity of the FBP1/β-catenin interaction, cells were co-infected with human cyclin D1 and FBP1 viruses. The choice of this cyclin was dictated by the fact that human cyclin D1 can form a complex with the Skp2 ubiquitin ligase complex (Skp1-Cul1-Skp2; Yu et al., 1998, Proc. Natl. Acad. Sci. U.S.A, 95:11324-9). Under the same conditions used to demonstrate the formation of the FBP1/β-catenin complex, cyclin D1 could not be co-immunoprecipitated with Flag-tagged FBP1, and anti-cyclin D1 antibodies were unable to co-immunoprecipitate FBP1 (FIG. 35B, lanes 1-3). Co-expression of Skp1 (FIG. 35B, lanes 4-6) or Cdk4 with FBP1 and cyclin D1 did not stimulate the association of cyclin D1 with FBP1.

Mammalian expression plasmids carrying HA-tagged β-catenin and Flag-tagged FBP1 (wild type or mutant) were then co-transfected in human 293 cells. β-catenin was detected in anti-Flag immunoprecipitates when co-expressed with either wild type or (ΔF)FBP1 mutant (FIG. 35C, lanes 4-6), confirming the presence of a complex formed between β-catenin and FBP1 in human cells.

7.2.2 F-box Deleted FBP1 Mutant Stabilizes β-Catenin In Vivo

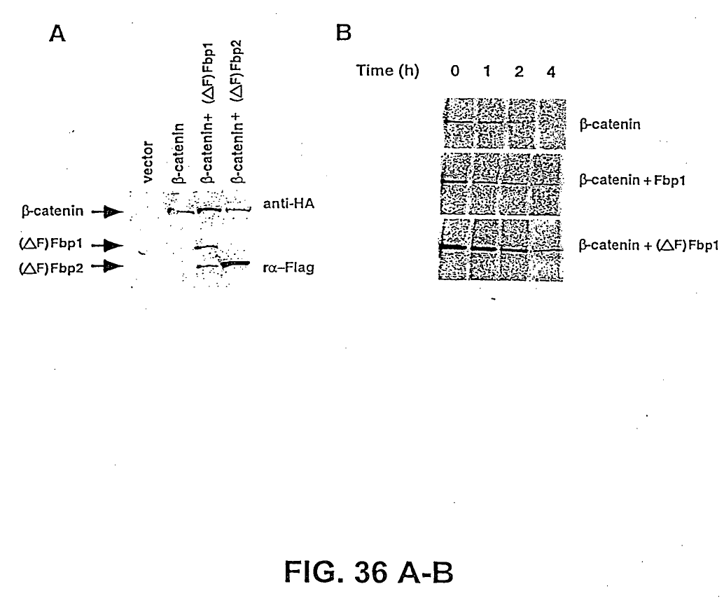

The association of (ΔF)FBP1 to β-catenin suggested that (ΔF)FBP1 might act as a dominant negative mutant in vivo by being unable to bind Skp1/Cul1 complex, on the one hand, while retaining the ability to bind β-catenin, on the other. HA-tagged β-catenin was co-expressed together with Flag-tagged (ΔF)FBP1 or with another F-box deleted FBP, (ΔF)FBP2. FBP2 was also obtained with our screening for Skp1-interactors; and, like FBP1, contains several WD-40 domains. The presence of (ΔF)FBP1 specifically led to the accumulation of higher quantities of β-catenin (FIG. 36A). To determine whether this accumulation was due to an increase in β-catenin stability, we measured the half-life of β-catenin using pulse chase analysis. Human 293 cells were transfected with HA-tagged β-catenin alone or in combination with the wild type or mutant FBP1. While wild type Fpb1 had little effect on the degradation of β-catenin, the F-box deletion mutant prolonged the half life of β-catenin from 1 to 4 hours (FIG. 36B).

FBP1 is also involved in CD4 degradation induced by the HIV-1 Vpu protein (Margottin et al., supra). It has been shown that Vpu recruits FBP1 to DC4 and (ΔF) FBP1 inhibits Vpu-mediated CD4 regulation. In addition, FBP1-ubiquitin ligase complex also controls the stability of IKBαa (Yaron et al., 1998, Nature, 396: 590). Thus, the interactions between FBP1 and β-catenin, Vpu protein, CD4, and IKBαa are potential targets that can be used to screen for agonists, antagonists, ligands, and novel substrates using the methods of the present invention.

8. Example Methods for Identifying p27 as a Substrate of the FBP Skp2

Degradation of the mammalian G1 cyclin-dependent kinase (Cdk) inhibitor p27 is required for the cellular transition from quiescence to the proliferative state. The ubiquitination and degradation of p27 depend upon its phosphorylation by cyclin/Cdk complexes. Skp2, an F-box protein essential for entry into S phase, specifically recognizes p27 in a phosphorylation-dependent manner. Furthermore, both in vivo and in vitro, Skp2 is a rate-limiting component of the machinery that ubiquitinates and degrades phosphorylated p27. Thus, p27 degradation is subject to dual control by the accumulation of both Skp2 and cyclins following mitogenic stimulation.

This Example discloses novel assays that have been used to identify the interaction of Skp2 and p27 in vitro. First, an in vitro ubiquitination assay performed using p27 as a substrate is described. Second, Skp2 is depleted from cell extracts using anti-Skp2 antibody, and the effect on p27 ubiquitin ligase activity is assayed. Purified Skp2 is added back to such immunodepleted extracts to restore p27 ubiquitination and degradation. Also disclosed is the use of a dominant negative mutant, (AF)Skp2, which interferes with p27 ubiquitination and degradation.

The assays described herein can be used to test for compounds that inhibit cell proliferation. The assays can be carried out in the presence or absence of molecules, compounds, peptides, or other agents described in Section 5.5. Agents that either enhance or inhibit the interactions or the ubiquitination activity can be identified by an increase or decrease the formation of a final product are identified. Such agents can be used, for example, to inhibit Skp2-regulated p27 ubiquitination and degradation in vivo. Molecules identified by these assays are potentially useful drugs as therapeutic agents against cancer and proliferative disorders.

Dominant negative mutants, for example the mutant (ΔF)Skp2, and antisense oligos targeting SKP2, mRNA interfere with p27 ubiquitination and degradation, and can be used in gene therapies against cancer. The assays described herein can also be used to identify novel substrates of the novel FBP proteins, as well as modulators of novel ubiquitin ligase complex—substrate interactions and activities.

8.1 Materials and Methods for Identification of p27 as a Skp2 Substrate

Protein extraction for in vitro ubiquitination assay Approx. 4 ml of HeLa S3 cell pellet were suspended in 6 ml of ice-cold buffer consisting of 20 mM Tris-HCl (pH 7.2), 2 mM DTT, 0.25 mM EDTA, 10 μg/ml leupeptin, and 10 μg/ml pepstatin. The suspension was transferred to a cell nitrogen-disruption bomb (Parr, Moline, Ill., cat #4639) that had been rinsed thoroughly and chilled on ice before use. The bomb chamber was connected to a nitrogen tank and the pressure was brought slowly to 1000 psi. The chamber was left on ice under the same pressure for 30 minutes and then the pressure was released slowly. The material was transferred to an Eppendorf tube and centrifuged in a microcentrifuge at 10,000 g for 10 minutes. The supernatant (S-10) was divided into smaller samples and frozen at −80° C. This method of extract preparation based on the use of a cell nitrogen-disruption bomb extract preserves the activity to in vitro ubiquitinate p27 better than the method previously described (Pagano et al., 1995, Science 269:682-685).

Reagents and antibodies Ubiquitin aldehyde (Hershko & Rose, 1987, Proc. Natl. Acad. Sci. USA 84:1829-33), methyl-ubiquitin (Hershko & Heller, 1985, Biochem. Biophys. Res. Commun. 128:1079-86) and p13 beads (Brizuela et al., 1987, EMBO J. 6:3507-3514) were prepared as described. β, γ-imidoadenosine-50-triphosphate (AMP-PNP), staurosporine, hexokinase, and deoxy-glucose were from Sigma; lovastatine obtained from Merck; flavopiridol obtained from Hoechst Marion Roussel. The phospho-site p27 specific antibody was generated in collaboration with Zymed Inc. by injecting rabbits with the phospho-peptide NAGSVEQT*PKKPGLRRRQT (SEQ ID NO: 85), corresponding to the carboxy terminus of the human p27 with a phosphothreonine at position 187 (T*). The antibody was then purified from serum with two rounds of affinity chromatography using both phospho- and nonphospho-peptide chromatography. All the other antibodies are described in Section 6.1.

Immunodepletion Assays For immunodepletion assays, 3 μl of an Skp2 antiserum was adsorbed to 15 μl Affi-Prep Protein-A beads (BioRad), at 4° C. for 90 min. The beads were washed and then mixed (4° C., 2 hours) with 40 μl of HeLa extract (approximately 400 μg of protein). Beads were removed by centrifugation and supernatants were filtered through a 0.45-μ Microspin filter (Millipore). Immunoprecipitations and immunoblots were performed as described (M. Pagano, et al., 1995, supra. Rabbit polyclonal antibody against purified GST-Skp2 was generated, affinity-purified (AP) and characterized as described (M. Pagano, in Cell Cycle-Materials and Methods, M. Pagano Ed. (Springer, N.Y., 1995), chap. 24; E. Harlow and D. Lane, in Using antibodies. A Laboratory Manual (Cold Spring Harbor Laboratory, Cold Spring Harbor, N.Y., 1998), in collaboration with Zymed Inc. (cat # 51-1900). Monoclonal antibodies (Mabs) to human Cul1, and cyclin E, (Faha et al., 1993, J. of Virology 67:2456); AP rabbit antibodies to human p27, Skp1 (Latres et al., 1999, Oncogene 18:849), Cdk2 (Pagano, et al., 1992, Science 255:1144) and phospho-site p27 specific antibody. Mab to cyclin B was from Santa Cruz (cat # sc-245); Mabs to p21 (cat # C24420) and p27 (cat # K25020) Transduction lab; anti-Flag rabbit antibody from Zymed (cat # 71-5400). An AP goat antibody to an N-terminal Skp2 peptide (Santa Cruz, cat # sc-1567) was used.

Construction of Skp2 F-box mutant (ΔF)Skp2 mutant was obtained by removing a DNA fragment (nucleotides 338-997) with BspEI and XbaI restriction enzymes, and replacing it with a PCR fragment containing nucleotides 457 to 997. The final construct encoded a protein lacking residues 113-152.

Recombinant proteins cDNA fragments encoding the following human proteins: Flag-tagged FBP1, Flag-tagged (ΔF)FBP1, Flag-tagged FBP3a, Skp2, HA-tagged Cul1, HA-tagged Cul2, β-catenin, His-tagged cyclin D1, Skp1, His-tagged Skp1, His-tagged Elongin C were inserted into the baculovirus expression vector pBacpak-8 (Clonetech) and cotransfected into Sf9 cells with linearized baculovirus DNA using the BaculoGold transfection kit (Pharmingen). Baculoviruses expressing human His-tagged cyclin E and HA-tagged Cdk2 were supplied by D. Morgan (Desai, 1992, Molecular Biology of the Cell 3: 571). Recombinant viruses were used to infect 5B cells and assayed for expression of their encoded protein by immunoblotting as described above. His-proteins were purified with Nickel-agarose (Invitrogen) according to the manufacturer's instructions. The different complexes were formed by co-expression of the appropriate baculoviruses and purified by nickel-agarose chromatography, using the His tag at the 5′ of Skp1 and cyclin E. Unless otherwise stated, recombinant proteins were added to incubations at the following amounts: cyclin E/Cdk2, ˜0.5 pmol; Skp1, ˜0.5 pmol; Skp2, ˜0.1 pmol; FBP1, ˜0.1 pmol; FBP3a, 0.1 pmol, Cul1, ˜0.1 pmol. The molar ratio of Skp1/Skp2, Skp1/FBP1, Skp1/FBP3a, and Skp1/Cul1 in the purified preparations was ˜5.

Extract preparation and cell synchronization, Transient transfections, Immunoprecipitation and Immunoblotting Methods were carried out as described in Section 6.1, supra.

8.2 Results

8.2.1 p27 In Vitro Ubiquitination Assay

In an exemplary in vitro ubiquitination assay, logarithmically growing, HeLa-S3 cells were collected at a density of 6×105 cells/ml. Cells are arrested in G1 by 48-hour treatment with 70 μM lovastatin as described (O'Connor &. Jackman, 1995 in Cell Cycle-Materials and Methods, M. Pagano, ed., Springer, N.Y., chap. 6). 1 μl of in vitro translated [35S]p27 is incubated at 30° C. for different times (0-75 minutes) in 10 μl of ubiquitination mix containing: 40 mM Tris pH 7.6, 5 mM MgCl₂, 1 mM DTT, 10% glycerol, 1 μM ubiquitin aldehyde, 1 mg/ml methyl ubiquitin, 10 mM creatine phosphate, 0.1 mg/ml creatine phosphokinase, 0.5 mM ATP, 1 μM okadaic acid, 20-30 μg HeLa cell extract. Ubiquitin aldehyde can be added to the ubiquitination reaction to inhibit the isopeptidases that would remove the chains of ubiquitin from p27. Addition of methyl ubiquitin competes with the ubiquitin present in the cellular extracts and terminates p27 ubiquitin chains. Such chains appear as discrete bands instead of a high molecular smear. These shorter polyubiquitin chains have lower affinity for the proteasome and therefore are more stable. Reactions are terminated with Laemmli sample buffer containing β-mercaptoethanol and the products can be analyzed on protein gels under denaturing conditions.

Polyubiquitinated p27 forms are identified by autoradiography. p27 degradation assay is performed in a similar manner, except that (i) Methylated ubiquitin and ubiquitin aldehyde were omitted; (ii) The concentration of HeLa extract is approximately 7 μg/μ1; (iii) Extracts are prepared by hypotonic lysis (Pagano et al., 1995, Science 269:682), which preserves proteasome activity better than the nitrogen bomb disruption procedure. In the absence of methyl ubiquitin, p27 degradation activity, instead of p27 ubiquitination activity, can be measured.

The samples are immunoprecipited with an antibody to p27 followed by a subsequent immunoprecipitation with an anti-ubiquitin antibody and run on an 8% SDS gel. The high molecular species as determined by this assay are ubiquitinated. As a control, a p27 mutant lacking all 13 lysines was used. This mutant form of p27 is not ubiquitinated and runs at higher molecular weight on the 8% SDS gel.

8.2.2 p27-Skp2 Interaction Assays and p27-Skp2 Immunodepletion Assay

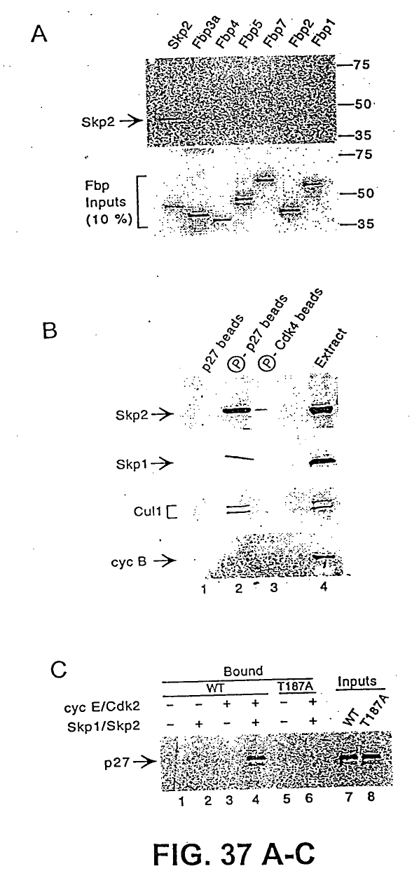

The recruitment of specific substrates by yeast and human FBPs to Skp1/cullin complexes is phosphorylation-dependent. Accordingly, peptides derived from IκBα and β-catenin bind to FBP1 specifically and in a phosphorylation-dependent manner (Yaron, 1998, Nature 396: 590; Winston et al., 1999, Genes Dev. 13: 270). A p27 phospho-peptide with a phosphothreonine at position 187 was assayed for its ability to bind to human FBPs, including Skp2 and the FBP1, FBP2, FBP3a, FBP4, FBP5, FBP6, and FBP7, isolated by using a 2-hybrid screen using Skp1 as bait, as described in Section 6, above. Four of these FBPs contain potential substrate interaction domains, such as WD-40 domains in FBP1 and FBP2, and leucine-rich repeats in Skp2 and FBP3a. The phospho-p27 peptide was immobilized to Sepharose beads and incubated with these seven in vitro translated FBPs (FIG. 37A). Only one FBP, Skp2, was able to bind to the phospho-T187 p27 peptide. Then, beads linked to p27 peptides (in either phosphorylated or unphosphorylated forms) or with an unrelated phospho-peptide were incubated with HeLa cell extracts. Proteins stably associated with the beads were examined by immunoblotting. Skp2 and its associated proteins, Skp1 and Cul1, were readily detected as proteins bound to the phospho-p27 peptide but not to control peptides (FIG. 37B).

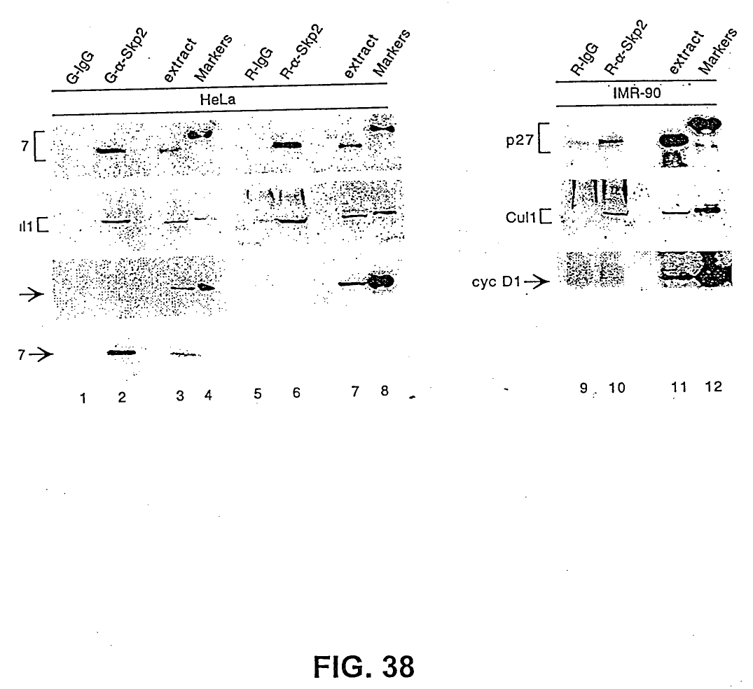

To further study p27 association to Skp2, in vitro translated p27 was incubated with either Skp1/Skp2 complex, cyclin E/Cdk2 complex, or the combination of both complexes under conditions in which p27 is phosphorylated on T187 by cyclin E/Cdk2 (Montagnoli, A., et al., 1999, Genes & Dev 13: 1181). Samples were then immunoprecipitated with an anti-Skp2 antibody. p27 was co-immunoprecipitated with Skp2 only in the presence of cyclin E/Cdk2 complex (FIG. 37C). Notably, under the same conditions, a T187-to-alanine p27 mutant, p27(T187A), was not co-immunoprecipitated by the anti-Skp2 antibody. Finally, we tested Skp2 and p27 association in vivo. Extracts from HeLa cells and IMR90 human diploid fibroblasts were subjected to immunoprecipitation with two different antibodies to Skp2 and then immunoblotted. p27 and Cul1, but not cyclin D1 and cyclin B1, were specifically detected in Skp2 immunoprecipitates (FIG. 38). Importantly, using a phospho-T 187 site p27 specific antibody we demonstrated that the Skp2-bound p27 was phosphorylated on T187 (FIG. 38, lane 2, bottom panel). Furthermore, an anti-peptide p27 antibody specifically co-immunoprecipitated Skp2. These results indicate that the stable interaction of p27 with Skp2 was highly specific and dependent upon phosphorylation of p27 on T187.

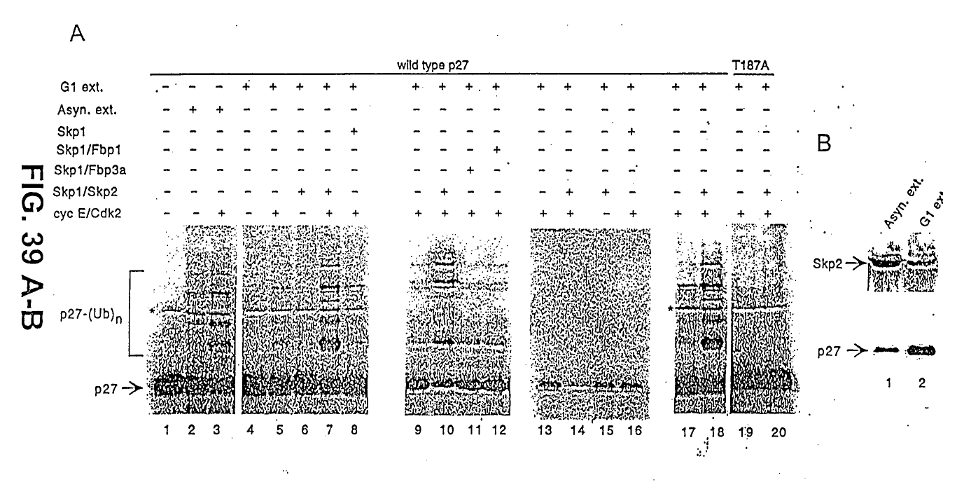

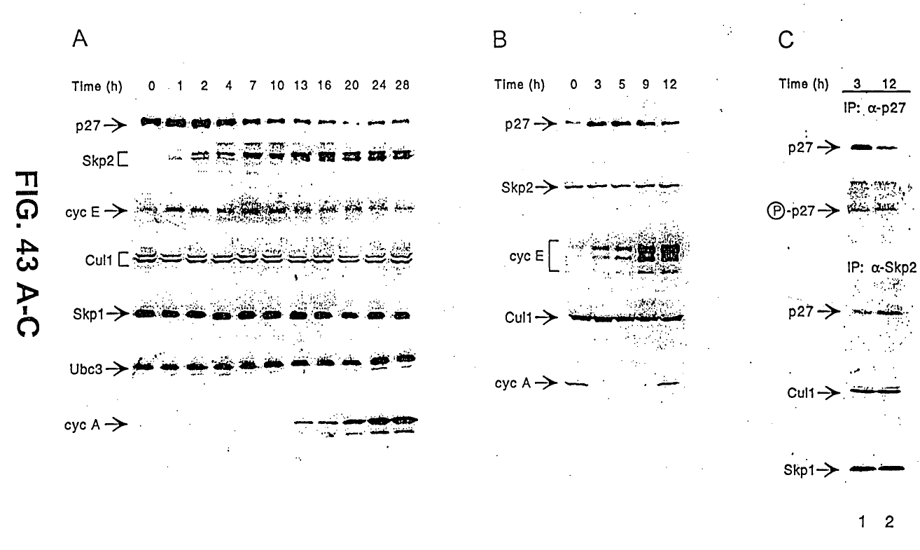

A cell-free assay for p27 ubiquitination which faithfully reproduced the cell cycle stage-specific ubiquitination and degradation of p27 has been developed (Montagnoli et al., supra). Using this assay, a p27-ubiquitin ligation activity is higher in extracts from asynchronously growing cells than in those from G1-arrested cells (FIG. 39A, lanes 2 and 4). In accordance with previous findings (Montagnoli, A., et al., supra), the addition of cyclin E/Cdk2 stimulated the ubiquitination of p27 in both types of extracts (FIG. 39A, lanes 3 and 5). However, this stimulation was much lower in extracts from G1-arrested cells than in those from growing cells, suggesting that in addition to cyclin E/Cdk2, some other component of the p27-ubiquitin ligation system is rate-limiting in G1. This component could be Skp2 since, in contrast to other SCF subunits, its levels are lower in extracts from G1 cells than in those from asynchronous cells and are inversely correlated with levels of p27 (FIGS. 39B and 43). Skp2 was thus tested to determine if it is a rate-limiting component of a p27 ubiquitin ligase activity. The addition of recombinant purified Skp1/Skp2 complex alone to G1 extracts did not stimulate p27 ubiquitination significantly (FIG. 39A, lane 6). In contrast, the combined addition of Skp1/Skp2 and cyclin E/Cdk2 complexes strongly stimulated p27 ubiquitination in G1 extracts (FIG. 39A, lane 7). Similarly, the combined addition of Skp1/Skp2 and cyclin E/Cdk2 strongly stimulated p27 proteolysis as measured by a degradation assay (FIG. 39A, lanes 13-16). Since the Skp1/Skp2 complex used for these experiments was isolated from insect cells co-expressing baculovirus His-tagged-Skp1 and Skp2 (and co-purified by nickel-agarose chromatography), it was possible that an insect-derived F-box protein co-purified with His-Skp1 and was responsible for the stimulation of p27 ubiquitination in G1 extracts. This possibility was eliminated by showing that the addition of a similar amount of His-tagged-Skp 1, expressed in the absence of Skp2 in insect cells and purified by the same procedure, did not stimulate p27 ubiquitination in the presence of cyclin E/Cdk2 (FIG. 39A, lane 8). Furthermore, we found that neither FBP1 nor FBP3a could replace Skp2 for the stimulation of p27-ubiquitin ligation in G1 extracts (FIG. 39A, lanes 9-12). Stimulation of p27-ubiquitination in G1 extracts by the combined addition of Skp1/Skp2 and cyclin E/Cdk2 could be observed only with wild-type p27, but not with the p27(T 187A) mutant (lanes 17-20), indicating that phosphorylation of p27 on T187 is required for the Skp2-mediated ubiquitination of p27. These findings indicated that both cyclin E/Cdk2 and Skp1/Skp2 complexes are rate-limiting for p27 ubiquitination and degradation in the G1 phase.

To further investigate the requirement of Skp2 for p27 ubiquitin ligation, Skp2 was specifically removed from extracts of asynchronously growing cells by immunodepletion with an antibody to Skp2. The immunodepletion procedure efficiently removed most of Skp2 from these extracts and caused a drastic reduction of p27-ubiquitin ligation activity (FIG. 40A, lane 4) as well as of p27 degradation activity. This effect was specific as shown by the following observations: (i) Similar treatment with pre-immune serum did not inhibit p27-ubiquitination (FIG. 40A, lane 3); (ii) Pre-incubation of anti-Skp2 antibody with recombinant GST-Skp2 (lane 5), but not with a control protein (lane 4), prevented the immunodepletion of p27-ubiquitination activity from extracts; (iii) p27ubiquitinating activity could be restored in Skp2-depleted extracts by the addition of His-Skp1/Skp2 complex (FIG. 40B, lane 3) but not His-Skp1 (lane 2), His-Skp1/Cul1 complex (lane 4), or His-Skp1/FBP1.

We then immunoprecipitated Skp2 from HeLa extracts and tested whether this immunoprecipitate contained a p27 ubiquitinating activity. The anti-Skp2 beads, but not a immunoprecipitate made with a pre-immune (PI) serum, was able to induce p27 ubiquitination in the presence of cyclin E/Cdk2 (FIG. 40C, lanes 2 and 3). The addition of purified recombinant E1 ubiquitin-activating enzyme, and purified recombinant Ubc3 did not greatly increase the ability of the Skp2 immunoprecipitate to sustain p27 ubiquitination, (FIG. 40C, lane 5), likely due to the presence of both proteins in the rabbit reticulocyte lysate used for p27 in vitro translation.

8.2.3 F-BOX Deleted SKP2Mutant Stabilzes p27 In Vivo

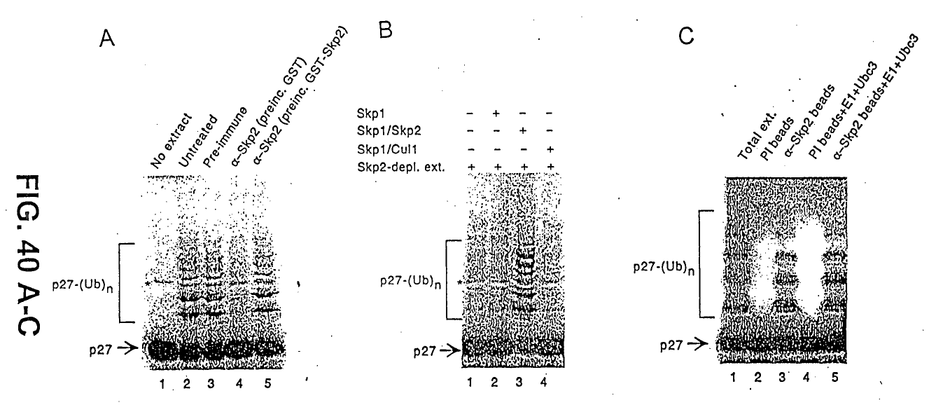

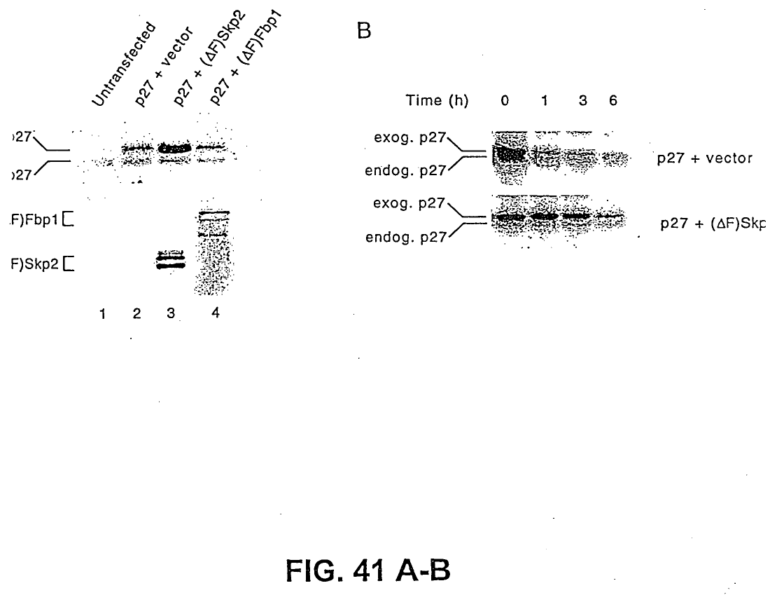

Skp2 also targets p27 for ubiquitin-mediated degradation in vivo. The F-box-deleted FBP1 mutant, (ΔF)FBP1, acts in vivo as a dominant negative mutant, most likely because without the F-box is unable to bind Skp1/Cul1 complex but retains the ability to bind its substrates. Therefore, once expressed in cells, (ΔF)Fb sequesters β-catenin and IKBa and causes their stabilization. An F-box deleted Skp2 mutant, (ΔF)Skp2, was constructed. p27 was expressed in murine cells either alone or in combination with (ΔF)Skp2 or (ΔF)FBP1 (see FIG. 41). The presence of (ΔF)Skp2 led to the accumulation of higher quantities of p27. To determine whether this accumulation was due to an increase in p27 stability, the half-life of p27 was measured using pulse chase analysis (for details, see Section 8, above). Indeed, (ΔF)Skp2 prolonged p27 half-life from less than 1 hour to ˜3 hours. Since in these experiments the efficiency of transfection was approximately 10%, (ΔF)Skp2 affected only the stability of co-expressed human exogenous p27, but not of murine endogenous p27.

8.2.4 SKP2 Antisense Experiments

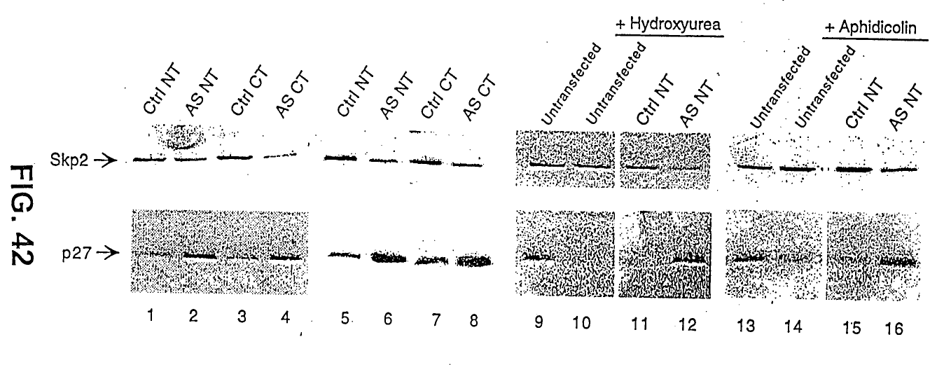

SKP2 mRNA was targeted with antisense oligonucleotides to determine whether a decrease in Skp2 levels would influence the abundance of endogenous p27. Two different antisense oligos, but not control oligodeoxynucleotides induced a decrease in Skp2 protein levels (FIG. 42). Concomitant with the Skp2 decrease, there was a substantial increase in the level of endogenous p27 protein. Similar results were obtained with cells blocked at the G1/S transition with hydroxyurea or aphidicolin treatment (lanes 9-16). Thus, the effect of the SKP2 antisense oligos on p27 was not a secondary consequence of a possible block in G1 due to the decrease in Skp2 levels.

Antisense experiments were performed as described in (Yu, 1998, Proc. Natl. Acad. Sci. U.S.A. 95: 11324). Briefly, four oligodeoxynucleotides that contain a phosphorothioate backbone and C-5 propyne pyrimidines were synthesized (Keck Biotechnology Resource Laboratory at Yale University): (1) 5′-CCTGGGGGATGTTCTCA-3′ (SEQ ID NO: 86) (the antisense direction of human Skp2 cDNA nucleotides 180-196); (2) 5′-GGCTTCCGGGCATTTAG-3′ (SEQ ID NO: 87) [the scrambled control of (1)]; (3) 5′-CATCTGGCACGATTCCA-3′ (SEQ ID NO: 88) (the antisense direction of Skp2 cDNA nucleotides 1137-1153); (4) 5′-CCGCTCATCGTATGACA-3′ (89) [the scrambled control for (3)]. The oligonucleotides were delivered into HeLa cells using Cytofectin GS (Glen Research) according to the manufacturers instructions. The cells were then harvested between 16 and 18 hours postransfection.

9. Example Method for Identifying Cks1 as a Mediator of the FBP Skp2/p27 Interaction

As stated in Example 8, p27 is recognized by Skp2 in a phosphorylation-dependent manner for entry into S phase and Skp2 is a rate-limiting component of the machinery that ubiquitinates and degrades phosphorylated p27. This Example discloses novel assays that have been used to identify the interactions of Cks1 with Skp2 and Cks1 with p27 in vitro and in a purified system. First, extracts of HeLa cells are fractionated and the activitiy of the fractions to promote the ligation of p27 is tested. Second, identification of Cks1 as the factor required for p27-ubiquitin ligation is confirmed with use of recombinant Cks1. Third, identification of Cks1's involvement in the p27-ubiquitin ligation after p27 is phosphorylated. Fourth, Cks1 increases the binding of Skp2 to p27. Fifth, Cks1 binds to Skp2. Sixth, Cks1 binds to the C-terminus of p27.

The assays described herein can be used to test for compounds that inhibit cell proliferation. The assays can be carried out in the presence or absence of molecules, compounds, peptides, or other agents described in Section 5.5. Agents that either enhance or inhibit the interactions or the ubiquitination activity can be identified by an increase or decrease the formation of a final product are identified. Such agents can be used, for example, to inhibit Skp2-regulated p27 ubiquitination and degradation in vivo. Molecules identified by these assays are potentially useful drugs as therapeutic agents against cancer and proliferative disorders.

Dominant negative mutants and antisense mRNA, oligos targeting the gene for Cks1, interfere with p27 ubiquitination and degradation, and can be used in gene therapies against cancer. The assays described herein can also be used to identify additional novel substrates of the novel FBP proteins, as well as additional modulators of novel ubiquitin ligase complex—substrate interactions and activities.

9.1 Materials and Methods for Identifying Cks1 AS a Mediator of the FBP Skp2/p27 Interaction

Proteins His₆-tagged p27 and Cdc34 were expressed in E. coli and purified by nickel-agarose chromatography. Cks2 and β13^{Suc1} were expressed in bacteria and purified by gel filtration chromatography. His₆-Skp1/Skp2, His₆-Skp1/P-TrCP, His₆-cyclin E/Cdk2, and His₆-Cul-1/ROC1 were produced by co-infection of 5B insect cells with baculoviruses encoding the corresponding proteins and were purified by nickel-agarose chromatography as described previously (Montagnoli, et al., 1999, Genes & Dev. 13:1501; Carrano, et al., 1999, Nat. Cell Biol. 1:193). The approximate concentrations of recombinant proteins in these preparations were (in pmole/μl): Skp1, 5; Skp2, 0.5; Cul-1, 4; ROC1, 1; cyclin E, 8; Cdk2, 1.5. Purified recombinant human Nedd8 was the generous gift of C. Pickart, and purified recombinant human Cks1 was the generous gift of S. Reed. Purified GST-IκBα(1-154) and its constitutively active kinase IKKβ^{S177E,S181E} were generously provided by Z.-Q. Pan. ³⁵S-labeled p27, Skp2 and Cks proteins were prepared by in vitro transcription-translation, using the TnT Quick kit (Promega) and ³⁵S-methionine (Amersham).

Purification of Nedd8-conjugating enzymes Purified recombinant human Nedd8 was the generous gift of C. Pickart. A mixture of Nedd8-conjugating enzymes (E1-like APP-BP1-Uba3 heterodimer and E2-like Ubc12: Osaka, et al., 1998, Genes Dev. 12:2263; Gong, L., Yeh, E. T., 1999, J. Biol. Chem. 274:12036) was co-purified from lysates of rabbit reticulocytes by a “covalent affinity” chromatography procedure similar to that used for the purification of E2s (Hershko, et al., 1983, J. Biol. Chem. 258:8206), except that unfractionated reticulocyte lysate was applied to a column of GST-Nedd8-Sepharose (5 mg/ml). Following a wash with 1M KC1, all proteins bound to immobilized Nedd8 by thiolester linkages were co-eluted with a solution containing 20 mM DTT. The DTT eluate was concentrated by ultrafiltration to approx. 1/10 of the original volume of reticulocyte lysate. This preparation had strong activity in the ligation of Nedd8 to Cul-1, without any detectable hydrolase activity that removes Nedd8 from Cul-1.

Purification of the factor required for p27-ubiquitin ligation A frozen pellet from 50 g of HeLa S3 cells (National Cell Culture Center) was disrupted by a nitrogen cell disruption bomb (Parr, Moline, Ill.) as described Montagnoli, et al., 1999, Genes & Dev. 13:1181, except that the buffer also contained 10 μg/ml chymostatin and 5 μg/ml aprotinin. The extract was centrifuged at 15,000×g for 20 min and the supemantants were centrifuged again at 100,000×g for 60 min. The supernatant was subjected to fractionation on DEAE-cellulose as described (Hershko, et al., 1983, J. Biol. Chem. 258:8206), except that 2,500 mg of protein was loaded on 250 ml of resin. The fraction not adsorbed to the resin (Fraction 1) was collected and was concentrated by centrifuge ultrafiltration to approx. 10 mg/ml. Fraction 1 (100 mg of protein) was subjected to heat-treatment at 90° C. for 10 minutes. The sample was allowed to stay on ice for 30 min, and then the precipitate was removed by centrifugation (10,000×g, 15 min). Approximately 99% of protein was removed by heat-treatment. The supernatant was concentrated by ultrafiltration and then was applied to a MonoS HR 5/5 column (Pharmacia) equilibrated with 50 mM Tris-HCl, 1 mM DTT and 0.1% (w/v) Brij-35 (Boehringer). The column was washed with 15 ml of the above buffer and was then eluted with a gradient of 0-200 mM NaCl. Activity in column fractions was followed by the p27-ubiquitin ligation assay in the presence of purified SCF^{Skp2} components (see below). The peak fractions of activity eluted at around 30-40 mM NaCl. The peak containing factor activity was pooled, concentrated by centrifuge ultrafiltration and was subjected to the final step of gel filtration chromatography on Superdex-75 HR 10/30 column (Pharmacia) equilibrated with 20 mM Tris-HCl (pH 7.2), 150 mM NaCl, 1 mM DTT and 01% Brij-35. Samples of 0.5 ml were collected at a flow rate of 0.4 ml/min. Column fractions were concentrated to a volume of 50 μl by centrifuge ultrafiltration (Centricon-10, Amicon). Samples of 0.004 μl of column fractions were assayed for activity to stimulate p27-ubiquitin ligation. Results were quantified by phosphorimager analysis and were expressed as the percentage of ³⁵S-p27 converted to ubiquitin conjugates. Arrows at top indicate the elution position of molecular mass marker proteins (kDa).

Mass spectrometric sequencing The 10-kDa protein from the last step of purification was excised and digested in gel as described (Shevchenko, et al., 1996, Anal. Cham. 68:850. Mass spectrometric analysis was performed on a Sciex QSTAR mass spectrometer (MDS-Sciex, Concord, ON, Canada). A tryptic peptide at mass 2163.5 was fragmented from doubly and triply charged species to yield a complete match to residues 5-20 of human Cks1.

Assay of p27-ubiquitin ligation. Unless otherwise stated, the reaction mixture contained in a volume of 10 μl: 40 mM Tris-HCl (pH 7.6), 5 mM MgCl₂, 1 mM DTT, 10% (v/v) glycerol, 10 mM phosphocreatine, 100 μg/ml creatine phosphokinase, 0.5 mM ATP, 1 mg/ml soybean trypsin inhibitor, 1 μM ubiquitin aldehyde, 1 mg/ml methylated ubiquitin, 1 pmol E1, 50 pmol Cdc34, 0.25 μl Skp2/Skp1, 0.25 μl Cul-1/ROC1, 0.1 μl cyclin E/Cdk2, 0.5 μl of ³⁵S-p27 and additions as specified. Following incubation at 30° C. for 60 minutes, samples were subjected to SDS-polyacrylamide gel electrophoresis and autoradiography. The ligation of IκBα to ubiquitin was assayed as described (Chen, et al., 2000, J. Biol. Chem. 275:15432), except that baculovirus-expressed, purified Skp1/β-TrCP was used (5 pmol Skp1, ˜1 pmol P-TrCP).

Preparation of ³²P labeled purified p27 and assay of its ubiquitinylation. Purified p27 (0.18 μg) was incubated (60 minutes at 30° C.) with Cdk2/cyclin E (0.25 μl) in a reaction mixture containing in a volume of 10 μl: 50 mM Tris-HCl (pH 7.6), 5 mM MgCl₂, 1 mM DTT, 10% glycerol, 1 mg/ml soybean trypsin inhibitor, 1 μM okadaic acid and 100 μM [³²P-γ-]ATP (˜50 μCi). This preparation is referred to as “³²P-p27”. The ligation of p27 to MeUb was assayed as described above, with the following changes: ³⁵S-p27 was replaced by ³²P-p27, the concentration of unlabeled ATP was increased to 2 mM (for more complete isotopic dilution of labeled ATP present in the preparation of ³²P-p27) and okadaic acid (1 μM) was added.

Assay of binding of p27 to Skp2/Skp1 The reaction mixture contained in a volume of 10 μl: 40 mM Tris-HCl (pH 7.6), 2 mg/ml bovine serum albumin, 1 μl ³⁵S-p27, 1 μl Cdk2/cyclin E, 1 μl Skp2/Skp1, as well as MgCl₂, ATP, DTT, phosphocreatine and creatine phosphokinase at concentrations similar to those described above for p27-ubiquitin ligation assay. Following incubation at 30° C. for 30 min, 6 μl of Affi-prep-Protein A beads (BioRad) to which polyclonal rabbit antibody against full length Skp2 (Carrano, et al., 1999, Nat. Cell Biol. 1:193) had been covalently linked by dimethyl pimelimidate (Harlow, E. & Lane, D., 1998, in Antibodies. A Laboratory Manual (eds. Harlow, E. & Lane, D.), Cold Spring Harb. LabPress, Cold Spring Harbor, N.Y.) was added. The samples were rotated with the anti-Skp2-Protein A beads at 4° C. for 2 hours, and then the beads were washed 4 times with 1-ml portions of RIPA buffer (Harlow, E. & Lane, D., 1998, in Antibodies. A Laboratory Manual (eds. Harlow, E. & Lane, D.), Cold Spring Harb. LabPress, Cold Spring Harbor, N.Y.). Following elution with SDS electrophoresis sample buffer, the samples were subjected to SDS-polyacrylamide gel electrophoresis and autoradiography.

9.2 Results

9.2.1 The Factor from Fraction 1 is a Protein

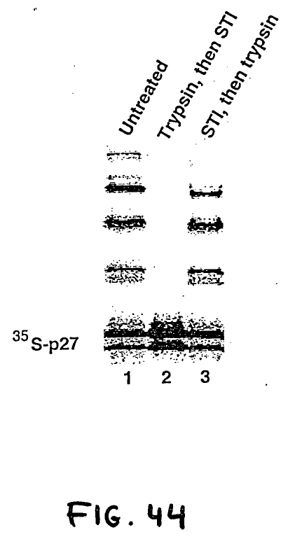

The activity of Fraction 1 is not destroyed by heating at 90° C. However, the active factor is a protein, as indicated by the observation that incubation of heat-treated Fraction 1 with trypsin completely destroyed its activity (FIG. 44, lane 2). Heat-treated Fraction 1 (˜0.1 mg/ml) was incubated at 37° C. for 60 min with 50 mM Tris-HCl (pH 8.0) either in the absence (lane 1) or in the presence of 0.6 mg/ml of TPCK-treated trypsin (Sigma T8642) (lane 2). Trypsin action was terminated by the addition of 2 mg/ml of soybean trypsin inhibitor (STI). In lane 3, STI was added 5 min prior to a similar incubation with trypsin. Subsequently, samples corresponding to ˜50 ng of heat-treated Fraction 1 were assayed for the stimulation of p27-ubiquitin ligation. Incubation of Fraction 1 with trypsin is terminated by the addition of excess soybean trypsin inhibitor (STI), to prevent proteolytic damage to the other components of the system, added following trypsin treatment. STI indeed efficiently blocks trypsin action as is shown in a control experiment in which STI is added to heated Fraction 1 prior to incubation with trypsin (FIG. 44, lane 3). In this incubation, there is no significant decrease in p27-ubiquitin ligation.

9.2.2 The Factor from Fraction 1 is not Nedd8

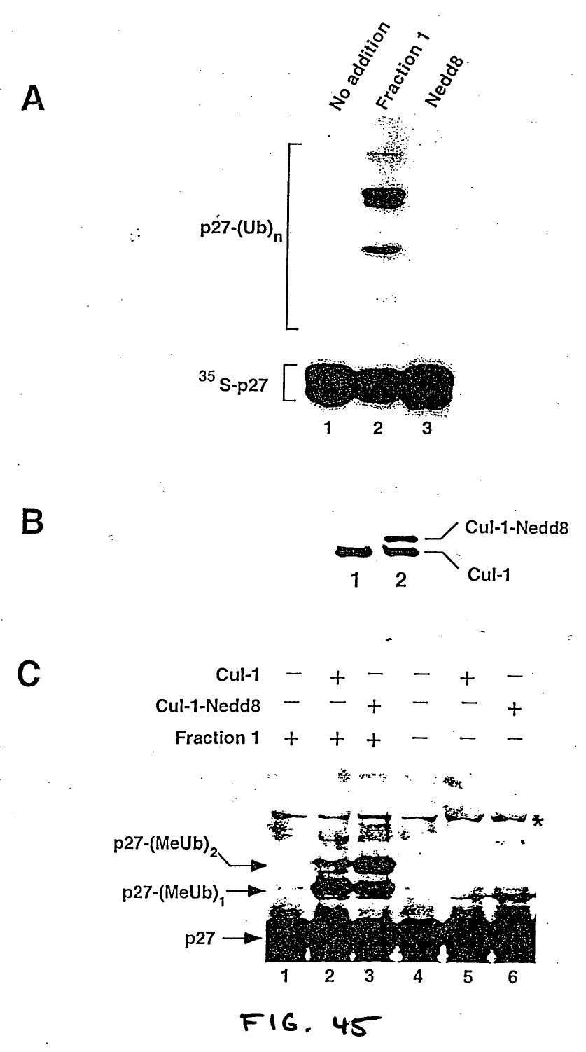

Podust et al. (Podust, et al., 2000, Proc. Natl. Acad. Sci. U.S.A. 97:4579) have reported that the ligation of p27 to ubiquitin requires Fraction 1, and have suggested that Nedd8 is the active component in Fraction 1. Nedd8 (called Rub-1 in yeast) is a highly conserved ubiquitin-like protein that is ligated to different cullins, including Cul-1 (Yeh, et al., 2000, Gene 248:1). The ligation of Nedd8 to Cul-1 has been shown to stimulate, though not to be absolutely required for, the activity of the SCF^{β-TrCP} complex in the ligation of ubiquitin to IKBa (Furukawa, et al., 2000, Mol. Cell Biol. 20:8185; Read, et al., 2000, Mol. Cell Biol. 20:2326; Wu, et al., 2000, J. Biol. Chem 275:32317). Since ³⁵S-labeled p27 can be produced by in vitro translation in reticulocyte lysates, and since reticulocyte lysates contain the enzymes required for the ligation of Nedd8 to cullins (Osaka, et al., 1998, Genes Dev. 12:2549), it is possible that under these conditions Nedd8 could be ligated to Cul-1. However, recombinant purified Nedd8 does not replace the factor from Fraction 1 in promoting p27-ubiquitin ligation (FIG. 45A). Where indicated, ˜50 ng of heat-treated Fraction 1 or 100 ng of purified recombinant human Nedd8 are added to the p27-MeUb ligation assay. To further examine this problem, the enzymes that ligate Nedd8 to Cul-1 are purified by affinity chromatography on GST-Nedd8-Sepharose. Incubation of Cul-1 with Nedd8 and its purified conjugating enzymes convert about one-half of Cul-1 molecules to Nedd8-conjugated form that migrates slower in SDS-polyacrylamide gel electrophoresis (FIG. 45B). Ligation of Nedd8 to Cul-1. Cul-1/ROC1 (3 μl) is incubated with Nedd8 (10 μg) and purified Nedd8-conjugating enzymes (20 μl) in a 100-μl reaction mixture containing Tris (pH 7.6), MgCl₂, ATP, phosphocreatine, creatine phosphokinase, DTT, glycerol and STI at concentrations similar to those described for the p27-ubiquitin ligation assay. A control preparation of Cul1/ROC1 is incubated under similar conditions, but without Nedd8 conjugating enzymes. Following incubation at 30° C. for 2 hours, samples of control or Nedd8-modified preparations are separated on an 8% polyacrylamide-SDS gel and immunoblotted with an anti-Cul-1 antibody (Zymed). The slower migrating form indeed contains Nedd8 as is verified by immunoblotting with a specific antibody directed against Nedd8. The activity of these preparations of Nedd8-conjugated and unmodified Cul-1 in the p27 ubiquitinylation reaction is measured in the presence or absence of heat-treated Fraction 1. Bacterially expressed, purified p27 (20 ng) is used as the substrate rather than ³⁵S-labeled p27 translated in reticulocyte lysate, because reticulocyte lysates also contain the enzyme(s) that rapidly cleave(s) the amide linkage between Nedd8 and Cul-1. The ligation of p27 to MeUb occurrs at 30C for 60 minutes and is followed by separation on a 12.5% polyacrylamide-SDS gel, transfer to nitrocellulose, and immunoblotting with a monoclonal antibody directed against p27 (Transduction Laboratories). Using this purified system and in the presence of heat-treated Fraction 1, significant formation of mono-ubiquitinylated, and less of di-ubiquitiynylated derivatives of p27 is promoted by unmodified Cul-1 (FIG. 45C). With the purified system, conjugates with MeUb larger than the di-ubiquitinylated form are not observed, as opposed to the 4-5 conjugates observed with in vitro-translated ³⁵S-p27 (compare with FIG. 44). With Cul-1 conjugated to Nedd8, a modest stimulation in the ubiquitinylation of p27 is observed, with a special increase in the formation of the di-ubiquitin derivative (FIG. 45, lane 3). In different preparations of Cul-1, Nedd8 ligation increases the over-all rate of p27-ubiquitin ligation by 1.5-3 fold. The basal activity of p27-ubiquitin ligation observed with unmodified Cul-1 is not due to its significant modification by Nedd8 in insect cells, from which baculovirus-expressed Cul-1 was purified, because similar activity is observed with a mutant Cul-1 in which Lys720 at its specific Nedd8-ligation site (Yeh, et al., 2000, Gene 248:1) was changed to Arg. Other investigators have also observed that elimination of Nedd8 modification by a similar mutation significantly reduced, but did not abolish the activity of SFC^{β-TrCP} in the ubiqutinylation of IκBα (Furukawa, et al., 2000, Mol. Cell Biol. 20:8185; Read, et al., 2000, Mol. Cell Biol. 20:2326; Wu, et al., 2000, J. Biol. Chem 275:32317). Importantly, the supplementation of Fraction 1 is still required for p27-MeUb ligation even in the presence of Nedd8-modified Cul-1 (FIG. 45, lanes 5 and 6). Similar results are obtained when MeUb is replaced by native ubiquitin, except that in the latter case high molecular weight polyubiquitin derivatives of p27 are formed. Thus, the data does not support the conclusions of Podust et al. (Podust et al., 2000, Proc. Natl. Acad. Sci. U.S.A. 97:4579) that the active component in Fraction 1 is Nedd8.

9.2.3 Purification of the Factor and its Identification as Cks1

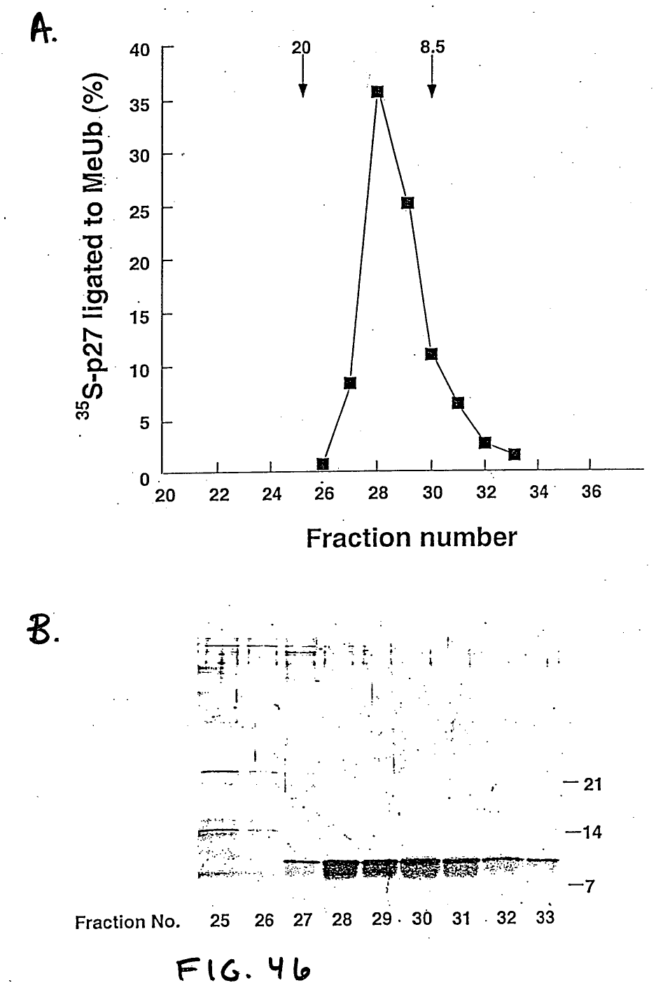

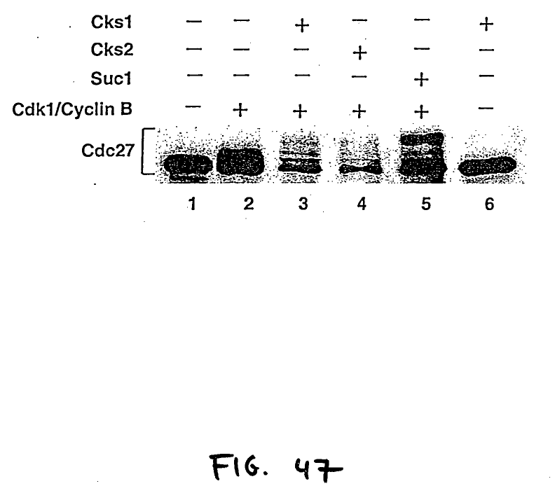

The factor from fraction 1 is purified. FIG. 46A shows the last step of purification on a gel filtration column. The peak of active material from the MonoS step was applied to a Superdex 75 HR 10/30 column (Pharmacia) equilibrated with 20 mM Tris-HCl (pH 7.2), 150 mM NaCl, 1 mM DTT and 01% Brij-35. Samples of 0.5 ml were collected at a flow rate of 0.4 ml/min. Column fractions were concentrated to a volume of 50 μl by centrifuge ultrafiltration (Centricon-10, Amicon). Samples of 0.004 μL of column fractions were assayed for activity to stimulate p27-ubiquitin ligation. Results were quantified by phosphorimager analysis and were expressed as the percentage of ³⁵S-p27 converted to ubiquitin conjugates. Arrows at top indicate the elution position of molecular mass marker proteins (kDa). Activity eluted as a sharp peak at an apparent molecular mass of approx. 10 kDa. Electrophoresis of samples of 2.5 μl from the indicated fractions of the Superdex 75 column on a 16% polyacrylamide-SDS gel and silver staining of column fractions show a single protein of approx. 10 kDa (FIG. 46B). Numbers on the right indicate the migration position of molecular mass marker proteins (kDa). Elution of the ˜10 kDa protein peak coincided with the elution of the peak of activity in fractions 27-28. However, a similar-sized protein continues to be eluted in fractions 30-31, where activity declines markedly. To identify the protein(s), samples from fraction 28 (peak of activity) and fraction 31, subsequent to the peak of activity, are subjected to mass spectrometric sequencing of tryptic peptides. A tryptic peptide of the sequence QIYYSDKYDDEEFEYR, corresponding to amino acid residues 5-20 of human Cks1, is detected in the 10 kDa protein of both fractions. The reason for the difference in the activity of the Cks1 protein in these different fractions is not known. Possibly, the Cks1 protein in fraction 31 is a denatured comformer that may have altered exclusion properties in the gel filtration column.

9.2.4 Activity of Cks1/Suc Proteins

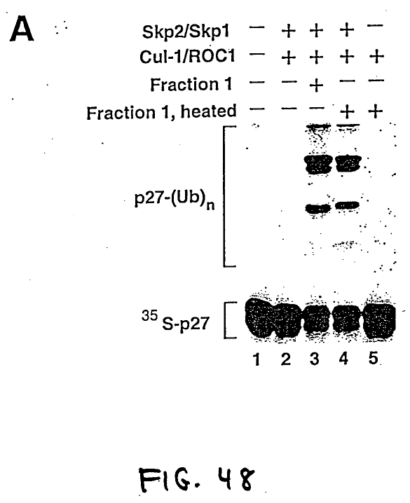

To address whether all Cks/Suc1 proteins used in this study were functional, we have examined their action in promoting multi-phosphorylation of cyclosome/APC by protein kinase Cdk1/cyclinB was examined (Patra, D. & Dunphy, W. G., 1998, Genes Dev. 12:2549; Shteinberg, M. & Hershko, A., 1999, Biochem. Biophys. Res. Commun. 257:12). Cyclosomes from S-phase HeLa cells were partially purified (Yudkovsky, et al., 2000, Biochem. Biophys. Res. Commun. 271:299) and incubated with 500 units of Suc1-free Cdk1/cyclin B (Shteinberg, M. & Hershko, A., 1999, Biochem. Biophys. Res. Commun. 257:12), as described (Yudkovsky, et al., 2000, Biochem. Biophys. Res. Commun. 271:299). Where indicated, 10 ng/μl of the corresponding Cks/Suc1 protein was supplemented. The samples were subjected to immunoblotting with a monoclonal antibody directed against human Cdc27 (Transduction Laboratories). As shown in FIG. 47 the Cdk1-catalyzed hyperphosphorylation of Cdc27, a subunit of the cyclosome/APC, is markedly stimulated by all three recombinant Cks/Suc1 proteins. This is indicated by the decrease in the unphosphorylated form of Cdc27 and its conversion to several hyperphosphorylated forms that migrate slower in SDS-polyacrylamide gel electrophoresis (FIG. 47, lanes 3-5) This large electrophoretic shift, promoted by all recombinant Cks/Suc1 proteins, requires the action of protein kinase Cdk1/cyclin B (FIG. 47, lane 6). All three bacterially expressed Cks/Suc1 proteins used are at least 95% homogeneous, as indicated by SDS-polyacrylamide gel electrophoresis and Coomassie staining.

9.2.5 Confirmation that the Factor Required for p27-Ubiquitin Ligation is Cks1

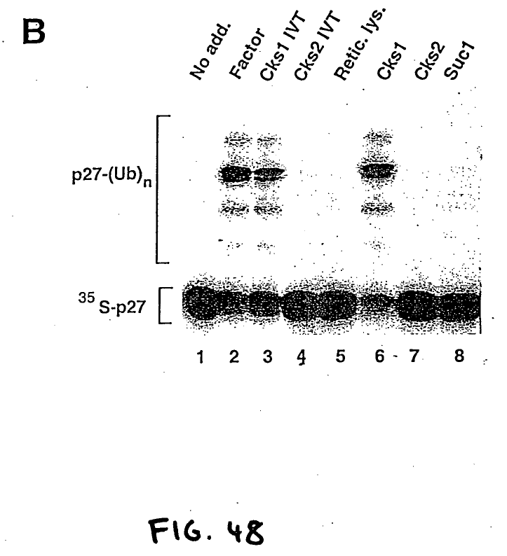

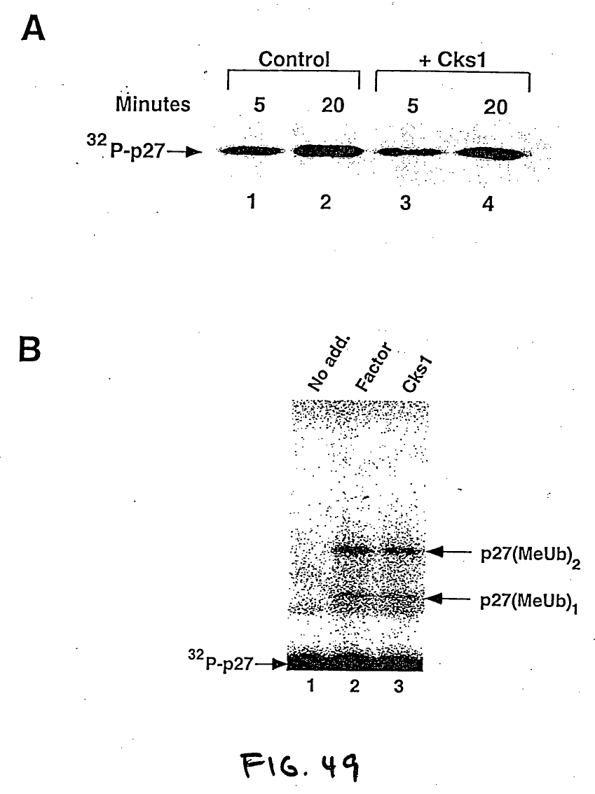

Cks1 produced by in vitro translation (FIG. 48B, lane 3) or bacterially expressed, purified Cks1 (FIG. 48B, lane 6) effectively replaced the factor in this reaction. This action is found to be specific for Cks1 and is not shared by other members of the Cks/Suc1 family of proteins. Human Cks2, which is 81% identical and 90% similar to Cks1, as well as the fission yeast homologue, Suc1, are completely inactive in this reaction, either when produced by in vitro translation (FIG. 48B, lane 4) or as bacterially expressed purified proteins (FIG. 48B, lanes 7 and 8) Purified recombinant Cks2 and Suc1 do not stimulate p27-ubiquitin ligation even when added at up to 50-fold higher concentrations despite their being functional, as demonstrated by their ability to promote the multi-phosphorylation of Cdc27 by Cdk1. The combined evidence thus strongly indicates that the action of Cks1 in p27-ubiquitin ligation is specific and is not shared by other members of this protein family.

9.2.6 Cks1 Promotes the Ligation of Ubiquitin to P27

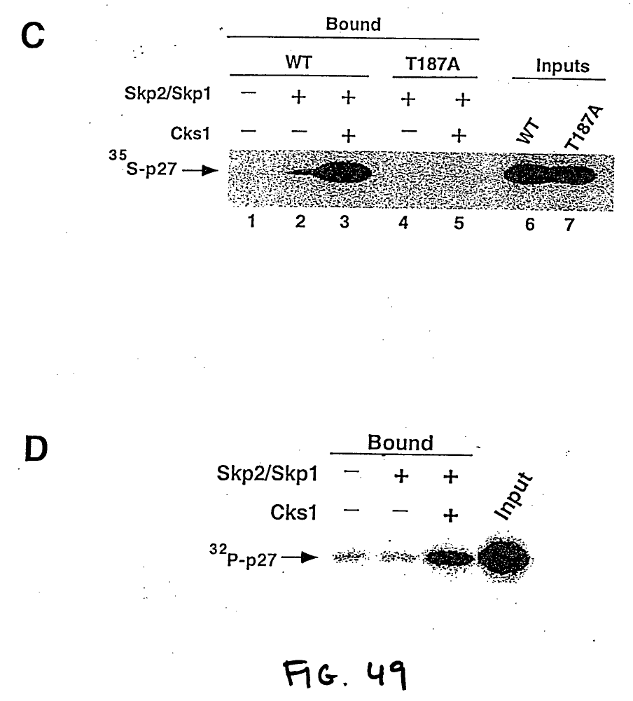

Cks1 does not seem to be required for the action of all mammalian SCF complexes. In the well-characterized case of SCF^{β-TrCP}, the purified complex carries out robust ubiquitinylation of IκB in vitro (Tan, et al., 1999, Mol. Cell 3:527). Furthermore, the addition of Cks1 had no observable influence on the rate of the ligation of ubiquitin to phosphorylated IκBα by purified SCF^{β-TrCP}. It seemed more likely that Cks1 is specifically involved either in the action of the SCF^{Skp2} complex or in some other process necessary for p27-ubiquitin ligation. Since p27 has to be phosphorylated on Thr-187 by Cdk2 for recognition by the SCF^{Skp2} complex (Carrano, et al., 1999, Mat. Cell Biol. 1:1993; Tsvetkov, et al., 1999, Current Biology 661) and since Cks proteins may stimulate the protein kinase activity of some, but not all, Cdk/cyclin complexes (Reynard, et al., 2000, Mol. Cell Biol. 20:5858), it seems possible that Cks1 stimulates the phosphorylation of p27 by Cdk2. However, as shown in (FIG. 49A) p27 is rapidly phosphorylated by Cdk2/cyclin E in the absence of Cks1, and the addition of Cks1 has no significant influence on this process. The conclusion that Cks1 acts at a step subsequent to the phosphorylation of p27 is corroborated by the finding that when purified p27 is first phosphorylated by incubation with Cdk2/cyclin E and ³²[P-γ]_ATP, its subsequent ligation to MeUb still requires Cks1 (FIG. 49B) Therefore, Cks1 greatly stimulates the binding of phosphorylated p27 to Skp2.

9.2.7 Cks1 Affects the Binding of Phosphorylated p27 to Skp2

Whether the step affected by Cks1 is the binding of phosphorylated p27 to Skp2 was assessed. Skp2/Skp1 complex was used instead of Skp2, because in the absence of Skp1, recombinant Skp2 is not expressed abundantly in insect cells in a soluble form. Previously small, but significant binding of ³⁵S-labeled, in vitro-translated p27 to Skp2/Skp1 was detected (by immunoprecipitation with an antibody directed against Skp2), which is dependent upon its phosphorylation on Thr-187 by Cdk2/cyclin E (Carrano, et al., 1999, Nat. Cell Biol 1:193). Using a similar procedure, the binding of p27 to Skp2/Skp1 is greatly stimulated by Cks1 (FIG. 49C, lanes 2 and 3). This action requires the phosphorylation of p27 on Thr-187, since binding of the non-phosphorylatable mutant Thr-187-Ala did not occur even in the presence of Cks1 (FIG. 49C, lanes 4 and 5). To examine whether this action of Cks1 also occurs in a completely purified system devoid of reticulocyte lysate present in preparations of in vitro-translated p27, a similar experiment is performed with bacterially expressed, purified p27 that is phosphorylated by ³²[P-γ] ATP. In this case there is some non-specific binding of phosphorylated p27 to anti-Skp2-Protein A beads in the absence of Skp2. Still, a marked stimulation of the specific binding of ³²P-p27 to Skp2/Skp1 by Cks1 is observed (FIG. 49D) Therefore, Cks1 greatly stimulates the binding of phosphorylated p27 to Skp2.

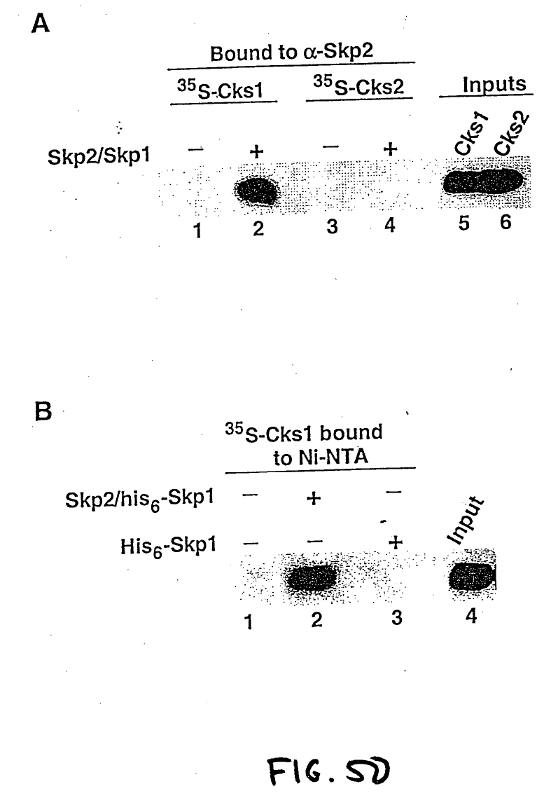

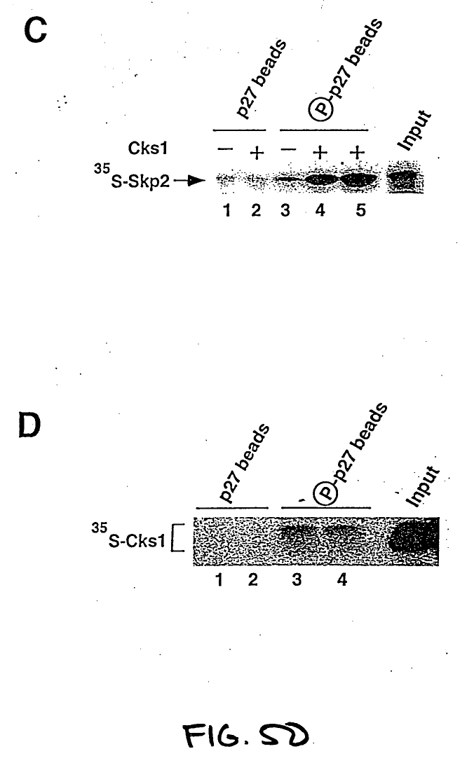

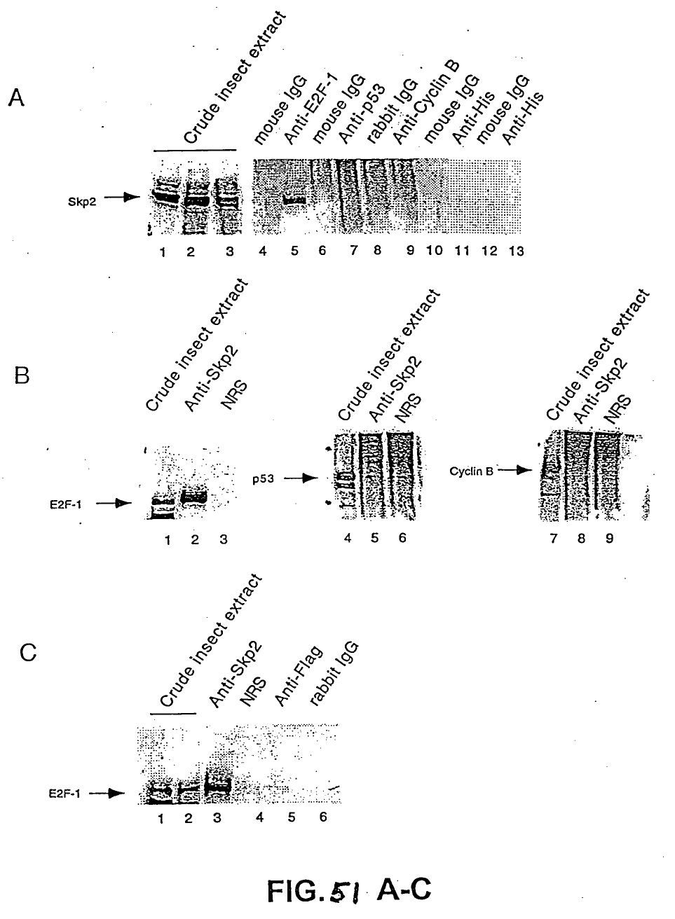

As shown in FIG. 50A, a strong binding of ³⁵S-Cks1 to the Skp2/Skp1 complex was observed. Under similar conditions, no binding of ³⁵S-Cks2 to Skp2/Skp1 was seen. Since in these experiments Skp2/Skp1 complex is used (because of the lack of recombinant native Skp2), it is examined whether Cks1 may bind to Skp1 in the absence of Skp2. In the experiment shown in FIG. 50B, ³⁵S-Cks1 is incubated with either His₆-Skp1 or with Skp2/His₆-Skp1 complex, and then binding to Ni-NTA-agarose beads is estimated. A strong binding of Cks1 to Skp2/His₆-Skp1 but not to His₆-Skp1 was observed. Thus, human Cks1 specifically binds to the Skp2/Skp1 complex, likely through the Skp2 protein.

The results presented herein demonstrate that the binding of Skp2 to phosphopeptide-Sepharose beads (but not to control beads that contained an identical but unphosphorylated p27-derived peptide) is greatly increased by Cks1 (FIG. 50C). These findings indicate that binding to this phosphopetide can serve as a valid tool to study Cks1-assisted Skp2-p27 interaction. Using the same p27-derived peptide beads, significant binding of ³⁵S-Cks1 to phosphorylated p27 peptide, but not to unphosphorylated p27 peptide is observed FIG. 50D. These findings indicate that Cks1 binds directly to phospho-Thrl87 of p27 and demonstrate that the presence of Cdk2/cyclin E is not obligatory for the binding of Skp2 to phosphorylated p27.

10. Example Assay to Identify an FBP Interaction With a Cell Cycle Regulatory Protein (e.g., SKP2 with E2F)

The following study was conducted to identify novel substrates of the known FBP, Skp2.

As shown in FIG. 44, E2F-1, but not other substrates of the ubiquitin pathway assayed, including p53 and Cyclin B, physically associates with Skp2. Extracts of insect cells infected with baculoviruses co-expressing Skp2 and E2F-1, (lanes 1,4 and 5), or Skp2 and hexa-histidine p53 (His-p53) (lanes 2,6,7,10 and 11), or Skp2 and His-Cyclin B (lanes 3,8,9,12, and 13) were either directly immunoblotted with an anti-serum to Skp2 (lanes 1-3) or first subjected to immunoblotted with an anti-serum to Skp2 (lanes 1-3) or first subjected to immunoprecipitation with the indicated antibodies and then immunoblotted with an anti-serum to Skp2 (lanes 4-13). Antibodies used in the immunoprecipitations are: normal purified mouse immunoglobulins (IgG) (lane 4,6,10 and 12), purified mouse monoclonal anti-E2F-1 antibody (KH-95, from Santa Cruz) (lane 5), purified mouse monoclonal anti-p53 antibody (DO-1, from Oncogene Science) (lane 7), purified rabbit IgG (lane 8), purified rabbit polyclonal anti-Cyclin B antibody (lane 9), purified mouse monoclonal anti-His antibody (clone 34660, from Qiagen) (lanes 11 and 13).

As shown in FIG. 44B, Skp2 physically associates with E2F-1 but not with other substrates of the ubiquitin pathway (p53 and Cyclin B). Extracts of insect cells infected with baculoviruses co-expressing Skp2 and E2F-1 (lanes 1-3), or Skp2 and His-p53 (lanes 4-6), or Skp2 and His-Cyclin B (lanes 7-9) were either directly immunoblotted with antibodies to the indicated proteins (lanes 1,4 and 7) or first subjected to immunoprecipitation with the indicated anti-sera and then immunoblotted with antibodies to the indicated proteins (lanes 2,3,5,6,8 and 9). Anti-sera used in the immunoprecipitations are: anti-Skp2 serum (lanes 2,5 and 8), and normal rabbit serum (NRS) (lane 3,6 and 9).

As shown in FIG. 44C, E2F-1 physically associates with Skp2 but not with another F-box protein (FBP1). Extracts of insect cells infected with baculoviruses co-expressing Skp2 and E2F-1 (lanes 1,3 and 4), or Flag-tagged-FBP1 and E2F-1 (lanes 2,5 and 6) were either directly immunoblotted with a mouse monoclonal anti-E2F-1 antibody (lanes 1 and 2) or first subjected to immunoprecipitation with the indicated antibodies and then immunoblotted with a mouse monoclonal anti-E2F-1 antibody (lanes 3-6). Antibodies used in the immunoprecipitations are: anti-Skp2 serum (lanes 3), NRS (lane 4), purified rabbit polyclonal anti-Flag (lane 5), purified rabbit IgG (lane 6).

The methodology used in this example can also be applied to identify novel substrates of any FBP, including, but not limited to, the FBPs of the invention, such as FBP1, FBP2, FBP3a, FBP3b, FBP4, FBP5, FBP6, FBP7, FBP8, FBP9, FBP10, FBP11, FBP12, FBP13, FBP14, FBP15, FBP16, FBP17, FBP18, FBP19, FBP20, FBP21, FBP22, FBP23, FBP24, and FBP25.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein are incorporated herein by reference for all purposes.

1-9. (canceled)

10. A method for treating a proliferative disorder in a patient comprising administering to a patient in need thereof a compound that modulates the interaction of p27 and skp2.

11. The method of claim 10 in which the compound inhibits the interaction of p27 and skp2.

12. The method of claim 10 in which the compound enhances the interaction of p27 and skp2.

13. The method of claim 10 wherein the interaction of p27 and skp2 is measured by measuring binding of p27 and skp2.

14. The method of claim 10 wherein the interaction of p27 and skp2 is measured by measuring p27 ubiquitination.

15. The method of claim 10 wherein the interaction of p27 and skp2 is measured by measuring p27 degradation.

16. The method of claim 10 wherein the interaction of p27 and skp2 is measured by measuring cell cycle activity.

17. The method of claim 12 wherein the proliferative disorder is a degenerative disorder, growth deficiency, hypoproliferative disorder, physical trauma, lesion, wound, or nervous system disorder.

18. A method for preventing a proliferative disorder in an individual comprising administering to an individual in need thereof a compound that modulates the interaction of p27 and skp2.

19. A method for treating a patient with a proliferative disorder by administering to a patient in need thereof a compound that increases ubiquitination of p27.

20. A method for treating a patient with a proliferative disorder by administering to a patient in need thereof a compound that increases degradation of p27.

21. The method of claims 10 and 18-20 wherein the compound is selected from the group consisting of a small molecule, peptide, antibody, antisense molecule, and a ribozyme.

22. The method of claims 10, 19 and 20 wherein the proliferative disorder is an inflammatory disorder.

23. The method of claims 10, 19 and 20 wherein the proliferative disorder is a fibroblast proliferative disorder.

24. The method of claims 10, 19 and 20 wherein the proliferative disorder is a T-cell proliferative disorder.

25. The method of claims 10, 12, 19, and 20 wherein the proliferative disorder is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, acute lymphocytic leukemia, acute myelocytic leukemia, chronic leukemia, polycythemia vera, Hodgkin's disease lymphoma, non-Hodgkin's disease lymphoma, multiple myeloma, Waldenstrom's macroglobulinemia, or heavy chain disease.
